SEQUENCE LISTING (396 sequences)

SEQ ID NO: 1 - protein, 149 aa, Escherichia coli
MKLLKVAAIAAIFSGSALAGVVPQYGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 2 - protein, 151 aa, Escherichia coli
MKNKLLFMMLTILGAPGIAAAAGYDLANSETNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 3 - DNA, 57 bp, Artificial Sequence
  Description of Artificial Sequence Synthetic primer
ggggatccgaattcgtctgagctgaacatttaccagtacggtggcaagcttgcggcc

SEQ ID NO: 4 - DNA, 57 bp, Artificial Sequence
  Description of Artificial Sequence Synthetic primer
ggggatccgaattcgtctgcacttgctctgcaaactgatgcccgtaagcttgcggcc

SEQ ID NO: 5 - DNA, 57 bp, Artificial Sequence
  Description of Artificial Sequence Synthetic primer
ggggatccgaattcgaactcctccgtcaacgtgactcaggttggcaagcttgcggcc

SEQ ID NO: 6 - DNA, 57 bp, Artificial Sequence
  Description of Artificial Sequence Synthetic primer
ggggatccgaattcgtttggtaacaacgcgaccgctcatcagtacaagcttgcggcc

SEQ ID NO: 7 - DNA, 60 bp, Artificial Sequence
  Description of Artificial Sequence Synthetic primer
ggggatccgaattcgccgtctgagctgaacatttaccagtacggtggcaagcttgcggcc

SEQ ID NO: 8 - DNA, 60 bp, Artificial Sequence
  Description of Artificial Sequence Synthetic primer
ggggatccgaattcgtctgcacttgctctgcaaactgatgcccgtcggaagcttgcggcc

SEQ ID NO: 9 - DNA, 60 bp, Artificial Sequence
  Description of Artificial Sequence Synthetic primer
ggggatccgaattcgaactcctccgtcaacgtgactcaggttggcccgaagcttgcggcc

SEQ ID NO: 10 - DNA, 60 bp, Artificial Sequence
  Description of Artificial Sequence Synthetic primer
ggggatccgaattcgtttggtaacaacgcgaccgctcatcagtaccggaagcttgcggcc

SEQ ID NO: 11 - protein, 10 aa, Artificial Sequence
  Description of Artificial Sequence Synthetic peptide
SELNIYQYGG

SEQ ID NO: 12 - protein, 10 aa, Artificial Sequence
  Description of Artificial Sequence Synthetic peptide
SALALQTDAR

SEQ ID NO: 13 - protein, 10 aa, Artificial Sequence
  Description of Artificial Sequence Synthetic peptide
NSSVNVTQVG

SEQ ID NO: 14 - protein, 10 aa, Artificial Sequence
  Description of Artificial Sequence Synthetic peptide
FGNNATAHQY

SEQ ID NO: 15 - protein, 11 aa, Artificial Sequence
  Description of Artificial Sequence Synthetic peptide
PSELNIYQYGG

SEQ ID NO: 16 - protein, 11 aa, Artificial Sequence
  Description of Artificial Sequence Synthetic peptide
SALALQTDARR

SEQ ID NO: 17 - protein, 11 aa, Artificial Sequence
  Description of Artificial Sequence Synthetic peptide
NSSVNVTQVGP

SEQ ID NO: 18 - protein, 11 aa, Artificial Sequence
  Description of Artificial Sequence Synthetic peptide
FGNNATAHQYR

SEQ ID NO: 19 - DNA, 57 bp, Artificial Sequence
  Description of Artificial Sequence Synthetic primer
ggggatccgaattcgaatcaggcagccataattggtcaagctgggaagcttgcggcc

SEQ ID NO: 20 - DNA, 57 bp, Artificial Sequence
  Description of Artificial Sequence Synthetic primer
ggggatccgaattcgaatagtgctcagttacggcagggaggctcaaagcttgcggcc

SEQ ID NO: 21 - DNA, 57 bp, Artificial Sequence
  Description of Artificial Sequence Synthetic primer
ggggatccgaattcgaaaacggcaattgtagtgcagagacagtcgaagcttgcggcc

SEQ ID NO: 22 - DNA, 57 bp, Artificial Sequence
  Description of Artificial Sequence Synthetic primer
ggggatccgaattcgtcgcaaatggctattcgcgtgacacaacgtaagcttgcggcc

SEQ ID NO: 23 - DNA, 60 bp, Artificial Sequence
  Description of Artificial Sequence Synthetic primer
ggggatccgaattcgaatcaggcagccataattggtcaagctgggcggaagcttgcggcc

SEQ ID NO: 24 - DNA, 60 bp, Artificial Sequence
  Description of Artificial Sequence Synthetic primer
ggggatccgaattcgaatagtgctcagttacggcagggaggctcaccgaagcttgcggcc

SEQ ID NO: 25 - DNA, 60 bp, Artificial Sequence
  Description of Artificial Sequence Synthetic primer
ggggatccgaattcgaaaacggcaattgtagtgcagagacagtcgccgaagcttgcggcc

SEQ ID NO: 26 - DNA, 60 bp, Artificial Sequence
  Description of Artificial Sequence Synthetic primer
ggggatccgaattcgtcgcaaatggctattcgcgtgacacaacgtcggaagcttgcggcc

SEQ ID NO: 27 - protein, 10 aa, Artificial Sequence
  Description of Artificial Sequence Synthetic peptide
NQAAIIGQAG

SEQ ID NO: 28 - protein, 10 aa, Artificial Sequence
  Description of Artificial Sequence Synthetic peptide
NSAQLRQGGS

SEQ ID NO: 29 - protein, 10 aa, Artificial Sequence
  Description of Artificial Sequence Synthetic peptide
KTAIVVQRQS

SEQ ID NO: 30 - protein, 10 aa, Artificial Sequence
  Description of Artificial Sequence Synthetic peptide
SQMAIRVTQR

SEQ ID NO: 31 - protein, 11 aa, Artificial Sequence
  Description of Artificial Sequence Synthetic peptide
NQAAIIGQAGR

SEQ ID NO: 32 - protein, 11 aa, Artificial Sequence
  Description of Artificial Sequence Synthetic peptide
NSAQLRQGGSP

SEQ ID NO: 33 - protein, 11 aa, Artificial Sequence
  Description of Artificial Sequence Synthetic peptide
KTAIVVQRQSP

SEQ ID NO: 34 - protein, 11 aa, Artificial Sequence
  Description of Artificial Sequence Synthetic peptide
SQMAIRVTQRR

SEQ ID NO: 35 - protein, 13 aa, Artificial Sequence
  Description of Artificial Sequence Synthetic peptide
RRRSELNIYQYGG

SEQ ID NO: 36 - protein, 13 aa, Artificial Sequence
  Description of Artificial Sequence Synthetic peptide
PPPSELNIYQYGG

SEQ ID NO: 37 - protein, 16 aa, Artificial Sequence
  Description of Artificial Sequence Synthetic peptide
RRRSELNIYQYGGRRR

SEQ ID NO: 38 - protein, 16 aa, Artificial Sequence
  Description of Artificial Sequence Synthetic peptide
PPPSELNIYQYGGPPP

SEQ ID NO: 39 - protein, 16 aa, Artificial Sequence
  Description of Artificial Sequence Synthetic peptide
PPPSELNIYQYGGRRR

SEQ ID NO: 40 - protein, 13 aa, Artificial Sequence
  Description of Artificial Sequence Synthetic peptide
SELNIYQYGGRRR

SEQ ID NO: 41 - protein, 13 aa, Artificial Sequence
  Description of Artificial Sequence Synthetic peptide
SELNIYQYGGPPP

SEQ ID NO: 42 - protein, 10 aa, Artificial Sequence
  Description of Artificial Sequence Synthetic peptide
SEKNKYQYGG

SEQ ID NO: 43 - protein, 10 aa, Artificial Sequence
  Description of Artificial Sequence Synthetic peptide
SELNKYKYGG

SEQ ID NO: 44 - protein, 10 aa, Artificial Sequence
  Description of Artificial Sequence Synthetic peptide
SELNKKQYGG

SEQ ID NO: 45 - protein, 10 aa, Artificial Sequence
  Description of Artificial Sequence Synthetic peptide
KELNIYQYGK

SEQ ID NO: 46 - protein, 10 aa, Artificial Sequence
  Description of Artificial Sequence Synthetic peptide
KELNKYQYGK

SEQ ID NO: 47 - protein, 13 aa, Artificial Sequence
  Description of Artificial Sequence Synthetic peptide
RRRSALALQTDAR

SEQ ID NO: 48 - protein, 13 aa, Artificial Sequence
  Description of Artificial Sequence Synthetic peptide
PPPSALALQTDAR

SEQ ID NO: 49 - protein, 13 aa, Artificial Sequence
  Description of Artificial Sequence Synthetic peptide
SALALQTDARRRR

SEQ ID NO: 50 - protein, 13 aa, Artificial Sequence
  Description of Artificial Sequence Synthetic peptide
SALALQTDARPPP

SEQ ID NO: 51 - protein, 16 aa, Artificial Sequence
  Description of Artificial Sequence Synthetic peptide
RRRSALALQTDARRRR

SEQ ID NO: 52 - protein, 16 aa, Artificial Sequence
  Description of Artificial Sequence Synthetic peptide
PPPSALALQTDARPPP

SEQ ID NO: 53 - protein, 16 aa, Artificial Sequence
  Description of Artificial Sequence Synthetic peptide
PPPSALALQTDARRRR

SEQ ID NO: 54 - protein, 12 aa, Artificial Sequence
  Description of Artificial Sequence Synthetic peptide
KSALAKQTDARK

SEQ ID NO: 55 - protein, 12 aa, Artificial Sequence
  Description of Artificial Sequence Synthetic peptide
KSALALQTDARK

SEQ ID NO: 56 - protein, 10 aa, Artificial Sequence
  Description of Artificial Sequence Synthetic peptide
SKLKLQTDAR

SEQ ID NO: 57 - protein, 10 aa, Artificial Sequence
  Description of Artificial Sequence Synthetic peptide
SALALKKDAR

SEQ ID NO: 58 - protein, 10 aa, Artificial Sequence
  Description of Artificial Sequence Synthetic peptide
SALKLKTDAR

SEQ ID NO: 59 - protein, 13 aa, Artificial Sequence
  Description of Artificial Sequence Synthetic peptide
RRRKTAIVVQRQS

SEQ ID NO: 60 - protein, 13 aa, Artificial Sequence
  Description of Artificial Sequence Synthetic peptide
PPPKTAIVVQRQS

SEQ ID NO: 61 - protein, 16 aa, Artificial Sequence
  Description of Artificial Sequence Synthetic peptide
RRRKTAIVVQRQSPPP

SEQ ID NO: 62 - protein, 16 aa, Artificial Sequence
  Description of Artificial Sequence Synthetic peptide
RRRKTAIVVQRQSRRR

SEQ ID NO: 63 - protein, 16 aa, Artificial Sequence
  Description of Artificial Sequence Synthetic peptide
PPPKTAIVVQRQSPPP

SEQ ID NO: 64 - protein, 13 aa, Artificial Sequence
  Description of Artificial Sequence Synthetic peptide
KTAIVVQRQSRRR

SEQ ID NO: 65 - protein, 13 aa, Artificial Sequence
  Description of Artificial Sequence Synthetic peptide
KTAIVVQRQSPPP

SEQ ID NO: 66 - protein, 10 aa, Artificial Sequence
  Description of Artificial Sequence Synthetic peptide
KTKIKVQRQS

SEQ ID NO: 67 - protein, 10 aa, Artificial Sequence
  Description of Artificial Sequence Synthetic peptide
KTAKVKQRQS

SEQ ID NO: 68 - protein, 10 aa, Artificial Sequence
  Description of Artificial Sequence Synthetic peptide
KTAKKVQRQS

SEQ ID NO: 69 - protein, 12 aa, Artificial Sequence
  Description of Artificial Sequence Synthetic peptide
KKTAIVVQRQSK

SEQ ID NO: 70 - protein, 12 aa, Artificial Sequence
  Description of Artificial Sequence Synthetic peptide
KKTAIKVQRQSK

SEQ ID NO: 71 - protein, 13 aa, Artificial Sequence
  Description of Artificial Sequence Synthetic peptide
PPPSQMAIRVTQR

SEQ ID NO: 72 - protein, 13 aa, Artificial Sequence
  Description of Artificial Sequence Synthetic peptide
RRRSQMAIRVTQR

SEQ ID NO: 73 - protein, 13 aa, Artificial Sequence
  Description of Artificial Sequence Synthetic peptide
SQMAIRVTQRPPP

SEQ ID NO: 74 - protein, 13 aa, Artificial Sequence
  Description of Artificial Sequence Synthetic peptide
SQMAIRVTQRRRR

SEQ ID NO: 75 - protein, 16 aa, Artificial Sequence
  Description of Artificial Sequence Synthetic peptide
PPPSQMAIRVTQRPPP

SEQ ID NO: 76 - protein, 16 aa, Artificial Sequence
  Description of Artificial Sequence Synthetic peptide
RRRSQMAIRVTQRRRR

SEQ ID NO: 77 - protein, 16 aa, Artificial Sequence
  Description of Artificial Sequence Synthetic peptide
PPPSQMAIRVTQRRRR

SEQ ID NO: 78 - protein, 10 aa, Artificial Sequence
  Description of Artificial Sequence Synthetic peptide
SQMKKRVTQR

SEQ ID NO: 79 - protein, 10 aa, Artificial Sequence
  Description of Artificial Sequence Synthetic peptide
SQKAKRVTQR

SEQ ID NO: 80 - protein, 10 aa, Artificial Sequence
  Description of Artificial Sequence Synthetic peptide
SQMAIRKTKR

SEQ ID NO: 81 - protein, 12 aa, Artificial Sequence
  Description of Artificial Sequence Synthetic peptide
KSQMAIRVTQRK

SEQ ID NO: 82 - protein, 16 aa, Artificial Sequence
  Description of Artificial Sequence Synthetic peptide
RRRKTAIVVQRQSPPP

SEQ ID NO: 83 - protein, 12 aa, Artificial Sequence
  Description of Artificial Sequence Synthetic peptide
KSQMAKRVTQRK

SEQ ID NO: 84 - protein, 20 aa, Artificial Sequence
  Description of Artificial Sequence Synthetic peptide
RRRRRKTAIVVQRQSPPPPP

SEQ ID NO: 85 - protein, 18 aa, Artificial Sequence
  Description of Artificial Sequence Synthetic peptide
RRRKTAIVVQRQSPPPPP

SEQ ID NO: 86 - protein, 18 aa, Artificial Sequence
  Description of Artificial Sequence Synthetic peptide
RRRRRKTAIVVQRQSPPP

SEQ ID NO: 87 - protein, 16 aa, Artificial Sequence
  Description of Artificial Sequence Synthetic peptide
GGGKTAIVVQRQSPPP

SEQ ID NO: 88 - protein, 16 aa, Artificial Sequence
  Description of Artificial Sequence Synthetic peptide
RRRKTAIVVQRQSGGG

SEQ ID NO: 89 - protein, 14 aa, Artificial Sequence
  Description of Artificial Sequence Synthetic peptide
RRKTAIVVQRQSPP

SEQ ID NO: 90 - protein, 12 aa, Artificial Sequence
  Description of Artificial Sequence Synthetic peptide
RKTAIVVQRQSP

SEQ ID NO: 91 - protein, 15 aa, Artificial Sequence
  Description of Artificial Sequence Synthetic peptide
KETAAAKFERQHMDS

SEQ ID NO: 92 - DNA, 57 bp, Artificial Sequence
  Description of Artificial Sequence Synthetic primer
ggccgcaagcttgccaccgtactggtaaatgttcagctcagacgaattcggatcccc

SEQ ID NO: 93 - DNA, 57 bp, Artificial Sequence
  Description of Artificial Sequence Synthetic primer
ggccgcaagcttacgggcatcagtttgcagagcaagtgcagacgaattcggatcccc

SEQ ID NO: 94 - DNA, 57 bp, Artificial Sequence
  Description of Artificial Sequence Synthetic primer
ggccgcaagcttcgactgtctctgcactacaattgccgttttcgaattcggatcccc

SEQ ID NO: 95 - DNA, 66 bp, Artificial Sequence
  Description of Artificial Sequence Synthetic primer
ggggatccgaattcgcgccgtcggtctgagctgaacatttaccagtacggtggcaagcttgcggcc

SEQ ID NO: 96 - DNA, 66 bp, Artificial Sequence
  Description of Artificial Sequence Synthetic primer
ggccgcaagcttgccaccgtactggtaaatgttcagctcagaccgacggcgcgaattcggatcccc

SEQ ID NO: 97 - DNA, 66 bp, Artificial Sequence
  Description of Artificial Sequence Synthetic primer
ggggatccgaattcgccgccaccatctgagctgaacatttaccagtacggtggcaagcttgcggcc

SEQ ID NO: 98 - DNA, 66 bp, Artificial Sequence
  Description of Artificial Sequence Synthetic primer
ggccgcaagcttgccaccgtactggtaaatgttcagctcagatggtggcggcgaattcggatcccc

SEQ ID NO: 99 - DNA, 75 bp, Artificial Sequence
  Description of Artificial Sequence Synthetic primer
ggggatccgaattcgcgccgtcgctctgagctgaacatttaccagtacggtggccgccgtcgcaagcttgcggcc

SEQ ID NO: 100 - DNA, 75 bp, Artificial Sequence
  Description of Artificial Sequence Synthetic primer
ggggatccgaattcgccgccaccatctgagctgaacatttaccagtacggtggcccaccaccgaagcttgcggcc

SEQ ID NO: 101 - DNA, 75 bp, Artificial Sequence
  Description of Artificial Sequence Synthetic primer
ggggatccgaattcgccgccaccatctgagctgaacatttaccagtacggtggccgccgtcgcaagcttgcggcc

SEQ ID NO: 102 - DNA, 75 bp, Artificial Sequence
  Description of Artificial Sequence Synthetic primer
ggccgcaagcttgcgacggcggccaccgtactggtaaatgttcagctcagatggtggcggcgaattcggatcccc

SEQ ID NO: 103 - DNA, 66 bp, Artificial Sequence
  Description of Artificial Sequence Synthetic primer
ggggatccgaattcgtctgagctgaacatttaccagtacggtggccgccgtcggaagcttgcggcc

SEQ ID NO: 104 - DNA, 66 bp, Artificial Sequence
  Description of Artificial Sequence Synthetic primer
ggccgcaagcttccgacggcggccaccgtactggtaaatgttcagctcagacgaattcggatcccc

SEQ ID NO: 105 - DNA, 66 bp, Artificial Sequence
  Description of Artificial Sequence Synthetic primer
ggggatccgaattcgtctgagctgaacatttaccagtacggtggcccgccaccaaagcttgcggcc

SEQ ID NO: 106 - DNA, 57 bp, Artificial Sequence
  Description of Artificial Sequence Synthetic primer
ggggatccgaattcgtctgagaaaaacaaataccagtacggtggcaagcttgcggcc

SEQ ID NO: 107 - DNA, 66 bp, Artificial Sequence
  Description of Artificial Sequence Synthetic primer
ggccgcaagctttggtggcgggccaccgtactggtaaatgttcagctcagacgaattcggatcccc

SEQ ID NO: 108 - DNA, 57 bp, Artificial Sequence
  Description of Artificial Sequence Synthetic primer
ggggatccgaattcgtctgagctgaacaaatacaaatacggtggcaagcttgcggcc

SEQ ID NO: 109 - DNA, 57 bp, Artificial Sequence
  Description of Artificial Sequence Synthetic primer
ggccgcaagcttgccaccgtatttgtatttgttcagctcagacgaattcggatcccc

SEQ ID NO: 110 - DNA, 57 bp, Artificial Sequence
  Description of Artificial Sequence Synthetic primer
ggggatccgaattcgtctgagctgaacaaaaagcagtacggtggcaagcttgcggcc

SEQ ID NO: 111 - DNA, 57 bp, Artificial Sequence
  Description of Artificial Sequence Synthetic primer
ggccgcaagcttgccaccgtactgctttttgttcagctcagacgaattcggatcccc

SEQ ID NO: 112 - DNA, 63 bp, Artificial Sequence
  Description of Artificial Sequence Synthetic primer
ggggatccgaattcgaaatctgagctgaacatttaccagtacggtggcaagaagcttgcggcc

SEQ ID NO: 113 - DNA, 63 bp, Artificial Sequence
  Description of Artificial Sequence Synthetic primer
ggccgcaagcttcttgccaccgtactggtaaatgttcagctcagatttcgaattcggatcccc

SEQ ID NO: 114 - DNA, 57 bp, Artificial Sequence
  Description of Artificial Sequence Synthetic primer
ggggatccgaattcgaaagagctgaacaaataccagtacggtaaaaagcttgcggcc

SEQ ID NO: 115 - DNA, 57 bp, Artificial Sequence
  Description of Artificial Sequence Synthetic primer
ggccgcaagctttttaccgtactggtatttgttcagctctttcgaattcggatcccc

SEQ ID NO: 116 - DNA, 66 bp, Artificial Sequence
  Description of Artificial Sequence Synthetic primer
ggggatccgaattcgcgccgtcggtctgcacttgctctgcaaactgatgcccgtaagcttgcggcc

SEQ ID NO: 117 - DNA, 66 bp, Artificial Sequence
  Description of Artificial Sequence Synthetic primer
ggggatccgaattcgccgccaccatctgcacttgctctgcaaactgatgcccgtaagcttgcggcc

SEQ ID NO: 118 - DNA, 66 bp, Artificial Sequence
  Description of Artificial Sequence Synthetic primer
ggccgcaagcttacgggcatcagtttgcagagcaagtgcagaccgacggcgcgaattcggatcccc

SEQ ID NO: 119 - DNA, 66 bp, Artificial Sequence
  Description of Artificial Sequence Synthetic primer
ggggatccgaattcgtctgcacttgctctgcaaactgatgcccgtcgccgtcggaagcttgcggcc

SEQ ID NO: 120 - DNA, 66 bp, Artificial Sequence
  Description of Artificial Sequence Synthetic primer
ggccgcaagcttccgacggcgacgggcatcagtttgcagagcaagtgcagacgaattcggatcccc

SEQ ID NO: 121 - DNA, 66 bp, Artificial Sequence
  Description of Artificial Sequence Synthetic primer
ggggatccgaattcgtctgcacttgctctgcaaactgatgcccgtccgccaccaaagcttgcggcc

SEQ ID NO: 122 - DNA, 66 bp, Artificial Sequence
  Description of Artificial Sequence Synthetic primer
ggccgcaagctttggtggcggacgggcatcagtttgcagagcaagtgcagacgaattcggatcccc

SEQ ID NO: 123 - DNA, 75 bp, Artificial Sequence
  Description of Artificial Sequence Synthetic primer
ggggatccgaattcgcgccgtcggtctgcacttgctctgcaaactgatgcccgtcgccgtcggaagcttgcggcc

SEQ ID NO: 124 - DNA, 75 bp, Artificial Sequence
  Description of Artificial Sequence Synthetic primer
ggccgcaagcttccgacggcgacgggcatcagtttgcagagcaagtgcagaccgacggcgcgaattcggatcccc

SEQ ID NO: 125 - DNA, 75 bp, Artificial Sequence
  Description of Artificial Sequence Synthetic primer
ggggatccgaattcgccgccaccatctgcacttgctctgcaaactgatgcccgtccgccaccaaagcttgcggcc

SEQ ID NO: 126 - DNA, 75 bp, Artificial Sequence
  Description of Artificial Sequence Synthetic primer
ggccgcaagctttggtggcggacgggcatcagtttgcagagcaagtgcagatggtggcggcgaattcggatcccc

SEQ ID NO: 127 - DNA, 75 bp, Artificial Sequence
  Description of Artificial Sequence Synthetic primer
ggggatccgaattcgcgccgtcggtctgcacttgctctgcaaactgatgcccgtccgccaccaaagcttgcggcc

SEQ ID NO: 128 - DNA, 66 bp, Artificial Sequence
  Description of Artificial Sequence Synthetic primer
ggggatccgaattcgaaatctgcacttgctaaactgcaaactgatgcccgtaaaaagcttgcggcc

SEQ ID NO: 129 - DNA, 63 bp, Artificial Sequence
  Description of Artificial Sequence Synthetic primer
ggggatccgaattcgaaatctgcacttgctctgcaaactgatgcccgtaaaaagcttgcggcc

SEQ ID NO: 130 - DNA, 57 bp, Artificial Sequence
  Description of Artificial Sequence Synthetic primer
ggggatccgaattcgtctaagcttaaactgcaaactgatgcccgtaagcttgcggcc

SEQ ID NO: 131 - DNA, 75 bp, Artificial Sequence
  Description of Artificial Sequence Synthetic primer
ggccgcaagctttggtggcggacgggcatcagtttgcagagcaagtgcagaccgacggcgcgaattcggatcccc

SEQ ID NO: 132 - DNA, 57 bp, Artificial Sequence
  Description of Artificial Sequence Synthetic primer
ggggatccgaattcgtctgcacttgctctgaagaaagatgcccgtaagcttgcggcc

SEQ ID NO: 133 - DNA, 57 bp, Artificial Sequence
  Description of Artificial Sequence Synthetic primer
ggccgcaagcttacgggcatctttcttcagagcaagtgcagacgaattcggatcccc

SEQ ID NO: 134 - DNA, 57 bp, Artificial Sequence
  Description of Artificial Sequence Synthetic primer
ggggatccgaattcgtctgcacttaaactgaaaactgatgcccgtaagcttgcggcc

SEQ ID NO: 135 - DNA, 66 bp, Artificial Sequence
  Description of Artificial Sequence Synthetic primer
ggggatccgaattcgcgccgtcggaaaacggcaattgtagtgcagagacagtcgaagcttgcggcc

SEQ ID NO: 136 - DNA, 66 bp, Artificial Sequence
  Description of Artificial Sequence Synthetic primer
ggccgcaagcttcgactgtctctgcactacaattgccgttttccgacggcgcgaattcggatcccc

SEQ ID NO: 137 - DNA, 66 bp, Artificial Sequence
  Description of Artificial Sequence Synthetic primer
ggggatccgaattcgccgccaccaaaaacggcaattgtagtgcagagacagtcgaagcttgcggcc

SEQ ID NO: 138 - DNA, 66 bp, Artificial Sequence
  Description of Artificial Sequence Synthetic primer
ggccgcaagcttcgactgtctctgcactacaattgccgtttttggtggcggcgaattcggatcccc

SEQ ID NO: 139 - DNA, 66 bp, Artificial Sequence
  Description of Artificial Sequence Synthetic primer
ggggatccgaattcgaaaacggcaattgtagtgcagagacagtcgcgccgtcggaagcttgcggcc

SEQ ID NO: 140 - DNA, 66 bp, Artificial Sequence
  Description of Artificial Sequence Synthetic primer
ggccgcaagcttccgacggcgcgactgtctctgcactacaattgccgttttcgaattcggatcccc

SEQ ID NO: 141 - DNA, 66 bp, Artificial Sequence
  Description of Artificial Sequence Synthetic primer
ggggatccgaattcgaaaacggcaattgtagtgcagagacagtcgccgccaccaaagcttgcggcc

SEQ ID NO: 142 - DNA, 66 bp, Artificial Sequence
  Description of Artificial Sequence Synthetic primer
ggccgcaagctttggtggcggcgactgtctctgcactacaattgccgttttcgaattcggatcccc

SEQ ID NO: 143 - DNA, 75 bp, Artificial Sequence
  Description of Artificial Sequence Synthetic primer
ggggatccgaattcgcgccgtcggaaaacggcaattgtagtgcagagacagtcgcgccgtcggaagcttgcggcc

SEQ ID NO: 144 - DNA, 75 bp, Artificial Sequence
  Description of Artificial Sequence Synthetic primer
ggccgcaagcttccgacggcgcgactgtctctgcactacaattgccgttttccgacggcgcgaattcggatcccc

SEQ ID NO: 145 - DNA, 75 bp, Artificial Sequence
  Description of Artificial Sequence Synthetic primer
ggggatccgaattcgccgccaccaaaaacggcaattgtagtgcagagacagtcgccgccaccaaagcttgcggcc

SEQ ID NO: 146 - DNA, 75 bp, Artificial Sequence
  Description of Artificial Sequence Synthetic primer
ggccgcaagctttggtggcggcgactgtctctgcactacaattgccgtttttggtggcggcgaattcggatcccc

SEQ ID NO: 147 - DNA, 75 bp, Artificial Sequence
  Description of Artificial Sequence Synthetic primer
ggggatccgaattcgcgccgtcggaaaacggcaattgtagtgcagagacagtcgccgccaccaaagcttgcggcc

SEQ ID NO: 148 - DNA, 75 bp, Artificial Sequence
  Description of Artificial Sequence Synthetic primer
ggccgcaagctttggtggcggcgactgtctctgcactacaattgccgttttccgacggcgcgaattcggatcccc

SEQ ID NO: 149 - DNA, 57 bp, Artificial Sequence
  Description of Artificial Sequence Synthetic primer
ggggatccgaattcgaaaaaaaagattgtagtgcagagacagtcgaagcttgcggcc

SEQ ID NO: 150 - DNA, 57 bp, Artificial Sequence
  Description of Artificial Sequence Synthetic primer
ggccgcaagcttcgactgtctctgcactacaatcttttttttcgaattcggatcccc

SEQ ID NO: 151 - DNA, 57 bp, Artificial Sequence
  Description of Artificial Sequence Synthetic primer
ggggatccgaattcgaaaacggcaaagattaaacagagacagtcgaagcttgcggcc

SEQ ID NO: 152 - DNA, 57 bp, Artificial Sequence
  Description of Artificial Sequence Synthetic primer
ggccgcaagcttcgactgtctctgtttaatctttgccgttttcgaattcggatcccc

SEQ ID NO: 153 - DNA, 57 bp, Artificial Sequence
  Description of Artificial Sequence Synthetic primer
ggggatccgaattcgaaaacggcaaaaaaggtgcagagacagtcgaagcttgcggcc

SEQ ID NO: 154 - DNA, 57 bp, Artificial Sequence
  Description of Artificial Sequence Synthetic primer
ggccgcaagcttcgactgtctctgcaccttttttgccgttttcgaattcggatcccc

SEQ ID NO: 155 - DNA, 63 bp, Artificial Sequence
  Description of Artificial Sequence Synthetic primer
ggggatccgaattcgaagaaaacggcaattgtagtgcagagacagtcgaagaagcttgcggcc

SEQ ID NO: 156 - DNA, 63 bp, Artificial Sequence
  Description of Artificial Sequence Synthetic primer
ggccgcaagcttcttcgactgtctctgcactacaattgccgttttcttcgaattcggatcccc

SEQ ID NO: 157 - DNA, 66 bp, Artificial Sequence
  Description of Artificial Sequence Synthetic primer
ggggatccgaattcgaagaaaacggcaattaaagtagtgcagagacagtcgaagaagcttgcggcc

SEQ ID NO: 158 - DNA, 66 bp, Artificial Sequence
  Description of Artificial Sequence Synthetic primer
ggccgcaagcttcttcgactgtctctgcactactttaattgccgttttcttcgaattcggatcccc

SEQ ID NO: 159 - DNA, 66 bp, Artificial Sequence
  Description of Artificial Sequence Synthetic primer
ggggatccgaattcgcgccgtcggtcgcaaatggctattcgcgtgacacaacgtaagcttgcggcc

SEQ ID NO: 160 - DNA, 66 bp, Artificial Sequence
  Description of Artificial Sequence Synthetic primer
ggccgcaagcttacgttgtgtcacgcgaatagccatttgcgaccgacggcgcgaattcggatcccc

SEQ ID NO: 161 - DNA, 66 bp, Artificial Sequence
  Description of Artificial Sequence Synthetic primer
ggggatccgaattcgccgccaccatcgcaaatggctattcgcgtgacacaacgtaagcttgcggcc

SEQ ID NO: 162 - DNA, 66 bp, Artificial Sequence
  Description of Artificial Sequence Synthetic primer
ggccgcaagcttacgttgtgtcacgcgaatagccatttgcgatggtggcggcgaattcggatcccc

SEQ ID NO: 163 - DNA, 66 bp, Artificial Sequence
  Description of Artificial Sequence Synthetic primer
ggggatccgaattcgtcgcaaatggctattcgcgtgacacaacgtcgccgtcggaagcttgcggcc

SEQ ID NO: 164 - DNA, 66 bp, Artificial Sequence
  Description of Artificial Sequence Synthetic primer
ggccgcaagcttccgacggcgacgttgtgtcacgcgaatagccatttgcgacgaattcggatcccc

SEQ ID NO: 165 - DNA, 66 bp, Artificial Sequence
  Description of Artificial Sequence Synthetic primer
ggggatccgaattcgtcgcaaatggctattcgcgtgacacaacgtccgccaccaaagcttgcggcc

SEQ ID NO: 166 - DNA, 66 bp, Artificial Sequence
  Description of Artificial Sequence Synthetic primer
ggccgcaagctttggtggcggacgttgtgtcacgcgaatagccatttgcgacgaattcggatcccc

SEQ ID NO: 167 - DNA, 75 bp, Artificial Sequence
  Description of Artificial Sequence Synthetic primer
ggggatccgaattcgcgccgtcggtcgcaaatggctattcgcgtgacacaacgtcgccgtcggaagcttgcggcc

SEQ ID NO: 168 - DNA, 75 bp, Artificial Sequence
  Description of Artificial Sequence Synthetic primer
ggggatccgaattcgccgccaccatcgcaaatggctattcgcgtgacacaacgtccgccaccaaagcttgcggcc

SEQ ID NO: 169 - DNA, 75 bp, Artificial Sequence
  Description of Artificial Sequence Synthetic primer
ggccgcaagcttccgacggcgacgttgtgtcacgcgaatagccatttgcgaccgacggcgcgaattcggatcccc

SEQ ID NO: 170 - DNA, 75 bp, Artificial Sequence
  Description of Artificial Sequence Synthetic primer
ggggatccgaattcgcgccgtcggtcgcaaatggctattcgcgtgacacaacgtccgccaccaaagcttgcggcc

SEQ ID NO: 171 - DNA, 75 bp, Artificial Sequence
  Description of Artificial Sequence Synthetic primer
ggccgcaagctttggtggcggacgttgtgtcacgcgaatagccatttgcgaccgacggcgcgaattcggatcccc

SEQ ID NO: 172 - DNA, 57 bp, Artificial Sequence
  Description of Artificial Sequence Synthetic primer
ggggatccgaattcgtcgcaaatgaagaaacgcgtgacacaacgtaagcttgcggcc

SEQ ID NO: 173 - DNA, 57 bp, Artificial Sequence
  Description of Artificial Sequence Synthetic primer
ggccgcaagcttacgttgtgtcacgcgtttcttcatttgcgacgaattcggatcccc

SEQ ID NO: 174 - DNA, 57 bp, Artificial Sequence
  Description of Artificial Sequence Synthetic primer
ggggatccgaattcgtcgcaaaaagctaaacgcgtgacacaacgtaagcttgcggcc

SEQ ID NO: 175 - DNA, 57 bp, Artificial Sequence
  Description of Artificial Sequence Synthetic primer
ggccgcaagcttacgttgtgtcacgcgtttagctttttgcgacgaattcggatcccc

SEQ ID NO: 176 - DNA, 57 bp, Artificial Sequence
  Description of Artificial Sequence Synthetic primer
ggggatccgaattcgtcgcaaatggctattcgcaaggtgaaacgtaagcttgcggcc

SEQ ID NO: 177 - DNA, 57 bp, Artificial Sequence
  Description of Artificial Sequence Synthetic primer
ggccgcaagcttacgtttcaccttgcgaatagccatttgcgacgaattcggatcccc

SEQ ID NO: 178 - DNA, 63 bp, Artificial Sequence
  Description of Artificial Sequence Synthetic primer
ggggatccgaattcgaaatcgcaaatggctattcgcgtgacacaacgtaaaaagcttgcggcc

SEQ ID NO: 179 - DNA, 63 bp, Artificial Sequence
  Description of Artificial Sequence Synthetic primer
ggggatccgaattcgaaatcgcaaatggctaaacgcgtgacacaacgtaaaaagcttgcggcc

SEQ ID NO: 180 - DNA, 63 bp, Artificial Sequence
  Description of Artificial Sequence Synthetic primer
ggccgcaagctttttacgttgtgtcacgcgaatagccatttgcgatttcgaattcggatcccc

SEQ ID NO: 181 - DNA, 34 bp, Artificial Sequence
  Description of Artificial Sequence Synthetic primer
aagaattcgcgtcgccgccgtcgcaaaacggcaa

SEQ ID NO: 182 - DNA, 28 bp, Artificial Sequence
  Description of Artificial Sequence Synthetic primer
aataaagcttcggtggcggcgactgtct

SEQ ID NO: 183 - DNA, 34 bp, Artificial Sequence
  Description of Artificial Sequence Synthetic primer
aataaagcttaggcggcggtggcggcgactgtct

SEQ ID NO: 184 - DNA, 28 bp, Artificial Sequence
  Description of Artificial Sequence Synthetic primer
aagaattcgcgccgtcgcaaaacggcaa

SEQ ID NO: 185 - DNA, 39 bp, Artificial Sequence
  Description of Artificial Sequence Synthetic primer
aagaattcgggcggtggcaaaacggcaattgtagtgcag

SEQ ID NO: 186 - DNA, 38 bp, Artificial Sequence
  Description of Artificial Sequence Synthetic primer
aatgaagcttgccgccacccgactgtctctgcactaca

SEQ ID NO: 187 - DNA, 66 bp, Artificial Sequence; identical to SEQ ID NO: 135
  Description of Artificial Sequence Synthetic primer

SEQ ID NO: 188 - DNA, 29 bp, Artificial Sequence
  Description of Artificial Sequence Synthetic primer
aagaattcgcgtcgcaaaacggcaattgt

SEQ ID NO: 189 - DNA, 27 bp, Artificial Sequence
  Description of Artificial Sequence Synthetic primer
aatgaagctttggcggcgactgtctct

SEQ ID NO: 190 - protein, 5 aa, Artificial Sequence
  Description of Artificial Sequence Synthetic peptide
RRRRR

SEQ ID NO: 191 - DNA, 30 bp, Artificial Sequence
  Description of Artificial Sequence Synthetic primer
aagaattcgcgcaaaacggcaattgtagtg

SEQ ID NO: 192 - DNA, 26 bp, Artificial Sequence
  Description of Artificial Sequence Synthetic primer
aatgaagcttcggcgactgtctctgc

SEQ ID NO: 193 - protein, 14 aa, Artificial Sequence
  Description of Artificial Sequence Synthetic peptide
SQMAIRRVTQRPPP

SEQ ID NO: 194 - protein, 13 aa, Artificial Sequence
  Description of Artificial Sequence Synthetic peptide
SQMAIRVTQRRRR

SEQ ID NO: 195 - protein, 8 aa, Escherichia coli
NSALALQT

SEQ ID NO: 196 - protein, 7 aa, Escherichia coli
TAIVVQR

SEQ ID NO: 197 - protein, 6 aa, Escherichia coli
GGVVIA

SEQ ID NO: 198 - protein, 4 aa, Escherichia coli
VVIA

SEQ ID NO: 199 - protein, 4 aa, Escherichia coli
AIVV

SEQ ID NO: 200 - DNA, 456 bp, Escherichia coli
atgaaacttttaaaagtagcagcaattgcagcaatcgtattctccggtagcgctctggcaggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtactaa

SEQ ID NO: 201 - DNA, 455 bp, Escherichia coli
atgaaaaacaaattgttatttatgatgttaacaatactgggtgcgcctgggattgcagccgcagcaggttatgatttagctaattcagaatataacttcgcggtaaatgaattgagtaagtcttcatttaatcaggcagccataattggtcaagctgggactaataatagtgctcagttacggcagggaggctcaaaacttttggcggttgttgcgcaagaaggtagtagcaccgggcaaagattgaccagacaggagattataaaccttgcatatattgatcaggcgggcagtgccaacgatgccagtatttcgcaaggtgcttatggtaatactgcgatgattatccagaaaggttctggtaataaagcaaatattacacagtatggtactcaaaaaacggcaattgtagtgcagagacagtcgcaatggctattcgcgtgacacaacgttaa

SEQ ID NO: 202 - protein, 22 aa, Escherichia coli
LRQGGSKLLAVVAQEGSSNRAK

SEQ ID NO: 203 - protein, 20 aa, Escherichia coli
LRQGGSKLLAVVAQEGSSNR

SEQ ID NO: 204 - protein, 20 aa, Escherichia coli
QGGSKLLAVVAQEGSSNRAK

SEQ ID NO: 205 - protein, 151 aa, Artificial Sequence
  Description of Artificial Sequence Synthetic polypeptide
MKLLKVAAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 206 - protein, 151 aa, Artificial Sequence
  Description of Artificial Sequence Synthetic polypeptide
MKNKLLFMMLTILGAPGIAAAAGYDLANSEYNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 207 - protein, 131 aa, Artificial Sequence
  Description of Artificial Sequence Synthetic polypeptide
GVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 208 - protein, 131 aa, Artificial Sequence
  Description of Artificial Sequence Synthetic polypeptide
AAGYDLANSEYNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 209 - protein, 8 aa, Escherichia coli
ALKKQTDA

SEQ ID NO: 210 - protein, 8 aa, Escherichia coli
AIVVQTQS

SEQ ID NO: 211 - protein, 8 aa, Escherichia coli
GGNSALAL

SEQ ID NO: 212 - protein, 8 aa, Escherichia coli
SQRQKVIA

SEQ ID NO: 213 - protein, 9 aa, Escherichia coli
NSSVNVTQV

SEQ ID NO: 214 - protein, 8 aa, Escherichia coli
SELNIYQY

SEQ ID NO: 215 - protein, 8 aa, Escherichia coli
TAIVVQRQ

SEQ ID NO: 216 - protein, 5 aa, Artificial Sequence
  Description of Artificial Sequence Synthetic peptide
PPPPP

SEQ ID NO: 217 - protein, 5 aa, Artificial Sequence
  Description of Artificial Sequence Synthetic peptide
AIVVQ

SEQ ID NO: 218 - protein, 8 aa, Artificial Sequence
  Description of Artificial Sequence Synthetic peptide
QRQVVIAT

SEQ ID NO: 219 - protein, 15 aa, Artificial Sequence
  Description of Artificial Sequence Synthetic peptide
XXXSEGGGSEGGGXX

SEQ ID NO: 220 - protein, 6 aa, Escherichia coli
LAVVAQ

SEQ ID NO: 221 - protein, 16 aa, Escherichia coli
GGSKLLAVVAQEGSSN

SEQ ID NO: 222 - protein, 9 aa, Escherichia coli
KLLAVVAQE

SEQ ID NO: 223 - protein, 8 aa, Escherichia coli
KTAIVVQR

SEQ ID NO: 224 - protein, 17 aa, Escherichia coli
TQKTAIVVQRQSQMAIR

SEQ ID NO: 225 - protein, 24 aa, Listeria monocytogenes
MKKIMLVITLILVSPIAQQTEAKD

SEQ ID NO: 226 - protein, 27 aa, Artificial Sequence
  Description of Artificial Sequence Synthetic peptide
RRRNQQNYQQYSQNGNQQQGNNRYPPP

SEQ ID NO: 227 - protein, 27 aa, Artificial Sequence
  Description of Artificial Sequence Synthetic peptide
RRRNQQNYQQYSQNGNQQQGNNRYPPP

SEQ ID NO: 228 - protein, 27 aa, Artificial Sequence
  Description of Artificial Sequence Synthetic peptide
RRRISESTHNTNNANVTSADALIKPPP

SEQ ID NO: 229 - protein, 27 aa, Artificial Sequence
  Description of Artificial Sequence Synthetic peptide
RRRISESTHNTNNANVTSADALIKPPP

SEQ ID NO: 230 - protein, 31 aa, Bacillus anthracis
MKKRKVLIPLMALSTILVSSTGNLEVIQAEV

SEQ ID NO: 231 - protein, 29 aa, Listeria monocytogenes
MNMKKATIAATAGIAVTAFAAPTIASAST

SEQ ID NO: 232 - protein, 54 aa, Listeria monocytogenes
MQKTRKERILEALQEEKKNKKSKKFKTGATIAGVTAIATSITVPGIEVIVSADE

SEQ ID NO: 233 - protein, 28 aa, Bacillus anthracis
MKKLKMASCALVAGLMFSGLTPNAFAED

SEQ ID NO: 234 - protein, 31 aa, Staphylococcus aureus
MAKKFNYKLPSMVALTLVGSAVTAHQVQAAE

SEQ ID NO: 235 - protein, 59 aa, Listeria monocytogenes
MTDKKSENQTEKTETKENKGMTRREMLKLSAVAGTGIAVGATGLGTILNVVDQVDKALT

SEQ ID NO: 236 - protein, 53 aa, Bacillus subtillis
MAYDSRFDEWVQKLKEESFQNNTFDRRKFIQGAGKIAGLGLGLTIAQSVGAFG

SEQ ID NO: 237 - protein, 29 aa, Lactococcus lactis
MKKKIISAILMSTVILSAAAPLSGVYADT

SEQ ID NO: 238 - protein, 5 aa, Listeria monocytogenes
TEAKD

SEQ ID NO: 239 - protein, 5 aa, Lactococcus lactis
VYADT

SEQ ID NO: 240 - protein, 5 aa, Bacillus anthracis
IQAEV

SEQ ID NO: 241 - protein, 5 aa, Listeria monocytogenes
ASAST

SEQ ID NO: 242 - protein, 5 aa, Listeria monocytogenes
VSADE

SEQ ID NO: 243 - protein, 5 aa, Bacillus anthracis
AFAED

SEQ ID NO: 244 - protein, 5 aa, Staphylococcus aureus
VQAAE

SEQ ID NO: 245 - protein, 5 aa, Listeria monocytogenes
DKALT

SEQ ID NO: 246 - DNA, 84 bp, Artificial Sequence
  Description of Artificial Sequence Synthetic oligonucleotide
gcatgctccctatcagtgatagagattgacatccctatcagtgatagagatactgagcacatcagcaggacgcactgaccagga

SEQ ID NO: 247 - DNA, 286 bp, Artificial Sequence
  Description of Artificial Sequence Synthetic polynucleotide
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccata

SEQ ID NO: 248 - DNA, 90 bp, Artificial Sequence
  Description of Artificial Sequence Synthetic oligonucleotide
ccatcgaatggctgaaatgagctgttgacaattaatcatccggctcgtataatgtgtggaattgtgagcggataacaatttcacacagga

SEQ ID NO: 249 - DNA, 102 bp, Artificial Sequence
  Description of Artificial Sequence Synthetic polynucleotide
gcatgcacagataaccatctgcggtgataaattatctctggcggtgttgacataaataccactggcggttataatgagcacatcagcagggtatgcaaagga

SEQ ID NO: 250 - protein, 20 aa, Escherichia coli
GTQKTAIVVQRQSQMAIRVT

SEQ ID NO: 251 - protein, 150 aa, Shigella sp.
MKLLKVAAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVQVGFGNNATAHQY

SEQ ID NO: 252 - protein, 149 aa, Artificial Sequence
  Description of Artificial Sequence Synthetic polypeptide
MKLLKVAAIAAIVFSGSALAGVVPQYGGGNHGGGGNNSGPNSELNIYQYGGGNSALVLQTDARNSDLTITQHGGGNGADVGQGSDDSSIDLTQRGFGNSATLDQWNGKNSEMTVKQFGGGNGAAVDQTASNSSVNVQVGFGNNATAHQY

SEQ ID NO: 253 - protein, 150 aa, Artificial Sequence; identical to SEQ ID NO: 251
  Description of Artificial Sequence Synthetic polypeptide

SEQ ID NO: 254 - protein, 150 aa, Artificial Sequence; identical to SEQ ID NO: 251
  Description of Artificial Sequence Synthetic polypeptide

SEQ ID NO: 255 - protein, 151 aa, Artificial Sequence
  Description of Artificial Sequence Synthetic polypeptide
MKLLKVAAIAAIVFSGSALAGVVPQYGGGGGNHGGGGNNSGPNSELNIYQYGGGNSALALQADARNSDLTITQHGGGNGADVGQGSDDSSIDLTQRGFGNSATLDQWNGKDSHMTVKQFGGGNGAAVDQTASNSTVNVQVGFGNNATAHQY

SEQ ID NO: 256 - protein, 151 aa, Salmonella typhimurium
MKLLKVAAFAAIVVSGSALAGVVPQWGGGGNHNGGGNSSGPDSTLSIYQYGSANAALALQSDARKSETTITQSGYGNGADVGQGADNSTIELTQNGFRNNATIDQWNAKNSDITVGQYGGNNAALVNQTASDSSVMVRQVGFGNNATANQY

SEQ ID NO: 257 - protein, 151 aa, Salmonella enterica; identical to SEQ ID NO: 256

SEQ ID NO: 258 - protein, 151 aa, Artificial Sequence; identical to SEQ ID NO: 256
  Description of Artificial Sequence Synthetic polypeptide

SEQ ID NO: 259 - protein, 150 aa, Artificial Sequence; identical to SEQ ID NO: 251
  Description of Artificial Sequence Synthetic consensus peptide

SEQ ID NO: 260 - protein, 151 aa, Salmonella typhimurium; identical to SEQ ID NO: 256

SEQ ID NO: 261 - protein, 151 aa, Salmonella enterica; identical to SEQ ID NO: 256

SEQ ID NO: 262 - protein, 151 aa, Salmonella enterica; identical to SEQ ID NO: 256

SEQ ID NO: 263 - protein, 151 aa, Salmonella enterica; identical to SEQ ID NO: 256

SEQ ID NO: 264 - protein, 151 aa, Salmonella enterica; identical to SEQ ID NO: 256

SEQ ID NO: 265 - protein, 151 aa, Salmonella enterica; identical to SEQ ID NO: 256

SEQ ID NO: 266 - protein, 151 aa, Salmonella enterica; identical to SEQ ID NO: 256

SEQ ID NO: 267 - protein, 151 aa, Salmonella enterica; identical to SEQ ID NO: 256

SEQ ID NO: 268 - protein, 151 aa, Salmonella enterica; identical to SEQ ID NO: 256

SEQ ID NO: 269 - protein, 151 aa, Salmonella enterica; identical to SEQ ID NO: 256

SEQ ID NO: 270 - protein, 151 aa, Salmonella enterica; identical to SEQ ID NO: 256

SEQ ID NO: 271 - protein, 152 aa, Escherichia coli
MKLLKVAAIAAIVFSGSALAGVVPQYGGGGGNHGGGGNNSGPNSELNIYQYGGGNSALALQADARNSDLTITQHGGGNGADVGQGSDDSSIDLTQRGFGNSATLDQWNGKDSHMTVKQFGGGNGAAVDQTASNSTVNVTQVGFGNNATAHQY

SEQ ID NO: 272 - protein, 152 aa, Escherichia coli; identical to SEQ ID NO: 271

SEQ ID NO: 273 - protein, 152 aa, Escherichia coli; identical to SEQ ID NO: 271

SEQ ID NO: 274 - protein, 152 aa, Escherichia coli; identical to SEQ ID NO: 271

SEQ ID NO: 275 - protein, 152 aa, Escherichia coli; identical to SEQ ID NO: 271

SEQ ID NO: 276 - protein, 152 aa, Escherichia coli; identical to SEQ ID NO: 271

SEQ ID NO: 277 - protein, 151 aa, Escherichia coli
MKLLKVAAIAAIVFSGSALAGVVPQYGGGGNHGGGGNNSGPNSELNIYQYGGGNSALALQADARNSDLTITQHGGGNGADVGQGSDDSSIDLTQRGFGNSATLDQWNGKDSTMTVKQFGGGNGAAVDQTASNSSVNVTQVGFGNNATAHQY

SEQ ID NO: 278 - protein, 152 aa, Escherichia coli
MKLLKVAAIAAIVFSGSALAGVVPQYGGGGGNHGGGGNNSGPNSELNIYQYGGGNSALALQADARNSDLTITQHGGGNGADVGQGSDDSSIDLTQRGFGNSATLDQWNGKDSTMTVKQFGGGNGAAVDQTASNSSVNVTQVGFGNNATAHQY

SEQ ID NO: 279 - protein, 151 aa, Escherichia coli; identical to SEQ ID NO: 277

SEQ ID NO: 280 - protein, 151 aa, Escherichia coli; identical to SEQ ID NO: 277

SEQ ID NO: 281 - protein, 151 aa, Escherichia coli; identical to SEQ ID NO: 277

SEQ ID NO: 282 - protein, 152 aa, Escherichia coli
MKLLKVAAIAAIVFSGSALAGVVPQYGGGGGNHGGGGNNSGPNSELNIYQYGGGNSALAQQADARNSDLTITQHGGGNGADVGQGSDDSSIDLTQRGFGNSATLDQWNGKDSTMTVKQFGGGNGAAVDQTASNSSVNVTQVGFGNNATAHQY

SEQ ID NO: 283 - protein, 152 aa, Escherichia coli; identical to SEQ ID NO: 282

SEQ ID NO: 284 - protein, 151 aa, Escherichia coli; identical to SEQ ID NO: 205

SEQ ID NO: 285 - protein, 151 aa, Escherichia coli; identical to SEQ ID NO: 205

SEQ ID NO: 286 - protein, 151 aa, Escherichia coli; identical to SEQ ID NO: 205

SEQ ID NO: 287 - protein, 151 aa, Shigella boydii; identical to SEQ ID NO: 205

SEQ ID NO: 288 - protein, 151 aa, Shigella boydii; identical to SEQ ID NO: 205

SEQ ID NO: 289 - protein, 151 aa, Escherichia coli; identical to SEQ ID NO: 205

SEQ ID NO: 290 - protein, 151 aa, Escherichia coli; identical to SEQ ID NO: 205

SEQ ID NO: 291 - protein, 151 aa, Escherichia coli; identical to SEQ ID NO: 205

SEQ ID NO: 292 - protein, 151 aa, Escherichia coli; identical to SEQ ID NO: 205

SEQ ID NO: 293 - protein, 151 aa, Escherichia coli; identical to SEQ ID NO: 205

SEQ ID NO: 294 - protein, 151 aa, Escherichia coli; identical to SEQ ID NO: 205

SEQ ID NO: 295 - protein, 151 aa, Escherichia coli; identical to SEQ ID NO: 205

SEQ ID NO: 296 - protein, 151 aa, Escherichia coli; identical to SEQ ID NO: 205

SEQ ID NO: 297 - protein, 151 aa, Escherichia coli; identical to SEQ ID NO: 205

SEQ ID NO: 298 - protein, 151 aa, Escherichia coli; identical to SEQ ID NO: 205

SEQ ID NO: 299 - protein, 151 aa, Escherichia coli; identical to SEQ ID NO: 205

SEQ ID NO: 300 - protein, 151 aa, Escherichia coli; identical to SEQ ID NO: 205

SEQ ID NO: 301 - protein, 151 aa, Escherichia coli
MKLLKVEAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 302 - protein, 150 aa, Shigella sonnei
MKLLKVAAIAAIVFSGSALAGVVPQYGGGNHGGGGNNSGPNSELNIYQYGGGNSALVLQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 303 - protein, 150 aa, Shigella sonnei; identical to SEQ ID NO: 302

SEQ ID NO: 304 - protein, 138 aa, Salmonella enterica
MRVKHAVVLLMLFSPLTWAGNMTFQFRNPNFGGNPNNGSFLLNSAQAQNSYKDPAYDNDFGIETPSALDNFTQAIQSQILGGLLTNINTGKPGRMVTNDFIIDIANRDGQLQLNVTDRKTGRTSTIEVSGLQTQSTDF

SEQ ID NO: 305 - protein, 138 aa, Salmonella enterica; identical to SEQ ID NO: 304

SEQ ID NO: 306 - protein, 138 aa, Salmonella enterica; identical to SEQ ID NO: 304

SEQ ID NO: 307 - protein, 138 aa, Salmonella enterica; identical to SEQ ID NO: 304

SEQ ID NO: 308 - protein, 138 aa, Shigella flexneri
MRVKHAVVLLMLISPLSWAGTMTFQFRNPNFGGNPNNGAFLLNSAQAQNSYKDPSYNDDFGIETPSALDNFTQAIQSQILGGLLSNINTGKPGRMVTNDYIVDIANRDGQLQLNVTDRKTGQTSTIQVSGLQNNSTDF

SEQ ID NO: 309 - protein, 138 aa, Shigella sonnei; identical to SEQ ID NO: 308

SEQ ID NO: 310 - protein, 138 aa, Shigella flexneri; identical to SEQ ID NO: 308

SEQ ID NO: 311 - protein, 138 aa, Shigella dysenteriae
MRVKHAVVLLMLISPLSWAGTMTFQFRNPNFGGNPNNGAFLLNSAQAQNSYKDPSYNDDFGIETPSALDNFTQAIQSQILGGLLSNINTGKPGRMVTNDYIVDIANRDGQLQLNMTDRKTGQTSTIQVSGLQNNSTDF

SEQ ID NO: 312 - protein, 131 aa, Salmonella enterica
MKRYLTWIVAAELLFATGNLHANEVEVEVPGLLTDHTVSSIGHEFYRAFSDKWESEYTGNLTINERPSARWGSWITITVNQDVIFQTFLFPMKRDFEKTVVFALAQTEEALNRRQIDQTLLSTSDLARDEF

SEQ ID NO: 313 - protein, 131 aa, Salmonella enterica; identical to SEQ ID NO: 312

SEQ ID NO: 314 - protein, 131 aa, Salmonella enterica; identical to SEQ ID NO: 312

SEQ ID NO: 315 - protein, 131 aa, Salmonella enterica; identical to SEQ ID NO: 312

SEQ ID NO: 316 - protein, 131 aa, Salmonella enterica; identical to SEQ ID NO: 312

SEQ ID NO: 317 - protein, 129 aa, Shigella flexneri
MKRYLRWIVAAEFLFAAGNLHAVEVEVPGLLTDHTVSSIGHDFYRAFSDKWESDYTGNLTINERPSARWGSWITITVNQDVIFQTFLFPLKRDSEKTVVFALIQTEEALNRRQINQALLSTDDLAHDEF

SEQ ID NO: 318 - protein, 129 aa, Shigella flexneri; identical to SEQ ID NO: 317

SEQ ID NO: 319 - protein, 129 aa, Shigella flexneri; identical to SEQ ID NO: 317

SEQ ID NO: 320 - protein, 129 aa, Shigella dysenteriae
MKRYLRWIVAAEFLFAAGNLHAVEVEVPGLLTDHTVSSIGHDFYRAFSDKWESDYTGNLTINERPSARWGSWITITVNQDVIFQTFLFPLKRDFEKTVVFALIQTEEALNRRQINQALLSTGDLAHGEF

SEQ ID NO: 321 - protein, 129 aa, Shigella sonnei
MKRYLRWIVAAEFLFAAGNLHAVEVEVPGLLTDHTVSSIGHDFYRAFSDKWESDYTGNLTINERPSARWGSWITITVNQDVIFQTFLFPLKRDFEKTVVFALIQTEEALNRRQINQALLSTGDLAHDEF

SEQ ID NO: 322 - protein, 160 aa, Escherichia coli
MYDQVQGDNMKNKLLFMMLTILGAPGIAAAAGYDLANSEYNFAVNELSKSSFNQAAIIGQAGTNNSAQLRQGGSKLLAVVAQEGSSNRAKIDQTGDYNLAYIDQAGNANDASISQGAYGNTAMIIQKGSGNKANITQYGTQKTAIVVQRQSQMAIRVTQR

SEQ ID NO: 323 - protein, 160 aa, Escherichia coli; identical to SEQ ID NO: 322

SEQ ID NO: 324 - protein, 160 aa, Escherichia coli; identical to SEQ ID NO: 322

SEQ ID NO: 325 - protein, 160 aa, Escherichia coli; identical to SEQ ID NO: 322

SEQ ID NO: 326 - protein, 160 aa, Escherichia coli; identical to SEQ ID NO: 322

SEQ ID NO: 327 - protein, 151 aa, Escherichia coli; identical to SEQ ID NO: 206

SEQ ID NO: 328 - protein, 91 aa, Escherichia coli
QAGTNNSAQLRQGGSKLLAVVAQEGSSNRAKIDQTGDYNLAYIDQAGSANDASISQGAYGNTAMIIQKGSGNKANITQYGTQKTAIVVQRQ

SEQ ID NO: 329 - protein, 151 aa, Escherichia coli; identical to SEQ ID NO: 206

SEQ ID NO: 330 - protein, 151 aa, Escherichia coli; identical to SEQ ID NO: 206

SEQ ID NO: 331 - protein, 151 aa, Escherichia coli; identical to SEQ ID NO: 206

SEQ ID NO: 332 - protein, 151 aa, Escherichia coli; identical to SEQ ID NO: 206

SEQ ID NO: 333 - protein, 151 aa, Escherichia coli; identical to SEQ ID NO: 206

SEQ ID NO: 334 - protein, 151 aa, Escherichia coli; identical to SEQ ID NO: 206

SEQ ID NO: 335 - protein, 151 aa, Escherichia coli; identical to SEQ ID NO: 206

SEQ ID NO: 336 - protein, 151 aa, Escherichia coli; identical to SEQ ID NO: 206

SEQ ID NO: 337 - protein, 151 aa, Escherichia coli; identical to SEQ ID NO: 206

SEQ ID NO: 338 - protein, 151 aa, Escherichia coli; identical to SEQ ID NO: 206

SEQ ID NO: 339 - protein, 151 aa, Escherichia coli; identical to SEQ ID NO: 206

SEQ ID NO: 340 - protein, 151 aa, Escherichia coli; identical to SEQ ID NO: 206

SEQ ID NO: 341 - protein, 151 aa, Escherichia coli; identical to SEQ ID NO: 206

SEQ ID NO: 342 - protein, 151 aa, Escherichia coli; identical to SEQ ID NO: 206

SEQ ID NO: 343 - protein, 151 aa, Escherichia coli; identical to SEQ ID NO: 206

SEQ ID NO: 344 - protein, 151 aa, Escherichia coli; identical to SEQ ID NO: 206

SEQ ID NO: 345 - protein, 151 aa, Escherichia coli; identical to SEQ ID NO: 206

SEQ ID NO: 346 - protein, 151 aa, Escherichia coli; identical to SEQ ID NO: 206

SEQ ID NO: 347 - protein, 151 aa, Shigella sonnei
MKNKLLFMMLTILGAPGIAAAAGYDLANSEYNFAVNELSKSSFNQAAIIGQAGTNNSTQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 348 - protein, 151 aa, Shigella sonnei; identical to SEQ ID NO: 347

SEQ ID NO: 349 - protein, 160 aa, Escherichia coli
MYDQVQGDNMKNKLLFMMLTILGAPGIAAAAGYDLANSEYNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 350 - protein, 160 aa, Escherichia coli; identical to SEQ ID NO: 349

SEQ ID NO: 351 - protein, 160 aa, Escherichia coli; identical to SEQ ID NO: 349

SEQ ID NO: 352 - protein, 160 aa, Escherichia coli; identical to SEQ ID NO: 349

SEQ ID NO: 353 - protein, 160 aa, Escherichia coli; identical to SEQ ID NO: 349

SEQ ID NO: 354 - protein, 160 aa, Escherichia coli; identical to SEQ ID NO: 349

SEQ ID NO: 355 - protein, 160 aa, Escherichia coli; identical to SEQ ID NO: 349

SEQ ID NO: 356 - protein, 160 aa, Escherichia coli; identical to SEQ ID NO: 349

SEQ ID NO: 357 - protein, 160 aa, Escherichia coli; identical to SEQ ID NO: 349

SEQ ID NO: 358 - protein, 160 aa, Escherichia coli; identical to SEQ ID NO: 349

SEQ ID NO: 359 - protein, 160 aa, Escherichia coli; identical to SEQ ID NO: 349

SEQ ID NO: 360 - protein, 160 aa, Escherichia coli; identical to SEQ ID NO: 349

SEQ ID NO: 361 - protein, 160 aa, Escherichia coli; identical to SEQ ID NO: 349

SEQ ID NO: 362 - protein, 160 aa, Escherichia coli
MYDQVQGDNMKNKLLFMMLTILGAPGIAAAAGYDLANSEYNFAVNELSKSSFNQAAIIGQAGTNNSAQLRQGGSKLLTVVAQEGSSNRAKIDQTGDYNLAYIDQAGSANDASISQGAYGNTAMIIQKGSGNKANITQYGTQKTAIVVQRQSQMAIRVTQR

SEQ ID NO: 363 - protein, 160 aa, Escherichia coli; identical to SEQ ID NO: 362

SEQ ID NO: 364 - protein, 151 aa, Shigella flexneri
MKNKLLFMMLTILGAPGIAAAAGYDLANSEYNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAVVVQRQSQMAIRVTQR

SEQ ID NO: 365 - protein, 151 aa, Shigella flexneri; identical to SEQ ID NO: 364

SEQ ID NO: 366 - protein, 151 aa, Shigella flexneri; identical to SEQ ID NO: 364

SEQ ID NO: 367 - protein, 151 aa, Shigella flexneri; identical to SEQ ID NO: 364

SEQ ID NO: 368 - protein, 151 aa, Shigella flexneri
MKNKLLFMMLTILGAPGIAAAAGYDLANSEYNFAVNELSKSSFNQAAIIGQAGTNNSAPLRQGGSKLLAVVAQEGSSNRAKIDQTGDYNLAYIDQAGSANDASISQGAYGNTAMIIQKGSGNKANITQYGTQKTAVVVQRQSQMAIRVTQR

SEQ ID NO: 369 - protein, 151 aa, Shigella flexneri; identical to SEQ ID NO: 368

SEQ ID NO: 370 - protein, 151 aa, Salmonella enterica
MKNKLLFMMLTILGAPGIATATNYDLARSEYNFAVNELSKSSFNQAAIIGQVGTDNSARVRQEGSKLLSVISQEGGNNRAKVDQAGNYNFAYIEQTGNANDASISQSAYGNSAAIIQKGSGNKANITQYGTQKTAVVVQKQSHMAIRVTQR

SEQ ID NO: 371 - protein, 151 aa, Salmonella enterica; identical to SEQ ID NO: 370

SEQ ID NO: 372 - protein, 151 aa, Salmonella enterica; identical to SEQ ID NO: 370

SEQ ID NO: 373 - protein, 151 aa, Salmonella enterica; identical to SEQ ID NO: 370

SEQ ID NO: 374 - protein, 151 aa, Salmonella enterica; identical to SEQ ID NO: 370

SEQ ID NO: 375 - protein, 151 aa, Salmonella enterica; identical to SEQ ID NO: 370

SEQ ID NO: 376 - protein, 151 aa, Salmonella enterica; identical to SEQ ID NO: 370

SEQ ID NO: 377 - protein, 151 aa, Salmonella enterica; identical to SEQ ID NO: 370

SEQ ID NO: 378 - protein, 151 aa, Salmonella enterica; identical to SEQ ID NO: 370

SEQ ID NO: 379 - protein, 151 aa, Salmonella enterica
MKNKLLFMMLTILGAPGIATATNYDLARSEYNFAVNELSKSSFNQAAIIGQVGTDNSARVRQEGSKLLSVISQEGENNRAKVDQAGNYNFAYIEQTGNANDASISQSAYGNSAAIIQKGSGNKANITQYGTQKTAVVVQKQSHMAIRVTQR

SEQ ID NO: 380 - protein, 151 aa, Salmonella enterica; identical to SEQ ID NO: 379

SEQ ID NO: 381 - protein, 151 aa, Salmonella enterica; identical to SEQ ID NO: 379

SEQ ID NO: 382 - protein, 151 aa, Salmonella enterica; identical to SEQ ID NO: 379

SEQ ID NO: 383 - protein, 151 aa, Salmonella enterica; identical to SEQ ID NO: 379

SEQ ID NO: 384 - protein, 151 aa, Salmonella enterica; identical to SEQ ID NO: 379

SEQ ID NO: 385 - protein, 6 aa, Escherichia coli
SALALQ

SEQ ID NO: 386 - protein, 6 aa, Escherichia coli
SELNIY

SEQ ID NO: 387 - protein, 5 aa, Escherichia coli
NSSVN

SEQ ID NO: 388 - protein, 5 aa, Artificial Sequence
  Description of Artificial Sequence Synthetic peptide
LPXTG

SEQ ID NO: 389 - protein, 6 aa, Escherichia coli
NNATAH

SEQ ID NO: 390 - protein, 5 aa, Escherichia coli
TAIVV

SEQ ID NO: 391 - protein, 8 aa, Escherichia coli
SQMAIRTV

SEQ ID NO: 392 - protein, 6 aa, Escherichia coli
AAIIGQ

SEQ ID NO: 393 - protein, 6 aa, Escherichia coli
SAQLRQ

SEQ ID NO: 394 - protein, 8 aa, Escherichia coli
NSDLTITQ

SEQ ID NO: 395 - protein, 5 aa, Unknown
  Description of Unknown Beta-amyloid recognition peptide
KLVFF

SEQ ID NO: 396 - protein, 6 aa, Homo sapiens
VQIVYK

(19) United States
(12) Patent Application Publication
(10) Pub. No.: US 2012/0301433 A1
(43) Pub. Date: Nov. 29, 2012

(54) BACTERIOPHAGES EXPRESSING AMYLOID PEPTIDES AND USES THEREOF

(75) Inventors: Timothy Kuan-Ta Lu, Charlestown, MA (US); Susan Lindquist, Chestnut Hill, MA (US); Rajaraman Krishnan, Quincy, MA (US); James Collins, Newton Center, MA (US); Charles W. O'Donnell, Somerville, MA (US); Bonnie Berger Leighton, Newtonville, MA (US); Srinivas Devadas, Lexington, MA (US)

(73) Assignees: WHITEHEAD INSTITUTE FOR BIOMEDICAL RESEARCH, Cambridge, MA (US); MASSACHUSETTS INSTITUTE OF TECHNOLOGY, Cambridge, MA (US); TRUSTEES OF BOSTON UNIVERSITY, Boston, MA (US)

(21) Appl. No.: 13/387,888

(22) PCT Filed: Jul. 29, 2010
(86) PCT No.: PCT/US10/43770

§ 371 Date: Aug. 8, 2012

Related U.S. Application Data
(60) Provisional application No. 61/229,703, filed on Jul. 29, 2009.
(60) Provisional application No. 61/233,697, filed on Aug. 13, 2009.

(51) Int. Cl. C12N 7/01; C12S 9/00; A61K 35/76

(52) U.S. Cl. 424/93.2; 435/235.1; 435/264

(57) ABSTRACT

The present invention generally relates to engineered bacteriophages which express amyloid peptides for the modulation (e.g. increase or decrease) of protein aggregates and amyloid formation. In some embodiments, the engineered bacteriophages express anti-amyloid peptides for inhibiting protein aggregation and amyloid formation, which can be useful in the treatment and prevention of and bacterial infections and biofilms. In some embodiments, the engineered bacteriophages express amyloid peptides for promoting amyloid formation, which are useful for increasing amyloid formation such as promoting bacterial biofilms. Other aspects relate to methods to inhibit bacteria biofilms, and methods for the treatment of amyloid related disorders, e.g., Alzheimer's disease using an anti-amyloid peptide engineered bacteriophages. Other aspects of the invention relate to engineered bacteriophages to express the amyloid peptides on the bacteriophage surface and/or secrete the amyloid peptides, e.g., anti-amyloid peptides and pro-amyloid peptides, and uses thereof for modulation protein aggregates and amyloid formation.

### CROSS REFERENCE TO RELATED APPLICATIONS

This application claims benefit under 35 U.S.C. 119(e) of U.S. Provisional Patent Application Ser. No. 61/229,703 filed Jul. 29, 2009, and U.S. Provisional Patent Application Ser. No. 61/233,697 filed Aug. 13, 2009, the contents of each are incorporated herein in their entirety by reference.

### GOVERNMENT SUPPORT

This invention was made with government support under R01 GM 025874-29 and OD003644 awarded by the National Institites of Health (NIH). The Government has certain rights to the invention.

### FIELD OF THE INVENTION

The present invention relates to the field of treatment and prevention of bacteria and bacterial infections. In particular, the present invention relates to engineered bacteriophages that have been engineered to express and secrete amyloid peptides, including anti-amyloid peptides and pro-amyloid peptides.

### BACKGROUND OF THE INVENTION

Bacterial biofilms are sources of contamination that are difficult to eliminate in a variety of industrial, environmental and clinical settings. Biofilms are polymer structures secreted by bacteria to protect bacteria from various environmental attacks, and thus result also in protection of the bacteria from disinfectants and antibiotics. Biofilms can be found on any environmental surface where sufficient moisture and nutrients are present. Bacterial biofilms are associated with many human and animal health and environmental problems. For instance, bacteria form biofilms on implanted medical devices, e.g., catheters, heart valves, joint replacements, and damaged tissue, such as the lungs of cystic fibrosis patients. Bacteria in biofilms are highly resistant to antibiotics and host defenses and consequently are persistent sources of infection.

Biofilms also contaminate surfaces such as water pipes and the like, and render also other industrial surfaces hard to disinfect. For example, catheters, in particular central venous catheters (CVCs), are one of the most frequently used tools for the treatment of patients with chronic or critical illnesses and are inserted in more than 20 million hospital patients in the USA each year. Their use is often severely compromised as a result of bacterial biofilm infection which is associated with significant mortality and increased costs. Catheters are associated with infection by many biofilm forming organisms such as Staphylococcus epidermidis, Staphylococcus aureus, Pseudomonas aeruginosa, Enterococcus faecalis and Candida albicans which frequently result in generalized blood stream infection. Approximately 250,000 cases of CVC-associated bloodstream infections occur in the US each year with an associated mortality of 12%-25% and an estimated cost of treatment per episode of approximately $25,000. Treatment of CVC-associated infections with conventional antimicrobial agents alone is frequently unsuccessful due to the extremely high tolerance of biofilms to these agents. Once CVCs become infected the most effective treatment still involves removal of the catheter, where possible, and the treatment of any surrounding tissue or systemic infection using antimicrobial agents. This is a costly and risky procedure and re-infection can quickly occur upon replacement of the catheter.

Bacteriophages (often known simply as “phages”) are viruses that grow within bacteria. The name translates as “eaters of bacteria” and reflects the fact that as they grow, the majority of bacteriophages kill the bacterial host in order to release the next generation of bacteriophages. Naturally occurring bacteriophages are incapable of infecting anything other than specific strains of the target bacteria, undermining their potential for use as control agents.

Bacteriophages (phage) and their therapeutic uses have been the subject of much interest since they were first recognized early in the 20th century. Lytic bacteriophages are viruses that infect bacteria exclusively, replicate, disrupt bacterial metabolism and destroy the cell upon release of phage progeny in a process known as lysis. These bacteriophages have very effective antibacterial activity and in theory have several advantages over antibiotics. Most notably they replicate at the site of infection and are therefore available in abundance where they are most required; no serious or irreversible side effects of phage therapy have yet been described and selecting alternative phages against resistant bacteria is a relatively rapid process that can be carried out in days or weeks.

Bacteriophages have been reported to be used to sanitize surfaces that may be contaminated with bacteria, as discussed in for example, U.S. Pat. No. 6,699,701. Also, systems using bacteriophages that encode enzymes that attack certain biofilm components have been described. Other examples of lytic enzymes, such as dispersin encoded by bacteriophages that have been used to destroy bacteria have been reported in U.S. Pat. No. 6,335,012 and U.S. Patent Application Publication No. 2005/0004030.

For example, PCT Publication No. WO 2004/062677 discusses a method of treating bacterial biofilm using a bacteriophage capable of infecting the bacteria within the biofilm and wherein the bacteriophage also encodes a polysaccharide lyase enzyme that is capable of degrading polysaccharides in the biofilm. In one embodiment, additional enzyme is absorbed on the surface of the phage.

However, even when the phage of WO 2004/062677 is delivered with an enzyme mixture or with an enzyme “associated” or “absorbed” on the surface of the first phage dose, the method requires that after the initial administration, the phage released from the destroyed bacteria must “find” and infect at least one additional bacterium to enable it to continue to degrade polysaccharides in the biofilm. Therefore, WO 2004/062677 specifically discusses the benefits of using multiple dosages of phage administration to enhance the results (see, e.g., page 14, lines 6-10). Such multiple administration is not always possible or practical. Additionally, WO 2004/062677 describes use of modified phages to degrade polysaccharides in the biofilm once it has formed. There is no discussion, teaching or suggestion of uses of bacteriophages which prevent the formation or maintenance of the biofilm. Moreover, bacterial infections can persist and propagate if surrounded by a biofilm, and use of bacteriophage to effectively reduce bacterial infections can be limited by the requirement for the bacteriophage to find and infect bacteria before it can destroy the surrounding biofilm, providing a formidable obstacle when the bacterial concentration in the biofilm is low or when most of the bacteria have been destroyed and some bacterial isolates are still protected by a large mass of biofilm.

The Eastern European research and clinical trials, particularly in treating human diseases, such as intestinal infections, have apparently concentrated on use of naturally occurring phages and their combined uses (Lorch, A. (1999), “Bacteriophages: An alternative to antibiotics?” Biotechnology and Development Monitor, No. 39, p. 14-17). Bacteriophage have also been used in the past for treatment of plant diseases, such as fireblight as described in U.S. Pat. No. 4,678,750. Non-engineered bacteriophages have been used as carriers to deliver antibiotics (such as chloroamphenicol) (Yacoby et al., Antimicrobial agents and chemotherapy, 2006; 50; 2087-2097), which suggest attaching aminoglycosides antibiotics, such as chloroamphenicol, to the outside of filamentous non-engineered bacteriophage (Yacoby et al., Antimicrobial agents and chemotherapy, 2007; 51; 2156-2163). Bacteriophages have also been engineered to express lethal cell death genes Gef and ChpBK (Westwater et al., 2003, Antimicrobial agents and chemotherapy, 47; 1301-1307).

There are amyloids found in humans, yeast, and bacteria. Curli protein in E. coli constitute amyloids (Chapman, et al. Role of Escherichia coli curli operons in directing amyloid fiber formation. Science 295, 851-855, (2002). There is a lack of effective treatments for diseases which involve amyloidosis. Small-molecule inhibition of amyloids is hard to achieve since protein-protein interfaces need to be disrupted (Arkin et al., Small-molecule inhibitors of protein-protein interactions: progressing towards the dream. Nat Rev Drug Discov 3, 301-317 (2004)). Peptide-based inhibitors of amyloids are difficult to deliver to sites of disease or to bacterially infected surfaces which are difficult to access by conventional routes of administration.

Therefore, there is a need for improved compositions and methods to prevent the formation and maintenance of bacterial biofilms.

### SUMMARY OF THE INVENTION

The present invention relates in part to compositions and methods to inhibit or disrupt the formation of, or maintenance of protein aggregates. One aspect of the present invention is directed to engineered bacteriophages expressing at least one anti-amyloid peptide which inhibits or disrupts the formation or maintenance of protein aggregates, in particular high order aggregates which comprise at least two different polypeptides. In some embodiments, the anti-amyloid peptide which inhibits or disrupts the formation of, or maintenance of protein aggregates is expressed on the surface of the bacteriophage, and in some embodiments the anti-amyloid peptide is released from the bacteria infected with the bacteriophage, for example by secretion or release at the time of bacterial lysis.

Accordingly, one aspect of the present invention relates to the engineered bacteriophages as discussed herein which express an anti-amyloid peptide which inhibit or disrupt the formation or maintenance of protein aggregates. In one embodiment, an engineered bacteriophage which expresses an anti-amyloid peptide is termed an “anti-amyloid peptide engineered bacteriophage” or simply as an “engineered bacteriophage” herein and inhibits the formation of protein aggregates which comprise of two or more different polypeptides, e.g., “higher order aggregates” which are protein aggregates formed by a first polypeptide which acts as a seed for the formation of an aggregate comprising at least in part, a second polypeptide.

In alternative embodiments and one aspect of the present invention relates to the engineered bacteriophages as discussed herein which express an amyloid peptide which promotes the formation or maintenance of protein aggregates. In one embodiment, an engineered bacteriophage which expresses an amyloid which promotes the formation of protein aggregrated is termed an “amyloid peptide engineered bacteriophage” or “pro-amyloid peptide engineered bacteriophage” and promotes or increases the formation of protein aggregates which comprise of two or more different polypeptides, e.g., “higher order aggregates” which are protein aggregates formed by a first polypeptide which acts as a seed for the formation of an aggregate comprising at least in part, a second polypeptide. In some embodiments, a pro-amyloid peptide engineered bacteriophage can be used to promote or increase bacteria and/or promote the formation of a bacterial biofilms in environmental, industrial, and clinical settings by administering a composition comprising at least one pro-amyloid engineered bacteriophage as discussed herein. Pro-amyloid peptides are useful in circimstsances where it is desirable to encourage biofilm formation, such as for example but not limited to, establishing microbial biofilms for remediation, microbial fuel cells, “beneficial” biofilms that block “harmful” biofilms from forming on important surfaces, etc).

In some embodiments, an anti-amyloid peptide expressed by an anti-amyloid peptide engineered bacteriophage as disclosed herein is a peptide derived from a first amyloidogenic polypeptide or a second amyloidogenic polypeptide which makes up a high order aggregate. In some embodiments, an anti-amyloid peptide expressed by an anti-amyloid peptide engineered bacteriophage as disclosed herein is a CsgA or a CsgB peptide. In some embodiments, an anti-amyloid peptide engineered bacteriophage can be used to inhibit bacteria and/or removing bacterial biofilms in environmental, industrial, and clinical settings by administering a composition comprising at least one engineered bacteriophage as discussed herein.

One advantage of the anti-amyloid peptide engineered bacteriophage as disclosed herein is to prevent the self-aggregation of anti-amyloid peptides. For example, one of the major problems associated with use of anti-amyloid peptides for therapeutic or other purposes (e.g. anti-amyloid peptides administered by themselves or in a pharmaceutical composition) is their tendency to self-aggregate. Thus, the inventors have demonstrated that by placing the anti-amyloid peptides on the surface of a bacteriophage capsid, it provides a structure for anti-amyloid peptide spacing and prevents aggregation of the anti-amyloid peptides, as well as provides a convenient way to synthesize a lot of anti-amyloid peptides and deliver them to inhibit amyloid formation or inhibit amyloid maintenance.

The inventors also demonstrated that an anti-amyloid peptide engineered bacteriophage as disclosed herein can reduce the number of bacteria in a population of bacteria. Accordingly, the inventors have developed a modular design strategy in which bacteriophages are engineered to have enhanced ability to inhibit and kill bacteria which produce biofilms by expressing an anti-amyloid peptide which blocks amyloid formation or inhibits or disrupts the formation or maintenance of protein aggregates, such as curli amyloid present in bacterial biofilms.

In some embodiments, a bacteriophage can be engineered or modified to express at least one anti-amyloid peptide. In some embodiments, an anti-amyloid peptide engineered bacteriophage can be further modified to also express a biofilm degrading enzyme, such as dispersin B (DspB), an enzyme that hydrolyzes β-1,6-N-acetyl-D-glucosamine, according to the methods as disclosed in U.S. patent application Ser. Nos. 12/337,677 and 11/662,551 and International Application WO06/137847 which are incorporated herein in their entirety by reference.

Also discussed herein is the generation of a diverse library of anti-amyloid peptide engineered bacteriophages described herein, such as a library of anti-amyloid peptide engineered bacteriophages which are capable of inhibiting the formation or maintenance of amyloid formation, for example, for reducing biofilm produced by a wide variety of bacterial strains.

Bacteriophages (often known simply as “phages”) are viruses that grow within bacteria. The name translates as “eaters of bacteria” and reflects the fact that as they grow, the majority of bacteriophages kill the bacterial host in order to release the next generation of bacteriophages. Accordingly, the replication of anti-amyloid peptide engineered bacteriophages with subsequent bacterial lysis and expression of an anti-amyloid peptide renders this a two-pronged attack strategy for inhibiting amyloid formation in bacterial biofilm, as well as killing bacteria and eliminating bacterial populations, and/or removing bacterial biofilms in environmental, industrial, and clinical settings.

Bacteriophages and their therapeutic uses have been the subject of much interest since they were first recognized early in the 20th century. Lytic bacteriophages are viruses that infect bacteria exclusively, replicate, disrupt bacterial metabolism and destroy the cell upon release of phage progeny in a process known as lysis. These bacteriophages have very effective antibacterial activity and in theory have several advantages over antibiotics. Most notably they replicate at the site of infection and are therefore available in abundance where they are most required; no serious or irreversible side effects of phage therapy have yet been described and selecting alternative phages against resistant bacteria is a relatively rapid process that can be carried out in days or weeks.

Bacteriophage have been used in the past for treatment of plant diseases, such as fireblight as described in U.S. Pat. No. 4,678,750. Also, bacteriophages have been previously used to destroy biofilms (e.g., U.S. Pat. No. 6,699,701). In addition, systems using natural bacteriophages that encode biofilm destroying enzymes in general have been described. Examples of lytic enzymes encoded by bacteriophages that have been used as enzyme dispersion to destroy bacteria have been reported (U.S. Pat. No. 6,335,012 and U.S. Patent Application Publication No. 2005/0004030 which is incorporated herein by reference). The Eastern European research and clinical trials, particularly in treating human diseases, such as intestinal infections, has apparently concentrated on use of naturally occurring phages and their combined uses (Lorch, A. (1999), “Bacteriophages: An alternative to antibiotics?” Biotechnology and Development Monitor, No. 39, p. 14-17).

For example, PCT Publication No. WO 2004/062677 and U.S. patent application Ser. No. 10/541,716 provides a method of treating bacterial biofilm, wherein the method comprises use of a first bacteriophage that is capable of infecting a bacterium within said biofilm, and a first polysaccharide lyase enzyme that is capable of degrading a polysaccharide within said biofilm. However, other studies have reported that addition of alginate lyase to established P. aeruginosa biofilm caused no observable detachment of biofilm and the use of lyases would not be optimal for biofilm treatment (Christensen et al., 2001). International Patent Application WO/2006/137847, which are incorporated herein by reference, describes a bacteriophage that expresses a biofilm degrading enzyme attached to its surface.

However, one of the key problem associated with the use of bacteriophages as potential therapeutics are their inability to access bacteria protected by the biofilm barrier. Accordingly, one aspect of the present invention overcomes this problem by providing an anti-amyloid peptide engineered bacteriophage that encodes an anti-amyloid peptide or portion thereof that is displayed on the surface of the phage. Consequently, in such embodiments, the anti-amyloid peptide engineered bacteriophage has an active anti-amyloid peptide on its surface that will inhibit the formation or maintenance of the biofilm by inhibiting curli amyloid formation. Thereafter, when the anti-amyloid peptide engineered bacteriophage encounters a bacterial cell, the phage will replicate. After the phage enters the cell for replication in addition to the normal phage components that are needed for replication in the cell, there will also be the anti-amyloid peptide and a moiety, typically a capsid protein or a capsid attaching part of such capsid protein, fused to the anti-amyloid peptide for attaching the anti-amyloid peptide to the phage surface. Thus after the multiplication and lysis of the cell by the phage a new generation of these anti-amyloid peptide engineered bacteriophage are produced. These in turn will inhibit biofilm maintenance and/or formation or maintenance and can replicate in subsequent bacterial cells thus creating a continuous system for inhibition of biofilm formation and maintenance. Each new generation of anti-amyloid peptide engineered bacteriophage carries the anti-amyloid peptide allowing the anti-amyloid peptide engineered bacteriophage to attack the biofilm from outside, by the inhibition of curli amyloid formation and maintenance, and lyse the bacteria from inside, by the action of anti-amyloid peptide engineered bacteriophage infecting the bacterium, multiplification of the phage, and consequent cell lysis.

In some embodiments, a moiety can be used to direct and attach the anti-amyloid peptide to the surface of an anti-amyloid peptide engineered bacteriophage according to the present invention include, for example, moieties that are commonly used in the phage display techniques well known to one skilled in the art. For example, the anti-amyloid peptide can be part of the other part of a fusion protein, wherein the other part of the fusion protein is part of the surface of the phage such as the capsid, for example, a 10B capsid protein. For example, the 10B capsid protein makes up about 10% of the capsid protein of T7 phage. Proteases can be displayed on the surface of the phage as described by Atwell S and Wells J A (Selection for improved subtiligases by phage display. Proc Natl Acad Sci USA. 1999.96(17):9497-502). Atwell and Wells describe a system where about 16-17 amino acids of active sites of the protease were displayed on the phage and showed protease activity. Accordingly, one useful amino acids sequence is signal peptide-XXX-SEGGGSEGGG-XX (SEQ ID NO: 219) (X is optional, or any amino acid). Another example of useful moieties is a xylan binding domain of xylanase (Miyakubo H, Sugio A, Kubo T, Nakai R, Wakabayashi K, Nakamura S. Phage display of xylan-binding module of xylanase J from alkaliphilic Bacillus sp. strain 41M-1. Nucleic Acids Symp Ser. 2000. (44):165-6). In Miyakubo et al., the moiety displayed on the phage was not the active site of the enzyme but the substrate binding site of the enzyme, which also retained its capacity to bind the substrate. Accordingly, one aspect of the present invention provides an anti-amyloid peptide engineered bacteriophage which can continuously inhibit the formation and/or maintenance of a bacterial biofilm and uses thereof for inhibiting the formation or maintenance of a biofilm.

In addition to displaying at least one anti-amyloid peptide on the surface of an anti-amyloid peptide engineered bacteriophage, the phage may also encode an anti-amyloid peptide that is not displayed on the surface.

One aspect of the present invention describes an anti-amyloid peptide engineered bacteriophage for inhibiting the formation of a biofilm or inhibiting the maintenance of a biofilm, wherein essentially one dosage or round of infection by the anti-amyloid peptide engineered bacteriophage is sufficient to allow complete inhibition of biofilm formation, because the infected bacteria will produce anti-amyloid peptide engineered bacteriophages that contain the anti-amyloid peptides either on their surface or are expressed and released (by lysis or secretion) from the bacteria. This allows replenishment of the anti-amyloid peptide engineered bacteriophage so they can continue to inhibit biofilm formation even in the absence of immediately infectable bacteria in the environment. This solves a requirement for persistent re-application which can be a problem by previously described phage systems. For example, even when the phage of WO 2004/062677 is delivered with an enzyme mixture or with an enzyme associated on the surface of the first phage dose, the method requires that after the initial administration, the phage released from the destroyed bacteria must find and infect at least one additional bacterium to enable it to continue to degrade the biofilm. Therefore, WO 2004/062677 specifically discusses the need for using multiple dosages of phage administration to enhance the results.

Another aspect of the present invention relates to the development of a diverse library of anti-amyloid peptide engineered bacteriophage. By multiplying within the bacterial population and hijacking the bacterial machinery, use of an anti-amyloid peptide engineered bacteriophage achieves high local concentrations of both the lytic phage and the anti-amyloid peptide in the zone of the bacterial population, even with small initial phage inoculations. Thus, the present invention is suitable for delivery of the anti-amyloid engineered bacteriophage at bacterial infection where are difficult to reach or get access to.

The inventors have demonstrated that an anti-amyloid peptide engineered bacteriophage as disclosed herein is faster and has increased efficiency of killing bacteria, such as bacteria in biofilms as compared to use of a non-engineered bacteriophage alone (i.e. a bacteriophage which is not an engineered bacteriophage) (See FIG. 3). Thus, the inventors have demonstrated a significant and surprising improvement of such an anti-amyloid peptide engineered bacteriophage as disclosed herein over the combined use of non-engineered bacteriophages as therapies described in prior art. Specifically, the inventors have also demonstrated that use of such an anti-amyloid peptide engineered bacteriophage as disclosed herein is very effective at reducing the number of antibiotic resistant bacterial cells which can develop in the presence of sub-inhibitory antimicrobial drug concentrations.

One aspect of the present invention relates to engineering or modification of any bacteriophage strain or species to generate an anti-amyloid peptide engineered bacteriophage disclosed herein. For example, an anti-amyloid peptide engineered bacteriophage can be engineered from any bacteriophage known by a skilled artisan. For example, in one embodiment, the bacteriophage is a lysogenic bacteriophage, for example but not limited to a M13 bacteriophage. In another embodiment, the bacteriophage is a lytic bacteriophage such as, but not limited to T7 bacteriophage. In another embodiment, the bacteriophage is a phage K or a Staphyloccocus phage K for use against bacterial infections of methicillin-resistant S. aureus.

One aspect of the present invention relates to an anti-amyloid peptide engineered bacteriophage which is an anti-amyloid peptide engineered lysogenic M13 bacteriophage comprising a nucleic acid operatively linked to a promoter, such as a M13 promoter, wherein the nucleic acid encodes at least one anti-amyloid peptide, such as a CsgA or a CsgB anti-amyloid peptide, where a CsgA peptide is selected from SEQ ID NOs: 11-18 or SEQ ID NOs: 35-58 or variants or modified variants thereof, and a CsgB peptide is selected from SEQ ID NOs: 27-34 or SEQ ID NOs: 59-90, or variants or modified variants thereof. In some embodiments, the CsgA peptide is a Class III CsgA peptide, e.g., selected from SEQ ID NO: 52 or 53, and the CsgB peptide is a Class III Csg III peptide, e.g., selected from SEQ ID NO: 61-65.

In some embodiments, an anti-amyloid peptide engineered bacteriophage as disclosed herein is an anti-amyloid peptide engineered lysogenic M13 bacteriophage comprising a nucleic acid operatively linked to a promoter, such as a M13 promoter, wherein the nucleic acid encodes at least one anti-amyloid peptide such as an anti-amyloid peptide, selected from the group of SEQ ID NOs: 11-18 or 27 to 90, or variants or modified variants thereof.

In some embodiments, an anti-amyloid peptide engineered bacteriophage as disclosed herein is an anti-amyloid peptide engineered lysogenic M13 bacteriophage comprising a nucleic acid operatively linked to a promoter, such as a M13 promoter, wherein the nucleic acid encodes at least one anti-amyloid peptide selected from the group of SEQ ID NOs: 12, 16, 29 and 33. In some embodiments, an anti-amyloid peptide engineered bacteriophage is an engineered lysogenic M13 bacteriophage comprising a nucleic acid operatively linked to a promoter, such as a M13 promoter, wherein the nucleic acid encodes at least one anti-amyloid peptide selected from the CsgA III of peptides (SEQ ID NO: 52-53), or from the CsgAIIb peptide class (SEQ ID NOs:35, 36, 39-41, 45, 49-51), or from the CsgAIIa peptide group (SEQ ID NO: 11 and 12) or from the CsgAI group (SEQ ID NOs: 42, 44, 46, 57 and 58).

In another embodiment, an anti-amyloid peptide engineered bacteriophage as disclosed herein is an engineered lysogenic M13 bacteriophage comprising a nucleic acid operatively linked to a promoter, such as a M13 promoter, wherein the nucleic acid encodes at least one antimicrobial agent such as an anti-amyloid peptide, selected from the CsgBIII group (SEQ ID NOs: 61-65) or from the CsgBIIb peptide group (SEQ ID NOs: 59, 60, 69, 75, 81, 93 and 94) or from the CsgBIIa group (SEQ ID NO: 29) or from CsgBI peptide group (SEQ ID NOs: 66-68 and 70-72).

In a preferred embodiment, the anti-amyloid peptide engineered bacteriophage is an engineered lysogenic M13 bacteriophage comprising a nucleic acid operatively linked to a promoter, such as a M13 promoter, wherein the nucleic acid encodes at least one anti-amyloid peptide, selected from the CsgAIII group of peptides (SEQ ID NO: 52, 53) or CsgBIII peptides (SEQ ID NOs: 61-65).

Another aspect of the present invention relates to an anti-amyloid peptide engineered bacteriophage which is an anti-amyloid peptide engineered lytic T7 bacteriophage comprising a nucleic acid operatively linked to a promoter, such as a T7 promoter, wherein the nucleic acid encodes at least one anti-amyloid peptide, such as a CsgA or a CsgB anti-amyloid peptide, where a CsgA peptide is selected from SEQ ID NOs: 11-18 or SEQ ID NOs: SEQ ID NOs: 35-58 or variants or modified variants thereof, and a CsgB peptide is selected from SEQ ID NOs: 27-34 or SEQ ID NOs: 59-90, or variants or modified variants thereof.

In some embodiments, an anti-amyloid peptide engineered bacteriophage as disclosed herein is an anti-amyloid peptide engineered lytic T7 bacteriophage comprising a nucleic acid operatively linked to a promoter, such as a promoter, such a T7 promoter, wherein the nucleic acid encodes at least one anti-amyloid peptide such as an anti-amyloid peptide, selected from the group of SEQ ID NOs: 35-90, or variants or modified variants thereof.

In some embodiments, an anti-amyloid peptide engineered bacteriophage as disclosed herein is an anti-amyloid peptide engineered lytic T7 bacteriophage comprising a nucleic acid operatively linked to a promoter, such as a T7 promoter, wherein the nucleic acid encodes at least one anti-amyloid peptide selected from the group of SEQ ID NOs: 12, 16, 29 and 33. In some embodiments, an anti-amyloid peptide engineered bacteriophage is an engineered lytic T7 bacteriophage comprising a nucleic acid operatively linked to a promoter, such as a T7 promoter, wherein the nucleic acid encodes at least one anti-amyloid peptide selected from the CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58).
In another embodiment, an anti-amyloid peptide engineered bacteriophage as disclosed herein is an engineered lytic T7 bacteriophage comprising a nucleic acid operatively linked to a promoter, such as a T7 promoter, wherein the nucleic acid encodes at least one antimicrobial agent such as an anti-amyloid peptide, selected from the CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72).

In a preferred embodiment, the anti-amyloid peptide engineered bacteriophage is an engineered lytic T7 bacteriophage comprising a nucleic acid operatively linked to a promoter, such as a T7 promoter, wherein the nucleic acid encodes at least one anti-amyloid peptide, selected from the CsgAIII group of peptides (SEQ ID NO: 52-53) or CsgBIII peptides (SEQ ID NOs: 61-65).

In some embodiments of the invention, an anti-amyloid engineered bacteriophage is administered in combination with an additional antimicrobial agent, thus allowing a reduction in the amount of such additional antimicrobial agent as compared to if the antimicrobial agent were used separately (i.e. a decrease in dose of antimicrobial agent required to effectively treat a subject suffering from an infection). For example, in some embodiments, administering an anti-amyloid peptide engineered bacteriophage in combination with an additional antimicrobial agent allows a reduction in the dose of either the antimicrobial agent or both, or a reduction in the duration or frequency of treatment. In some embodiments, a reduction is about at least 10%, or about at least 20%, or about at least 30%, or about at least 40%, or about at least 50% or more than 50% of the dose of antimicrobial agent as compared to the dose of an antimicrobial agent without the presence of an anti-amyloid peptide engineered bacteriophage.

Another aspect of the present invention relates to a method to inhibit or eliminate a bacterial infection comprising administering to a surface infected with bacteria an anti-amyloid peptide engineered bacteriophage comprising a nucleic acid operatively linked to a bacteriophage promoter, wherein the nucleic acid encodes at least one anti-amyloid peptide, such as a CsgA peptide and/or a CsgB peptide, including but not limited to SEQ ID NO:11-18 or 35-58 (i.e. CsgA peptides) SEQ ID NOs: 27-34 or 59-90 (i.e. CsgB peptides) and SEQ ID NOs: 53-90 (modified CsgA and CsgB peptides). In some embodiments, the present invention relates to a method to inhibit or eliminate a bacterial infection comprising administering to a surface infected with bacteria an anti-amyloid peptide engineered bacteriophage comprising a nucleic acid operatively linked to a bacteriophage promoter, wherein the nucleic acid encodes at least one anti-amyloid peptide, such as one selected from the CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58) or from the CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72).

In some embodiments, the method can also optimally include administering at least one additional agent, such as an additional antimicrobial agent or other agent which inhibits fiber assembly.

In some embodiments of all aspects described herein, a bacteriophage useful in the methods disclosed herein and used to generate an anti-amyloid peptide engineered bacteriophage is any bacteriophage know by a skilled artisan. A non-limiting list of examples of bacteriophages which can be used are disclosed in Table 9 herein. In one embodiment, the bacteriophage is a lysogenic bacteriophage such as, for example a M13 lysogenic bacteriophage. In alternative embodiments, a bacteriophage useful in all aspects disclosed herein is a lytic bacteriophage, for example but not limited to a T7 lytic bacteriophage. In one embodiment, a bacteriophage useful in all aspects disclosed herein is a SP6 bacteriophage or a phage K, or a staphylococcus phage K bacteriophage.

In some embodiments, administration of any anti-amyloid peptide engineered bacteriophage as disclosed herein can occur substantially simultaneously with any additional agent, such as an additional antimicrobial agent or another agent which inhibits fiber assembly. In alternative embodiments, the administration of an anti-amyloid peptide engineered bacteriophage can occur prior to the administration of at least one additional antimicrobial agent and/or agent which inhibits fiber assembly. In other embodiments, the administration of an additional antimicrobial agent or agent which inhibits fiber assembly occurs prior to the administration of an anti-amyloid peptide engineered bacteriophage.

In some embodiments, additional antimicrobial agents which can be administered in combination with an anti-amyloid peptide engineered bacteriophage as disclosed herein include, for example but not limited to, antimicrobial agents selected from a group comprising ciproflaxacin, levofloxacin, and ofloxacin, gatifloxacin, norfloxacin, lomefloxacin, trovafloxacin, moxifloxacin, sparfloxacin, gemifloxacin, pazufloxacin or variants or analogues thereof. In some embodiments, an antimicrobial agents useful in the methods as disclosed herein is ofloxacin or variants or analogues thereof. In some embodiments, antimicrobial agents useful in the methods as disclosed herein are aminoglycoside antimicrobial agents, for example but not limited to, antimicrobial agents selected from a group consisting of amikacin, gentamycin, tobramycin, netromycin, streptomycin, kanamycin, paromomycin, neomycin or variants or analogues thereof. In some embodiments, an antimicrobial agent useful in the methods as disclosed herein is gentamicin or variants or analogues thereof. In some embodiments, antimicrobial agents useful in the methods as disclosed herein are β-lactam antibiotic antimicrobial agents, such as for example but not limited to, antimicrobial agents selected from a group consisting of penicillin, ampicillin, penicillin derivatives, cephalosporins, monobactams, carbapenems, β-lactamase inhibitors or variants or analogues thereof. In some embodiments, an antimicrobial agent useful in the methods as disclosed herein is ampicillin or variants or analogues thereof.

Another aspect of the present invention relates to a composition comprising a lysogenic M13 anti-amyloid peptide engineered bacteriophage comprising a nucleic acid operatively linked to a M13 promoter, wherein the nucleic acid encodes at least one anti-amyloid peptide, for example selected from SEQ ID NO: 11-18 (CsgA peptides, see Table 3), or SEQ ID NO: 27-34 (CsgB peptides, see Table 4) or SEQ ID NO: 35-90 (modified CsgA or CsgB peptides, see Table 5). In some embodiments, the present invention provides a composition comprising at least one lysogenic M13 anti-amyloid peptide engineered bacteriophage comprising a nucleic acid operatively linked to a M13 promoter, wherein the nucleic acid encodes at least one anti-amyloid peptide, for example selected from the CsgAIII group of peptides (SEQ ID NO: 52, 53), or from the CsgBIII group of peptides (SEQ ID NOs: 61-65). In some embodiments, the anti-amyloid peptide expressed by the lysogenic M13 anti-amyloid peptide engineered bacteriophage is selected from at least one of the following from the group of: CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58) or from the CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72).

Another aspect of the present invention relates to a composition comprising a lytic T7 anti-amyloid peptide engineered bacteriophage comprising a nucleic acid operatively linked to a T7 promoter, wherein the nucleic acid encodes at least one anti-amyloid peptide, for example selected from SEQ ID NO: 11-18 (CsgA peptides, see Table 3), or SEQ ID NO:27-34 (CsgB peptides, see Table 4) or SEQ ID NO: 35-90 (modified CsgA or CsgB peptides, see Table 5). In some embodiments, the present invention provides a composition comprising at least one lytic T7 anti-amyloid peptide engineered bacteriophage comprising a nucleic acid operatively linked to a promoter, such as a T7 promoter, wherein the nucleic acid encodes at least one anti-amyloid peptide, for example selected from the CsgAIII group of peptides (SEQ ID NO: 52, 53), or from the CsgBIII group of peptides (SEQ ID NOs: 61-65). In some embodiments, the anti-amyloid peptide expressed by the lysogenic M13 anti-amyloid peptide engineered bacteriophage is selected from at least one of the following from the group of: CsgAIIb peptide group (SEQ ID NOs: 35, 36, 39-41, 45, 49-51), or from the CsgAIIa peptide group (SEQ ID NO: 11 and 12) or from the CsgAI group (SEQ ID NOs: 42, 44, 46, 57 and 58) or from the CsgBIIb peptide group (SEQ ID NOs: 59, 60, 69, 75, 81, 93 and 94) or from the CsgBIIa group (SEQ ID NO: 29) or from CsgBI peptide group (SEQ ID NOs: 66-68 and 70-72).

In some embodiments, a composition comprising an anti-amyloid peptide engineered bacteriophage can further comprise an additional agent, such as for example an antimicrobial agent or an agent which inhibits fiber aggregation such as, for example but not limited to, quinolone antimicrobial agents and/or aminoglycoside antimicrobial agents and/or β-lactam antimicrobial agent, for example, but not limited to, antimicrobial agents selected from a group comprising ciproflaxacin, levofloxacin, and ofloxacin, gatifloxacin, norfloxacin, lomefloxacin, trovafloxacin, moxifloxacin, sparfloxacin, gemifloxacin, pazufloxacin, amikacin, gentamycin, tobramycin, netromycin, streptomycin, kanamycin, paromomycin, neomycin, penicillin, ampicillin, penicillin derivatives, cephalosporins, monobactams, carbapenems, β-lactamase inhibitors or variants or analogues thereof.

In some embodiments, the composition comprises at least one anti-amyloid peptide engineered bacteriophage as disclosed herein.

Another aspect of the present invention relates to a kit comprising a lysogenic M13 anti-amyloid peptide engineered bacteriophage comprising the nucleic acid operatively linked to a promoter, such as a M13 promoter, wherein the nucleic acid encodes at least one anti-amyloid peptide, for example selected from SEQ ID NO: 11-18 (CsgA peptides, see Table 3), or SEQ ID NO:27-34 (CsgB peptides, see Table 4) or SEQ ID NO: 53-90 (modified CsgA or CsgB peptides, see Table 5).

Another aspect of the present invention relates to a kit comprising a lytic T7 anti-amyloid peptide engineered bacteriophage comprising the nucleic acid operatively linked to a T7 promoter, wherein the nucleic acid encodes at least one at least one anti-amyloid peptide, for example selected from SEQ ID NO: 11-18 (CsgA peptides, see Table 3), or SEQ ID NO:27-34 (CsgB peptides, see Table 4) or SEQ ID NO: 35-90 (modified CsgA or CsgB peptides, see Table 5).

Another aspect the invention provides compositions and methods for identifying anti-amyloid peptides that inhibit amyloid formation or maintenance. In such an embodiment, an anti-amyloid peptide can be identified using a computational method as described in Example 4. The computational method comprises predicting amyloid fiber structures, and constructing point mutations to identify potential residues (“hits”) essential to enhancing or inhibiting fiber formation. The “hits” can then be confirmed by mutation experiments as described in Example 4. In another embodiment, phage can be engineered to express a candidate anti-amyloid peptide. In some embodiments the candidate anti-amyloid peptide is derived from an amyloidogenic polypeptide, as disclosed herein. In some embodiments the candidate anti-amyloid peptide is a modified version of a peptide derived from an amyloidogenic polypeptide. In some embodiments the candidate anti-amyloid peptide has a random sequence. In some embodiments, a collection or plurality of engineered phage that collectively express a plurality of candidate anti-amyloid peptides (e.g., peptides derived from an amyloidogenic polypeptide, modified versions thereof, or random sequences, are provided). The collection could comprise, e.g., between about 10 and about 10⁸ or more different candidate anti-amyloid peptide sequences in various embodiments. The ability of the anti-amyloid peptide engineered phage expressing a candidate anti-amyloid peptide to inhibit amyloid formation or maintenance in vitro or in vivo is assessed, using, for example any method known to one of ordinary skill in the art or as disclosed herein in the Examples. An anti-amyloid peptide engineered phage expressing a candidate anti-amyloid peptide which significantly inhibits amyloid formation or maintenance can be assessed using the assay as described herein and those which inhibit bacterial infection and/or amyloid formation can be identified and selected. In some embodiments, the identified phage can be selected to be used as an anti-amyloid agent as disclosed herein. In some embodiments, the selected candidate anti-amyloid peptide encoded by such phage are used as anti-amyloid agents. For example, the present invention encompasses use of an anti-amyloid peptide engineered phage expressing a candidate anti-amyloid peptide to identify anti-amyloid peptides that inhibit formation of amyloids involved in disease such as Alzheimer's disease or other amyloid-associated diseases. Such anti-amyloid peptides can be selected and administered as a pharmaceutical composition for treatment and/or prophylaxis of the disease.

In some embodiments, any one of these anti-amyloid peptide engineered bacteriophages, used alone, or can be used in any combination. In some embodiments, an anti-amyloid peptide engineered bacteriophage as disclosed herein can also be used with at least one additional antimicrobial agent or an agent which inhibits amyloid aggregation.

In some embodiments, the methods and compositions as disclosed herein are administered to a subject. In some embodiments, the methods to inhibit or eliminate a bacterial infection comprising administering a composition comprising an anti-amyloid peptide engineered bacteriophage as disclosed herein to a subject, wherein the bacteria are present in the subject. In some embodiments, the subject is a mammal, for example, but not limited to a human. In some embodiments, the anti-amyloid peptide engineered bacteriophage inhibits bacterial infection by at least about 10%, or at least about 20%, or at least about 30%, or least about 40%, or at least about 50%, or at least about 60%, 70%, 80%, 90%, 95%, 99%, or greater than 99%, such as 100%.

In some embodiments, an anti-amyloid peptide engineered bacteriophage as disclosed herein can be used to reduce the number of bacteria as compared to use of a non-engineered bacteriophage. In some embodiments, an anti-amyloid peptide engineered bacteriophage as disclosed herein is useful in any combination to inhibit or eliminate a bacterial infection, such as for example inhibit or eliminate a bacteria present a biofilm.

Additionally, there are reports of modifying bacteriophages to increase their effectiveness of killing bacteria have also mainly focused on optimizing method to degrade bacteria biofilms, such as, for example introducing a lysase enzyme such as alginate lyse (discussed in International Application WO04/062677); or modifying bacteriophages to inhibit the cell which propagates the bacteriophage, such introducing a KIL gene such as the Holin gene in the bacteriophage (discussed in International Application WO02/034892 and WO04/046319), or introducing bacterial toxin genes such as pGef or ChpBK and Toxin A (discussed in U.S. Pat. No. 6,759,229 and Westwater et al., Antimicrobial agents and Chemotherapy, 2003., 47: 1301-1307). However, unlike the present invention the modified bacteriophages discussed in WO04/062677, WO02/034892, WO04/046319, U.S. Pat. No. 6,759,229 and Westwater et al., have not been modified to express anti-amyloid peptides to inhibit or disrupt the formation or maintenance of protein aggregates in the biofilms, nor to inhibit the formation or maintenance of higher order aggregates, (where high order aggregates comprises of two or more different polypeptides which are formed by a first polypeptide which seeds the formation of an aggregate comprising at least in part of a second polypeptide).

In some embodiments, a non-engineered bacteriophage can be used to block amyloid formation.

One aspect of the present invention relates to an engineered bacteriophage comprising a nucleic acid operatively linked to a promoter, wherein the nucleic acid encodes at least one anti-amyloid peptide.

In some embodiments, the anti-amyloid peptide is a peptide between at least 5 and 50 amino acid long whose sequence comprises at least 5 and no more than 50 contiguous amino acids of the sequence of a first amyloidogenic polypeptide which is capable of nucleating amyloid formation by a second amyloidogenic polypeptide. In some embodiments, the anti-amyloid peptide is a peptide between at least 5 and 50 amino acid long whose sequence comprises at least 5 and no more than 50 contiguous amino acids of the sequence of a second amyloidogenic polypeptide, wherein a second amyloidogenic polypeptide forms an amyloid formation with a first amyloidogenic polypeptide. In some embodiments, the anti-amyloid peptide is a peptide between least 8 and no more than 30 contiguous amino acids of the sequence of a first amyloidogenic polypeptide. In some embodiments, the anti-amyloid peptide is a peptide between least 8 and no more than 30 contiguous amino acids of the sequence of a second amyloidogenic polypeptide. In some embodiments, the first and second amyloidogenic polypeptides are no more than 50% identical.

In some embodiments, at least one of the amyloidogenic polypeptide is a component of a naturally occurring amyloid or a component of a high order aggregate comprising at least two different polypeptides.

In some embodiments, at least one of the amyloidogenic polypeptides is a component of a biofilm generated by a bacterium, for example a human or animal pathogenic bacteria. In some embodiments, the bacterium is a gram-negative bacterium, such as a gram-negative rod. In some embodiments, the bacterium is an enterobacterium, or alternatively, a member of a genus selected from Escherichia, Klebsiella, Salmonella, and Shigella.

In some embodiments, a first amyloidogenic polypeptide is a CsgB polypeptide, and the second amyloidogenic polypeptide is a CsgA polypeptide. In some embodiments, the first and second amyloidogenic polypeptides are a CsgB polypeptide and a CsgA polypeptide, respectively.

In some embodiments, an anti-amyloid peptide expressed by the bacteriophage is between 10 and 30 amino acids in length, or between 15 and 25 amino acids in length.

In some embodiments, the sequence of the anti-amyloid peptide comprises or consists of a sequence selected from SEQ ID NO: 1 or SEQ ID NO: 2 and orthologs thereof.

In some embodiments, the anti-amyloid peptide is CsgA peptide, for example, a CsgA peptide selected from the group comprising: SEQ ID NO; 11-18, CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58) or orthologs thereof. In some embodiments, the CsgA peptide is selected from the group comprising: SEQ ID NOs: 52 or 53) or orthologs thereof.

In some embodiments, the anti-amyloid peptide is a CsgB peptide, for example, a CsgB peptide is selected from the group comprising: SEQ ID NO; 27-34, CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72) or orthologs thereof. In some embodiments, the CsgB peptide is selected from the group comprising: SEQ ID NOs: 61-65 or orthologs thereof.

In some embodiments, the anti-amyloid peptide sequence differs by not more than 3 amino acid insertions, deletions, or substitutions from that of the peptides of SEQ ID NO; 11-18, CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58), or SEQ ID NO; 27-34, CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72).

In some embodiments, an anti-amyloid peptide sequence differs by not more than 4 amino acid insertions, deletions or substutions.

In some embodiments, the N- and C-termini of an anti-amyloid peptide sequence alter by not more than 4 amino acid insertions, deletions or substutions.

In some embodiments, the N- and C-termini of the anti-amyloid peptide sequence can vary in length, for example, between 1 and 10 amino acids in length, or for example, between 3 and 8 amino acids in length. In some embodiments, the N- and C-termini of the anti-amyloid peptide sequence can comprise at least one additional amino acid residue. In particular, the N-terminus of the anti-amyloid peptide sequence can be extended by at least 1, at least 2, or at least 3 or more arginine or other amino acid residues. The C-terminus of the anti-amyloid peptide sequence can be extended by at least 1, at least 2, or at least 3 or more proline residues.

In some embodiments, an anti-amyloid peptide is expressed on the surface of the engineered bacteriophage from which it is expressed. In some embodiments, an anti-amyloid peptide is released from a bacterial host cell infected by the engineered bacteriophage, for example, by lysis of the bacterial cell or alternatively, by secretion by the bacterial host cell. In such embodiments, where the anti-amyloid peptide is secreted from the cell, the nucleic acid encoding the anti-amyloid peptide agent also encodes a signal sequence, such as, for example, a secretory sequence. In some embodiments, the secretory sequence is cleaved from the anti-amyloid peptide or antimicrobial peptide as the peptide exits the bacteria cell.

Another apect of the present invention relates to a method to reduce protein aggregate formation in a subject comprising administering to a subject at least one bacteriophage comprising a nucleic acid operatively linked to a promoter, wherein the nucleic acid encodes at least one anti-amyloid peptide.

In some embodiments, the subject suffers or is at risk of amyloid associated disorder. In some embodiments, the subject suffers from or is at increased risk of an infection by a bacterium, for example, a bacterium is associated with biofilm formation.

In some embodiments of this aspect and all aspects as disclosed herein, the subject is a mammal, such as a human.

In some embodiments, the method to reduce protein aggregate formation in a subject comprising administering to a subject at least one anti-amyloid peptide engineered bacteriophage as disclosed herein further comprises adding an additional agent to the subject.

In some embodiments, the anti-amyloid peptide inhibits the formation of at least one of the amyloidogenic polypeptides that is a component of a naturally occurring amyloid or a component of a high order aggregate comprising at least two different polypeptides. In some embodiments, the high order aggregate comprises a fiber. In some embodiments, the first amyloidogenic polypeptide is a CsgB polypeptide. In some embodiments, the second amyloidogenic polypeptide is a CsgA polypeptide.

In some embodiments, an anti-amyloid peptide expressed by the anti-amyloid peptide engineered bacteriophage varies in length, for example between 10 and 30 amino acids in length, or for example, between 15 and 25 amino acids in length. In some embodiments, an anti-amyloid peptide expressed by the anti-amyloid peptide engineered bacteriophage comprises or consists of a sequence of at least 8 contagious amino acids selected from any in SEQ ID NO: 1 or SEQ ID NO: 2 and orthologs thereof. In some embodiments, an anti-amyloid peptide expressed by the anti-amyloid peptide engineered bacteriophage is a CsgA peptide, such as, for example, selected from the group comprising: SEQ ID NO; 11-18, CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58) or orthologs thereof. In some embodiments, an anti-amyloid peptide expressed by the anti-amyloid peptide engineered bacteriophage is a CsgA peptide is selected from the group comprising: SEQ ID NOs: 52, 53) or orthologs thereof. In some embodiments, an anti-amyloid peptide expressed by the anti-amyloid peptide engineered bacteriophage is a CsgB peptide, for example, selected from the group comprising: SEQ ID NO; 27-34, CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72) or orthologs thereof. In some embodiments, an anti-amyloid peptide expressed by the anti-amyloid peptide engineered bacteriophage is a CsgB peptide selected from the group comprising: SEQ ID NOs: 61-65 or orthologs thereof.

In some embodiments, a plurality of anti-amyloid peptide engineered bacteriophages are administered to a subject, and in some embodiments, each bacteriophage comprises a nucleic acid which encodes one or more different anti-amyloid peptides. In some embodiments, the plurality of bacteriophages express one or more different anti-amyloid peptides from the same amyloidogenic polypeptide or a different amyloidogenic polypeptide. In some embodiments, at least one bacteriophage in a plurality of bacteriophages express one or more anti-amyloid peptides from a first amyloidogenic polypeptide and at least one bacteriophage in a plurality of bacteriophages expresses one or more anti-amyloid peptides from a second amyloidogenic polypeptide, for example, where the first amyloidogenic polypeptide is a CsgA polypeptide and a second amyloidogenic polypeptide is a CsgB polypeptide.

Another aspect of the present invention provides a composition comprising an anti-amyloid peptide engineered bacteriophage as disclosed herein. In some embodiments, the composition further comprises a pharmaceutical acceptable carrier. In some embodiments, the composition further comprises an additional agent, for example, other anti-amyloid peptides or an agent which inhibits fiber aggregation.

Another aspect of the present invention relates to kits comprising an anti-amyloid peptide engineered bacteriophage as disclosed herein, where the anti-amyloid peptide engineered bacteriophage comprises a nucleic acid operatively linked to a promoter, wherein the nucleic acid encodes at least one anti-amyloid peptide.

Another aspect of the present invention relates to the use of any of the anti-amyloid peptide engineered bacteriophages as disclosed herein for reducing the formation or maintenance of protein aggregates. In some embodiments, the anti-amyloid peptide engineered bacteriophages are used to inhibit a naturally forming amyloid or a high order aggregate comprising of at least two different polypeptides. In such embodiments, a naturally forming amyloid comprises a first amyloidogenic polypeptide which is capable of nucleating amyloid formation by a second amyloidogenic polypeptide. In some embodiments, the anti-amyloid peptide engineered bacteriophages are used to inhibit a naturally forming amyloid or a high order aggregate in a subject, for example, an amyloid or protein aggregate produced as part of a bacterial biofilm.

### BRIEF DESCRIPTION OF FIGURES

FIG. 1 shows amyloid formation in the presence of T7 or M13mp18 bacteriophage. Varying pfu/ml were added to wells containing 5 μM of curli or NM. Unmodified T7 exhibited minimal inhibition of curli and Sup35-NM amyloid fiber assembly, while unmodified M13mp18 had moderately strong efficacy against both curli and Sup35-NM fiber formation.

FIG. 2 shows amyloid formation in the presence of engineered T7 bacteriophage which display selected curli-inhibiting peptides on their capsid proteins. Varying pfu/ml were added to wells containing curli. The numbers in the legend refer to anti-amyloid peptide engineered T7 expressing CsgA- or CsgB-peptides listed in Table 3 and Table 4. The most effective engineered bacteriophages were the ones with construct #27 (SEQ ID NO: 29), #18 (SEQ ID NO: 12), #22 (SEQ ID NO: 16), and #31 (SEQ ID NO: 33). Unmodified control T7select-415 bacteriophage are indicated by T7#2 in the legend.

FIG. 3A-3B shows amyloid-inhibiting peptides expressed on phage capsids to suppress in vitro amyloid fiber assembly. FIG. 3A shows that T7 phages expressing wild-type CsgA₄₃₋₅₂, CsgA₅₅₋₆₄, and CsgB₁₃₃₋₁₄₂ (T7-CsgA₄₃₋₅₂, T7-CsgA₅₅₋₆₄, and T7-CsgB₁₃₃₋₁₄₂, bold green lines) stimulated curli fiber assembly at concentrations below 10³ PFU/mL but blocked assembly at concentrations above 10³ PFU/mL with moderate efficacy (Class IIa). Three classes of recombinant phage expressing curli-inhibiting peptides were distinguishable based on minimal (Class I, black lines), moderate (Class IIb, blue lines), and strong inhibition of curli fiber assembly (Class III, red lines) (see Example 4 and Tables 7 and 8). FIG. 3B shows T7 phages expressing wild-type CsgA₅₅₋₆₄ (squares) and CsgB₁₃₃₋₁₄₂ (diamonds) seeded curli fiber assembly at 500 PFU/mL. CsgA seeded assembly is shown for comparison (triangles).

FIG. 4A-4B shows the polypeptide sequence of CsgA (SEQ ID NO:1) (FIG. 4A) and nucleic acid sequence encoding CsgA (SEQ ID NO:200) (FIG. 4B).

FIG. 5A-5B shows the polypeptide sequence of CsgA (SEQ ID NO:2) (FIG. 5A) and nucleic acid sequence encoding CsgB (SEQ ID NO:201) (FIG. 5B).

FIG. 6 shows a histogram of the effectiveness of the anti-amyloid peptide engineered bacteriophages at inhibiting the growth of E. coli biofilms. 1×10⁴ plaque forming units (PFU)/mL of anti-amyloid peptide engineered bacteriophages were used to inhibit biofilm grow for 36 hours. The level of biofilm biomass was determined with crystal violet staining followed by solubilization in acetic acid and measurement of optical density at 600 nm. Anti-amyloid peptide engineered bacteriophage which express peptide sequence #76 (SEQ ID NO: 62) shows much lower biofilm biomass compared with control phage (T7 with a control peptide), T7 wild-type, and no phage treatment. Also shown are anti-amyloid peptide engineered bacteriophage which express peptide sequences #17 (SEQ ID NO: 11), #18 (SEQ ID NO: 12) and #27 (SEQ ID NO: 29). Of note, these anti-amyloid peptide engineered bacteriophages which were tested are non-replicative, (i.e. they do not replicate within in the host bacterial cells) so the experiment indicates the inhibition of the biofilm formation by these peptides sequences; #76 (SEQ ID NO: 62), #17 (SEQ ID NO: 11), #18 (SEQ ID NO: 12) and #27 (SEQ ID NO: 29).

FIGS. 7A-7E shows an assay to identify anti-amyloid peptides which bind to CsgA and CsgB polypeptides (nucleating sequences in CsgA and CsgB). FIG. 7A shows a schematic of CsgA polypeptides or CsgB polypeptides binding to peptides located on a “dot” of an assay, where the dots are coated with individual anti-amyloid peptides or anti-amyloid peptide-engineered bacteriophages. Dots where aggregrates form identify anti-amyloid peptides which bind to CsgA or CsgB (i.e. can be peptides to the binding sites of CsgA or CsgB) and are effective at inhibiting formation of aggregrates, and dots where no aggregrates form identify anti-amyloid peptides which do not specifically bind CsgA or CsgB polypeptides, and are less effective at ihibiting the formation of curli agregrates. FIG. 7B shows hits (identified by the arrow) of high order protein aggregrate, thus identifying anti-amyloid peptide engineered bacteriophages which binds CsgA or CsgB polypeptides and thus is effective at inhibiting protein aggregrate formation. Relative fluorescence of Alexa-labelled full-length CsgA bound to peptide arrays demonstrate that nucleation of CsgA is facilitated by three peptides in CsgB (SEQ ID NOs: 250, 203 and 204) which contain hydrophobic residues (underlined in red). FIG. 7C shows wildtype (wt) bacteriophage has formation of protein aggregrates in the presence of CsgB (left, postive control), no agregrates in the absence of CsgB polypeptide (CsgB−) (middle, negative control) and absence of aggregrates in the presence of bacteriophage CsgBpΔAIVV (right). FIG. 7A-7C is an example of an assay which can be used to identify anti-amyloid peptide which inhibit aggregrate formation as disclosed herein. FIG. 7D shows CsgB binding sequences, SEQ ID NOs: 250, 202. FIG. 7E shows various concentrations of peptides bound to maleimide plates to faciliate in vitro assembly of soluble CsgA into amyloids as monitored by ThT fluorescence. CsgB₁₃₀₋₁₄₉ facilitates CsgA assembly (0.1 μM, 0.25 μM, and 0.5 μM shown) with a process similar to a seeded assembly (i.e., can be fitted with first order kinetics). CsgB₆₂₋₈₁ and CsgA alone show assembly with lag phases even at their highest concentrations (0.5 μM shown).

FIGS. 8A-8C shows a schematic of the alignment of segments of the amino acid sequences other biofilm polypeptides. FIG. 8A shows the amino acid sequences of these biofilm polypeptides are highly conserved and can be used to derive anti-amyloid peptides as disclosed herein. In some embodiments, the anti-amyloid peptides expressed by the anti-amyloid peptide engineered bacteriophages of the present invention can comprise a peptide derived from any one of sequences shown in FIG. 8A (SEQ ID NO: 251-259). FIGS. 8B and 8C show amino acid sequences of additional biofilm polypeptides which can be used to derive anti-amyloid peptides as disclosed herein. The each polypeptide sequence is identified by the GeneBank No followed by a “_” and a portion of name of the polypeptide (SEQ ID NOs: 260-384). Each Genebank sequence is incorporated herein in its entirety by reference.

FIGS. 9A-9B shows a histogram of the effect of the small molecule inhibitors DAPH-12, DAPH-6 and Amphotericin B (AmphB) to prevent formation of curli amyloid fibers. FIG. 9A shows % amyloid fiber formation in the presence of CsgA, and in the presence of increasing ratios (1:20, 1:10) of the inhibitors DAPH-6 or DAPH-12. DAPH-12 secetively inhibits Curli assembly. FIG. 9B shows % amyloid fiber formation in the presence of NM or CsgA, and in the presence of increasing ratios (1:0.5, 1:2, 1:4) of the inhibitor AmphB. AmphB does not inhibit Curli assembly.

FIG. 10A-10B shows characteristics of the assay to identify inhibition of curli formation using the anti-amyloid peptide engineered bacteriophages. FIG. 10A is a schematic of location of identified hits, and FIG. 10B shows increase in ThT fluorescence (i.e. protein aggregation formation) over time.

FIG. 11A-11B shows characteristics of the assay to identify inhibition of curli formation using the anti-amyloid peptide engineered bacteriophages. FIG. 11A shows a schematic of hits where protein aggregates have formed at specific locations in the assay. FIG. 11B shows a electron micrograph of an example of curli amyloid fibrils formed at locations where proteins aggregrates have formed.

FIG. 12A-12B shows amino acid sequence alignment and homology of CsgA and CsgB polypeptides. FIG. 12A shows the alignment of the polypeptide sequences of CsgA (SEQ ID NO: 205) and CsgB (SEQ ID NO: 206) with the N-terminal signal sequence. The signal sequences for CsgA and CsgB are shown in Bold. FIG. 12B shows the alignment of the polypeptide sequences of CsgA (SEQ ID NO: 207) and CsgB (SEQ ID NO: 208) without the N-terminal signal sequence. In both FIGS. 12A and 12B, the binding sequences in CsgB (SEQ ID NOs: 202 and 250) are underlined. Accordingly, an anti-amyloid peptide engineered bacteriophage as disclosed herein can comprise a fragment of at least 7 consecutive amino acids from SEQ ID NO: 202 or SEQ ID NO: 250.

FIG. 13A-13E shows amyloid-inhibiting peptides expressed on phage capsids to reduce biofilm formation, block mammalian cell invasion by E. coli, decrease colony growth, and affect colony morphology. FIG. 13A-13B shows curli-inhibiting phage suppressed biofilm formation based on crystal violet staining and quantification with optical density readings at 600 nm (OD₆₀₀ₙₘ). All OD₆₀₀ₙₘ data was normalized so that untreated biofilms had OD₆₀₀ₙₘ=1. T7-RRR-CsgB₁₃₃₋₁₄₂-PPP had the greatest efficacy against biofilm formation. 10⁹ PFU/mL of phage was used in each treatment well. FIG. 13C shows E. coli invasion of HEp-2 cells, as determined with a gentamicin protection assay, is decreased in the presence of T7-CsgB₁₃₃₋₁₄₂-PPP and T7-RRR-CsgB₁₃₃₋₁₄₂-PPP (SEQ ID NO: 61). FIG. 13D shows E. coli colony growth, measured by colony circumference, is retarded by knocking out csgA (green circles) or csgB (blue triangles) as well as by treating with T7-CsgA₄₃₋₅₂ (grey crosses) and T7-RRR-CsgB₁₃₃₋₁₄₂-PPP (red squares). E. coli colony growth for untreated cells is shown for reference (black diamonds). FIG. 13E shows knocking out csgB or treating E. coli with T7-RRR-CsgB₁₃₃₋₁₄₂-PPP results in the loss of rough morphologies and binding of Congo red seen with wild-type cells. Also, E. coli ΔcsgB and E. coli treated with T7-RRR-CsgB₁₃₃₋₁₄₂-PPP are mucoid compared with wild-type cells.

FIG. 14 shows the biofilm-inhibiting activity of the engineered phage, T7-RRR-CsgB₁₃₃₋₁₄₂-PPP (SEQ ID NO: 61). Crystal violet staining of E. coli biofilms shows a concentration-dependent effect for biofilm inhibition by T7-RRR-CsgB₁₃₃₋₁₄₂-PPP. T7med-RRR-CsgB₁₃₃₋₁₄₂-PPP, which expresses 5-15 peptides copies per phage, at a concentration of 10⁹ PFU/mL displayed the poorest biofilm inhibition.

FIG. 15 shows varying the amino acids flanking CsgB₁₃₃₋₁₄₂ modulated biofilm formation. Replacing the C-terminal prolines of T7-RRR-CsgB₁₃₃₋₁₄₂-PPP with glycines resulted in enhancement of biofilm formation rather than inhibition. Decreasing the number of C-terminal prolines reduced the efficacy of biofilm inhibition. Increasing the arginine and/or proline residues in T7-RRR-CsgB₁₃₃₋₁₄₂-PPP had only moderate effects.

FIG. 16 shows the efficacy of the peptide-displaying T7 bacteriophage to prevent biofilm formation on plastic pegs. Preincubation of biofilm pegs with phage followed by biofilm growth and crystal violet staining revealed that T7-RRR-CsgB₁₃₃₋₁₄₂-PPP and T7-RRR-CsgB₁₃₃₋₁₄₂-PPPPP were the most effective at blocking biofilm growth.

FIG. 17A-17C shows in vitro aggregation of amyloid-β can be inhibited by anti-curli phage. The major nucleating sequence of CsgB contains a sequence, AIVV (SEQ ID NO: 199), that when reversed, is also present within a nucleating sequence in amyloid-β (GGVVIA) (SEQ ID NO: 197). FIG. 17A shows, as monitored by ThT fluorescence, the T7-RRR-CsgB₁₃₃₋₁₄₂-PPP phage increased the lag phase of in vitro amyloid-13 assembly, while FIGS. 17B and 17C shows T7-con and T7-wt were ineffective at increasing the lag time of amyloid-β fiber assembly, respectively.

FIG. 18 shows site-specific mutations in CsgA and CsgB (SEQ ID NOs: 209-212) abolished curli formation as assayed by Congo red binding on agar plates.

FIG. 19 shows a schematic of AmyloidMutant identifying putative interactions between CsgA and CsgB confirmed by experimental mutational analysis. Putative combinations of CsgA and CsgB interactions were scored using a Boltzmann statistical mechanical scoring function, log-odds potentials derived from the Protein Data Bank, and an efficient dynamic programming algorithm. One of the highest scoring interactions was detected to be between CsgA₅₄₋₆₁ and CsgB₁₃₄₋₁₄₀ (NSALALQT/TAIVVQR) (SEQ ID NO:195/SEQ ID NO: 196), consistent with results from the peptide arrays, as long with other CsgA sequences.

FIG. 20A-20C shows the anti-amyloid peptide engineered bacteriophage can be used to efficiently suppress aggregation of another aggregation-prone system, the yeast prion Sup35-NM. Five anti-amyloid peptide engineered bacteriophages including the control were constructed with different inserts. The anti-amyloid peptide engineered bacteriophage #1316 has the insert RRR-NQQNYQQYSQNGNQQQGNNRY-PPP (SEQ ID NO: 226) (amino acids 9-29 of the NM prion domain). The anti-amyloid peptide engineered bacteriophage #1317 has the insert RRR-NQQNYQQYSQNGNQQQGNNRY-PPP-STOP (SEQ ID NO: 227) (amino acids 9-29 of the NM prion domain). The anti-amyloid peptide engineered bacteriophage #1318 has the insert RRR-ISESTHNTNNANVTSADALIK-PPP (SEQ ID NO: 228) (amino acids 220-240 of the NM prion domain). The anti-amyloid peptide engineered bacteriophage #1319 has the insert RRR-ISESTHNTNNANVTSADALIK-PPP-STOP (SEQ ID NO: 229) (amino acids 220-240 of the NM prion domain). T7 control phage is from the T7select415 kit with control insert. FIG. 20A shows the formation of Sup35-NM amyloid fiber assembly, as monitored by ThT fluorescence, in the presence of the anti-amyloid peptide engineered bacteriophages #1316 and #1318. The concentration of the anti-amyloid peptide engineered bacteriophages was normalized to 5×10⁸ PFU/mL. FIG. 20B shows formation of Sup35-NM amyloid fiber assembly, as monitored by ThT fluorescence, in the presence of a higher concentration of anti-amyloid peptide engineered bacteriophages #1316, #1317, #1318, #139, T7 control or T7 wild-type. The concentration of the anti-amyloid peptide engineered bacteriophages was normalized to 1.6×10¹⁰ PFU/mL. FIG. 20C is another set of experiment, similar to FIG. 20B, showing formation of Sup35-NM amyloid fiber assembly, as monitored by ThT fluorescence, in the presence of the anti-amyloid peptide engineered bacteriophages #1317, #139, T7 control or T7 wild-type. The concentration of the anti-amyloid peptide engineered bacteriophages was normalized to 1.6×10¹⁰ PFU/mL.

### DETAILED DESCRIPTION OF THE INVENTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. It is also understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention relates in part to compositions and method to inhibit or disrupt the formation or maintenance of protein aggregates. One aspect of the present invention is directed to engineered bacteriophages which express, either in the surface of the bacteriophage or is released (by lysis or secretion) one or more anti-amyloid peptides which inhibit or disrupt the formation or maintenance of protein aggregates.

Accordingly, one aspect of the present invention relates to the engineered bacteriophages as discussed herein which express an anti-amyloid peptide which inhibits or disrupts the formation or maintenance of protein aggregates. In one embodiment, an engineered bacteriophage which expresses an anti-amyloid peptide is termed an “anti-amyloid peptide engineered bacteriophage” herein and inhibits protein aggregates which comprise of two or more different polypeptides, e.g., “higher order aggregates” which are protein aggregates formed by a first polypeptide which seeds the formation of an aggregate comprising at least in part of a second polypeptide.

The present invention is based in part on the discovery that small anti-amyloid peptides sequences expressed from bacteriophages inhibit curli fiber formation. In some embodiments, the anti-amyloid peptides are peptide sequences of bacterial CsgB polypeptides. In some embodiments, the anti-amyloid peptides are peptide sequences of bacterial CsgA polypeptides. As described in the Examples, specific peptides within E. coli CsgB nucleated assembly of amyloid fibers and specific peptides within E. coli CsgA, or modified variants of the specific peptides when expressed on the surface of a bacteriophage can inhibit amyloid formation and inhibit bacteria. The results thus demonstrate that short peptide portions of bacterial biofilm forming proteins, lacking the context provided by some or all of the remainder of the full length polypeptide from which they were derived, inhibit the assembly of the full length polypeptides to form higher order aggregates, e.g., fibrils. Furthermore, these results show the anti-amyloid peptide can inhibit aggregate formation when the anti-amyloid peptide is expressed on the surface of the bacteriophage. Notably, the results demonstrate that anti-amyloid peptide engineered bacteriophages can be used to inhibit a first polypeptide that functions as a seed to nucleate the assembly of a second polypeptide with a distinct sequence. These anti-amyloid peptide engineered bacteriophages which express the anti-amyloid peptides, either on the surface of the bacteriophage or are released (i.e. by lysis or secretion), compositions comprising the anti-amyloid peptide engineered bacteriophages, and uses thereof are aspects of the invention.

In some embodiments, an anti-amyloid peptide expressed by an anti-amyloid peptide engineered bacteriophage as disclosed herein is a CsgA or a CsgB peptide. In some embodiments, an anti-amyloid peptide engineered bacteriophage can be used to inhibit bacteria and/or remove bacterial biofilms in environmental, industrial, and clinical settings by administering a composition comprising at least one engineered bacteriophage as discussed herein.

In particular, the inventors have engineered bacteriophages to express an anti-amyloid peptide, for example on the outside of the bacteriophage surface or to release the anti-amyloid peptide (by lysis or secretion). Such engineered bacteriophages are referred to herein as an “anti-amyloid peptide engineered bacteriophage”. In particular, the inventors have engineered bacteriophages to specifically express an anti-amyloid peptide, including but not limited to peptides derived from naturally occurring polypeptides to inhibit biofilm formation or maintenance and/or to allow for faster and more effective killing of bacteria in bacterial infections, such as bacterial infections comprising more than one different bacterial host species.

Accordingly, one aspect of the present invention generally relates to an anti-amyloid peptide engineered bacteriophage where the bacteriophage has been modified or engineered to express and/or secrete an anti-amyloid peptide. At least one, or any combination of different anti-amyloid peptide engineered bacteriophage can be used alone, or in any combination to inhibit bacterial biofilm formation or maintenance and/or to reduce, eliminate, or kill a bacterial infection or reduce or eliminate bacterial contamination. In some embodiments, an anti-amyloid peptide engineered bacteriophage can be used with an additional agent, such as the same or a different anti-amyloid agent which is expressed by the bacteriophage.

Accordingly, one aspect of the present invention relates to the use of an anti-amyloid peptide engineered bacteriophage in conjunction with (i.e. in combination with) at least one other agent, such as an anti-amyloid agent or agent which inhibits fiber aggregation.

One aspect of the present invention relates to a method to inhibit or disrupt the formation or maintenance of protein aggregates. Another aspect of the present invention relates to a method to eliminate or decrease protein aggregates in bacterial biofilms.

In particular, one aspect of the present invention relates to methods and compositions comprising an anti-amyloid peptide engineered bacteriophage to inhibit or disrupt the formation or maintenance of protein aggregates such that the bacteriophage can subsequently kill the bacteria and/or so that the bacteria are rendered more susceptible to other anti-bacterial agents or a subjects natural defenses and immune system.

Another aspect of the present invention relates to the use of an anti-amyloid peptide engineered bacteriophage to inhibit or disrupt the formation or maintenance of protein aggregates, wherein the aggregates, in some embodiments, comprise at least 2 different polypeptides, and more particularly comprise a first amyloidogenic polypeptide which forms a seed to nucleate aggregation of a second amyloidogenic polypeptide. In one embodiment of this aspect and all aspects described herein, an anti-amyloid peptide engineered bacteriophage can comprise at least one or more than one anti-amyloid peptide, such as for example, at least 2, at least 3, at least 4, at least 5, least 6, at least 7, at least 8, at least 9 or at least 10 or more different anti-amyloid peptides at any one time. In some embodiments, an anti-amyloid peptide engineered bacteriophage as disclosed herein can used in combination with at least one or more different anti-amyloid peptide engineered bacteriophages, for example an anti-amyloid peptide engineered bacteriophage as disclosed herein can used in combination with at least 2, 3, 4, 5, 6, 7, 8, 9 or 10 or more different anti-amyloid peptide engineered bacteriophages.

Provided herein are a plurality of anti-amyloid peptide engineered bacteriophages which express at least one or a plurality of anti-amyloid peptides, wherein the peptides are portions of a first amyloidogenic polypeptide that is prone to form aggregates with a second amyloidogenic polypeptide of different sequence under appropriate conditions. In some embodiments the first amyloidogenic polypeptide is any polypeptide that can form heteroaggregates comprised in part of a second amyloidogenic polypeptide. In some embodiments of interest the first and second amyloidogenic polypeptides are at least 70%, 80%, 85%, 90%, 95%, 96%, 97%, 98%, 99%, or 100% identical to polypeptides that assemble to form amyloids present in biofilms. In some embodiments of particular interest the first amyloidogenic polypeptide is a CsgB polypeptide and the second amyloidogenic polypeptide is a CsgA polypeptide. In some embodiments the first amyloidogenic polypeptide is any naturally occurring polypeptide wherein heteroaggregates formed in part from the polypeptide and/or in part from fragments of the polypeptide play a role in disease, e.g., in mammals such as humans, non-human primates, domesticated animals, rodents such as mice or rats, etc. In some embodiments the first polypeptide is at least 70%, 80%, 85%, 90%, 95%, 96%, 97%, 98%, 99%, or 100% identical to such a naturally occurring polypeptide.

In some aspects, the present invention relates to a composition comprising anti-amyloid peptide engineered bacteriophages. In some embodiments, a composition comprises a plurality of anti-amyloid peptide engineered bacteriophages, e.g., up to 10, 50, 100, 150, 200, 250, or more different anti-amyloid peptide engineered bacteriophages, each expressing the same or unique (i.e. different) anti-amyloid peptides. The sequences of the anti-amyloid peptides may collectively encompass between 20-100% of a complete polypeptide sequence, e.g., 30-100%, 40-100%, 50-100%, 60-100%, 70-100%, 80-100%, or 90-100% of the full length sequence of an amyloid polypeptide, such as CsgA (SEQ ID NO: 1) or CsgB (SEQ ID NO:2). The peptides may be, e.g., 6-12, 8-15, 10-20, 10-30, 20-30, 30-40, or 40-50 amino acids in length. In some embodiments, the peptides overlap in sequence by between, e.g., 1-25 residues, e.g., between 5-20 residues, or between 10-15 residues. In some embodiments, the peptides “scan” at least a portion of the polypeptide, i.e., the starting positions of the peptides with respect to the polypeptide are displaced from one another (“staggered”) by X residues where X is, for example, between 1-10 residues or between 1-6 residues or between 1-3 residues. In one embodiment, the starting positions of the peptides with respect to the polypeptide sequence are staggered by 1 amino acid. For example, a first peptide corresponds to amino acids 1-20; a second peptide corresponds to amino acids 2-21; a third peptide corresponds to amino acids 3-22, etc. In another embodiment, the starting positions of the peptides with respect to the polypeptide sequence are staggered by 2 amino acids. For example, a first peptide corresponds to amino acids 1-20; a second peptide corresponds to amino acids 3-22; a third peptide corresponds to amino acids 5-23, etc. The collection need not include a peptide that comprises the N-terminal or C-terminal amino acid(s) of the polypeptide. For example, a signal sequence could be omitted. The collection could span any N-terminal, C-terminal, or internal portion of the polypeptide. In some embodiments the peptides have a detectable label, a reactive moiety, a tag, a spacer, or a crosslinker linked thereto. The peptides need not all be the same length and need not all fall within any single range of lengths.

In certain embodiments of all aspects of the invention, an anti-amyloid peptide expressed by the anti-amyloid peptide engineered bacteriophage as disclosed herein is a fragment or peptide of a polypeptide that normally promotes formation of biofilms. In some embodiments, an anti-amyloid peptide expressed by the anti-amyloid peptide engineered bacteriophage is a peptide derived from a first or second amyloidogenic polypeptide, wherein the first or second amyloidogenic polypeptide are at least 70%, 80%, 85%, 90%, or 95% identical to polypeptides that assemble to form amyloids present in biofilms e.g., bacterial polypeptides that assemble to form amyloid fibers such as curli. Curli are the major proteinaceous component of a complex extracellular matrix produced by many bacteria, e.g., many Enterobacteriaceae such as E. coli and Salmonella spp. (Barnhart M M, Chapman M R. Annu Rev Microbiol., 60:131-47, 2006). Other biofilm-forming bacteria of interest include Klebsiella, Pseudomonas, Enterobacter, Serratia, Citrobacter, Proteus, Yersinia, Citrobacter, Shewanella, Agrobacter, Campylobacter, etc.

Curli fibers are involved in adhesion to surfaces, cell aggregation, and biofilm formation. Curli also mediate host cell adhesion and invasion, and they are potent inducers of the host inflammatory response. Curli exhibit structural and biochemical properties of amyloids, e.g., they are nonbranching, β-sheet rich fibers that are resistant to protease digestion and denaturation by 1% SDS and bind to amyloid-specific moieties such as thioflavin T, which fluoresces when bound to amyloid, and Congo red, which produces a unique spectral pattern (“red shift”) in the presence of amyloid. Polypeptides that assemble to form curli are of interest at least in part because of their association with animal and human disease. Bacterial polypeptides that promote formation of biofilms present in a variety of natural habitats are also of interest. For example, in a recent study bacteria producing extracellular amyloid adhesins were identified within several phyla: Proteobacteria (Alpha-, Beta-, Gamma- and Deltaproteobacteria), Bacteriodetes, Chloroflexi and Actinobacteria (Larsen, P., et al., Environ Microbiol., 9(12):3077-90, 2007). Particularly in drinking water biofilms, a high number of amyloid-positive bacteria were identified. Bacteria of interest may be gram-negative or gram-positive. In some embodiment bacteria of interest are rods. In some embodiments they are aerobic. In some embodiments they are facultative anaerobes or anaerobes.

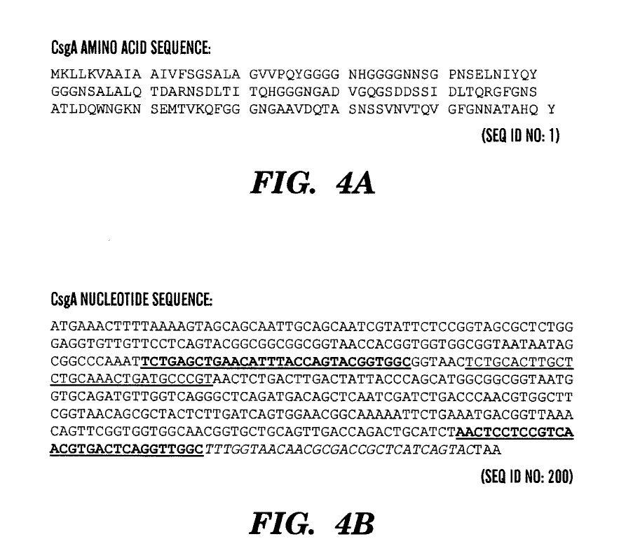

In nature, curli are assembled by a process in which the major curli subunit polypeptide, CsgA, is nucleated into a fiber by the minor curli subunit polypeptide, CsgB. CsgA and CsgB are about 30% identical at the amino acid level and contain five-fold internal symmetry characterized by conserved polar residues. The assembly process is believed to involve addition of soluble polypeptides to the growing fiber tip. Thus both subunits are incorporated into the fiber, although CsgA is the major protein constituent and CsgB is the nucleating, or seed forming polypeptide. In living bacteria, curli formation likely involves activities of several additional polypeptides encoded by other Csg genes (CsgD, CsgE, CsgF, CsgG), but these polypeptides are not required for curli formation in vitro. Sequences of CsgA and CsgB from a large number of bacteria have been identified. Exemplary CsgA and CsgB amino acid sequences are shown in FIGS. 4A (SEQ ID NO:1) and 5A (SEQ ID NO: 2), respectively. One of skill in the art will readily be able to find CsgA and CsgB sequences by searching databases such as GenBank publicly available through the National Center for Biotechnology Information (NCBI) (see ncbi.nlm.nih.gov), and there are computational methods for determining, and predicting anti-amyloid peptides to inhibit curli formation in the methods and bacteriophages as disclosed herein.

In one aspect of the present invention, an anti-amyloid peptide engineered bacteriophage as disclosed herein can comprise a nucleic acid encoding an anti-amyloid peptide, wherein the anti-amyloid peptide is derived from a CsgB polypeptide or a CsgA polypeptide. In another embodiment, an anti-amyloid peptide engineered bacteriophage as disclosed herein can comprise a nucleic acid encoding a fragment of a naturally occurring anti-amyloid agent. In other embodiments, an anti-amyloid peptide engineered bacteriophage as disclosed herein can comprise a nuclei acid encoding a fragment of a different Csg polypeptide selected from the group of CsgD, CsgE, CsgF, CsgG polypeptides.

In some embodiments of this aspect and all aspects described herein, an anti-amyloid peptide engineered bacteriophage as disclosed herein can comprise a nucleic acid encoding an anti-amyloid peptide such as, for example but it not limited to, at least one of the following different CsgA peptide is selected from SEQ ID NOs: 11-18 or SEQ ID NOs: 35-58 or variants or modified variants thereof, and a CsgB peptide is selected from SEQ ID NOs: 27-34 or SEQ ID NOs: 59-90, or variants or modified variants thereof.

In one embodiment of this aspect and all aspect described herein, an anti-amyloid peptide engineered bacteriophage can comprise at least 2, 3, 4, 5 or even more, for example 10 different nucleic acids which encode an anti-amyloid peptide, for example, 2, 3, 4, 5, 6, 7 or more of the anti-amyloid peptides encoded by nucleic acid sequences SEQ ID NO: 3-10, 19-26. In some embodiments, any or all different combinations of anti-amyloid peptides and be present in an anti-amyloid peptide engineered bacteriophage.

In another aspect of the present invention, an anti-amyloid peptide engineered bacteriophage can comprise at least one nucleic acid encoding an anti-amyloid agent which inhibits or blocks amyloid formation. In some embodiments of this aspect, and all other aspects described herein, such an anti-amyloid peptide expressed by an anti-amyloid peptide engineered bacteriophage which inhibits or blocks the formation of amyloids refers to any anti-amyloid peptide which inhibits the formation of amyloid aggregates by at least about 10% or at least about 15%, or at least about 20% or at least about 30% or at least about 50% or more than 50%, or any integer between 10% and 50% or more, as compared to the use of a control peptide (e.g. not an anti-amyloid peptide). Stated another way, the anti-amyloid peptide can reduce the presence of an amyloid aggregates by at least about 10% or at least about 15%, or at least about 20% or at least about 30% or at least about 50% or more than 50%, or any integer between 10% and 50% or more, as compared to the use of a control peptide is encompassed for use useful in the present invention.

In some embodiments, the reduction of the amount of amyloid formation or amyloid aggregates by the anti-amyloid peptide expressed by an anti-amyloid peptide engineered bacteriophage is a reduction of at least about 10%, or at least about 15%, or at least about 20%, or at least about 25%, or at least about 35%, or at least about 50%, or at least about 60%, or at least about 90% and all integers in between 10-90% of the amount of the amyloid deposits when compared to a similar amount of a bacteriophage which has not been engineered to express an anti-amyloid peptide.

The inventors have also demonstrated herein in Examples that an anti-amyloid peptide engineered bacteriophage which comprises at least one anti-amyloid peptide can decrease amyloid formation, for example inhibit in vitro and in vivo assembly of curli formation by bacteria.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “anti-amyloid peptide engineered bacteriophage” refers to a bacteriophage that have been genetically engineered to comprise a nucleic acid which encodes an anti-amyloid peptide, for example, the anti-amyloid peptide reduces a formation or inhibits the maintenance of protein aggregates comprised, in some embodiments, of at least two different polypeptides. Naturally, one can engineer a bacteriophage to comprise at least one nucleic acid which encodes more than one anti-amyloid peptide, for example, two or more anti-amyloid peptides which are fragments from the same polypeptide (such as CsgB) or to at least two polypeptides (such as CsgA and CsgB) which can be used in the methods and compositions as disclosed herein.

The term “engineered bacteriophage” as used herein refers to an anti-amyloid peptide engineered bacteriophage as this phrase is defined herein.

The term “higher ordered” refers to an aggregate of at least 10 polypeptide subunits, or in some embodiments at least 15 polypeptide subunits, or in some embodiments at least 25 polypeptide subunits and is meant to exclude the many proteins that are known to include polypeptide dimers, tetramers, or other small numbers of polypeptide subunits in an active complex, although the peptides and polypeptides may form such complexes as well. The term “higher-ordered aggregate” also is meant to exclude random agglomerations of denatured proteins that can form in non-physiological conditions. Higher ordered aggregates of interest herein are commonly referred to in scientific literature by terms such as “amyloid”, “amyloid fibers”, “amyloid fibrils”, or simply as “fibers” or “fibrils”, and those terms are used interchangeably herein. The term “higher-ordered aggregate” is also used interchangeably herein with the noun “aggregate”. Polypeptides that assemble to form amyloid fibers are referred to herein as “amyloidogenic”. It will be understood that many polypeptides that participate in formation of higher-ordered aggregates can exist in at least two conformational states, only one of which is typically found in the ordered aggregates or fibrils. Stated another way, high-ordered aggregates comprise aggregation-prone polypeptides which bind to other different aggregation-prone polypeptide to form a higher ordered aggregate, e.g., an aggregate referred to in the scientific literature by terms such as “amyloid,” “amyloid fibrils,” “fibrils” (also referred to as “fibers”) and “prions”. By “higher ordered” is meant an aggregate of at least 25 polypeptide subunits, and is meant to exclude the many proteins that are known to include polypeptide dimers, tetramers, or other small numbers of polypeptide subunits in an active complex, although the peptides and polypeptides may form such complexes as well. The term “higher-ordered aggregate” also is meant to exclude random agglomerations of denatured proteins that can form in non-physiological conditions. The term “higher-ordered aggregate” is used interchangeably herein with the term “aggregate” unless otherwise indicated.

The term “assembles” refers to the property of certain polypeptides to form ordered aggregates under appropriate conditions and is not intended to imply that the formation of higher ordered aggregates will occur under every concentration or every set of conditions. A peptide that, when present as part of a first polypeptide, can promote (e.g., accelerate or cause) assembly of a second polypeptide differing in sequence from the first polypeptide, so as to form fibers comprising both first and second polypeptides, is referred to herein as a “nucleating peptide” and its amino acid sequence will be referred to as a “nucleating sequence”. Also, “nucleating peptide” encompasses peptides that nucleate assembly of a polypeptide with other polypeptides identical in sequence. Curli are composed of polypeptides of different sequences (CsgA and CsgB) but many amyloids are composed of identical polypeptides. In some embodiments of the invention, a nucleating peptide is characterized in that its deletion (e.g., in part or in full) from a polypeptide significantly slows down or abolishes fiber assembly with a compatible polypeptide.

The term “naturally occurring amyloid” or “naturally forming amyloid” refers to formation of protein aggregates under a natural condition. In particular, the naturally occurring amyloid or the naturally forming amyloid comprises a first amyloidogenic polypeptide which is capable of functioning as a seed for nucleating amyloid formation by a second amyloidogenic polypeptide.

Amyloid fibers have a characteristic morphology under electron microscopy, are β-sheet rich, typically non-branching, and react characteristically with certain amyloid-specific dyes such as thioflavin T (ThT) and Congo red. Such dyes may be used to identify and/or detect amyloid fibers and thus serve as indicators of the formation or presence of such fibers in certain embodiments of the invention. In certain embodiments of interest herein, amyloid fibers are composed of two different polypeptide species, e.g., CsgA and CsgB. In some embodiments amyloid fibers are composed of more than two polypeptide species. The ratio of first polypeptide to second polypeptide in the fiber can vary. In some embodiments, the fiber is composed largely of the second amyloidogenic polypeptide. For example, in some embodiments the second polypeptide species constitutes at least 70%, at least 80%, at least 90%, or more of the fiber by weight, or, in some embodiments by number, of subunits. In other embodiments, the first polypeptide species constitutes at least 70%, at least 80%, at least 90%, or more of the fiber by weight, or, in some embodiments by number, of subunits. In one aspect, peptides that are derived from a first amyloidogenic polypeptide, and to which a second amyloidogenic polypeptide having a different sequence to the first amyloidogenic polypeptide binds to form a higher ordered aggregate are provided. In some embodiments the first and second polypeptides are at least 50%, 60%, 70%, 80%, 90%, or up to 95% identical. In some embodiments the first and second amyloidogenic polypeptides are no more than 50% identical, e.g., between 20% and 40% identical. In some embodiments, the presence of the first polypeptide or an aggregation domain derived from the first polypeptide greatly accelerates or is required for formation of an amyloid comprising the second polypeptide. Either or both of the polypeptides may contain multiple aggregation domains, which can be identical or different in sequence.

The term “amyloid associated disorder” is used interchangeably herein with the term “amyloidosis” and refers to any of a number of disorders which have as a symptom or as part of its pathology the accumulation or formation of plaques or amyloid plaques or amyloid protein aggregates in a specific tissue or a various different tissues. The abnormal protein aggregates, also called deposits are called “amyloid”, or “amyloid plaques” are extracellular deposits comprised mainly of proteinaceous fibrils. Generally, the fibrils are composed of a dominant protein or peptide; however, the plaque may also include additional components that are peptide or non-peptide molecules. These protein aggregates damage the tissues and interfere with the function of the involved organ. An amyloid associated disorder or amyloidosis occurs in multiple forms: spontaneous, hereditary, and also in some instances is a result from a cancer of the blood cells called myeloma. Hereditary amyloidosis is an inherited form, and in some occasions is transmitted as an autosomal dominant trait.

The term “AL amyloidosis” as used herein refers to the disease or disorder from AL amyloid deposits, or the formation of amyloid deposits comprising monoclonal immunoglobulin light chain.

An “amyloid component” is any molecular entity that is present in an amyloid plaque including antigenic portions of such molecules. Amyloid components include but are not limited to proteins, peptides, proteoglycans, and carbohydrates. A “specific amyloid component” refers to a molecular entity that is found primarily or exclusively in the amyloid plaque of interest.

The term “CsgA polypeptide” as used herein encompasses any polypeptide whose sequence comprises or consists of the sequence of a naturally occurring bacterial CsgA polypeptide (SEQ ID NO:1). The term also encompasses polypeptides that are variants of a polypeptide whose sequence comprises or consists of the sequence of a naturally occurring bacterial CsgA polypeptide, which are referred to as “CsgA polypeptide variants”. In some embodiments a CsgA polypeptide variant is at least 20%, 30%, 40%, 50%, 60%, 70%, 80%, 85%, 90%, 95%, 96%, 97%, 98%, 99%, or 100% identical to or similar to a naturally occurring CsgA polypeptide (SEQ ID NO:1) across the length of the CsgA polypeptide variant. In some embodiments, a CsgA polypeptide variant is at least 20%, 30%, 40%, 50%, 60%, 70%, 80%, 85%, 90%, 95%, 96%, 97%, 98%, 99%, or 100% identical to or similar to a half (or 50%) of the length of a naturally occurring CsgA polypeptide (SEQ ID NO:1).

In some embodiments a “CsgA peptide” is also used interchangeably herein as a “CsgA polypeptide fragment” is at least 5% or 10%, 20%, 30%, 40%, 50%, 60%, 70%, 80%, 90%, 95%, 98%, or 100% as long as a naturally occurring CsgA polypeptide. In some embodiments a CsgA peptide is at least 8-10 amino acids long. In some embodiments, a CsgA peptide is at least 8-10 amino acids long of a variant of a CsgA polypeptide. In some embodiments, a CsgA peptide is at least 8, 9, 10, 11, 12, 13, 14, 15, 16, 17, 18, 19, 20 or more than 20 amino acids long of a naturally occurring CsgA polypeptide or a variant of a CsgA polypeptide. In some embodiments, a CsgA peptide is at least 8, 9, 10, 11, 12, 13, 14, 15, 16, 17, 18, 19, 20 or more than 20 amino acids long of a naturally occurring CsgA polypeptide where at least one amino acid has been modified (i.e. by substitution, deletion or addition of an amino acid or amino acid analogue). In some embodiments, a CsgA peptide is at least 7, 8, 9, 10, 11, 12, 13, 14, 15, 16, 17, 18, 19, 20 or more than 20 amino acids long of a naturally occurring CsgA polypeptide where at least 1, 2, 3, 4, 5 or more than 5 amino acids has been modified (i.e. by substitution, deletion or addition of an amino acid or amino acid analogue). In some embodiments, a CsgA peptide is at least 7, 8, 9, 10, 11, 12, 13, 14, 15, 16, 17, 18, 19, 20 or more than 20 amino acids long of a naturally occurring CsgA polypeptide where at least 1, 2, 3, 4, 5 or more than 5 amino acids has been added to the N-terminus or C-terminus or both of the CsgA peptide. In some embodiments a CsgA polypeptide is wild type at one, more, or all of the following positions: 49, 54, 139, 144 (where amino acid numbering is based on the E. coli CsgA sequence). In some embodiments the CsgA polypeptide has a substitution at one or more of the foregoing positions.

The term “CsgB polypeptide” as used herein encompasses any polypeptide whose sequence comprises or consists of the sequence of a naturally occurring bacterial CsgB polypeptide (SEQ ID NO:2). The term also encompasses polypeptides that are variants of a polypeptide whose sequence comprises or consists of the sequence of a naturally occurring bacterial CsgB polypeptide. Such variants are referred to as “CsgB polypeptide variants”. In some embodiments a CsgB polypeptide variant is at least 30%, 40%, 50%, 60%, 70%, 80%, 85%, 90%, 95%, 96%, 97%, 98%, 99%, or 100% identical to or similar to a naturally occurring polypeptide across the length of the CsgB polypeptide variant (SEQ ID NO:2). In some embodiments, a CsgB polypeptide variant is at least 20%, 30%, 40%, 50%, 60%, 70%, 80%, 85%, 90%, 95%, 96%, 97%, 98%, 99%, or 100% identical to or similar to a half (or 50%) of the length of a naturally occurring CsgB polypeptide (SEQ ID NO:2).

In some embodiments a “CsgB peptide” is also used interchangeably herein as a “CsgB polypeptide fragment” is at least 5% or 10%, 20%, 30%, 40%, 50%, 60%, 70%, 80%, 90%, 95%, 98%, or 100% as long as a naturally occurring CsgB polypeptide. In some embodiments a CsgB peptide is at least 8-10 amino acids long. In some embodiments, a CsgB peptide is at least 8-10 amino acids long of a variant of a CsgB polypeptide. In some embodiments, a CsgB peptide is at least 8, 9, 10, 11, 12, 13, 14, 15, 16, 17, 18, 19, 20 or more than 20 amino acids long of a naturally occurring CsgB polypeptide or a variant of a CsgB polypeptide. In some embodiments, a CsgB peptide is at least 8, 9, 10, 11, 12, 13, 14, 15, 16, 17, 18, 19, 20 or more than 20 amino acids long of a naturally occurring CsgB polypeptide where at least one amino acid has been modified (i.e. by substitution, deletion or addition of an amino acid or amino acid analogue). In some embodiments, a CsgB peptide is at least 7, 8, 9, 10, 11, 12, 13, 14, 15, 16, 17, 18, 19, 20 or more than 20 amino acids long of a naturally occurring CsgB polypeptide where at least 1, 2, 3, 4, 5 or more than 5 amino acids has been modified (i.e. by substitution, deletion or addition of an amino acid or amino acid analogue). In some embodiments, a CsgB peptide is at least 7, 8, 9, 10, 11, 12, 13, 14, 15, 16, 17, 18, 19, 20 or more than 20 amino acids long of a naturally occurring CsgA polypeptide where at least 1, 2, 3, 4, 5 or more than 5 amino acids has been added to the N-terminus or C-terminus or both of the CsgB peptide. In some embodiments the CsgA or CsgB polypeptide variant lacks about 10-20 amino acids from the N-terminus, C-terminus, or both, as compared with a naturally occurring CsgA or CsgB polypeptide.

The term “anti-amyloid peptide” as used herein refers to any amyloid peptide which can inhibit the formation or maintenance of a high order aggregate. An anti-amyloid peptide is any peptide which results in inhibition of amyloid formation by at least about 30% or at least about 40%, or at least about 50% or at least about 60% or at least about 70% or more than 70%, or any integer between 30% and 70% or more, as compared to in the absence of the anti-amyloid peptide. The term anti-amyloid peptides encompasses all peptides that inhibit or reduce the formation or maintenance of protein aggregates, and are typically, for example but not limited to, short proteins, generally between 12 and 50 amino acids long, however larger proteins are also encompassed as anti-amyloid peptides in the present invention.

The term “pro-amyloid peptide” as used herein refers to any amyloid peptide which can increase the formation or promote the maintenance of a high order aggregate. A pro-amyloid peptide is any peptide which results in an increase in amyloid formation by at least about 10% or at least about 20% or at least about 30% or at least about 40%, or at least about 50% or at least about 60% or at least about 70% or more than 70%, or any integer between 10% and 70% or more, as compared to in the absence of the pro-amyloid peptide. The term pro-amyloid peptides encompasses all peptides that increase or promote the formation or maintenance of protein aggregates, and are typically, for example but not limited to, short proteins, generally between 12 and 50 amino acids long, however larger proteins are also encompassed as pro-amyloid peptides in the present invention.

The term “agent” as used herein and throughout the application is intended to refer to any means such as an organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. In some embodiments of interest, the term “agent” as used herein and throughtout the application can refer to an engineered bacteriopahge as disclosed herein.

The term “microorganism” includes any microscopic organism or taxonomically related macroscopic organism within the categories algae, bacteria, fungi, yeast and protozoa or the like. It includes susceptible and resistant microorganisms, as well as recombinant microorganisms. Examples of infections produced by such microorganisms are provided herein. In one aspect of the invention, an anti-amyloid peptide is used to target microorganisms in order to prevent and/or inhibit their growth, and/or for their use in the treatment and/or prophylaxis of an infection caused by the microorganism, for example multi-drug resistant microorganisms and/or gram-negative microorganisms.

The term “release” or “released” from the host cell means that the expressed anti-amyloid peptide is moved to the external of the bacterial cell.

The term “secretion” refers to the process of, elaborating and releasing agents or chemicals from a cell, or an agent expressed by the cell. In contrast to excretion, the substance may have a certain function, rather than being a waste product.

The term “infection” or “microbial infection” which are used interchangeably herein refers to in its broadest sense, any infection caused by a microorganism and includes bacterial infections, fungal infections, yeast infections and protozoal infections.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, whole blood, plasma, serum, urine, semen, saliva, aspirates, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tissue biopsies, scrapes (e.g. buccal scrapes), tumors, organs, and also samples of in vitro cell culture constituent. In some embodiments, where the sample is solid, it can be liquidized and homogenized into a liquid sample for use in the device and systems as disclosed herein. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. In some embodiments, the term “treatment” can also mean prolonging survival as compared to expected survival if not receiving treatment. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management. The subject is usually ill or injured, or at an increased risk of becoming ill relative to an average member of the population and in need of such attention, care, or management. Evidence of treatment may be clinical or sub-clinical. In some embodiments, treatment is prophylactic treatment. Prophylactic treatment refers to complete or partial prevention of development of high ordered aggregates, or prevention of a disease or disorder as a result of amyloid formation. The methods as disclosed herein can be used prophylatically, for example in instances where, a subject is susceptible for an amyloid related disorder, or likely to have amyloid formation, such as having or likely to have an infection with a species of bacteria which forms a biofilm. For example, microbial infections such as bacterial infections such those giving rise to biofilms can occur on any surface where sufficient moisture and nutrients are present. In some embodiments, preventive treatment can be used on a surface of implanted medical devices, such as catheters, heart valves and joint replacements. In particular, catheters are associated with infection by many biofilm forming organisms such as Staphylococcus epidermidis, Staphylococcus aureus, Pseudomonas aeruginosa, Enterococcus faecalis and Candida albicans which frequently result in generalized blood stream infection. In a subject identified to have a catheter infected with bacteria, such as for example, a bacterial infected central venous catheter (CVC), the subject can have the infected catheter removed and can be treated by the methods and compositions as disclosed herein comprising an engineered bacteriophage and anti-amyloid peptide to eliminate the bacterial infection. Furthermore, on removal of the infected catheter and its replacement with a new catheter, the subject can also be administered the compositions comprising engineered bacteriophages and anti-amyloid peptides as disclosed herein on a prophylaxis basis to prevent re-infection or the re-occurrence of the bacterial infection. Alternatively, a subject can be administered the compositions as disclosed herein comprising engineered bacteriophages and anti-amyloid peptides on a prophylaxis basis on initial placement of the catheter to prevent any antimicrobial infection such as a bacterial biofilm infection. The effect can be prophylactic in terms of completely or partially preventing a disease or sign or symptom thereof, and/or can be therapeutic in terms of a partial or complete cure of a disease.

As used herein, the term “effective amount” is meant an amount of an anti-amyloid peptide engineered bacteriophage effective to yield a desired decrease in amyloid amount, or a desired inhibition of amyloid formation or maintenance. In some embodiments, an effective amount of the anti-amyloid peptide engineered bacteriophage to reduce or inhibit amyloid formation or maintenance, is an amount of anti-amyloid peptide engineered bacteriophage which decreases the amount of amyloid, or inhibiting the formation of amyloid by a statistically significant amount as compared to in the absence of the anti-amyloid peptide engineered peptide. The term “effective amount” as used herein can also or alternately refer to that amount of composition comprising an anti-amyloid peptide engineered bacteriophage necessary to achieve the indicated effect, i.e. a reduction of the amount of amyloid, as a non-limiting example, a reduction in the amount of curli formation by bacteria, by at least 5%, at least 10%, by at least 20%, by at least 30%, at least 35%, at least 50%, at least 60%, at least 90% or any integer of a reduction of the amount of amyloid (e.g. curli amount by a bacteria) in 5% and 90% or more. As used herein, in some embodiments, the effective amount of an anti-amyloid peptide engineered bacteriophage as disclosed herein is the amount sufficient to inhibit the formation or inhibit the maintenance of amyloid, as a non-limiting example, an inhibition of the amount of curli formation by bacteria, by at least 5%, at least 10%, by at least 20%, by at least 30%, at least 35%, at least 50%, at least 60%, at least 90% or any integer of an inhibition of formation or maintenance of amyloid (e.g. curli formation or maintenance by a bacteria) in 5% and 90% or more. The “effective amount” or “effective dose” will, obviously, vary with such factors, in particular, the strain of bacteria being treated, the strain of bacteriophage being used, the genetic modification of the bacteriophage being used, the specific anti-amyloid peptide, as well as the particular condition being treated, the physical condition of the subject, the type of subject being treated, the duration of the treatment, the route of administration, the type of anti-amyloid peptide and/or enhancer of anti-amyloid peptide, the nature of concurrent therapy (if any), and the specific formulations employed, and the level of expression and level of secretion of the anti-amyloid peptide from the anti-amyloid peptide engineered bacteriophage components to each other. The term “effective amount” when used in reference to administration of the compositions comprising an anti-amyloid peptide engineered bacteriophage as disclosed herein to a subject refers to the amount of the compositions to reduce or stop at least one symptom of the disease or disorder, for example a symptom or disorder of the microorganism infection, such as bacterial infection. For example, an effective amount using the methods as disclosed herein would be considered as the amount sufficient to reduce a symptom of the disease or disorder of the bacterial infection by at least 10% or more. An effective amount as used herein would also include an amount sufficient to prevent or delay the development of a symptom of the disease, alter the course of a symptom disease (for example but not limited to, slow the progression of a symptom of the disease), or reverse a symptom of the disease. An effective amount as used herein also includes an amount sufficient to inhibit the biofilm formation or bacterial infection on a solid surface or in a fluid sample.

As used herein, the term “pharmaceutically acceptable carrier” means a pharmaceutically acceptable material, composition or vehicle, such as a liquid or solid filler, diluent, excipient, solvent, suspending agent or encapsulating material, involved in carrying or transporting the subject agents (i.e. anti-amyloid peptide engineered bacteriophages). The carrier can be liquid or solid and is selected with the planned manner of administration in mind. The carrier or excipient generally does not provide any pharmacological activity to the formulation, though it may provide chemical and/or biological stability, release characteristics, and the like. Exemplary formulations can be found, for example, in Remington's Pharmaceutical Sciences, 19^{th} Ed., Grennaro, A., Ed., 1995. The carrier or excipient can be used to carry the anti-amyloid peptide engineered bacteriophages from one organ, or portion of the body, to another organ, or portion of the body. Each carrier must be “acceptable” in the sense of being compatible with the other ingredients of the formulation and non injurious to the subject. The term “pharmaceutically acceptable carrier” is used interchangeably with a “pharmaceutical carrier”.

The terms “patient”, “subject” and “individual” are used interchangeably herein, and refer to an animal, particularly a human, to whom treatment including prophylaxis treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. Suitable mammals also include members of the orders Primates, Rodenta, Lagomorpha, Cetacea, Homo sapiens, Carnivora, Perissodactyla and Artiodactyla. Members of the orders Perissodactyla and Artiodactyla are included in the invention because of their similar biology and economic importance, for example but not limited to many of the economically important and commercially important animals such as goats, sheep, cattle and pigs have very similar biology and share high degrees of genomic homology.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “gene product(s)” as used herein refers to include RNA transcribed from a gene, or a polypeptide encoded by a gene or translated from RNA.

The terms “lower”, “reduce”, “reduction”, “decrease”, “inhibit”, “disrupt”, or “eliminate” are all used herein generally to mean a decrease by a statistically significant amount. The terms “inhibit” or “reduced” or “reduce” or “decrease” or “disrupt” or “eliminate” as used herein generally means to inhibit or decrease the amount of protein aggregation by a statistically significant amount relative to in the absence of an anti-amyloid peptide or anti-amyloid peptide engineered bacteriophage. The term “inhibition” or “inhibit” or “reduce” when referring to the activity of an anti-amyloid peptide or an anti-amyloid peptide engineered bacteriophage as disclosed herein refers to prevention of, or reduction in the rate of formation of, or the amount of amyloid. However, for avoidance of doubt, “inhibit” means statistically significant decrease in the amount of a targeted amyloid by at least about 10% as compared to in the absence of an anti-amyloid peptide, for example a decrease by at least about 20%, at least about 30%, at least about 40%, at least about 50%, or least about 60%, or least about 70%, or least about 80%, at least about 90% or more, up to and including a 100% inhibition, or any decrease in the amount of amyloid between 10-100% as compared to in the absence of an anti-amyloid peptide.

The terms “increased”, “increase” or “enhance” or “activate” or “promote” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “formation” used herein refers an appearance of protein aggregates, such as amyloid or amyloid-associated aggregates. The term “formation” also means an increase in the amount of amyloid aggregates. In some embodiments, the term “formation” refers to an appearance of a biofilm caused by bacterial infection. It can also mean an increase in the density or thickness of a biofilm.

The term “maintenance” used herein means keeping the amount of protein aggregates such as amyloid or amyloid-associated aggregates at a constant level. In some embodiments, the term “maintenance” means preventing development of biofilm resulted from bacterial infection.

The term “biofilm” used herein refers to an aggregation of microorganisms (e.g. bacteria) encapsulated in a polymeric matrix, such as amyloid plaque, and adherent to each other and/or to a surface of the host.

The term “nucleic acid” or “oligonucleotide” or “polynucleotide” used herein can mean at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O- and N-alkylated nucleotides, e.g. N6-methyl adenosine are suitable. The 2′ OH— group can be replaced by a group selected from H. OR, R. halo, SH, SR, NH₂, NHR, NR₂ or CN, wherein R is C—C6 alkyl, alkenyl or alkynyl and halo is F, Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

A “pharmaceutical composition” refers to a chemical or biological composition, including anti-amyloid peptide engineered bacteriophages or pro-amyloid peptide engineered bacteriophages suitable for administration to a mammalian individual. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

As used herein, the terms “administering,” and “introducing” are used interchangeably and refer to the placement of an anti-amyloid peptide engineered bacteriophage, or a pro-amyloid peptide engineered bacteriophage as disclosed herein onto the surface infected by bacteria or into a subject, such as a subject which is at risk of an amyloid associated disorder as disclosed herein, by any method or route which results in at least partial localization of an anti-amyloid peptide engineered bacteriophage at a desired site. The compositions as disclosed herein can be administered by any appropriate route which results in the effective reduction or inhibition of the growth of the bacteria. Administration also refers to placement of an anti-amyloid peptide engineered bacteriophage or pro-amyloid peptide engineered bacteriophage on a surface, or in a fluid sample, e.g. water.

The term “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically,” “peripheral administration” and “administered peripherally” as used herein mean the administration of a compound, bacteriophage, drug or other material other than directly into the central nervous system, such that it enters the animal's system and, thus, is subject to metabolism and other like processes, for example, subcutaneous administration.

The term “tissue” is intended to include intact cells, blood, blood preparations such as plasma and serum, bones, joints, muscles, smooth muscles, and organs.

The term “vectors” is used interchangeably with “plasmid” to refer to a nucleic acid molecule capable of transporting another nucleic acid to which it has been linked A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression encoding the DNA.

The terms “polypeptide” and “protein” are used interchangeably herein. A “peptide” is a relatively short polypeptide, typically between 2 and 60 amino acids in length, e.g., between 5 and 50 amino acids in length. Polypeptides (typically over 60 amino acids in length) and peptides described herein may be composed of standard amino acids (i.e., the 20 L-alpha-amino acids that are specified by the genetic code, optionally further including selenocysteine and/or pyrrolysine). Polypeptides and peptides may comprise one or more non-standard amino acids. Non-standard amino acids can be amino acids that are found in naturally occurring polypeptides, e.g., as a result of post-translational modification, and/or amino acids that are not found in naturally occurring polypeptides. Polypeptides and peptides may comprise one or more amino acid analogs known in the art can be used. Beta-amino acids or D-amino acids may be used. One or more of the amino acids in a polypeptide or peptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated may still be referred to as a “polypeptide”. Polypeptides may be purified from natural sources, produced in vitro or in vivo in suitable expression systems using recombinant DNA technology, synthesized through chemical means such as conventional solid phase peptide synthesis and/or using methods involving chemical ligation of synthesized peptides. The term “polypeptide sequence” or “peptide sequence” or “amino acid sequence” as used herein can refer to the polypeptide material itself or the peptide material itself and/or to the sequence information (i.e. the succession of letters or three letter codes used as abbreviations for amino acid names) that biochemically characterizes a polypeptide. Polypeptide sequences herein are presented in an N-terminal to C-terminal direction unless otherwise indicated.

The term “analog” as used herein refers to a composition that retains the same structure or function (e.g., binding to a receptor) as a polypeptide or nucleic acid herein. Examples of analogs include peptidomimetics, peptide nucleic acids, small and large organic or inorganic compounds, as well as derivatives and variants of a polypeptide or nucleic acid herein. The term “analog” as used herein of anti-amyloid peptide, such as an anti-amyloid peptide immunogens as disclosed herein, for example SEQ ID NOs: 11-18 and 27-90 or any peptide derived from SEQ ID NO:1 or 2 refers to a molecule similar in function to either the entire molecule of a fragment thereof. The term “analogue” is intended to include allelic, species and variants. Analogs typically differ from naturally occurring peptides at one or a few positions, often by virtue of conservative substitutions. Analogs typically exhibit at least 80 or 90% sequence identity with the natural peptides or the peptide sequence they are an analogue of. In some embodiments, analogs also include unnatural amino acids or modifications of N or C terminal amino acids. Examples of unnatural amino acids are acedisubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, δ-N-methylarginine. Fragments and analogs can be screened for prophylactic or therapeutic efficacy or ability to inhibit or reduce maintenance of amyloid formation as described herein in the Examples. The terms “analogs” and “analogues” are used interchangeably herein.

The term “variant” as used herein refers to any polypeptide or peptide differing from a naturally occurring polypeptide by amino acid insertion(s), deletion(s), and/or substitution(s), created using, e.g., recombinant DNA techniques. In some embodiments amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, tyrosine, asparagine, and glutamine. The positively charged (basic) amino acids include arginine, lysine and histidine. The negatively charged (acidic) amino acids include aspartic acid and glutamic acid. In some embodiments cysteine is considered a non-polar amino acid. In some embodiments insertions or deletions may range in size from about 1 to 20 amino acids, e.g., 1 to 10 amino acids. In some instances larger domains may be removed without substantially affecting function. In certain embodiments, the sequence of a variant can be obtained by making no more than a total of 1, 2, 3, 5, 10, 15, or 20 amino acid additions, deletions, or substitutions to the sequence of a naturally occurring polypeptide. In some embodiments, not more than 1%, 5%, 10%, or 20% of the amino acids in a peptide, polypeptide or fragment thereof are insertions, deletions, or substitutions relative to the original polypeptide. In some embodiments, guidance in determining which amino acid residues may be replaced, added, or deleted without eliminating or substantially reducing activities of interest, may be obtained by comparing the sequence of the particular polypeptide with that of orthologous polypeptides from other organisms and avoiding sequence changes in regions of high conservation or by replacing amino acids with those found in orthologous sequences since amino acid residues that are conserved among various species may more likely be important for activity than amino acids that are not conserved.

The term “derivative” as used herein refers to peptides which have been chemically modified by techniques such as adding additional side chains, ubiquitination, labeling, pegylation (derivatization with polyethylene glycol), and insertion, deletion or substitution of amino acids, including insertion, deletion and substitution of amino acids and other molecules (such as amino acid mimetics or unnatural amino acids) that do not normally occur in the peptide sequence that is basis of the derivative, for example but not limited to insertion of ornithine which do not normally occur in human proteins. The term “derivative” is also intended to encompass all modified variants of the anti-amyloid peptide, variants, functional derivatives, analogues and fragments thereof, as well as peptides with substantial identity as compared to the reference peptide to which they refer to. The term derivative is also intended to encompass aptamers, peptidomimetics and retro-inverso peptides of the reference peptide to which it refers to. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size.

Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative.

A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A. and Viscomi, G. C., J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides or amino acid residues, usually from about 75% to 99%, and more preferably at least about 98 to 99% of the nucleotides or amino acid residues. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan. Homologous sequences can be the same functional gene in different species.

The term “substantial identity” as used herein refers to two peptide sequences, when optimally aligned, such as by the programs GAP or BESTFIT using default gap weights, share at least about 65%, at least about 70%, at least about 80%, at least about 90% sequence identity, at least about 95% sequence identity or more (e.g., 99% sequence identity or higher). In some embodiments, residue positions which are not identical differ by conservative amino acid substitutions.

A “glycoprotein” as use herein is protein to which at least one carbohydrate chain (oligopolysaccharide) is covalently attached. A “proteoglycan” as used herein is a glycoprotein where at least one of the carbohydrate chains is a glycosaminoglycan, which is a long linear polymer of repeating disaccharides in which one member of the pair usually is a sugar acid (uronic acid) and the other is an amino sugar.

The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The term “statistically significant” or “significantly” refers to statistical significance and generally means a two standard deviation (2 SD) below normal, or lower, amount of the amyloid aggregates or incidence of biofilm formation caused by bacteria infection. The term refers to statistical evidence that there is a difference. It is defined as the probability of making a decision to reject the null hypothesis when the null hypothesis is actually true. The decision is often made using the p-value.

Other than in the operating examples, or where otherwise indicated, all numbers expressing quantities of ingredients or reaction conditions used herein should be understood as modified in all instances by the term “about.” The term “about” when used in connection with percentages can mean±1%.

The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Anti-Amyloid Peptides and Pro-Amyloid Peptides

Some aspects of the invention encompasses an anti-amyloid peptide engineered bacteriophage which express at least one anti-amyloid peptide that inhibit amyloid aggregation or express at least one variant of anti-amyloid peptides that inhibits amyloid aggregation.

One aspect of the present invention relates to anti-amyloid peptide engineered bacteriophages which express at least one anti-amyloid peptide whose sequence comprises or consists of a fragment of the sequence of a naturally occurring bacterial CsgA polypeptide or a CsgB polypeptide, and compositions and uses thereof. In another aspect, the present invention relates to anti-amyloid peptide engineered bacteriophages which express at least one anti-amyloid peptide whose sequence comprises or consists of a variant of a fragment of the sequence of a naturally occurring bacterial CsgA polypeptide or a CsgB polypeptide, and compositions and uses thereof.

In another aspect, the present invention relates to anti-amyloid peptide engineered bacteriophages which express at least one anti-amyloid peptide whose sequence comprises or consists of a fragment of the sequence of a variant of a CsgA polypeptide or a variant of a CsgB polypeptide, and compositions and uses thereof.

Such amyloid peptide, e.g, an anti-amyloid peptide or a pro-amyloid peptide expressed by an anti-amyloid peptide engineered bacteriophages or pro-amyloid peptide may bind to a polypeptide, e.g., a CsgA polypeptide, and where the amyloid peptide is a anti-amyloid peptide, prevent the CsgA polypeptide from being added to a growing aggregate or the anti-amyloid peptide can bind to polypeptides within a growing aggregate and thereby inhibit binding of additional polypeptides to the aggregate. An anti-amyloid peptide expressed by the bacteriophage is a moiety that inhibits or disrupts aggregate formation, e.g., fiber assembly. In alternative embodiments, where the amyloid peptide is a pro-amyloid peptide, the pro-amyloid peptide promotes addition of the CsgA polypeptide to a growing aggregate or the pro-amyloid peptide can bind to polypeptides within a growing aggregate and thereby increase the occurance of binding of additional polypeptides to the aggregate. A pro-amyloid peptide expressed by the bacteriophage is a moiety that increases aggregate formation, e.g., increases fiber assembly.

In some embodiments, an anti-amyloid peptide engineered bacteriophage as disclosed herein expresses an anti-amyloid peptide which inhibits amyloid formation on biofilms, where for example, the anti-amyloid is derived from, or is a modified version of a peptide derived from a polypeptide that promotes the formation of a biofilm. In some embodiments, an anti-amyloid peptide engineered bacteriophage as disclosed herein expresses an anti-amyloid peptide derived from a first or a second amyloidogenic polypeptide, where the first and second amyloidogenic polypeptides are at least 70%, 80%, 85%, 90%, or 95% identical to polypeptides that assemble to form amyloids present in biofilms e.g., bacterial polypeptides that assemble to form amyloid fibers such as curli. Curli are the major proteinaceous component of a complex extracellular matrix produced by many bacteria, e.g., many Enterobacteriaceae such as E. coli and Salmonella spp. (Barnhart M M, Chapman M R. Annu Rev Microbiol., 60:131-47, 2006). Other biofilm-forming bacteria of interest include Klebsiella, Pseudomonas, Enterobacter, Serratia, Citrobacter, Proteus, Yersinia, Citrobacter, Shewanella, Agrobacter, Campylobacter, etc. Curli fibers are involved in adhesion to surfaces, cell aggregation, and biofilm formation. Curli also mediate host cell adhesion and invasion, and they are potent inducers of the host inflammatory response. Curli exhibit structural and biochemical properties of amyloids, e.g., they are nonbranching, β-sheet rich fibers that are resistant to protease digestion and denaturation by 1% SDS and bind to amyloid-specific moieties such as thioflavin T, which fluoresces when bound to amyloid, and Congo red, which produces a unique spectral pattern (“red shift”) in the presence of amyloid. Polypeptides that assemble to form curli are of interest at least in part because of their association with animal and human disease. Bacterial polypeptides that promote formation of biofilms present in a variety of natural habitats are also of interest. For example, in a recent study bacteria producing extracellular amyloid adhesins were identified within several phyla: Proteobacteria (Alpha-, Beta-, Gamma- and Deltaproteobacteria), Bacteriodetes, Chloroflexi and Actinobacteria (Larsen, P., et al., Environ Microbiol., 9(12):3077-90, 2007). Particularly in drinking water biofilms, a high number of amyloid-positive bacteria were identified. Bacteria of interest may be gram-negative or gram-positive. In some embodiment bacteria of interest are rods. In some embodiments they are aerobic. In some embodiments they are facultative anaerobes or anaerobes.

In nature, curli are assembled by a process in which the major curli subunit polypeptide, CsgA, is nucleated into a fiber by the minor curli subunit polypeptide, CsgB. CsgA and CsgB are about 30% identical at the amino acid level and contain five-fold internal symmetry characterized by conserved polar residues. The assembly process is believed to involve addition of soluble polypeptides to the growing fiber tip. Thus both subunits are incorporated into the fiber, although CsgA is the major protein constituent and CsgB is the nucleating polypeptide. Sequences of CsgA and CsgB from a large number of bacteria have been identified. Exemplary CsgA and CsgB amino acid sequences are shown in FIGS. 4A (SEQ ID NO:1) and 5A (SEQ ID NO: 2), respectively. One of skill in the art will readily be able to find CsgA and CsgB sequences by searching databases such as GenBank publicly available through the National Center for Biotechnology Information (NCBI) (see ncbi.nlm.nih.gov), and they are encompassed for use in generating anti-amyloid peptides to inhibit curli formation in the methods and bacteriophages as disclosed herein.

The present invention is based in part on the discovery that small peptides of bacterial CsgB can be used to inhibit curli fiber formation. Further, it was found that these sequence elements mimic the in vivo assembly of curli fibers in that, peptides whose sequence is found within the sequence of CsgB or CsgA efficiently nucleated assembly of CsgA into amyloid. As described in the Examples, specific peptides within E. coli CsgB and CsgA inhibited amyloid fiber formation when they were expressed on the surface of bacteriophages. Accordingly, the inventors demonstrated that short peptide portions of bacterial biofilm forming proteins bind directly to full length polypeptides and inhibit form higher order aggregates, e.g., fibrils. Notably, the results demonstrate that specific anti-amyloid peptides can be expressed by a bacteriophage and effectively used to inhibit amyloid fiber assembly. These anti-amyloid peptide engineered bacteriophages, compositions comprising the anti-amyloid peptide engineered bacteriophages, and uses thereof are aspects of the invention.

The invention also provide a plurality of different anti-amyloid peptide engineered bacteriophages, and related compositions and methods disclosed herein, wherein anti-amyloid peptide engineered bacteriophages expresses at least one CsgB peptide and/or at least one CsgA peptide, as those terms are defined herein.

In some embodiments, an anti-amyloid peptide engineered bacteriophage expresses at least one CsgA peptide, which is a peptide whose sequence comprises a portion of a CsgA polypeptide sequence (SEQ ID NO:1) and/or expresses at least one CsgB peptide, which is a peptide whose sequence comprises a portion of CsgB polypeptide sequence (SEQ ID NO: 2). Examples of such peptide are listed in Tables 3 (SEQ ID NO: 11-18) and 4 respectively (SEQ ID NO: 27-34). Examples of variants of CsgA peptides include, but are not limited to SEQ ID NO: 35-58, and examples of variants of CsgB peptides include, but are not limited to SEQ ID NO: 59-90, as disclosed in Table 5.

In certain embodiments, in addition to a portion of a CsgA or CsgB polypeptide sequence, a CsgA peptide and/or CsgB peptide can further comprise one or more additional amino acids, e.g., one or more alanine or lysine residues (e.g., a double alanine tag, a double lysine tag, etc.), which may be located at the N- or C-terminus of the portion of the CsgA or CsgB sequence. Without limitation, such additional residues may be useful for expression and/or secretion of the anti-amyloid peptide (i.e. CsgA and/or CsgB peptide) or attaching the anti-amyloid peptides (i.e. CsgA and/or CsgB peptide) to the surface of the bacteriophage. Examples of such variant CsgA peptides and CsgB peptide which can be expressed by the bacteriophage are listed in Table 5 (SEQ ID NO: 35-90).

In some embodiments, a CsgA peptide and/or CsgB peptide can comprise a portion of a CsgA or CsgB polypeptide where at least one amino acid is modified (i.e. substituted or added or deleted). Without limitation, such modified amino acids enhance the efficacy of the anti-amyloid peptide to inhibit the formation or maintenance of amyloids. Examples of such variant CsgA peptides and CsgB peptide which can be expressed by the bacteriophage are listed in Table 5 (SEQ ID NO: 35-90).

In some embodiments an anti-amyloid peptide engineered bacteriophage encodes at least one anti-amyloid peptide, such as a CsgA peptide or a CsgB peptide, where a CsgA peptide is selected from SEQ ID NOs: 11-18 or SEQ ID NOs: 35-58 or variants or modified variants thereof, and a CsgB peptide is selected from SEQ ID NOs: 27-34 or SEQ ID NOs: 59-90, or variants or modified variants thereof.

In some embodiments an anti-amyloid peptide engineered bacteriophage encodes at least one anti-amyloid peptide, such as a CsgA peptide or a CsgB peptide, where a CsgA peptide is selected from the group of SEQ ID NOs: 83 to 130, or variants or modified variants thereof.

In some embodiments an anti-amyloid peptide engineered bacteriophage encodes at least one anti-amyloid peptide, such as a CsgA peptide or a CsgB peptide, where a CsgA peptide is selected from any of the group of SEQ ID NOs: 12, 16, 52 or 53 and the CsgB peptide is selected from any of SEQ ID NOs: 29, 33 or 61-65.

In some embodiments an anti-amyloid peptide engineered bacteriophage encodes at least one anti-amyloid peptide, such as a CsgA peptide or a CsgB peptide, where a CsgA peptide is selected from the CsgA III class of peptides (SEQ ID NO: 52-53), or from the CsgAIIb class of peptides (SEQ ID NOs:35, 36, 39-41, 45, 49-51), or from the CsgAIIa class of peptide (SEQ ID NO: 11 and 12) or from the CsgAI class of peptides (SEQ ID NOs: 42, 44, 46, 57 and 58).

In some embodiments an anti-amyloid peptide engineered bacteriophage encodes at least one anti-amyloid peptide, such as a CsgA peptide or a CsgB peptide, where a CsgA peptide is selected from selected from the CsgBIII class of peptides (SEQ ID NOs: 61-65) or from the CsgBIIb class of peptides (SEQ ID NOs: 59, 60, 69, 75, 81, 93 and 94) or from the CsgBIIa class of peptides (SEQ ID NO: 29) or from CsgBI class of peptides (SEQ ID NOs: 66-68 and 70-72).

In a preferred embodiment, an anti-amyloid peptide engineered bacteriophage encodes at least one anti-amyloid peptide, such as a CsgA peptide or a CsgB peptide, where a CsgA peptide is selected from the CsgAIII group of peptides (SEQ ID NO: 52, 53) or CsgBIII peptides (SEQ ID NOs: 61-65).

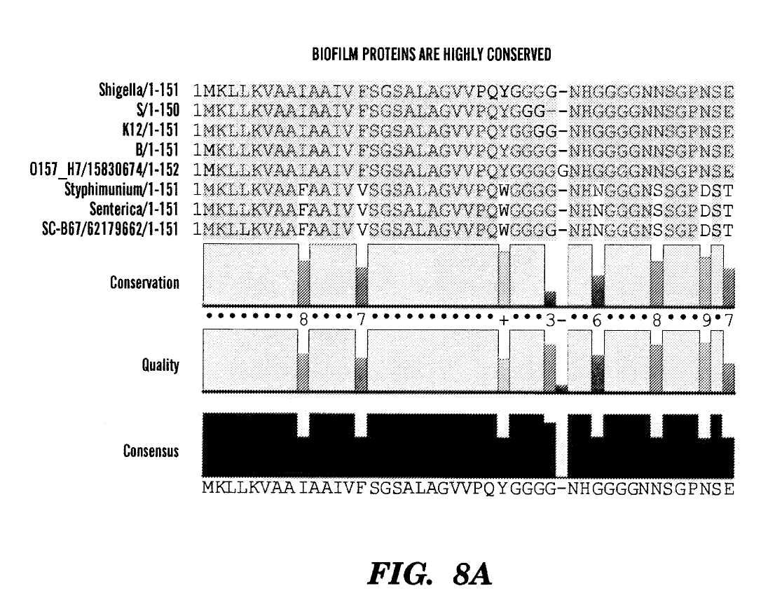

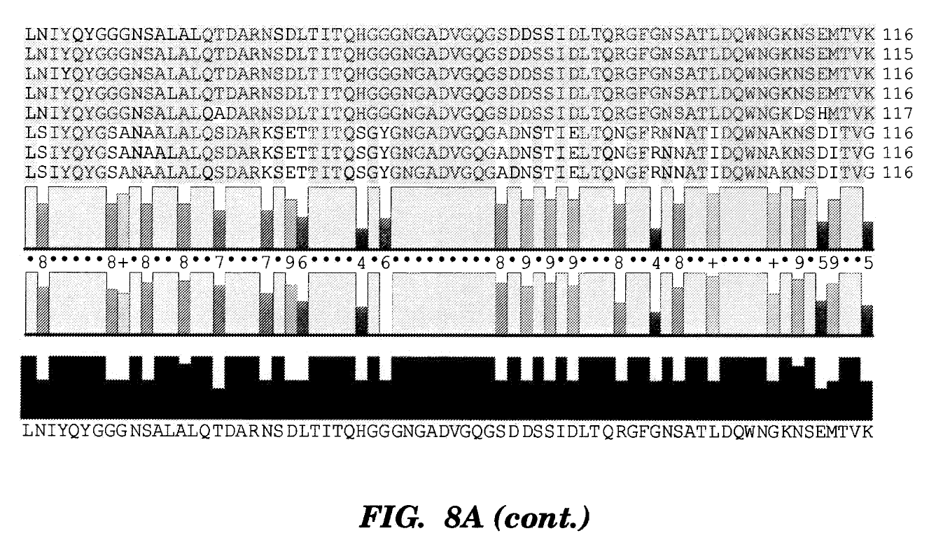

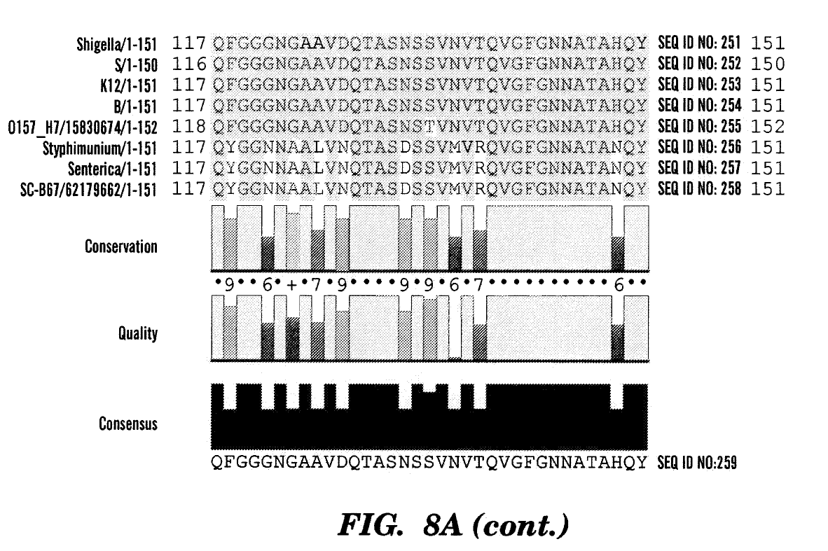

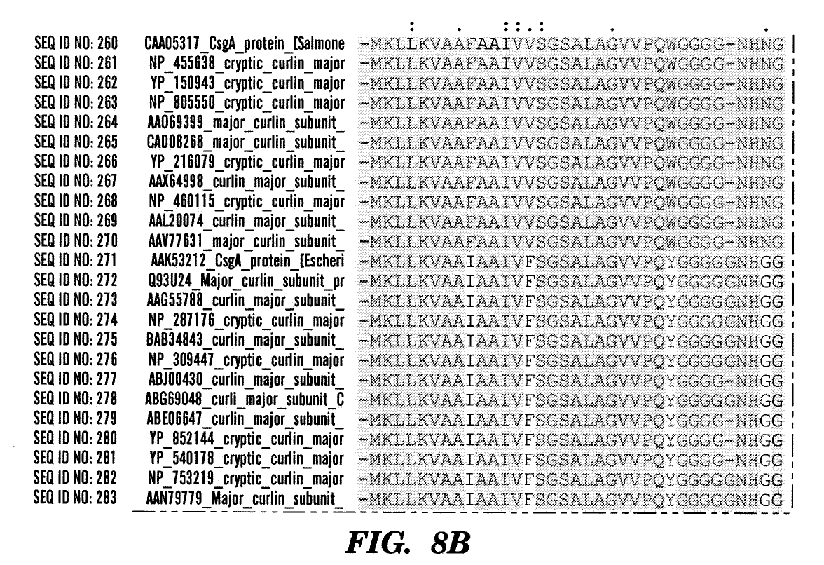

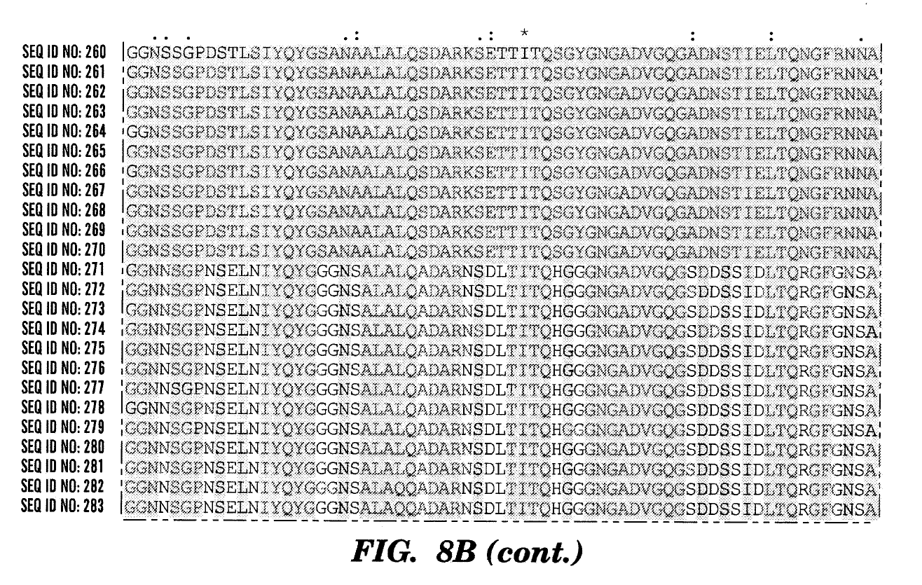

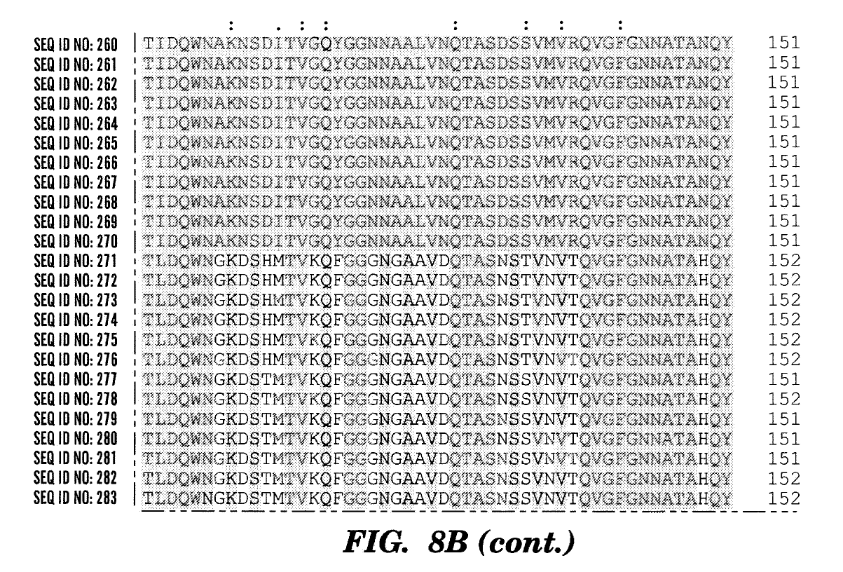

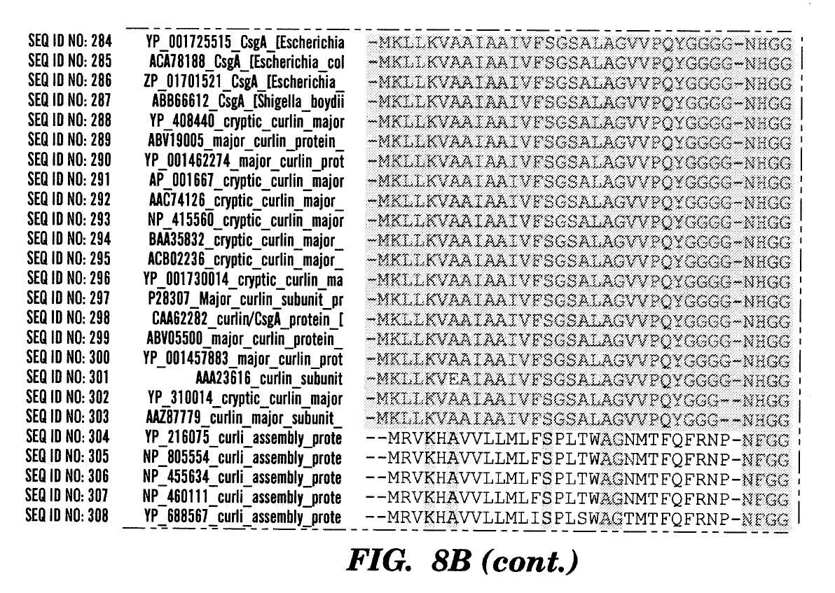

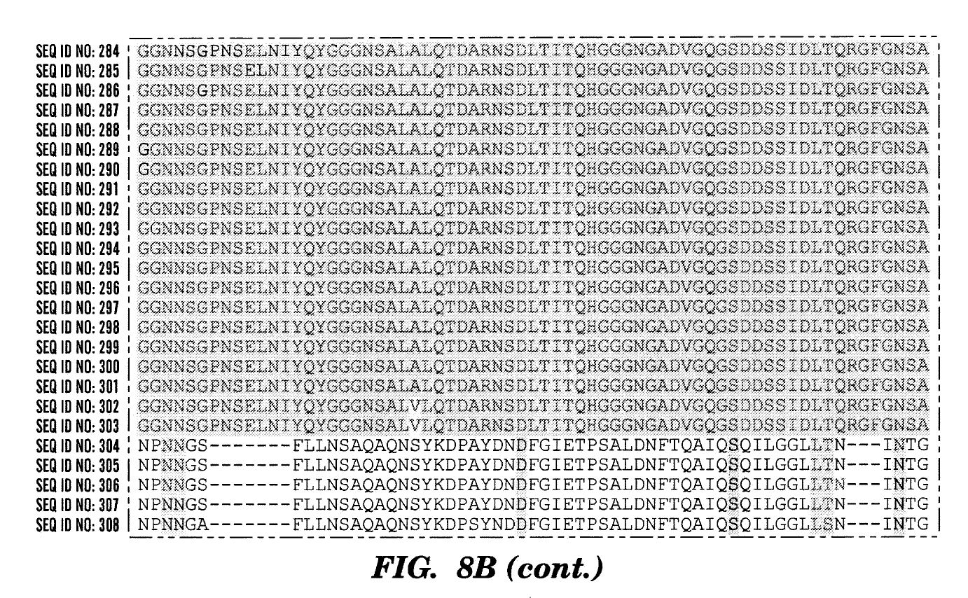

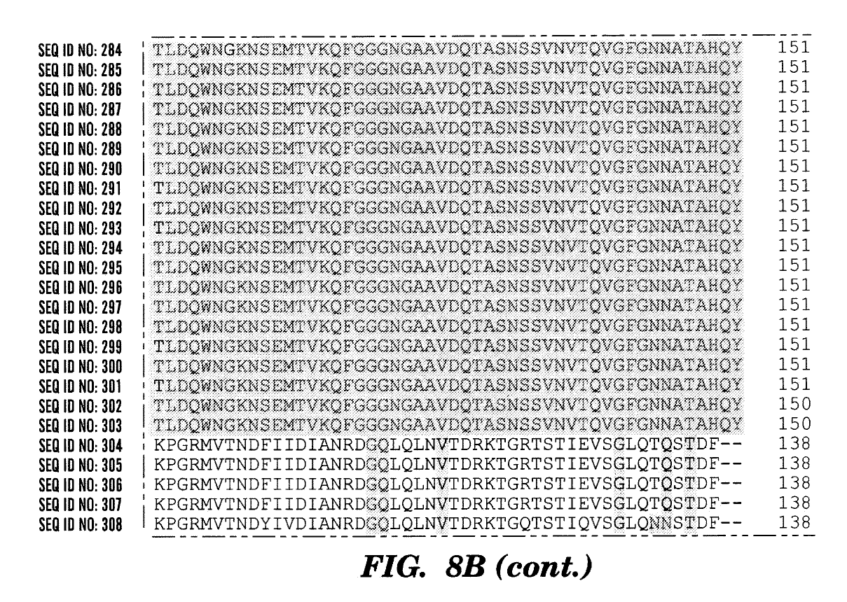

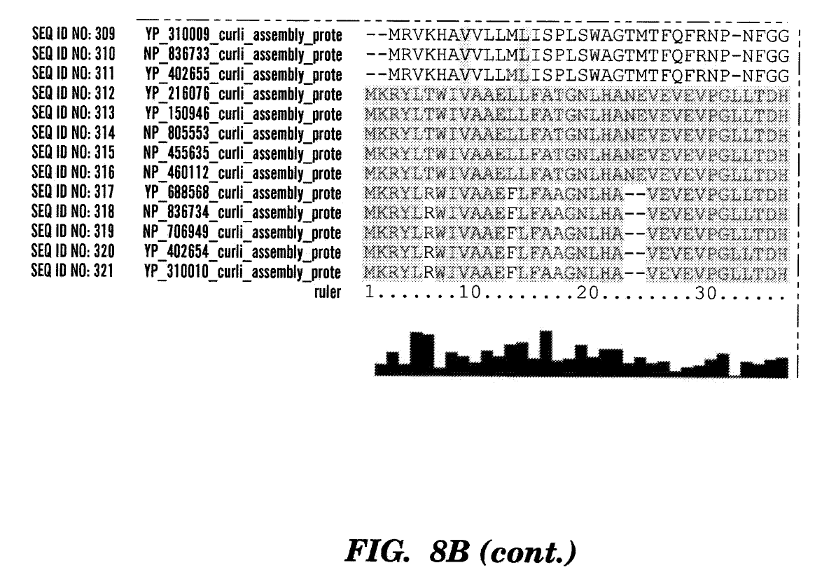

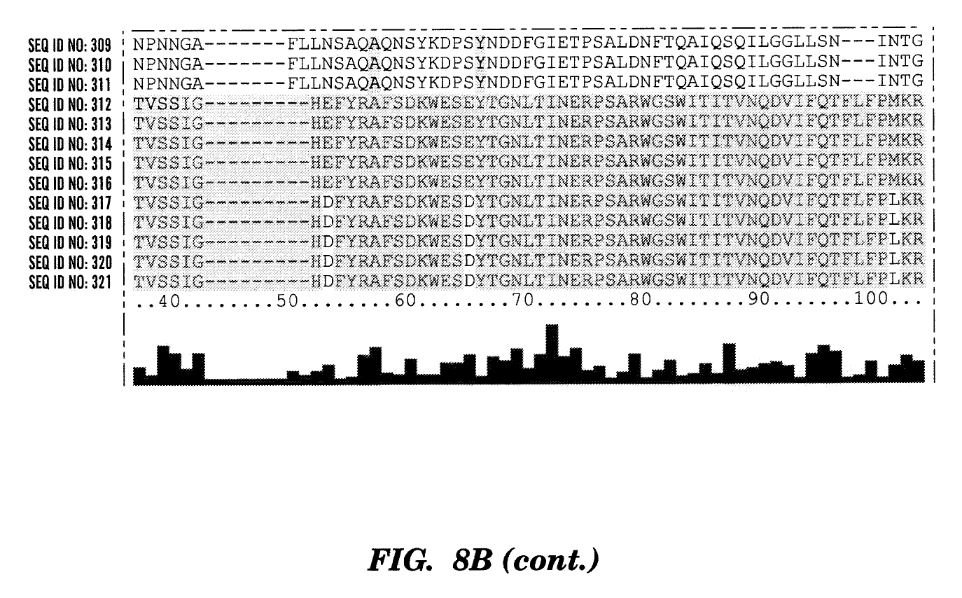

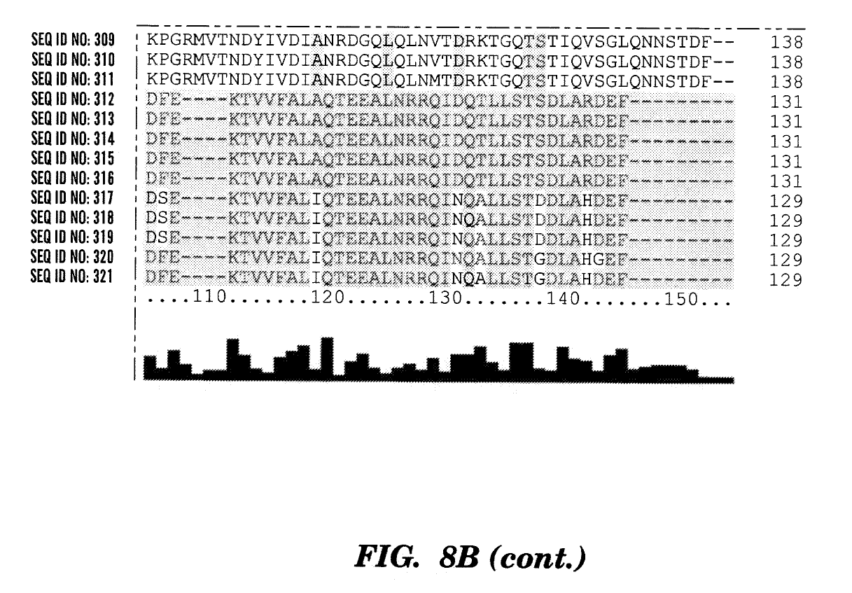

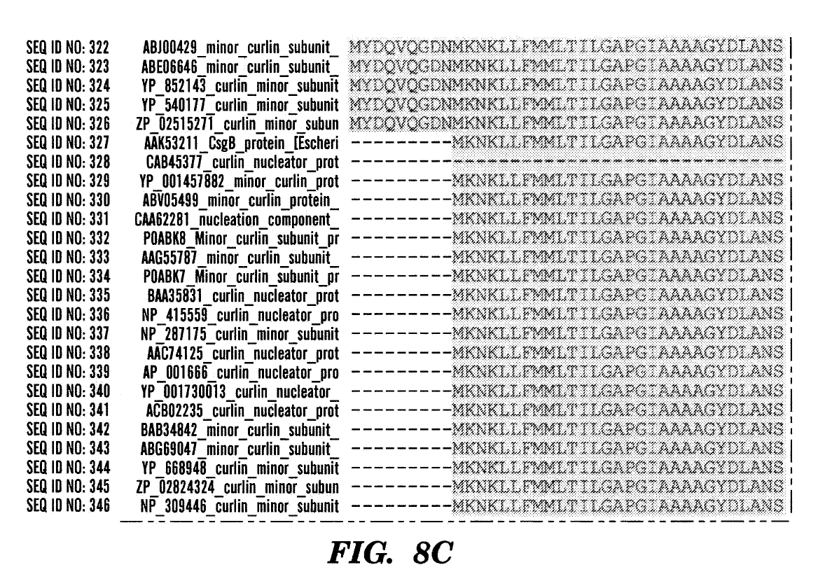

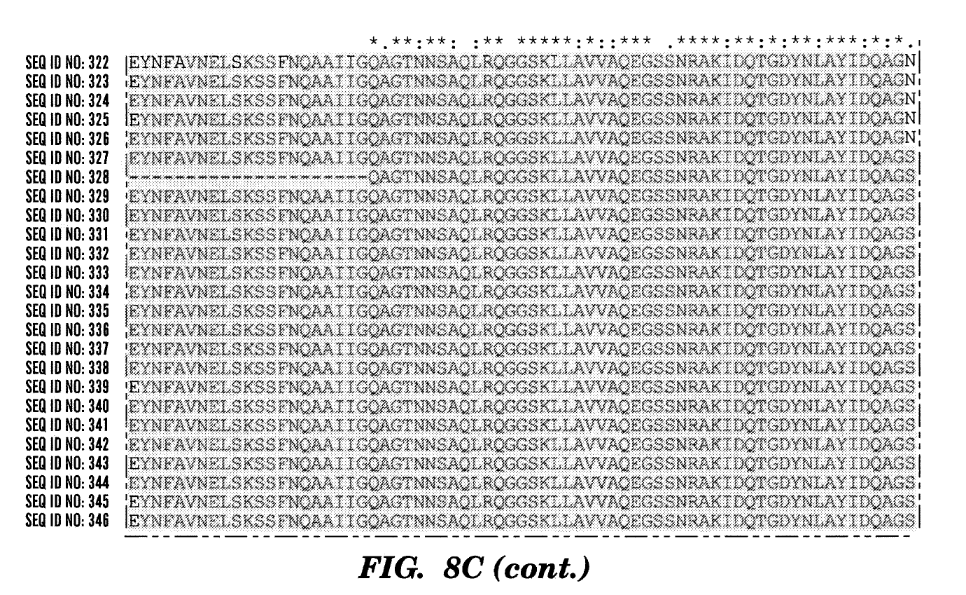

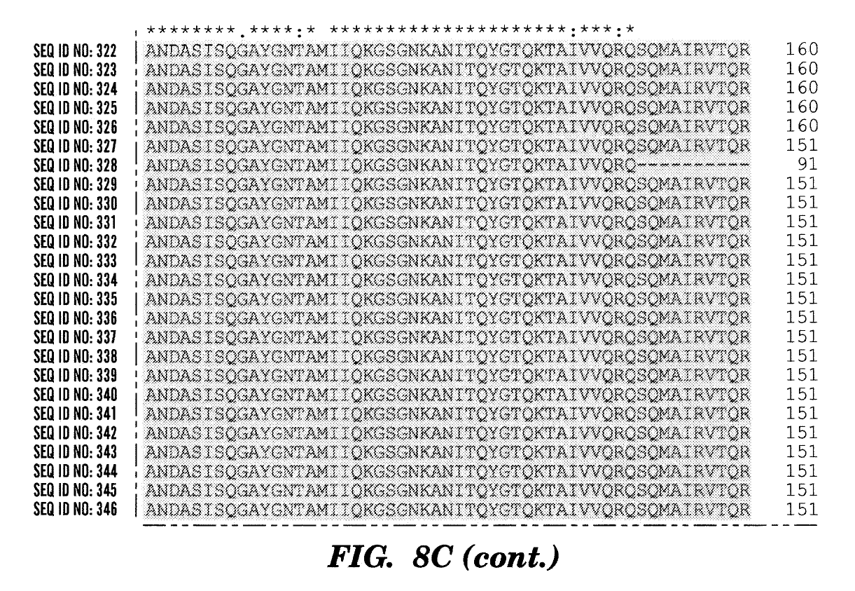

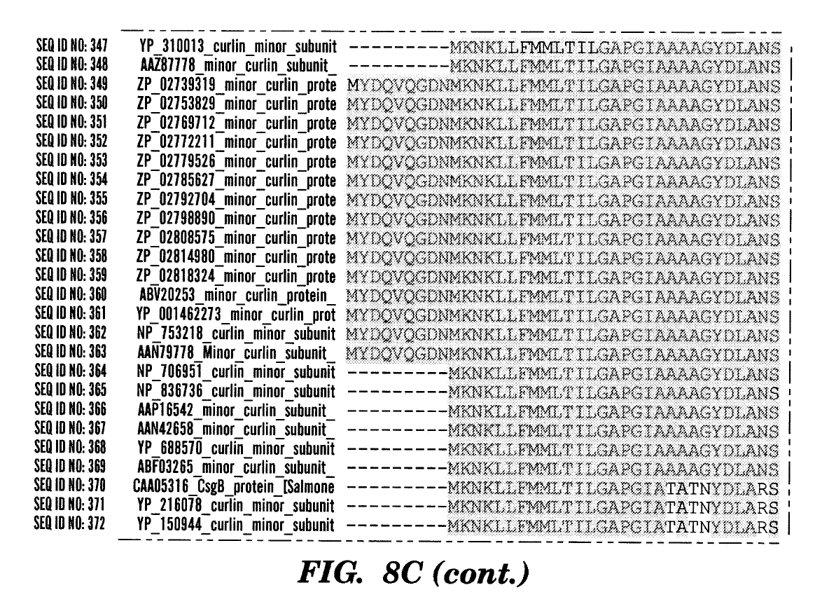

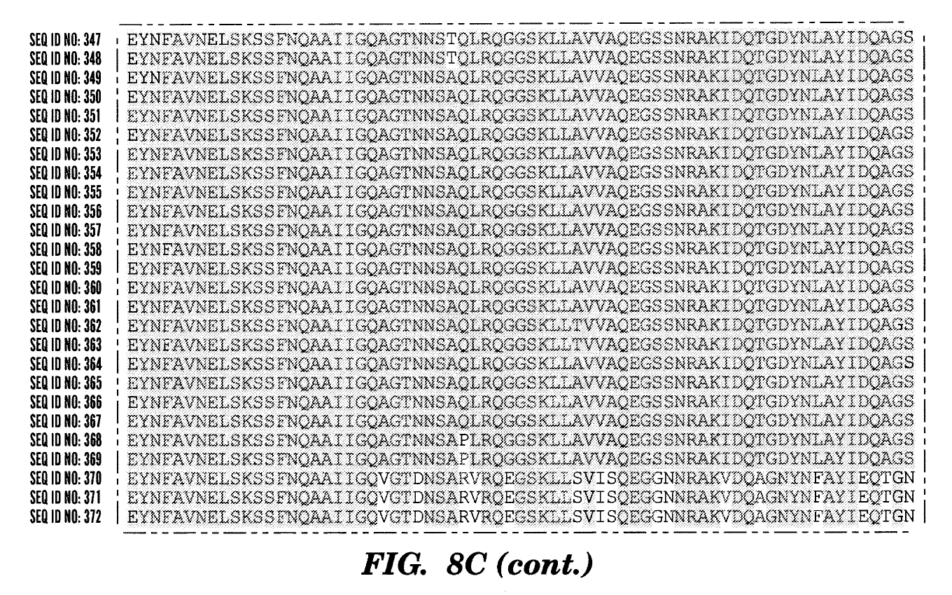

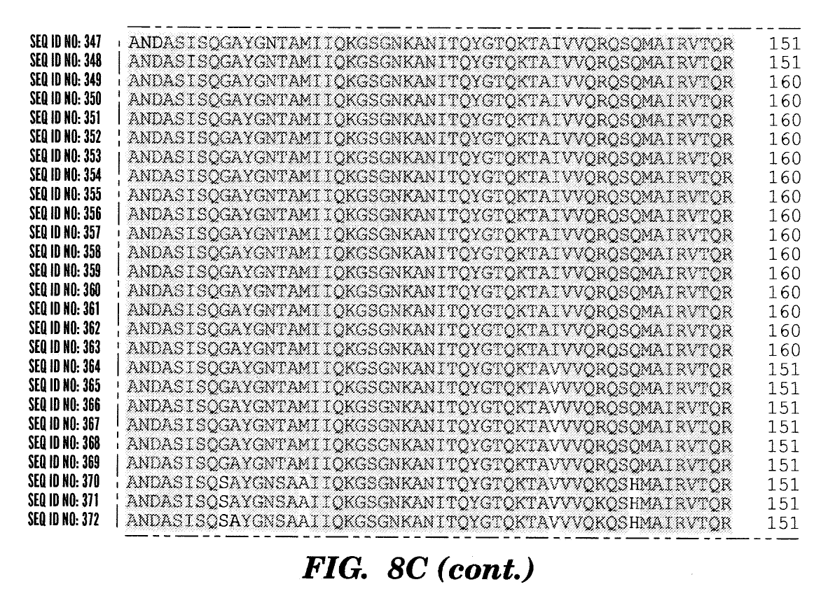

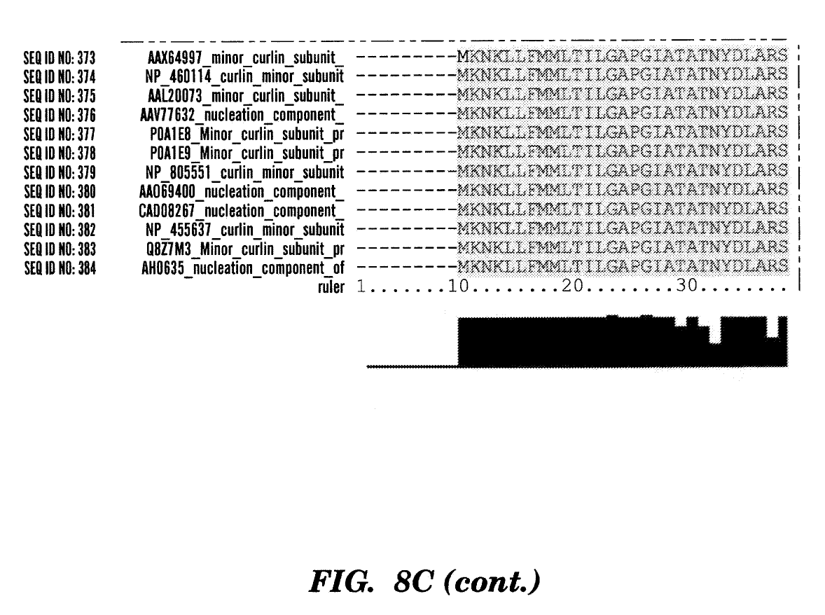

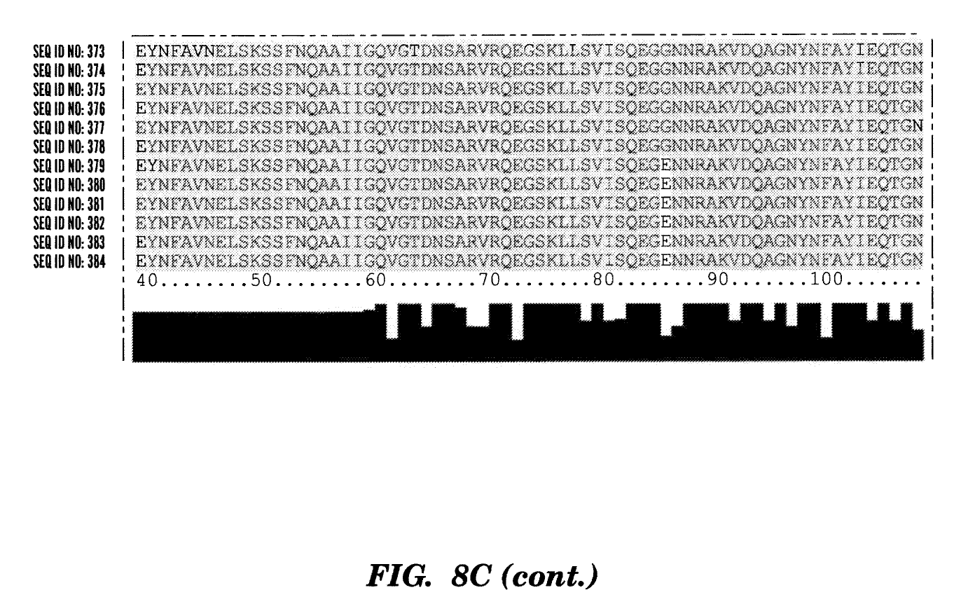

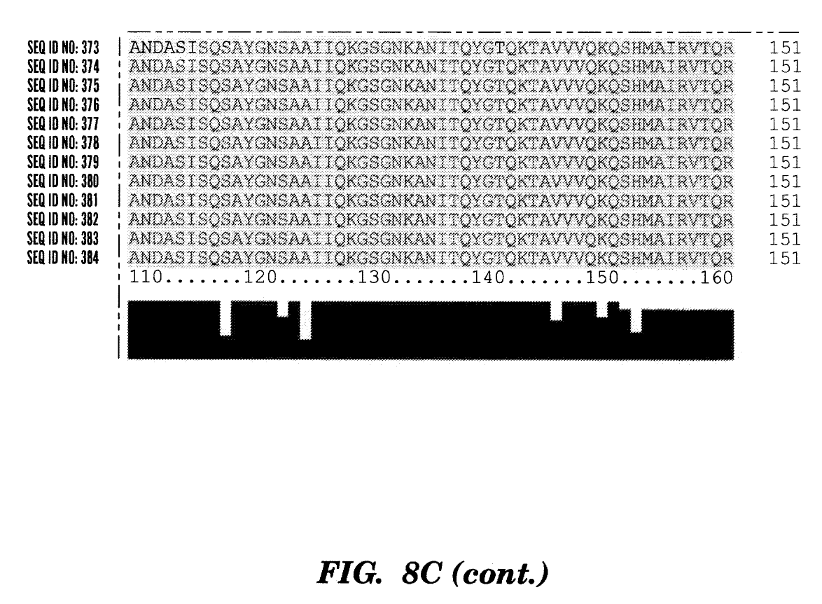

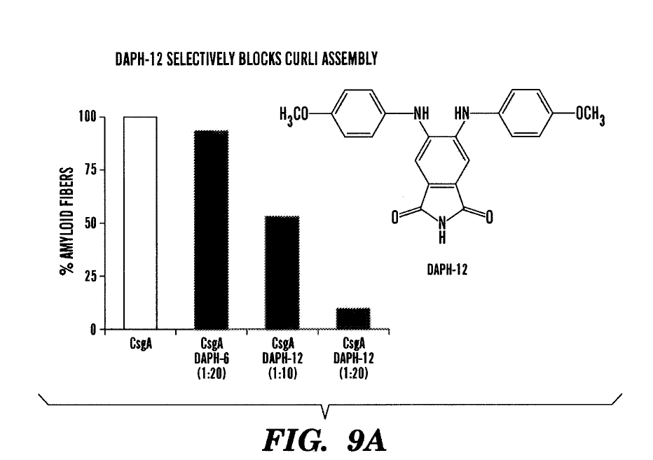

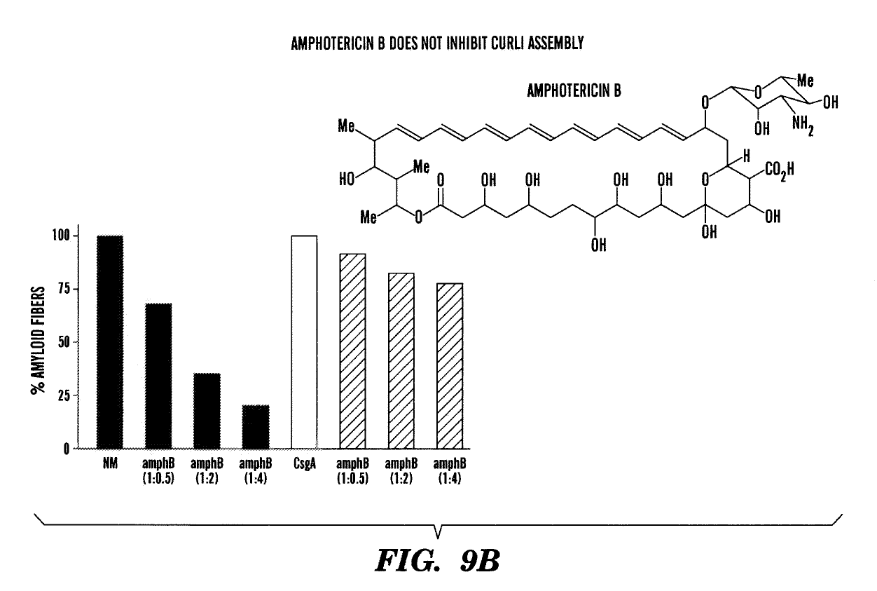

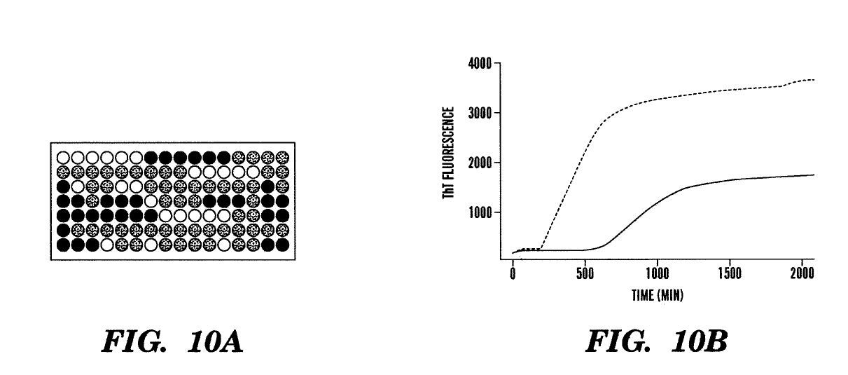

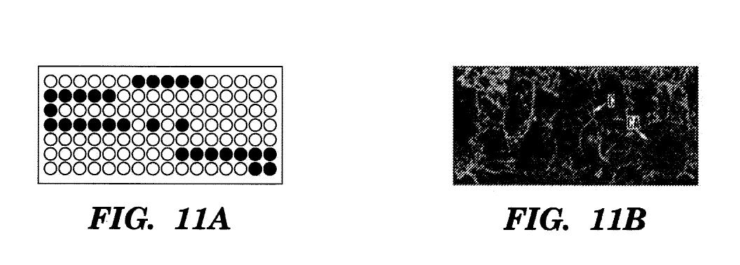

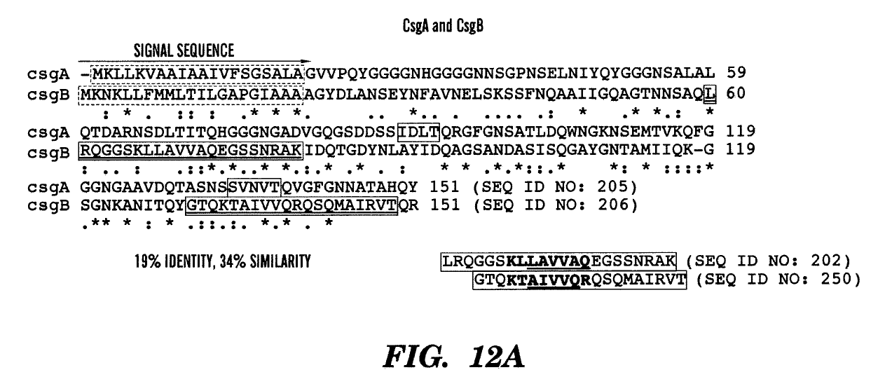

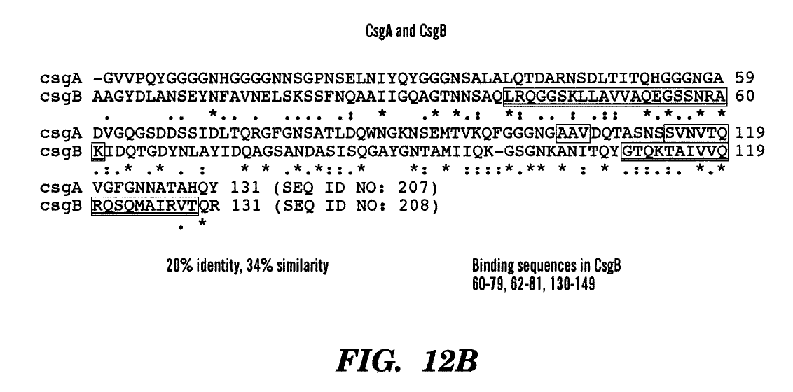

In some embodiments an anti-amyloid peptide engineered bacteriophage encodes at least one anti-amyloid peptide, wherein the anti-amyloid peptide comprises a fragment of at least 5, or at least 6 or at least 7 concecutive amino acids from SEQ ID NO: 1 or SEQ ID NO: 2. In other embodiments, an anti-amyloid peptide engineered bacteriophage encodes at least one anti-amyloid peptide, wherein the anti-amyloid peptide is derived from any of SEQ ID NOs: 61, 62, 63, 64 or 65, or any fragment of a protein involved in biofilm formation as shown in FIG. 8A. In other embodiments, an anti-amyloid peptide engineered bacteriophage encodes at least one anti-amyloid peptide, wherein the anti-amyloid peptide is derived from any polypeptide listed in FIG. 8B or 8C, or any fragment of a protein involved in biofilm formation as shown in FIG. 8B or 8C.

In addition to CsgA and CsgB, curli formation likely involves activities of several additional polypeptides encoded by other Csg genes (CsgD, CsgE, CsgF, CsgG) in living bacteria, but these polypeptides are not required for curli formation in vitro. Thus, in some embodiments, an anti-amyloid peptide engineered bacteriophage can encode at least a fragment of a different Csg polypeptide selected from the group comprising CsgD, CsgR, CsgF and CsgG polypeptides.

The invention also provides a composition comprising at least one anti-amyloid peptide engineered bacteriophage expressing at least one CsgA peptide and/or at least one CsgB peptide as disclosed herein.

The invention provides compositions comprising at least one anti-amyloid peptide engineered bacteriophage expressing any of the foregoing CsgA peptides or CsgB peptides.

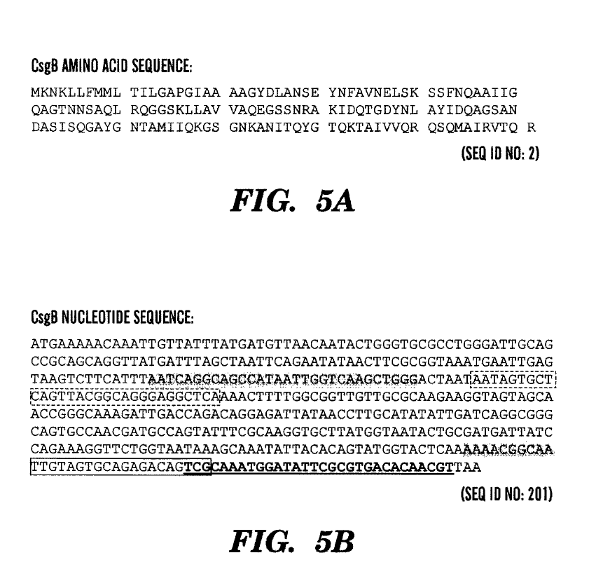

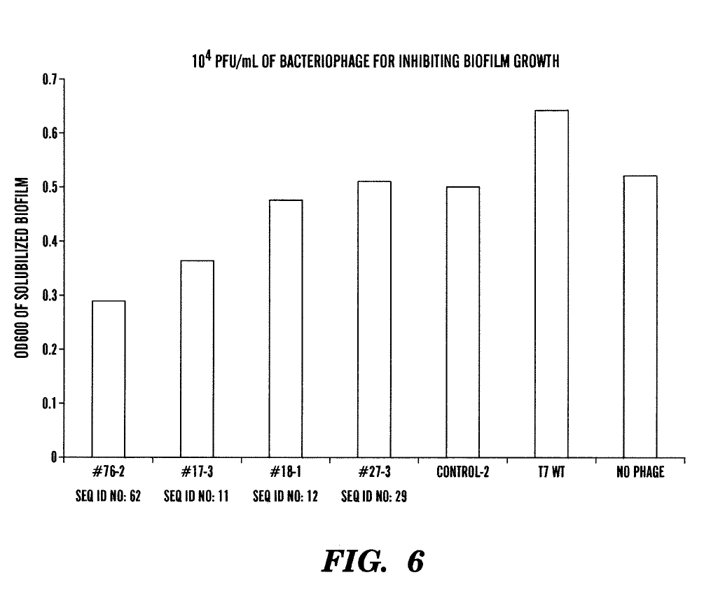

FIGS. 4A and 5A show certain CsgA and CsgB sequences of use in the present invention and accession numbers thereof. Anti-amyloid peptides encompassed to be expressed by the anti-amyloid peptide engineered bacteriophages comprise or consist of these amino acid sequences or portions thereof which are capable of nucleating aggregation of CsgA. It will be appreciated that peptides of interest can, in certain embodiments, encompass the minimal nucleating sequences and additional sequences on one or both ends.

Exemplary CsgB peptides to be expressed by an anti-amyloid peptide engineered bacteriophage have a sequence that comprises or consists of a sequence falling within amino acids 50-90 or 120-160 of E. coli CsgB, or within the corresponding amino acids within CsgB from other bacterial species. Exemplary CsgB peptide sequences include amino acids 55-75 or 125-155 of CsgB, or a portion of the afore-mentioned sequences. Specific examples of 25 amino acid CsgB peptides include, e.g., peptides having the sequence of amino acids 57-81, 58-82, 59-83, 60-84, 61-85, 62-86, 63-87, 125-149, 126-150, 127-151, 128-152, 129-153, 130-154, etc., of CsgB. Specific examples of 23 amino acid CsgB peptides include, e.g., peptides having the sequence of amino acids 58-80, 59-81, 60-82, 61-83, 62-84, 63-87, 127-149, 128-150, 129-151, 130-152, 131-153, 132-154, etc., of CsgB. Specific examples of 22 amino acid CsgB peptides include, e.g., peptides having the sequence of amino acids 59-80, 60-81, 61-82, 62-83, 129-150, 130-151, 131-152, etc., of CsgB. Specific examples of 21 amino acid CsgB peptides include, e.g., peptides having the sequence of amino acids 59-79, 60-80, 61-81, 62-82, 129-149, 130-150, 131-151, etc., of CsgB. Specific examples of 20 amino acid CsgB peptides include, e.g., peptides having the sequence of amino acids 60-79, 61-80, 62-81, 130-149, 131-150, etc., of CsgB polypeptide.

The following CsgB peptides to be expressed by a bacteriophage are exemplary: (i) LRQGGSKLLAVVAQEGSSNRAK (SEQ ID NO: 202) (CsgB 60-81); (ii) GTQKTAIVVQRQSQMAIRVT (SEQ ID NO: 250) (CsgB 130-149). In some embodiments a peptide comprises at least AIVVQ (SEQ ID NO: 228) and, optionally, one or more additional amino acids found in CsgB at locations N- or C-terminal to AIVVQ. In some embodiments a peptide comprises at least LAVVAQ (SEQ ID NO: 220) and, optionally, 1, 2, 3, 4, 5, 6, or more additional amino acids found in CsgB at locations N- or C-terminal to LAVVAQ (SEQ ID NO: 220), i.e., the peptide could be extended in either or both directions. For example, one such peptide is GGSKLLAVVAQEGSSN (SEQ ID NO: 221). Peptides can comprise KLLAVVAQE (SEQ ID NO: 222) or KTAIVVQR (SEQ ID NO: 223) and, optionally, one or more additional amino acids found in CsgB at locations N- or C-terminal to such peptides, i.e., the peptide could be extended in either or both directions by, for example, 1, 2, 3, 4, 5, or 6 amino acids. For example, one such peptide is TQKTAIVVQRQSQMAIR (SEQ ID NO: 224). In some embodiments a peptide is between 5 and 25 amino acids long, e.g., 6, 7, 8, 9, 10, 11, 12, 13, 14, 15, 16, 176, 18, 19, 20, 21, 22, 23, 24, or 25 amino acids long.

It will be appreciated that SEQ ID NOs: 1 and 2 are found in certain E. coli strains. Minor differences may be encountered in other E. coli strains or in CsgA and CsgB polypeptides from different bacterial genera. Peptides that are orthologs of the afore-mentioned peptides (SEQ ID NOs: 12, 16, 27-34 or SEQ ID NO: 52, 53, and 61-65) in any particular bacterial strain, species, genus, or family are encompassed to be expressed by the anti-amyloid peptide engineered bacteriophage. One of skill in the art will be able to identify such orthologs based on sequence comparisons. Also provided are variants of any of the afore-mentioned peptides (SEQ ID NO: 11-18, 27-90). In some embodiments, a variant of a particular CsgA peptide or CsgB peptide may have 1, 2, or 3 amino acid substitutions, additions, and/or deletions relative to the original peptide. In some embodiments a substitution is a conservative substitution. In some embodiments a polar or hydrophilic amino acid is added or substituted. Optionally the peptides further comprise a tag, detectable moiety, etc. CsgA and/or CsgB peptides may be tested using the methods described herein in the Examples to select those CsgA and/or CsgB peptides or variants or orthologs thereof that may be preferable for use in inhibiting amyloid formation or inhibition of protein aggregation in a subject. The optimal CsgA and/or CsgB peptide may differ depending on various factors such as the subject to be treated, the particular bacteria, or the type of amyloid formation, etc.

Each of the CsgA and/or CsgB peptides as described herein is encompassed for expression by an anti-amyloid peptide engineered bacteriophage. In some embodiments an anti-amyloid peptide engineered bacteriophage expresses at least one CsgA and/or CsgC and/or CsgD and/or CsgE and/or CsgF, and/or CsgB peptide.

Other anti-amyloid peptides are encompassed for use in anti-amyloid peptide engineered bacteriophages as disclosed herein. For example, without limitation, such anti-amyloid peptides include those disclosed in WO2008/033451, which is incorporated herein by reference. In other embodiments, amino acid sequences which can be used to derive anti-amyloid peptides include Self-Coalesces into Higher-Ordered AggreGates (SCHAG) sequences as that term is used in U.S. Ser. No. 11/004,418, which is incorporated herein by reference. By “SCHAG amino acid sequence” is meant any amino acid sequence which, when included as part or all of the amino acid sequence of a protein, can cause the protein to coalesce with like proteins into higher ordered aggregates commonly referred to in scientific literature by terms such as “amyloid,” “amyloid fibers,” “amyloid fibrils,” “fibrils,” or “prions.” It will be understood than many proteins that will self-coalesce into higher-ordered aggregates can exist in at least two conformational states, only one of which is typically found in the ordered aggregates or fibrils. The term “self-coalesces” refers to the property of the polypeptide such as those described herein or known in the art to form ordered aggregates with polypeptides having an identical amino acid sequence under appropriate conditions and is not intended to imply that the coalescing will naturally occur under every concentration or every set of conditions.

In certain embodiments the polypeptide is not Sup35 or a region thereof at least 40 amino acids long, e.g., the N, M, or NM domain. In some embodiments the polypeptide is not SEQ ID NO: 131 of PCT/US2006/022460 (WO 2006/135738). In certain embodiments the peptides are not derived from the foregoing polypeptides.

In other embodiments, an anti-amyloid peptide engineered bacteriophage can comprise a portions of a polypeptide that is prone to aggregation under appropriate conditions (i.e. an “aggregation-prone”) polypeptide. In one embodiment, the aggregation-prone polypeptide is a yeast or fungal prion protein. In another embodiment, the aggregation-prone polypeptide is a mammalian prion protein. In another embodiment, the aggregation-prone polypeptide is any polypeptide known to self-aggregate in vitro or in vivo. In one embodiment the polypeptide is any polypeptide that forms amyloid. In one embodiment the polypeptide is any polypeptide wherein aggregates formed from the polypeptide and/or from fragments of the polypeptide play a role in disease.

Polypeptides and diseases of interest include amyloid β protein, associated with Alzheimer's disease; immunoglobulin light chain fragments, associated with primary systemic amyloidosis; serum amyloid A fragments, associated with secondary systemic amyloidosis; transthyretin and transthyretin fragments, associated with senile systemic amyloidosis and familial amyloid polyneuropathy I; cystatin C fragments, associated with hereditary cerebral amyloid angiopathy; β2-microglobulin, associated with hemodialysis-related amyloidosis; apolipoprotein A-I fragments, associated with familial amyloid polyneuropathy II; a 71 amino acid fragment of gelsolin, associated with Finnish hereditary systemic amyloidosis; islet amyloid polypeptide fragments, associated with Type II diabetes; calcitonin fragments, associated with medullary carcinoma of the thyroid; prion protein and fragments thereof, associated with spongiform encephalopathies; atrial natriuretic factor, associated with atrial amyloidosis; lysozyme and lysozyme fragments, associated with hereditary non-neuropathic systemic amyloidosis; insulin, associated with injection-localized amyloidosis; and fibrinogen fragments, associated with hereditary renal amyloidosis. The polypeptide which can be used to derive an anti-amyloid peptide can be a full length polypeptide or a fragment thereof that self-assembles to form an aggregate.

Other anti-amyloid peptides to be expressed by anti-amyloid peptide engineered bacteriophages as disclosed herein can be derived from any amyloid protein or polypeptide or any polypeptide which makes up a high ordered aggregate as that term is defined herein. For example, high ordered aggregates which can be used to derived anti-amyloid peptides to be expressed by an anti-amyloid peptide engineered bacteriophages as disclosed include polypeptides such as Sup35 proteins, Ure2 proteins, New1 proteins, Rnq1 proteins, mammalian prion proteins, amyloid precursor protein, Aβ40, Aβ42, immunoglobulin (Ig) light chain, serum amyoid A, wild type or variant transthyretin, lysozyme, BnL, cystatin C, β2-microglobulin, apoliprotein A1, gelsolin or a mutant thereof, lactotransferrin, islet amyloid polypeptide, fibrinogen, prolactin, insulin, calcitonin, atrial natriuretic factor, α-synuclein, Huntingtin, superoxide dismutase, or α1-chymotrypsin.

One of skill in the art will readily be able to identify the full length sequences of these or any other aggregation-prone polypeptide which can be used to derive an anti-amyloid peptide to be expressed by an anti-amyloid peptide engineered bacteriophages as disclosed herein by reference to public databases as well as the scientific and patent literature. For example, the sequence of Sc Sup35 is provided in U.S. Ser. No. 11/004,418.

Aggregation domains of the yeast prion proteins Saccharomyces cerevisiae (Sc) Sup35 and Candida albicans (Ca) Sup 35 are useful to derive anti-amyloid peptides to be expressed by the anti-amyloid peptide engineered bacteriophages as disclosed herein. In some embodiments, a variety of peptides located between amino acids 1-40 of Sc Sup35 are capable of binding to full length Sc Sup35 (but not Ca Sup35) to form higher ordered aggregates, and thus are encompassed for use as anti-amyloid peptide to be expressed by an anti-amyloid peptide engineered bacteriophages as disclosed herein. In another embodiment, anti-amyloid peptide to be expressed by an anti-amyloid peptide engineered bacteriophages consists of amino acids 10-29 of Sc Sup35. In another embodiment, anti-amyloid peptide to be expressed by an anti-amyloid peptide engineered bacteriophages includes amino acids 69-76 of Ca Sup35 which is capable of binding to full length Ca Sup35 (but not to Sc Sup35) to form higher ordered aggregates.

In another aspect, a protein aggregation domain of an amyloid polypeptide is useful to derive an anti-amyloid peptide to be expressed by an anti-amyloid peptide engineered bacteriophages as described herein. A protein aggregation domain may be located N-terminal or C-terminal to an amyloid polypeptide of interest. A protein aggregration domain of an amyloid polypeptide is region of any polypeptide which contacts a second polypeptide to form a high order aggregate.

In some embodiments, an anti-amyloid peptide expressed by an anti-amyloid peptide engineered bacteriophage is a peptide derived from an amyloid polypeptide where there is a commercial, therapeutic, prophylactic or practical interest to prevent amyloid formation. Exemplary amyloid polypeptides from which an anti-amyloid peptide can be derived includes any polypeptide whose aggregation is associated with a mammalian disease or amyloid associated disorder.

The term “derived from as used herein means that the amyloid peptide, e.g, an anti-amyloid peptide or a pro-amyloid peptide is a fragment of” the polypeptide or is sufficiently similar in sequence to a fragment of the polypeptide to nucleate self-assembly of the polypeptide to form an aggregate.

The length of the fragment may be, e.g., between 10 amino acids up to the full length of the polypeptide, e.g., at least 10, 20, 50, 100, 200, 300, or 500 amino acids, etc., provided that the fragment contains a domain that mediates self-assembly to form higher ordered aggregates. The fragment may encompass between 20-100% of the total polypeptide sequence, e.g., 30-100%, 40-100%, 50-100%, 60-100%, 70-100%, 80-100%, or 90-100% of the total sequence.

A plurality of anti-amyloid peptide engineered bacteriophage, or pro-amyloid peptide engineered bacteriophage can comprise, e.g., up to 10, 50, 100, 150, 200, 250, or more different amyloid peptides, e.g, an anti-amyloid peptide or a pro-amyloid peptides. Collectively and as an illustrative example only, in various embodiments, anti-amyloid peptides of a plurality of different anti-amyloid peptide engineered bacteriophages can encompass between 20-100% of a total polypeptide sequence, e.g., 30-100%, 40-100%, 50-100%, 60-100%, 70-100%, 80-100%, or 90-100% of a polypeptide sequence from which the anti-amyloid peptides encoded by an anti-amyloid peptide engineered bacteriophages are derived.

In some embodiments, an anti-amyloid peptide encoded by an anti-amyloid peptide engineered bacteriophage can be, e.g., 6-12, 8-15, 10-20, 10-30, 20-30, 30-40, or 40-50 amino acids in length. In some embodiments, anti-amyloid peptides encoded by a plurality of anti-amyloid peptide engineered bacteriophages can overlap in sequence by between, e.g., 1-25 residues, e.g., between 5-20 residues, or between 10-15 residues. In some embodiments, an anti-amyloid peptide encoded by an anti-amyloid peptide engineered bacteriophage can “scan” at least a portion of the polypeptide, i.e., the starting positions of the peptides with respect to the polypeptide are displaced from one another (“staggered”) by X residues where X is, for example, between 1-10 residues or between 1-6 residues or between 1-3 residues. In one embodiment, the starting positions of anti-amyloid peptides encoded by a plurality of anti-amyloid peptide engineered bacteriophages with respect to the amyloid polypeptide sequence from which it is derived is staggered by 1 amino acid. For example, a first anti-amyloid peptide corresponds to amino acids 1-20; a second anti-amyloid peptide corresponds to amino acids 2-21; a third anti-amyloid peptide corresponds to amino acids 3-22, etc. In another embodiment, the starting positions of anti-amyloid peptides encoded by a plurality of anti-amyloid peptide engineered bacteriophages with respect to the amyloid polypeptide sequence from which it is derived is staggered by 2 amino acids. For example, a first anti-amyloid peptide corresponds to amino acids 1-20; a second anti-amyloid peptide corresponds to amino acids 3-22; a third anti-amyloid peptide corresponds to amino acids 5-23, etc.

A plurality of anti-amyloid peptides encoded by a plurality of anti-amyloid peptide engineered bacteriophages need not include the N-terminal or C-terminal amino acid of the amyloid polypeptide. In some embodiments, a plurality of anti-amyloid peptide encoded by an anti-amyloid peptide engineered bacteriophage can span any N-terminal, C-terminal, or internal portion of an amyloid polypeptide. The anti-amyloid peptides could include or further include a detectable label, a reactive moiety, a tag, a spacer, a crosslinker, etc. The anti-amyloid peptides encoded by a plurality of anti-amyloid peptide engineered bacteriophages need not all be the same length and need not all fall within any single range of lengths.

### Attachment or Expression of the Anti-Amyloid Peptide on the Surface of a Bacteriophage

In one embodiment, the invention provides a bacteriophage that has been genetically engineered to express at least one amyloid peptide, e.g, an anti-amyloid peptide or a pro-amyloid peptide on their surface. The theoretical boundaries of the expression of a amyloid peptide, e.g, an anti-amyloid peptide or a pro-amyloid peptide copy number per phage depend primarily on the size of the anti-amyloid peptide, and the type of bacteriophage and the number of capsid proteins per phage. Generally, the number of anti-amyloid peptides or pro-amyloid peptides displayed on the phage is dependent on the number of capsid protein of the phage. For example in T7, one can use one fusion protein in the case of a large amyloid peptide, e.g, an anti-amyloid peptide or a pro-amyloid peptide, or as many as 415 in the case of a small amyloid peptide, e.g, an anti-amyloid peptide or a pro-amyloid peptide. Preferably, each phage has multiple copies of the amyloid peptide, e.g, an anti-amyloid peptide or a pro-amyloid peptide on their surface. The phage can carry, for example, 1 copy, 2, 3, 4, 5, 6, 7, 8, 9, 10, 11, 12, 13, 14, 15, 16, 17, 18, 19, 20 copies, 20-50, 50-100, 100-200, 200-300, 300-400, 400-500 or more, of anti-amyloid peptide on their surface. Wild type T7 has a capsid that is composed of 10% of 10B, a small capsid protein. One can make a fusion protein with this capsid protein and the anti-amyloid peptide. For example, 10B plus about 40 to 50 amino acids encoding anti-amyloid peptide. In an alternative embodiment, one could theoretically replace every capsid protein provided the anti-amyloid peptide does not sterically hinder the capsid protein formation. Typically, the anti-amyloid peptide engineered bacteriophage carries at least about 5-15 copies of anti-amyloid peptide on its surface. For example, in one embodiment, the anti-amyloid peptide is a fusion protein with 10B capsid protein so it can be displayed on the phage surface.

The fusion protein could comprise a single amyloid peptide, e.g, an anti-amyloid peptide or a pro-amyloid peptide or a plurality of amyloid peptides, e.g, anti-amyloid peptides or pro-amyloid peptides, which could be the same or different in sequence. The amyloid peptides, e.g, an anti-amyloid peptide or a pro-amyloid peptide could be derived from a single bacterial polypeptide, e.g., E. coli CsgB, or from multiple different bacterial polypeptides. For example, a fusion protein could comprise a first anti-amyloid peptide derived from a first bacterial species or genus and a second anti-amyloid peptide derived from a second bacterial species or genus. Such anti-amyloid-capsid fusion proteins, and nucleic acids encoding such fusion proteins, are aspects of the invention.

Secretion of an Anti-Amyloid Peptide from the Host Bacterial Cell

In some embodiments, the amyloid peptide, e.g, an anti-amyloid peptide or a pro-amyloid peptide expressed from the host bacterial cell is released when the bacterial host cell lyses in the lytic cycle process of bacteriophage infection. In alternative embodiment, the expressed amyloid peptide, e.g, an anti-amyloid peptide or a pro-amyloid peptide is released from the bacterial host cell by the bacterial host cell via the secretory pathway. In such an embodiment, the amyloid peptide, e.g, an anti-amyloid peptide or a pro-amyloid peptide expressed from the bacteriophage-infected host bacterial cell also contains a signal peptide such as a secretory signal sequence. Such a secretory signal sequence allows intracellular transport of the amyloid peptide, e.g, an anti-amyloid peptide or a pro-amyloid peptide to the bacterial cell plasma membrane for its secretion from the bacteria. Accordingly, in such an embodiment, the expressed amyloid peptide, e.g, an anti-amyloid peptide or a pro-amyloid peptide is expressed as a pro-amyloid peptide comprising the signal sequence and an amyloid peptide, e.g, an anti-amyloid peptide or a pro-amyloid peptide, where the signal sequence is subsequently cleaved as the amyloid peptide, e.g, an anti-amyloid peptide or a pro-amyloid peptide is secreted from the host bacteria to render the mature amyloid peptide in its active form without the signal sequence. In some embodiments, multiple bacteriophage expressing an amyloid peptide, e.g, an anti-amyloid peptide or a pro-amyloid peptide at their surface are released following lysis of a bacterial cell infected by the bacterophage.

One particular benefit of an anti-amyloid peptide engineered bacteriophage expressing an anti-amyloid peptide, and a method of using it according to methods disclosed herein is the presence of the anti-amyloid in the immediate locality of the bacteriophage, thus the anti-amyloid peptide is released from bacterial host cells infected with the bacteriophage, via either lysis or being secreted, allowing the anti-amyloid peptide to inhibit the formation of, or maintainance of amyloid. Additionally, another advantage of delivering the anti-amyloid peptides by being expressed by a bacteriophage is that it enables the anti-amyloid peptides to come into contact with amyloids which may not be accessible using conventional methods, for example it allows the anti-amyloid peptides to be within the locality of biofilms in difficult to reach places due to the bacteria being located in a difficult to access location, such as a small space or between two pieces of material. As such, another advantage of the present invention is an improved genetically engineered bacteriophage which express anti-amyloid peptides within the near vicinity of amyloids, such as curli amyloid in biofilms produced by bacterial cells, which may not be accessible to anti-amyloid peptides delivered by other means.

### Signal Sequence:

Without wishing to be bound to theory, when proteins are expressed by a cell, including a bacterial cell, the proteins are targeted to a particular part in the cell or secreted from the cell. Thus, protein targeting or protein sorting is the mechanism by which a cell transports proteins to the appropriate positions in the cell or outside of it. Sorting targets can be the inner space of an organelle, any of several interior membranes, the cell's outer membrane, or its exterior via secretion. This delivery process is carried out based on information contained in the protein itself. Correct sorting is crucial for the cell; errors can lead to diseases.

With some exceptions, bacteria lack membrane-bound organelles as found in eukaryotes, but they may assemble proteins onto various types of inclusions such as gas vesicles and storage granules. Also, depending on the species of bacteria, bacteria may have a single plasma membrane (Gram-positive bacteria), or both an inner (plasma) membrane and an outer cell wall membrane, with an aqueous space between the two called the periplasm (Gram-negative bacteria). Proteins can be secreted into the environment, according to whether or not there is an outer membrane. The basic mechanism at the plasma membrane is similar to the eukaryotic one. In addition, bacteria may target proteins into or across the outer membrane. Systems for secreting proteins across the bacterial outer membrane may be quite complex and play key roles in pathogenesis. These systems may be described as type I secretion, type II secretion, etc.

In most Gram-positive bacteria, certain proteins are targeted for export across the plasma membrane and subsequent covalent attachment to the bacterial cell wall. A specialized enzyme, sortase, cleaves the target protein at a characteristic recognition site near the protein C-terminus, such as an LPXTG (SEQ ID NO: 197) motif (where X can be any amino acid), then transfers the protein onto the cell wall. An system analogous to sortase/LPXTG, termed exosortase/PEP-CTERM, is proposed to exist in a broad range of Gram-negative bacteria.

A. Secretion in Gram Negative Bacteria

By way of background but not wishing to be bound by theory, secretion is present in bacteria and archaea as well. ATP binding cassette (ABC) type transporters are common to all the three domains of life. The secretory system in bacteria, also referred to in the art as the “Sec system” is a conserved secretion system which generally requires the presence of an N-terminal signal peptide on the secreted protein. Gram negative bacteria have two membranes, thus making secretion topologically more complex. There are at least six specialized secretion systems (Type I-VI) in Gram negative bacteria.

1. Type I Secretion System (T1SS or TOSS):

It is similar to the ABC transporter, however it has additional proteins that, together with the ABC protein, form a contiguous channel traversing the inner and outer membranes of Gram-negative bacteria. It is a simple system, which consists of only three protein subunits: the ABC protein, membrane fusion protein (MFP), and outer membrane protein (OMP). Type I secretion system transports various molecules, from ions, drugs, to proteins of various sizes (20-900 kDa). The molecules secreted vary in size from the small Escherichia coli peptide colicin V, (10 kDa) to the Pseudomonas fluorescens cell adhesion protein LapA of 900 kDa. The best characterized are the RTX toxins and the lipases. Type I secretion is also involved in export of non-proteinaceous substrates like cyclic β-glucans and polysaccharides. Many secreted proteins are particularly important in bacterial pathogenesis. [Wooldridge K (2009). Bacterial Secreted Proteins: Secretory Mechanisms and Role in Pathogenesis. Caister Academic Press]

2. Type II Secretion System (T2SS):

Proteins secreted through the type II system, or main terminal branch of the general secretory pathway, depend on the Sec system for initial transport into the periplasm. Once there, they pass through the outer membrane via a multimeric complex of secretin proteins. In addition to the secretin protein, 10-15 other inner and outer membrane proteins compose the full secretion apparatus, many with as yet unknown function. Gram-negative type IV pili use a modified version of the type II system for their biogenesis, and in some cases certain proteins are shared between a pilus complex and type II system within a single bacterial species.

3. Type III Secretion System (T3SS or TTSS):

It is homologous to bacterial flagellar basal body. It is like a molecular syringe through which a bacterium (e.g. certain types of Salmonella, Shigella, Yersinia) can inject proteins into eukaryotic cells. The low Ca²⁺ concentration in the cytosol opens the gate that regulates T3SS. One such mechanism to detect low calcium concentration has been illustrated by the lcrV (Low Calcium Response) antigen utilized by Y. pestis, which is used to detect low calcium concentrations and elicits T3SS attachment. (Salyers et al, 2002; Bacterial Pathogenesis: A Molecular Approach, 2nd ed., Washington, D.C.: ASM Press)

4. Type IV Secretion System (T455 or TFSS):

It is homologous to conjugation machinery of bacteria (and archaeal flagella). It is capable of transporting both DNA and proteins. It was discovered in Agrobacterium tumefaciens, which uses this system to introduce the Ti plasmid and proteins into the host which develops the crown gall (tumor). [[Helicobactor pylori]] uses a type IV secretion system to deliver CagA into gastric epithelial cells. Bordetella pertussis, the causative agent of whooping cough, secretes the pertussis toxin partly through the type IV system. Legionella pneumophila, the causing agent of legionellosis (Legionnaires' disease) utilizes type IV secretion system, known as the icm/dot (intracellular multiplication/defect in organelle trafficking genes) system, to translocate numerous effector proteins into its eukaryotic host. (Cascales et al., (2003), Nat Rev Microbiol 1 (2): 137-149). The prototypic Type IV secretion system is the VirB complex of Agrobacterium tumefaciens (Christie et al. 2005; Ann Rev Microbiol 59: 451-485).

5. Type V Secretion System (T5SS):

Also know in the art as the “autotransporter system” (Thanassi, et al., 2005; Mol. Membrane. Biol. 22 (1): 63-72). type V secretion involves use of the Sec system for crossing the inner membrane. Proteins which use this pathway have the capability to form a beta-barrel with their C-terminus which inserts into the outer membrane, allowing the rest of the peptide (the passenger domain) to reach the outside of the cell. Often, autotransporters are cleaved, leaving the beta-barrel domain in the outer membrane and freeing the passenger domain.

6. Type VI Secretion System (T6SS):

Proteins secreted by the type VI system lack N-terminal signal sequences and therefore presumably do not enter the Sec pathway. (Pukatzki et al., (2006), PNAS 103 (5): 1528-33; Mougous et al., (2006) Science 312 (5779): 1526-30). Type VI secretion systems are now known to be widespread in Gram-negative bacteria. (Bingle et al., 2008; Curr. Opin. Microbiol. 11 (1): 3-8; Cascales E (2008), EMBO Reports 9 (8): 735-741).

7. Twin-Arginine Translocation:

Bacteria as well as mitochondria and chloroplasts also use many other special transport systems such as the twin-arginine translocation (Tat) pathway which, in contrast to Sec-depedendent export, transports fully folded proteins across the membrane. The signal sequence requires two consecutive arginines for targeting to this system.

8. Release of Outer Membrane Vesicles:

In addition to the use of the multiprotein complexes listed above, Gram-negative bacteria possess another method for release of material: the formation of outer membrane vesicles. [Chatterjee, et al., J. Gen. Microbiol.” “49”: 1-11 (1967); Kuehn et al., Genes Dev. 19(22):2645-55 (2005)]. Portions of the outer membrane pinch off, forming spherical structures made of a lipid bilayer enclosing periplasmic materials. Vesicles from a number of bacterial species have been found to contain virulence factors, some have immunomodulatory effects, and some can directly adhere to and intoxicate host cells. While release of vesicles has been demonstrated as a general response to stress conditions, the process of loading cargo proteins seems to be selective. [McBroom, et al., Mol. Microbiol. 63(2):545-58 (2007)]

B. Secretion in Gram Positive Bacteria

Proteins with appropriate N-terminal targeting signals are synthesized in the cytoplasm and then directed to a specific protein transport pathway. During, or shortly after its translocation across the cytoplasmic membrane, the protein is processed and folded into its active form. Then the translocated protein is either retained at the extracytoplasmic side of the cell or released into the environment. Since the signal peptides that target proteins to the membrane are key determinants for transport pathway specificity, these signal peptides are classified according to the transport pathway to which they direct proteins. Signal peptide classification is based on the type of signal peptidase (SPase) that is responsible for the removal of the signal peptide. The majority of exported proteins are exported from the cytoplasm via the general “Secretory (Sec) pathway”. Most well known virulence factors (e.g. exotoxins of Staphylococcus aureus, protective antigen of Bacillus anthracia, lysteriolysin O of Listeria monocytogenes) that are secreted by Gram-positive pathogens have a typical N-terminal signal peptide that would lead them to the Sec-pathway. Proteins that are secreted via this pathway are translocated across the cytoplasmic membrane in an unfolded state. Subsequent processing and folding of these proteins takes place in the cell wall environment on the trans-side of the membrane. In addition to the Sec system, some Gram-positive bacteria also contain the Tat-system that is able to translocate folded proteins across the membrane. Pathogenic bacteria may contain certain special purpose export systems that are specifically involved in the transport of only a few proteins. For example, several gene clusters have been identified in mycobacteria that encode proteins that are secreted into the environment via specific pathways (ESAT-6) and are important for mycobacterial pathogenesis. Specific ATP-binding cassette (ABC) transporters direct the export and processing of small antibacterial peptides called bacteriocins. Genes for endolysins that are responsible for the onset of bacterial lysis are often located near genes that encode for holin-like proteins, suggesting that these holins are responsible for endolysin export to the cell wall. [Wooldridge K (2009). Bacterial Secreted Proteins: Secretory Mechanisms and Role in Pathogenesis. Caister Academic Press]

In some embodiments, the signal sequence useful in the present invention is OmpA Signal sequence, however any signal sequence commonly known by persons of ordinary skill in the art which allows the transport and secretion of anti-amyloid peptide outside the bacteriophage infected cell are encompassed for use in the present invention.

Signal sequence that direct secretion of proteins from bacterial cells are well known in the art, for example as disclosed in International application WO2005/071088, which is herein incorporated in its entirety by reference.

For example, one can use some of the non-limited examples of signal peptide shown in Table 1 which can be attached to the amino-terminus or carboxyl terminus of the antimicrobial peptide to be expressed by the anti-amyloid peptide engineered bacteriophage. Attachment can be via fusion or chimera composition with selected anti-amyloid peptides resulting in the secretion from the bacterium infected with the anti-amyloid peptide engineered bacteriophage.

TABLE 1
Some exemplary signal peptides to direct secretion of an anti-amyloid peptide out of a
bacterial cell.

| | | Signal peptidase | | |
|---|---|---|---|---|
| Sectretion | Signal Peptide Amino Acid sequence | Site (cleavage site | | |
| Pathway | (NH₂-CO₂) | represented by ′) | Gene | Genus/Species |
| secA1 | MKKIMLVITLILVSPIAQQTEAKD | TEA′KD (SEQ | Hly (LLO) | Listeria |
| | (SEQ ID NO: 228) | ID NO: 238) | | monocytogenes |
| | MKKKIISAILMSTVILSAAAPLSGVYA | VYA′DT (SEQ | Usp45 | Lactococcus |
| | DT (SEQ ID NO: 229) | ID NO: 239) | | lactis |
| | MKKRKVLIPLMALSTILVSSTGNLEVI | IQA′EV (SEQ ID | Pag | Bacillus |
| | QAEV (SEQ ID NO: 230) | NO: 240) | (protective | anthracis |
| | | | antigen) | |
| secA2 | MNMKKATIAATAGIAVTAFAAPTIAS | ASA′ST (SEQ ID | Iap (invasion- | Listeria |
| | AST (SEQ ID NO: 231) | NO: 241) | associated | monocytogenes |
| | | | protein p60) | |
| | MQKTRKERILEALQEEKKNKKSKKF | VSA′DE (SEQ ID | NamA | Listeria |
| | KTGATIAGVTAIATSITVPGIEVIVSAD | NO: 242) | Imo2691 | monocytogenes |
| | E (SEQ ID NO: 232) | | (autolysin) | |
| | MKKLKMASCALVAGLMFSGLTPNAF | AFA′ED (SEQ ID | *BA_0281 | Bacillus |
| | AED (SEQ ID NO: 233) | NO: 243) | (NLP/P60 | anthracis |
| | | | family) | |
| | MAKKFNYKLPSMVALTLVGSAVTAH | VQA′AE (SEQ | * atl | Staphylococcus |
| | QVQAAE (SEQ ID NO: 234) | ID NO: 244) | (autolysin) | aureus |
| Tat | MTDKKSENQTEKTETKENKGMTRRE | DKA′LT (SEQ ID | Imo0367 | Listeria |
| | MLKLSAVAGTGIAVGATGLGTILNVV | NO: 245) | | monocytogenes |
| | DQVDKALT (SEQ ID NO: 235) | | | |
| | MAYDSRFDEWVQKLKEESFQNNTFD | | PhoD | Bacillus subtillis |
| | RRKFIQGAGKIAGLGLGLTIAQSVGA | | (alkaline | |
| | FG (SEQ ID NO: 236) | | phosphatase) | |

In alternative embodiments, one of ordinary skill in the art can use synthetic bacterial sequences, such as those discussed in Clérico et al., Biopolymers. 2008; 90(3):307-19, which is incorporated herein by reference. Alternatively, one can use methods to secrete peptides without the use of signal (or secretory) sequences, such as the methods disclosed in International Application WO2007/018853, which is incorporated herein by reference. Bacterial protein secretion is discussed in Driessen et al., Nat Struct Biol. 2001 June; 8(6):492-8, which is incorporated herein by reference. The localization of signal sequences, such as secretory signal sequences can be located anywhere on the peptide, so long as the signal is exposed on the peptide and its placement does not disrupt the inhibitory effect of the anti-amyloid peptide For example, it can be placed at the carboxy or amino terminus or even sometimes within the peptide, providing it satisfies the above conditions. Some signal sequences which can be used are disclosed in Table 7 of U.S. Pat. No. 6,072,039 which is incorporated herein in its entirety by reference.

### Modification of an Anti-Amyloid Peptide or Pro-Amyloid Engineered Bacteriophage

In another embodiment, an anti-amyloid peptide engineered bacteriophage can be further be modified to comprise nucleic acids which encode enzymes which assist in breaking down or degrading the biofilm matrix, for example any gene known as encoding a biofilm degrading enzyme by persons of ordinary skill in the art, such as, but not limited to Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In other embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase (see, e.g., Verhoef R. et al., Characterisation of a 1,4-beta-fucoside hydrolase degrading colanic acid, Carbohydr Res. 2005 Aug. 15; 340(11):1780-8), depolymerazing alginase, and DNase I, or combinations thereof, as disclosed in the methods as disclosed in U.S. patent application Ser. No. 11/662,551 and International Patent Application WO2006/137847 and provisional patent application 61/014,518, which are specifically incorporated herein in their entirety by reference.

In another embodiment, an anti-amyloid peptide engineered bacteriophage or a pro-amyloid engineered bacteriophage can be further be modified in a species-specific manner, for example, one can modify or select the bacteriophage on the basis for its infectivity of specific bacteria.

In another embodiment, an anti-amyloid peptide engineered bacteriophage or a pro-amyloid engineered bacteriophage can be further modified to comprise nucleic acids which encodes enzymes or sequences for other beneficial purposes such as, but not limited to, a fluorescent protein tag for visualization, or an aptamer for treatment of complications induced by amyloid-associated disorders.

A bacteriophage to be engineered or developed into an anti-amyloid peptide engineered bacteriophage or a pro-amyloid engineered bacteriophage can be any bacteriophage as known by a person of ordinary skill in the art. In some embodiments, an anti-amyloid peptide engineered bacteriophage is derived from any or a combination of bacteriophages listed in Tables 3-5.

In some embodiments, a bacteriophage which is engineered to become an anti-amyloid peptide engineered bacteriophage or a pro-amyloid engineered bacteriophage as disclosed herein is a lytic bacteriophage or lysogenic bacteriophage, or any bacteriophage that infects E. coli, P. aeriginosa, S. aureaus, E. facalis and the like. Such bacteriophages are well known to one skilled in the art and are listed in Tables 3-5, and include, but are not limited to, lambda phages, M13, T7, T3, and T-even and T-even like phages, such as T2, and T4, and RB69; also phages such as Pf1, Pf4, Bacteroides fragilis phage B40-8 and coliphage MS-2 can be used. For example, lambda phage attacks E. coli by attaching itself to the outside of the bacteria and injecting its DNA into the bacteria. Once injected into its new host, a bacteriophage uses E. coli's genetic machinery to transcribe its genes. Any of the known phages can be engineered to express an anti-amyloid peptide as described herein.

In some embodiments, bacteriophages which have been engineered to be more efficient cloning vectors or naturally lack a gene important in infecting all bacteria, such as male and female bacteria can be used to generate an anti-amyloid peptide engineered bacteriophage as disclosed herein. Typically, bacteriophages that have been engineered to lack genes for infecting all variants and species of bacteria can have reduced capacity to replicate in naturally occurring bacteria thus limiting the use of such phages in degradation of biofilm produced by the naturally occurring bacteria.

For example, the capsid protein of phage T7, gene 10, comes in two forms, the major product 10A (36 kDa) and the minor product 10B (41 kDa) (Condron, B. G., Atkins, J. F., and Gesteland, R. F. 1991. Frameshifting in gene 10 of bacteriophage T7. J. Bacteriol. 173:6998-7003). Capsid protein 10B is produced by frameshifting near the end of the coding region of 10A. NOVAGEN® modified gene 10 in T7 to remove the frameshifting site so that only 10B with the attached user-introduced peptide for surface display is produced (U.S. Pat. No. 5,766,905. 1998. Cytoplasmic bacteriophage display system, which is incorporated in its entirety herein by reference). The 10B-enzyme fusion product is too large to make up the entire phage capsid because the enzymes that are typically introduced into phages, such as T7, are large (greater than a few hundred amino acids). As a result, T7select 10-3b must be grown in host bacterial strains that produce wild-type 10A capsid protein, such as BLT5403 or BLT5615, so that enough 10A is available to be interspersed with the 10B-enzyme fusion product to allow replication of phage (U.S. Pat. No. 5,766,905. 1998. Cytoplasmic bacteriophage display system, which is incorporated in its entirety herein by reference). However, because most biofilm-forming E. coli do not produce wild-type 10A capsid protein, this limits the ability of T7select 10-3b displaying large enzymes on their surface to propagate within and lyse some important strains of E. coli. Accordingly, in some embodiments, the present invention provides genetically anti-amyloid peptide engineered bacteriophages that in addition to comprising a nucleic acid encoding an anti-amyloid peptide and being capable of expressing and secreting the gene product (i.e. the anti-amyloid peptide nucleic acid and/or antimicrobial protein or peptide), also express all the essential genes for virus replication in naturally occurring bacterial strains. In one embodiment, the invention provides an engineered T7select 10-3b phage that expresses both cellulase and 10A capsid protein.

It is known that wild-type T7 does not productively infect male (F plasmid-containing) E. coli because of interactions between the F plasmid protein PifA and T7 genes 1.2 or 10 (Garcia, L. R., and Molineux, I. J. 1995. Incomplete entry of bacteriophage T7 DNA into F plasmid-containing Escherichia coli. J. Bacteriol. 177:4077-4083.). F plasmid-containing E. coli infected by T7 die but do not lyse or release large numbers of T7 (Garcia, L. R., and Molineux, I. J. 1995. Incomplete entry of bacteriophage T7 DNA into F plasmid-containing Escherichia coli. J. Bacteriol. 177:4077-4083). Wild-type T3 grows normally on male cells because of T3's gene 1.2 product (Garcia, L. R., and Molineux, I. J. 1995, Id.). When T3 gene 1.2 is expressed in wild-type T7, T7 is able to productively infect male cells (Garcia, L. R., and Molineux, I. J. 1995. Id).

Because many biofilm-producing E. coli contain the F plasmid (Ghigo, et al., 2001. Natural conjugative plasmids induce bacterial biofilm development. Nature. 412:442-445), it is important, although not necessary, for an anti-amyloid peptide engineered bacteriophage to be able to productively infect also male cells. Therefore, in addition to an anti-amyloid peptide engineered bacteriophage expressing and secreting the anti-amyloid peptide, one can also engineer it to express the gene necessary for infecting the male bacteria. For example, one can use the modification described by Garcia and Molineux (Garcia, L. R., and Molineux, I. J. 1995. Incomplete entry of bacteriophage T7 DNA into F plasmid-containing Escherichia coli. J. Bacteriol. 177:4077-4083) to express T3 gene 1.2 in T7.

In some embodiments, an engineered anti-amyloid bacteriophage or a pro-amyloid engineered bacteriophage that lacks one or more genes important or essential for viral replication in a naturally occurring bacterial strain is administered or used together with a second bacteriophage that expresses all essential genes for virus replication in a naturally occurring bacterial strain. The second bacteriophage could be a non-engineered bacteriophage or a different engineered bacteriophage.

### Promoters for Expression of the Anti-Amyloid Peptide by an Anti-Amyloid Peptide Engineered Bacteriophage

In some embodiments, an anti-amyloid peptide or a pro-amyloid peptide can be attached to the surface of a bacteriophage by methods as disclosed herein and other methods known by an artisan of ordinary skill in the art. In all other embodiments all aspects described herein, an anti-amyloid peptide engineered bacteriophage can express an anti-amyloid peptide. In some embodiments, a pro-amyloid engineered bacteriophage can express a pro-amyloid peptide. In some embodiments, the expressed anti-amyloid peptide or pro-amyloid peptide is as a fusion protein to a coat protein to be on the surface of the bacteriophages, and in other embodiments, the anti-amyloid peptide or pro-amyloid peptide expressed by the bacteriophage is released from the bacteriophage (e.g. by lysis or secretion). In this aspect and all aspects as described herein, the anti-amyloid peptide or pro-amyloid peptide can be linked to a signal sequence (also known in the art as a signal peptide), such as a secretion sequence, allowing translocation of the anti-amyloid peptide, or pro-amyloid peptide to the bacterial cell surface or plasma membrane and secretion of the anti-amyloid peptide out or pro-amyloid peptide of the bacterial cell. An anti-amyloid peptide or pro-amyloid peptide which comprises a signal sequence allows it to be secreted from the host bacterial cell is referred to herein as a “secretable amyloid peptide”. In some embodiments, the signal sequence is a Omp secretion sequence. Thus, the nucleic acid encoding an amyloid peptide, e.g., an anti-amyloid peptide or a pro-amyloid peptide is operatively linked to the nucleic acid encoding the signal sequence.

In all aspects of the invention, gene expression from the nucleic acid encoding an amyloid peptide, e.g., an anti-amyloid peptide or a pro-amyloid peptide is regulated by a promoter to which the nucleic acid is operatively linked. In some embodiments, a promoter is a bacteriophage promoter. One can use any bacteriophage promoter known by one of ordinary skill in the art, for example but not limited to, any promoter listed in Table 10 or disclosed in world-wide web site “partsregistry.org/cgi/partsdb/pgroup.cgi?pgroup=other_regulator&show=1”.

In some embodiments, an amyloid peptide, e.g., an anti-amyloid peptide or a pro-amyloid peptide is a peptide as disclosed herein. In such embodiments a bacteriophage can be engineered to become an anti-amyloid peptide engineered bacteriophage or a pro-amyloid peptide engineered bacteriophage and, in some embodiments, to express a secretable form of an amyloid peptide, e.g., an anti-amyloid peptide or a pro-amyloid peptide. In some embodiments, a bacteriophage can be engineered to become an anti-amyloid peptide engineered bacteriophage to express an anti-amyloid peptide at the surface of the bacterophage. In some embodiments, the naturally occurring bacteriophage promoter is replaced in whole or in part with all or part of a heterologous promoter so that the bacteriophage and/or the bacteriophage infected-host cell expresses a high level of the secretable amyloid peptide, e.g., an anti-amyloid peptide or a pro-amyloid peptide. In some embodiments, a heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems, which are incorporated herein in their entirety by reference.

In some embodiments, a bacteriophage can be engineered as disclosed herein to express an amyloid peptide, e.g., an anti-amyloid peptide or a pro-amyloid peptide, under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al, which are all incorporated herein in their entirety by reference.

Other exemplary examples of promoter which can be used include, for example but not limited, Anhydrotetracycline(aTc) promoter, PLtetO-1 (Pubmed Nucleotide# U66309), Arabinose promoter (PBAD), IPTG inducible promoters PTAC (in vectors such as Pubmed Accession #EU546824), PTrc-2, Plac (in vectors such as Pubmed Accession #EU546816), PLlacO-1, PA1lacO-1, and Arabinose and IPTG promoters, such as Plac/ara-a. Examples of these promoters are as follows:

Anhydrotetracycline (aTc) promoter, such as PLtetO-1 (Pubmed Nucleotide# U66309): GCATGCTCCCTATCAGTGATAGAGATTGACATCCCTATCAGTGATAGAGATACTGAGCACATCAGCA GGACGCACTGACCAGGA (SEQ ID NO: 246); Arabinose promoter (PBAD): or modified versions which can be found at world-wide web site: partsregistry.org/wiki/index.php?title=Part:BBa_I13453″ AAGAAACCAATTGTCCATATTGCATCAGACATTGCCGTCACTGCGTCTTTTACTGGCTCTTCTCGCTA ACCAAACCGGTAACCCCGCTTATTAAAAGCATTCTGTAACAAAGCGGGACCAAAGCCATGACAAAA ACGCGTAACAAAAGTGTCTATAATCACGGCAGAAAAGTCCACATTGATTATTTGCACGGCGTCACAC TTTGCTATGCCATAGCATTTTTATCCATAAGATTAGCGGATCCTACCTGACGCTTTTTATCGCAACTC TCTACTGTTTCTCCATA (SEQ ID NO: 247); IPTG promoters: (i) PTAC (in vectors such as Pubmed Accession #EU546824, which is incorporated herein by reference), (ii) PTrc-2: CCATCGAATGGCTGAAATGAGCTGTTGACAATTAATCATCCGGCTCGTATAATGTGTGGAATTGTGA GCGGATAACAATTTCACACAGGA (SEQ ID NO: 248) and temperature sensitive promoters such as PLs1con, GCATGCACAGATAACCATCTGCGGTGATAAATTATCTCTGGCGGTGTTGACATAAATACCACTGGCG GTtATAaTGAGCACATCAGCAGG//GTATGCAAAGGA (SEQ ID NO: 249) and modified variants thereof.

### Modification of Engineered Bacteriophages.

In some embodiments of all aspects described herein, an anti-amyloid peptide engineered bacteriophage or pro-amyloid peptide engineered bacteriophage can also be designed for example, for optimal expression of amyloid peptide, e.g., an anti-amyloid peptide or a pro-amyloid peptide, or to delay cell lysis or using multiple phage promoters to allow for increased production of amyloid peptide, e.g., an anti-amyloid peptide or a pro-amyloid peptide, or for targeting multiple biofilm components with different amyloid peptide, e.g., with different an anti-amyloid peptides or different pro-amyloid peptides. In some embodiments, one can also target multi-species biofilm with a cocktail of different species-specific anti-amyloid peptide engineered bacteriophage or pro-amyloid peptide engineered bacteriophages, and combination therapy with other agents that are well known to one skilled in the art and phage to improve the efficacy of both types of treatment.

In some embodiments of all aspects described herein, an anti-amyloid peptide engineered bacteriophage can also be used together with other antibacterial or bacteriofilm degrading agents or chemicals such as EGTA, a calcium-specific chelating agent, effected the immediate and substantial detachment of a P. aeruginosa biofilm without affecting microbial activity, NaCl, CaCl₂ or MgCl₂, surfactants and urea.

Phage therapy or bacteriophage therapy has begun to be accepted in industrial and biotechnological settings. For example, the FDA has previously approved the use of phage targeted at Listeria monocytogenes as a food additive. Phage therapy has been used successfully for therapeutic purposes in Eastern Europe for over 60 years, and the development and use of phage therapy in clinical settings in Western medicine, in particular for treating mammals such as humans, is of great interest. In some embodiments of the invention, long-circulating phage that can avoid reticulo-endothelial (RES) clearance for increased in vivo efficacy are engineered to express anti-amyloid peptides. Accordingly, in all aspects described herein, the methods of the present invention are applicable to human treatment. A skilled artisan can also develop and carry out an appropriate clinical trial for use in clinical applications, such as therapeutic purposes as well as in human subjects. An anti-amyloid peptide engineered bacteriophage as disclosed herein is also expected to be effective in inhibiting formation and/or dispersing biofilms, including biofilms present in human organs, such as colon or lungs and other organs in a subject prone to bacterial infection associated with a bacterial biofilm.

Another aspect relates to a pharmaceutical composition comprising at least one anti-amyloid peptide engineered bacteriophage. In some embodiments of this and all aspects described herein, the composition comprising an anti-amyloid peptide engineered bacteriophage can be administered as a co-formulation with one or more other antimicrobial, non-antimicrobial or other therapeutic agents. In some embodiments, a pharmaceutical composition comprises at least one pro-amyloid peptide engineered bacteriophage.

In a further embodiment, the invention provides methods of administration of the compositions and/or pharmaceutical formulations comprising an anti-amyloid peptide engineered bacteriophage and include any means commonly known by persons skilled in the art. In some embodiments, the subject is any organism, including for example a mammalian, avian or plant. In some embodiments, the mammalian is a human, a domesticated animal and/or a commercial animal.

In one embodiment, the compositions and/or pharmaceutical formulations comprising an anti-amyloid peptide engineered bacteriophage or a pro-amyloid peptide engineered bacteriophage are administered into or onto solid surfaces, e.g. water pipes, water containers, catheters, fluid samples, food products and other surfaces infected by bacteria and susceptible to having a bacterial biofilm.

Non-lytic and non-replicative phage have been engineered to kill bacteria while minimizing endotoxin release. Accordingly, the present invention encompasses modification of an anti-amyloid peptide engineered bacteriophage or a pro-amyloid peptide engineered bacteriophage with minimal endotoxin release or toxin-free bacteriophage preparation.

The specificity of phage for host bacteria allows human cells as well as innocuous bacteria to be spared, potentially avoiding serious issues such as drug toxicity. Antibiotic therapy is believed to alter the microbial flora in the colon due to lack of target specificity, and in some instances allowing resistant C. difficile to proliferate and cause disease such as diarrhea and colitis.

For host specificity, if desired, a skilled artisan can generate a well-characterized library of anti-amyloid peptide engineered bacteriophages or pro-amyloid engineered bacteriophages, where specific anti-amyloid peptide engineered bacteriophage or pro-amyloid peptide engineered bacteriophage can be selected and for specific types of bacterial infection.

While one aspect of the present invention provides a method to increase (i.e. broadening) the ability of bacteriophages to target and be effective against multiple bacterial species, the diversity of bacterial infections may result in some instances where a single anti-amyloid peptide engineered bacteriophage as disclosed herein is not effective at killing or inhibiting biofilm formation or maintenance by all the different bacterial species in a given bacterial population. Thus, to circumvent this problem, one can administer a variety of different anti-amyloid peptide engineered bacteriophage to a bacterial population in order to be effective in killing or inhibiting biofilm formation or maintenance by all the different bacterial species in the heterogenous bacterial population. One can do this by having the same bacterial species expressing different anti-amyloid peptides, or alternatively, generating different an anti-amyloid peptide engineered bacteriophage from the same bacteriophage species expressing the same anti-amyloid peptide. In this way, one of ordinary skill in the art can use a combination of anti-amyloid peptide engineered bacteriophages as disclosed herein to be effective at killing or inhibiting biofilm formation or maintenance by a bacterial population comprising multiple different bacterial strains. Accordingly, in one embodiment, the invention provides use of a variety of different engineered bacteriophages in combination (i.e. a cocktail of engineered bacteriophages discussed herein) to cover a range of target bacteria.

One skilled in the art can generate a collection or a library of the anti-amyloid peptide engineered bacteriophages or pro-amyloid peptide engineered bacteriophages as disclosed herein by new cost-effective, large-scale DNA sequencing and DNA synthesis technologies. Sequencing technologies allows the characterization of collections of natural phage that have been used in phage typing and phage therapy for many years. Accordingly, a skilled artisan can use synthesis technologies as described herein to add different anti-amyloid peptides to produce a variety of new anti-amyloid peptide engineered bacteriophages.

Furthermore, rational engineering methods with new synthesis technologies can be employed to broaden an anti-amyloid peptide engineered bacteriophage host range. For example, a T7 anti-amyloid peptide engineered bacteriophage can be modified to express K1-5 endosialidase, allowing it to effectively replicate in E. coli that produce the K1 polysaccharide capsule. In some embodiments, the gene 1.2 from phage T3 can be used to extend an anti-amyloid peptide engineered bacteriophage to be able to transfect a host range to include E. coli that contain the F plasmid, thus demonstrating that multiple modifications of a phage genome can be done without significant impairment of the phage's ability to replicate. Bordetella bacteriophage use a reverse-transcriptase-mediated mechanism to produce diversity in host tropism which can also be used according to the methods of the present invention to create an anti-amyloid peptide engineered bacteriophage, and is lytic to the target bacterium or bacteria. The many biofilm-promoting factors required by E. coli K-12 to produce a mature biofilm are likely to be shared among different biofilm-forming bacterial strains and are thus also targets for an anti-amyloid peptide engineered bacteriophage as disclosed herein.

### Uses of the Engineered Bacteriophages

Accordingly, the inventors have demonstrated that an anti-amyloid peptide engineered bacteriophage as disclosed herein is effective at reducing amyloid formation, and decreasing the amyloid amount in biofilms produced by bacteria as compared to a bacteriophage which has not been engineered to express and secrete an anti-amyloid peptide.

The inventors have also discovered that an anti-amyloid peptide engineered bacteriophage can be adapted to express a variety of different anti-amyloid peptides, and can be further optionally modified, for example to express other biofilm-degrading enzymes to target a wide range of bacteria and bacteria biofilms. In some embodiments, an anti-amyloid peptide engineered bacteriophage can be used in combination with at least one other an anti-amyloid peptide engineered bacteriophage as disclosed herein, and optionally a different bacteriophage (engineered or non-engineered) or a different anti-amyloid peptide engineered bacteriophage, as well as a bacteriophage which is modified to express a therapeutic gene or a toxin gene or a biofilm degrading gene. Such bacteriophages are encompassed for use in the methods and compositions as disclosed herein.

In some embodiments, the anti-amyloid peptide engineered bacteriophages and methods and compositions provided herein can be used to inhibit biofilm formation or maintenance and/or that disrupt biofilms that have already formed. Such anti-amyloid peptide engineered bacteriophages and methods and compositions are useful for components of washes or disinfectant solutions (e.g., in combination with a suitable carrier such as water), to impregnate cleaning supplies such as sponges, wipes, or cloths, or as components of surface coatings (e.g., in combination with a suitable carrier such as a polymeric material or a carrier for slow release of the bacteriophage) for a variety of medical devices. Additionally, anti-amyloid peptide engineered bacteriophages and methods and compositions can be added to existing disinfectant or anti-microbial compositions. In certain embodiments, anti-amyloid peptide engineered bacteriophages and compositions thereof are useful as prophylactic or therapeutic agents in individuals who are susceptible to infection, infected (e.g., by biofilm-forming bacteria), and/or have an indwelling or implantable device, or are immunocompromised (e.g., individuals suffering from HIV, individuals taking immunosuppressive medication, or individuals with immune system deficiencies or dysfunction), or are allergic to antibiotics, or are hospitalized, or have an implanted prosthetic or medical device (e.g., an artificial heart valve, joint, stent, orthopedic appliance, etc.). Biofilms are often associated with cystic fibrosis, endocarditis, osteomyelitis, otitis media, urinary tract infections, oral infections, and dental caries, among other conditions. In some instances a biofilm-associated infection is a nosocomial infection. In some cases a biofilm-associated infection is a mixed infection, comprising multiple different microorganisms. In some cases an individual suffering from a biofilm-associated infection is at increased risk of contracting a second infection.

In some embodiments, an anti-amyloid peptide engineered bacteriophages and compositions thereof are useful as a component of a coating the surface of medical devices to prevent biofilm formation, for example, medical devices such as a catheter, stent, valve, pacemaker, conduit, cannula, appliance, scaffold, central line, IV line, pessary, tube, drain, trochar or plug, implant, a rod, a screw, or orthopedic or implantable prosthetic device or appliance. In some embodiments, the anti-amyloid peptide engineered bacteriophages can be coated on the surfaces of such medical devices such that they are slowly released from the surface. In another embodiment, an anti-amyloid peptide engineered bacteriophages and compositions thereof can be used as a component of a coating for a conduit, pipe lining, a reactor, filter, vessel, or equipment which comes into contact with a beverage or food, e.g., intended for human or animal consumption or treatment, or water or other fluid intended for consumption, cleaning, agricultural, industrial, or other use. In some embodiments an anti-amyloid peptide engineered bacteriophages and compositions thereof can be used as a component of a wound dressing, bandage, toothpaste, cosmetic, etc.

In another embodiment, an anti-amyloid peptide engineered bacteriophages and compositions thereof can be used to remove CsgA and/or CsgB polypeptides from a solution. The solution may be, e.g., water or a body fluid such as blood, plasma, serum, etc. The fluid is contacted with an anti-amyloid peptide engineered bacteriophage or compositions thereof. In some embodiments, the concentration of an anti-amyloid peptide engineered bacteriophage to be effective at inhibiting amyloid formation, for example, biofilm formation in solution is about at least 1×10² PFU/ml, or about at least 1×10³ PFU/ml, or about at least 1×10⁴ PFU/ml, or about at least 1×10⁵ PFU/ml, or about at least 1×10⁶ PFU/ml, or about at least 1×10² PFU/ml, or about at least 1×10⁸ PFU/ml, or about at least 1×10⁹ PFU/ml, or about at least 1×10¹⁰ PFU/ml, or more than about at least 1×10¹⁰ PFU/ml. In some embodiments, if the anti-amyloid peptide engineered bacteriophage is a non-relicating bacteriophage (i.e. does not infect cells and proliferate in the host bacteria via lysis), then the concentration of an anti-amyloid peptide engineered bacteriophage to be effective at inhibiting amyloid formation, for example, biofilm formation in solution is about at least 1×10⁷-1×10¹⁵ PFU/ml, for example, at least 1×10⁷ PFU/ml, or about at least 1×10⁸ PFU/ml, or about at least 1×10⁹ PFU/ml, or about at least 1×10¹⁰ PFU/ml, or about at least 1×10¹¹ PFU/ml, or about at least 1×10¹² PFU/ml, or about at least 1×10¹³ PFU/ml, or about at least 1×10¹⁴ PFU/ml, or about at least 1×10¹⁵ PFU/ml, or more than about at least 1×10¹⁵ PFU/ml.

In another embodiment, an anti-amyloid peptide engineered bacteriophages and compositions thereof can be used to decrease the presence of CsgA and/or CsgB polypeptides for waste clean-up, or sterilization purposes, or other industrial waste-management purposes.

In one embodiment, an anti-amyloid peptide engineered bacteriophages and compositions thereof are useful in a method to treat a subject either ex vivo or in vivo. In one embodiment, an anti-amyloid peptide engineered bacteriophage and a composition thereof can be used to inhibit protein aggregation or remove amyloids from a subject. In some embodiments, the subject is suffering from, or at risk of developing an amyloid associated disorder. In some embodiments, an anti-amyloid peptide engineered bacteriophages and compositions thereof are contacted with a blood product from the subject. In another embodiment, an anti-amyloid peptide engineered bacteriophages and compositions thereof are administered to a subject. In one embodiment an anti-amyloid peptide engineered bacteriophages and compositions thereof are contacted with the surface of an organ to be transplanted into a subject. The organ may be bathed in an anti-amyloid peptide engineered bacteriophages and compositions thereof prior to transplantation. In one embodiment, methods, anti-amyloid peptide engineered bacteriophages and compositions thereof can be used to remove protein aggregates and/or amyloids from a body fluid in a subject undergoing dialysis.

In some embodiments, the concentration of anti-amyloid peptide engineered bacteriophage for treatment of a subject to remove amyloid plaques in solution for example, remove amyloid formation from a biological sample (such as blood or other biological solution) can be about at least 1×10⁷-1×10¹⁵ PFU/ml, for example, at least 1×10⁷ PFU/ml, or about at least 1×10⁸ PFU/ml, or about at least 1×10⁹ PFU/ml, or about at least 1×10¹⁰ PFU/ml, or about at least 1×10¹¹ PFU/ml, or about at least 1×10¹² PFU/ml, or about at least 1×10¹³ PFU/ml, or about at least 1×10¹⁴ PFU/ml, or about at least 1×10¹⁵ PFU/ml, or more than about at least 1×10¹⁵ PFU/ml.

In some embodiments, where an anti-amyloid peptide engineered bacteriophage is used to treat a subject, the dose is at least 1×10⁷ PFU/ml or in some embodiments higher than 1×10⁷ PFU/ml. In some embodiments, where an anti-amyloid peptide engineered bacteriophage is used to treat a subject, such as a human subject with amyloidoses, an anti-amyloid peptide engineered bacteriophage can be administered multiple times (i.e. repeated doses). Should the bacteriophage/peptide/amyloid plaque complex to be immunogenic, then repeated dosing with the anti-amyloid peptide engineered bacteriophage would result in the plaques being cleared from the system. Typically, anti-amyloid peptide engineered bacteriophage is used to treat a subject or administered to a subject are non-relicating bacteriophages. Such bacteriophages are known to one of ordinary skill in the art and are disclosed herein.

In some embodiments, where an engineered bacteriophage express an amyloid peptide which promotes the formation or maintenance of protein aggregates, such a pro-amyloid peptide engineered bacteriophage can be used to promote or increase the formation of protein aggregates which comprise of two or more different polypeptides, e.g., “higher order aggregates”, for example, which are useful to promote or increase bacteria and/or promote the formation of a bacterial biofilms in environmental, industrial, and clinical settings by administering a composition comprising at least one pro-amyloid engineered bacteriophage as discussed herein. Pro-amyloid peptides are useful in circimstsances where it is desirable to encourage biofilm formation, such as for example but not limited to, establishing microbial biofilms for remediation, microbial fuel cells, “beneficial” biofilms that block “harmful” biofilms from forming on important surfaces, etc).

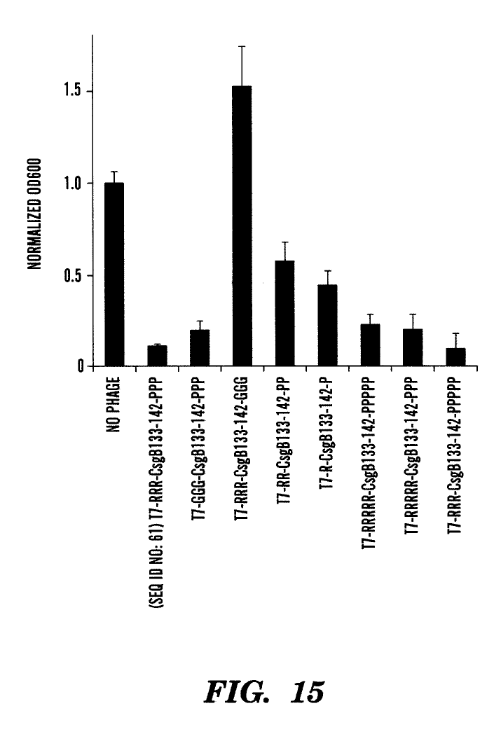

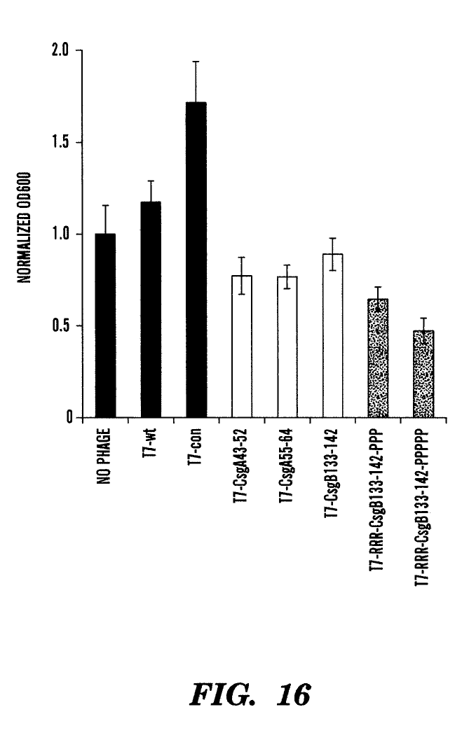

Accordingly, in some applications, it is beneficial to encourage and stimulate biofilm formation. For example, as described in Journal of Bioscience and Bioengineering Volume 101, Issue 1, January 2006, Pages 1-8, “Biofilm formation by B. subtilis and related species permits the control of infection caused by plant pathogens, the reduction of mild steel corrosion, and the exploration of novel compounds” (which is incorporated herein in its entirety by reference). Moreover, biofilms can be useful in environmental remediation such as cleaning wastewater, remediation of toxic compounds in contaminated soil or groundwater, and microbial leaching of inorganic materials. In these cases, the biofilm provides a stable environment where bacteria can metabolize toxic compounds or process chemicals for useful industrial purposes (see world wide web at: cs.montana.edu/ross/personal/intro-biofilms-s3.htm). Accordingly, the pro-amyloid peptide engineered bacteriophage, e.g., a bacteriophage expressing T7-RRR-CsgB(133-142)-GGG (see FIG. 15 in the Examples) can be used to promote the formation of bacteria biofilms for remediateion purposes, industrial purposes and clean-up purposes, controlling harmful or pathogenic bacterial infections and the like. Biofilm formation is also beneficial in symbiotic plant root nodules where the bacteria provide nitrogen fixation capabilities for plants see world wide web at: sysbio.org/research/bsi/biofilm/glucosemetabolism.stm). In other situations, biofilms may be used to house bacteria as environmental biosensors to detect environmental toxins or changes in environmental conditions. Finally, it can be beneficial to establish “good” biofilms in industrial settings that will not corrode pipes and will prevent “bad” biofilms from forming, since the “bad” biofilms can lead to corrosion and biofouling that is unwanted.

Biofilms can be used to create microbial fuel cells to produce energy from sustainable sources (Biosensors and Bioelectronics 22 (2007) 1672-1679). In these cases, biofilms can form on the electrodes or other materials to produce electrons or other forms of energy. Accordingly, the pro-amyloid peptide engineered bacteriophage, e.g., a bacteriophage expressing T7-RRR-CsgB(133-142)-GGG (see FIG. 15 in the Examples) can be used to promote the formation of bacteria biofilms for formation of environmental biosensors, detection of environmental conditions and toxins as well as reducing pathogenic biofilms such as biofouling, and for promoting biofilms in microbial fuel cells and the like.

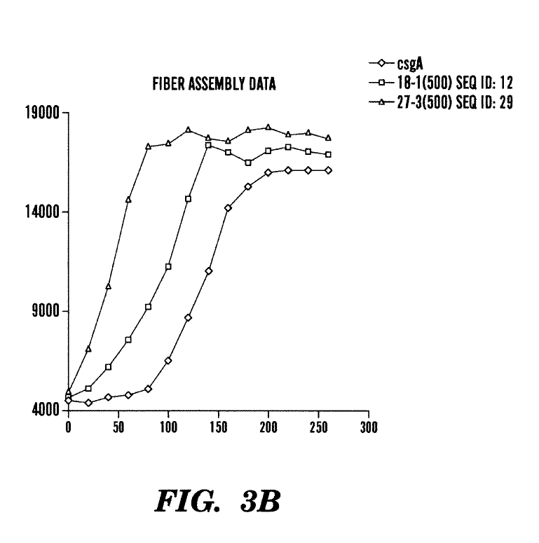

In some embodiments, engineered phage that express at least one pro-amyloid peptides can be also used to stimulate amyloid assembly and biofilm formation. As shown in FIG. 3B, bacteriophages phages that expressed the native CsgA or CsgB sequences lack the C- and N-terminal “beta-breaking” residues, such as arginines (R) and/or prolines (P) at the N- and C-terminal respectively, and have demonstrated to nucleate amyloid formation at low doses, such as bacteriophages expressing SEQ ID NO: 12 and SEQ ID NO: 29 as shown in FIG. 3B. Moreover, as shown in FIG. 15, T7-RRR-CsgB(133-142)-GGG actually stimulated rather than inhibited biofilm formation, demonstrating that at least one glycine residue, or at least 2, or at least about 3 or at least about 4 or more glycine residies at the C-terminus of the peptide can promote amyloid formation and increase the biofilm formation.

Thus, in some embodiments, pro-amyloid peptide engineered bacteriophage, e.g., a bacteriophage expressing T7-RRR-CsgB(133-142)-GGG, or non-modified CsgA and CsgB peptides lacking N- and C-terminal arginines and prolines (See FIG. 3B), can be used to induce amyloid assembly at low phage concentrations. Additionally, pro-amyloid peptide engineered bacteriophages which express amyloid peptides comprising non-beta-breaker amino acids (such as glycine) added to the C-terminal or N-terminal of the amyloidogenic or amyloid-nucleating domains can assist in biofilm formation, e.g., a bacteriophage expressing T7-RRR-CsgB(133-142)-GGG has been used to empirically demonstrate that particular amyloid peptides can stimulate amyloid formation and can lead to stimulation of biofilm formation (see FIG. 15).

### Bacterial Infections

One aspect of the present invention relates to the use of the methods and compositions comprising an anti-amyloid peptide engineered bacteriophage to inhibit the growth and/or kill (or reduce the cell viability) of a microorganism, such as a bacteria. In some embodiments, a pro-amyloid peptide engineered bacteriophage as disclosed herein can be used to increase bacteria infection or increase the amount of biofilm of bacteria. In some embodiments of this aspect and all aspects described herein, a microorganism is a bacterium. In some embodiments, the bacteria are gram positive or gram negative bacteria. In some embodiments, the bacteria are bacterium resistant to at least one drug. In further embodiments, the bacteria are polymyxin-resistant bacterium. In some embodiments, the bacterium is a persister bacteria. Examples of gram-negative bacteria are for example, but not limited to P. aeruginosa, A. bumannii, Salmonella spp, Klebsiella pneumonia, Shigeila spp. and/or Stenotrophomonas maltophilia. In one embodiment, the bacteria to be targeted using the phage of the invention include E. coli, S. epidermidis, Yersina pestis and Pseudomonas fluorescens.

In some embodiments, the methods and compositions as disclosed herein can be used to kill or reduce the viability of a bacterium, for example a bacterium such as, but not limited to: Bacillus cereus, Bacillus anbhracis, Bacillus cereus, Bacillus anthracis, Clostridium botulinum, Clostridium difficle, Clostridium tetani, Clostridium perfringens, Corynebacteria diptheriae, Enterococcus (Streptococcus D), Lieteria monocytogenes, Pneumoccoccal infections (Streptococcus pneumoniae), Staphylococcal infections and Streptococcal infections; Gram-negative bacteria including Bacteroides, Bordetella pertussis, Brucella, Campylobacter infections, enterohaemorrhagic Escherichia coli (EHEC/E. coli 0157:17), enteroinvasive Escherichia coli (EIEC), enterotoxigenic Escherichia coli (ETEC), Haemophilus influenzae, Helicobacter pylori, Klebsiella pneumoniae, Legionella spp., Moraxella catarrhalis, Neisseria gonnorrhoeae, Neisseria meningitidis, Proteus spp., Pseudomonas aeruginosa, Salmonella spp., Shigella spp., Vibrio cholera and Yersinia; acid fast bacteria including Mycobacterium tuberculosis, Mycobacterium avium-intracellulare, Myobacterium johnei, Mycobacterium leprae, atypical bacteria, Chlamydia, Myoplasma, Rickettsia, Spirochetes, Treponema pallidum, Borrelia recurrentis, Borrelia burgdorfii and Leptospira icterohemorrhagiae, Actinomyces, Nocardia, P. aeruginosa, A. bumannii, Salmonella spp., Klebsiella pneumonia, Shigeila spp. and/or Stenotrophomonas maltophilia and other miscellaneous bacteria.

Bacterial infections include, but are not limited to, infections caused by Bacillus cereus, Bacillus anbhracis, Bacillus cereus, Bacillus anthracis, Clostridium botulinum, Clostridium difficle, Clostridium tetani, Clostridium perfringens, Corynebacteria diptheriae, Enterococcus (Streptococcus D), Lieteria monocytogenes, Pneumoccoccal infections (Streptococcus pneumoniae), Staphylococcal infections and Streptococcal infections/Gram-negative bacteria including Bacteroides, Bordetella pertussis, Brucella, Campylobacter infections, enterohaemorrhagic Escherichia coli (EHEC/E. coli 0157:17) enteroinvasive Escherichia coli (EIEC), enterotoxigenic Escherichia coli (ETEC), Haemophilus influenzae, Helicobacter pylori, Klebsiella pneumoniae, Legionella spp., Moraxella catarrhalis, Neisseria gonnorrhoeae, Neisseria meningitidis, Proteus spp., Pseudomonas aeruginosa, Salmonella spp., Shigella spp., Vibrio cholera and Yersinia; acid fast bacteria including Mycobacterium tuberculosis, Mycobacterium avium-intracellulare, Myobacterium johnei, Mycobacterium leprae, atypical bacteria, Chlamydia, Myoplasma, Rickettsia, Spirochetes, Treponema pallidum, Borrelia recurrentis, Borrelia burgdorfii and Leptospira icterohemorrhagiae and other miscellaneous bacteria, including Actinomyces and Nocardia.

In some embodiments, the microbial infection is caused by gram-negative bacterium, for example, P. aeruginosa, A. bumannii, Salmonella spp, Klebsiella pneumonia, Shigeila spp. and/or Stenotrophomonas maltophilia. Examples of microbial infections include bacterial wound infections, mucosal infections, enteric infections, septic conditions, pneumonia, trachoma, onithosis, trichomoniasis and salmonellosis, especially in veterinary practice.

Examples of infections caused by P. aeruginosa include: A) Nosocomial infections; 1. Respiratory tract infections in cystic fibrosis patients and mechanically-ventilated patients; 2. Bacteraemia and sepsis; 3, Wound infections, particularly in burn wound patients; 4. Urinary tract infections; 5. Post-surgery infections on invasive devises 5. Endocarditis by intravenous administration of contaminated drug solutions; 7, Infections in patients with acquired immunodeficiency syndrome, cancer chemotherapy, steroid therapy, hematological malignancies, organ transplantation, renal replacement therapy, and other situations with severe neutropenia. B) Community-acquired infections; 1. Community-acquired respiratory tract infections; 2. Meningitis; 3. Folliculitis and infections of the ear canal caused by contaminated waters; 4. Malignant otitis externa in the elderly and diabetics; 5. Osteomyelitis of the caleaneus in children; Eye infections commonly associated with contaminated contact lens; 6. Skin infections such as nail infections in people whose hands are frequently exposed to water; 7. Gatrointestinal tract infections; 8. Muscoskeletal system infections.

Examples of infections caused by A. baumannii include: A) Nosocomial infections 1. Bacteraemia and sepsis, 2. respiratory tract infections in mechanically ventilated patients; 3. Post-surgery infections on invasive devices; 4. wound infectious, particularly in burn wound patients; 5. infection in patients with acquired immunodeficiency syndrome, cancer chemotherapy, steroid therapy, hematological malignancies, organ transplantation, renal replacement therapy, and other situations with severe neutropenia; 6. urinary tract infections; 7. Endocarditis by intravenous administration of contaminated drug solutions; 8. Cellulitis. B) Community-acquired infections: a. community-acquired pulmonary infections; 2. Meningitis; Cheratitis associated with contaminated contact lens; 4. War-zone community-acquired infections. C) Atypical infections: 1. Chronic gastritis.

Examples of infections caused by Stenotrophomonas maltophilia include Bacteremia, pneumonia, meningitis, wound infections and urinary tract infections. Some hospital breaks are caused by contaminated disinfectant solutions, respiratory devices, monitoring instruments and ice machines. Infections usually occur in debilitated patients with impaired host defense mechanisms.

Examples of infections caused by Klebsiella pneumoniae include community-acquired primary lobar pneumonia, particularly in people with compromised pulmonary function and alcoholics. It also caused wound infections, soft tissue infections and urinary tract infections.

Examples of infections caused by Salmonella app. are acquired by eating contaminated food products. Infections include enteric fever, enteritis and bacteremia.

Examples of infections caused by Shigella spp. include gastroenteristis (shigellosis).

The methods and compositions as disclosed herein comprising an anti-amyloid peptide engineered bacteriophage can also be used in various fields as where antiseptic treatment or disinfection of materials it required, for example, surface disinfection.

The methods and compositions as disclosed herein comprising an anti-amyloid peptide engineered bacteriophage can be used to treat microorganisms infecting a cell, group of cells, or a multi-cellular organism.

In one embodiment, an anti-amyloid peptide engineered bacteriophage as described herein can be used to reduce the rate of proliferation and/or growth of microorganisms. In some embodiments, the microorganism are either or both gram-positive or gram-negative bacteria, whether such bacteria are cocci (spherical), rods, vibrio (comma shaped), or spiral.

Of the cocci bacteria, micrococcus and staphylococcus species are commonly associated with the skin, and Streptococcus species are commonly associated with tooth enamel and contribute to tooth decay. Of the rods family, bacteria Bacillus species produce endospores seen in various stages of development in the photograph and B. cereus cause a relatively mild food poisoning, especially due to reheated fried food. Of the vibrio species, V. cholerae is the most common bacteria and causes cholera, a severe diarrhea disease resulting from a toxin produced by bacterial growth in the gut. Of the spiral bacteria, rhodospirillum and Treponema pallidum are the common species to cause infection (e.g., Treponema pallidum causes syphilis). Spiral bacteria typically grow in shallow anaerobic conditions and can photosynthesize to obtain energy from sunlight.

Moreover, the present invention relates to the use of an anti-amyloid peptide engineered bacteriophage, or a composition comprising an anti-amyloid peptide engineered bacteriophage to reduce the rate of growth and/or kill either gram positive, gram negative, or mixed flora bacteria or other microorganisms. In one embodiment, a composition consists essentially of an anti-amyloid peptide engineered bacteriophage as disclosed herein for the use to reduce the rate of growth and/or kill either gram positive, gram negative, or mixed flora bacteria or other microorganisms. In another embodiment, the composition contains at least one anti-amyloid peptide engineered bacteriophage as disclosed herein for the use to reduce the rate of growth and/or kill either gram positive, gram negative, or mixed flora bacteria or other microorganisms.

Such bacteria are for example, but are not limited to, listed in Table 2. Further examples of bacteria are, for example but not limited to Baciccis Antracis; Enterococcus faecalis; Corynebacterium; diphtheriae; Escherichia coli; Streptococcus coelicolor; Streptococcus pyogenes; Streptobacillus “oniliformis; Streptococcus agalactiae; Streptococcus pneurmoniae; Salmonella typhi; Salmonella paratyphi; Salmonella schottmulleri; Salmonella hirshieldii; Staphylococcus epidermidis; Staphylococcus aureus; Klebsiella pzeumoniae; Legionella pneumophila; Helicobacter pylori; Mycoplasma pneumonia; Mycobacterium tuberculosis; Mycobacterium leprae; Yersinia enterocolitica; Yersinia pestis; Vibrio cholerae; Vibrio parahaemolyticus; Rickettsia prowozekii; Rickettsia rickettsii; Rickettsia akari; Clostridium difficile; Clostridium tetani; Clostridium perfringens; Clostridianz novyii; Clostridianz septicum; Clostridium botulinum; Legionella pneumophila; Hemophilus influenzue; Hemophilus parainfluenzue; Hemophilus aegyptus; Chlamydia psittaci; Chlamydia trachonZatis; Bordetella pertcsis; Shigella spp.; Campylobacter jejuni; Proteus spp.; Citrobacter spp.; Enterobacter spp.; Pseudomonas aeruginosa; Propionibacterium spp.; Bacillus anthracia; Pseudomonas syringae; Spirrilum minus; Neisseria meningitidis; Listeria monocytogenes; Neisseria gonorrheae; Treponema pallidum; Francisella tularensis; Brucella spp.; Borrelia recurrentis; Borrelia hermsii; Borrelia turicatue; Borrelia burgdorferi; Mycobacterium avium; Mycobacterium smegmatis; Methicillin-resistant Staphyloccus aureus; Vanomycin-resistant enterococcus; and multi-drug resistant bacteria (e.g., bacteria that are resistant to more than 1, more than 2, more than 3, or more than 4 different drugs).

TABLE 2
Examples of bacteria.

| | | |
|---|---|---|
| Staphyloccocus aureus | Nisseria menigintidis | Helicbacter pylori |
| Bacillus anthracis | Nisseria gonerrhoeae | Legionella |
| Bacillus cereus | Vibrio cholerae | pnemophilia |
| Bacillus subtillis | Escherichia coli K12 | Borrelia burgdorferi |
| Streptococcus phemonia | Bartonella henselae | Ehrlichia chaffeensis |
| Streptococcus pyogenes | Haemophilus | Treponema pallidum |
| Clostridium tetani | influenzae | Chlamydia |
| Listeria monocytogenes | Salmonella typhi | trachomatis |
| Mycobacterium | Shigella dysentriae | |
| tuberculosis | Yerinisa pestis | |
| Staphyloccocus | Pseudomona | |
| epidermidis | aeruginosa | |

In some embodiments, an anti-amyloid peptide engineered bacteriophage as described herein can be used to treat an already drug resistant bacterial strain such as Methicillin-resistant Staphylococcus aureus (MRSA) or Vancomycin-resistant enterococcus (VRE) of variant strains thereof.

In some embodiments, the present invention also contemplates the use and methods of use of an anti-amyloid peptide engineered bacteriophage as described herein in all combinations with other agents, such as other anti-amyloid peptides and/or antibiotics to fight gram-positive bacteria that maintain resistance to certain drugs.

In some embodiments, an anti-amyloid peptide engineered bacteriophage as disclosed herein can be used to treat infections, for example bacterial infections and other conditions such as urinary tract infections, ear infections, sinus infections, bacterial infections of the skin, bacterial infections of the lungs, sexually transmitted diseases, tuberculosis, pneumonia, lyme disease, and Legionnaire's disease. Thus any of the above conditions and other conditions resulting from a microorganism infection, for example a bacterial infection or a biofilm can be prevented or treated by the compositions of the invention herein.

### Biofilms

Another aspect of the present invention relates to the use of an anti-amyloid peptide engineered bacteriophage to eliminate or reduce a bacterial biofilm, for example a bacterial biofilm in a medical, or industrial, or biotechnological setting. Alternatively, in some embodiments, the use of a pro-amyloid peptide engineered bacteriophage can be used to increase the bacteria biofilm, for example to promote the formation of bacteria biofilms for formation of environmental biosensors, detection of environmental conditions and toxins as well as reducing pathogenic biofilms such as biofouling, and for promoting biofilms in microbial fuel cells, to promote good bacteria to compete out harmful or pathogenic bacteria, and other such applications for promoting biofilm formation using engineered bacteriophages espressing pro-amyloid peptides.

For instance, some bacteria, including P. aeruginosa, actively form tightly arranged multi-cell structures in vivo known as biofilm. The production of biofilm is important for the persistence of infectious processes such as seen in pseudomonal lung-infections in patients with cystic fibrosis and diffuse panbronchiolitis and many other diseases. A bioflim is typically resistant to phagocytosis by host immune cells and the effectiveness of antibiotics at killing bacteria in biofilm structures can be reduced by 10 to 1000 fold. Bioflim production and arrangement is governed by quorum sensing systems. The disruption of the quorum sensing system in bacteria such as P. aeruginosa is an important anti-pathogenic activity as it disrupts the biofilm formation and also inhibits alginate production

### Pharmaceutical Formulations and Compositions

The anti-amyloid peptide engineered bacteriophage or pro-amyloid engineered bacteriophages as disclosed herein can be formulated in combination with one or more pharmaceutically acceptable agents. In some embodiments, combinations of different an anti-amyloid peptide engineered bacteriophages or pro-amyloid engineered bacteriophages can be tailored to be combined, where different anti-amyloid peptide engineered bacteriophages or pro-amyloid engineered bacteriophages are designed to target different (or the same) species of microorganisms or bacteria, which contribute towards morbidity and mortality. A pharmaceutically acceptable composition comprising an an anti-amyloid peptide engineered bacteriophage as disclosed herein, are suitable for internal administration to an animal, for example human.

In some embodiments, an anti-amyloid peptide engineered bacteriophage or pro-amyloid engineered bacteriophages as disclosed herein can be used for industrial sterilizing, sterilizing chemicals such as detergents, disinfectants, and ammonium-based chemicals (e.g. quaternary ammonium compounds such as QUATAL, which contains 10.5% N-alkyldimethyl-benzlammonium HCl and 5.5% gluteraldehyde as active ingredients, Ecochimie Ltée, Quebec, Canada), and can be used in concurrently with, or prior to or after the treatment or administration of an anti-amyloid peptide or agent which inhibits fiber association. Such sterilizing chemicals are typically used in the art for sterilizing industrial work surfaces (e.g. in food processing, or hospital environments), and are not suitable for administration to an animal.

In some embodiments, an anti-amyloid peptide engineered bacteriophage as disclosed herein can be used for household cleaning and sterilizing purposes. The anti-amyloid peptide engineered bacteriophage can be used in combination with other cleaning and sterilizing chemicals, e.g. detergents or disinfectants, or it can be administered before, after or concurrently with administration of other antibacterial agents capable of assisting in biofilm dispersion.

In another aspect of the present invention relates to a pharmaceutical composition comprising an anti-amyloid peptide engineered bacteriophage and a pharmaceutically acceptable excipient. Suitable carriers for the an anti-amyloid peptide engineered bacteriophage of the invention, and their formulations, are described in Remington's Pharmaceutical Sciences, 16^{th} ed., 1980, Mack Publishing Co., edited by Oslo et al. Typically an appropriate amount of a pharmaceutically acceptable salt is used in the formulation to render the formulation isotonic. Examples of the carrier include buffers such as saline, Ringer's solution and dextrose solution. The pH of the solution is preferably from about 5 to about 8, and more preferably from about 7.4 to about 7.8. Further carriers include sustained release preparations such as semipermeable matrices of solid hydrophobic polymers, which matrices are in the form of shaped articles, e.g. liposomes, films or microparticles. It will be apparent to those of skill in the art that certain carriers can be more preferable depending upon for instance the route of administration and concentration of an anti-amyloid peptide engineered bacteriophage being administered.

Administration to human can be accomplished by means determined by the underlying condition. For example, if an anti-amyloid peptide engineered bacteriophage is to be delivered into lungs of an individual, inhalers can be used. Such formulations can also include freeze-dried powders of the engineered bacteriophage, for example, for administration of the anti-amyloid peptide engineered bacteriophage to a subject by dry-powder inhalers or reconstitution for nebulization. If the composition is to be delivered into any part of the gut or colon, coated tablets, suppositories or orally administered liquids, tablets, caplets and so forth can be used. A skilled artisan will be able to determine the appropriate way of administering the phages of the invention in view of the general knowledge and skill in the art.

Compounds as disclosed herein, can be used as a medicament or used to formulate a pharmaceutical composition with one or more of the utilities disclosed herein. They can be administered in vitro to cells in culture, in vivo to cells in the body, or ex vivo to cells outside of a subject that can later be returned to the body of the same subject or another subject. Such cells can be disaggregated or provided as solid tissue in tissue transplantation procedures.

Compositions comprising at least one anti-amyloid peptide engineered bacteriophage or pro-amyloid engineered bacteriophages as disclosed herein can be used to produce a medicament or other pharmaceutical compositions. Use of the compositions as disclosed herein comprising an anti-amyloid peptide engineered bacteriophage can further comprise a pharmaceutically acceptable carrier. The composition can further comprise other components or agents useful for delivering the composition to a subject are known in the art. Addition of such carriers and other components to the agents as disclosed herein is well within the level of skill in this art.

In some embodiments, the composition comprising an anti-amyloid peptide engineered bacteriophage is a composition for sterilization of a physical object that is infected with bacteria, such as sterilization of hospital equipment, industrial equipment, medical devices and food products. In another embodiment, a composition comprising an anti-amyloid peptide engineered bacteriophage is a pharmaceutical composition useful to treat a bacterial infection in a subject, for example a human or animal subject.

In some embodiments, a pharmaceutical composition comprising an anti-amyloid peptide engineered bacteriophage as disclosed herein can be administered as a formulation adapted for passage through the blood-brain barrier or direct contact with the endothelium, for example where the anti-amyloid peptide inhibits the formation or maintenance of β-amyloid plaques in Alzheimer's disease. In some embodiments, the pharmaceutical composition comprising an anti-amyloid peptide engineered bacteriophage can be administered as a formulation adapted for systemic delivery. In some embodiments, the compositions can be administered as a formulation adapted for delivery to specific organs, for example but not limited to the liver, bone marrow, or systemic delivery.

Alternatively, pharmaceutical compositions comprising an anti-amyloid peptide engineered bacteriophage or pro-amyloid engineered bacteriophages can be added to the culture medium of cells ex vivo. In addition to an anti-amyloid peptide engineered bacteriophage or pro-amyloid engineered bacteriophages, such compositions can contain pharmaceutically-acceptable carriers and other ingredients or agents known to facilitate administration and/or enhance uptake (e.g., saline, dimethyl sulfoxide, lipid, polymer, affinity-based cell specific-targeting systems). In some embodiments, a pharmaceutical composition can be incorporated in a gel, sponge, or other permeable matrix (e.g., formed as pellets or a disk) and placed in proximity to the endothelium for sustained, local release. The composition comprising an anti-amyloid peptide engineered bacteriophage can be administered in a single dose or in multiple doses which are administered at different times.

Pharmaceutical compositions comprising an anti-amyloid peptide engineered bacteriophage or pro-amyloid engineered bacteriophage can be administered to a subject by any known route. By way of example, a composition comprising an anti-amyloid peptide engineered bacteriophage can be administered by a mucosal, pulmonary, topical, or other localized or systemic route (e.g., enteral and parenteral). The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

Pharmaceutical compositions can also optionally comprise include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (i.e., adjuvants), or targeting carries to target the immunogenic peptide to specific target cells or target organs, for example the bone marrow as a target organ or plasma cells as target cells.

For parenteral administration, the immunogenic peptide of the present invention can be administered as injectable dosages of a solution or suspension of the substance in a physiologically acceptable diluent with a pharmaceutical carrier which can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25, 3521-24 (1995); Cevc et al., Biochem. Biophys. Acta 1368, 201-15 (1998)).

Suitable choices in amounts and timing of doses, formulation, and routes of administration can be made with the goals of achieving a favorable response in the subject with a bacterial infection or infection with a microorganism, for example, a favorable response is killing or elimination of the microorganism or bacteria, or control of, or inhibition of growth of the bacterial infection in the subject or a subject at risk thereof (i.e., efficacy), and avoiding undue toxicity or other harm thereto (i.e., safety). Therefore, “effective” refers to such choices that involve routine manipulation of conditions to achieve a desired effect or favorable response.

A bolus of the pharmaceutical composition comprising an anti-amyloid peptide engineered bacteriophage can be administered to a subject over a short time, such as once a day is a convenient dosing schedule. Alternatively, the effective daily dose can be divided into multiple doses for purposes of administration, for example, two to twelve doses per day. Dosage levels of active ingredients in a pharmaceutical composition can also be varied so as to achieve a transient or sustained concentration of the composition in the subject, especially in and around the area of the bacterial infection or infection with a microorganism, and to result in the desired therapeutic response or protection. It is also within the skill of the art to start doses at levels lower than required to achieve the desired therapeutic effect and to gradually increase the dosage until the desired effect is achieved.

The effective amount of a pharmaceutical composition comprising an anti-amyloid peptide engineered bacteriophage or pro-amyloid engineered bacteriophage to be administered to a subject is dependent upon factors known to a persons of ordinary skill in the art such as bioactivity and bioavailability of the anti-amyloid peptide (e.g., half-life in the body, stability, and metabolism of the engineered bacteriophage); chemical properties of the anti-amyloid peptide (e.g., molecular weight, hydrophobicity, and solubility); route and scheduling of administration, and the like. It will also be understood that the specific dose level of the composition comprising an anti-amyloid peptide engineered bacteriophage as disclosed herein to be achieved for any particular subject can depend on a variety of factors, including age, gender, health, medical history, weight, combination with one or more other drugs, and severity of disease, and bacterial strain or microorganism the subject is infected with, such as infection with multi-resistant bacterial strains.

The term “treatment”, with respect to treatment of a bacterial infection or bacterial colonization, inter alia, preventing the development of the disease, or altering the course of the disease (for example, but not limited to, slowing the progression of the disease), or reversing a symptom of the disease or reducing one or more symptoms and/or one or more biochemical markers in a subject, preventing one or more symptoms from worsening or progressing, promoting recovery or improving prognosis, and/or preventing disease in a subject who is free therefrom as well as slowing or reducing progression of existing disease.

### Treatment Regimes.

### Therapeutic Use

### Selection of Subjects Administered a Composition Comprising an Engineered Bacteriophage

In some embodiments, an anti-amyloid peptide engineered bacteriophage and compositions thereof are useful in a method to treat a subject with an amyloid associated disease or disorder, which include for example but are not limited to, amyloid-related diseases, Alzheimer's Disease, Down's syndrome, vascular dementia or cognitive impairment, type II diabetes mellitus, amyloid A (reactive), secondary amyloidosis, familial mediterranean fever, familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heredity cerebral hemorrhage with amyloidosis (Icelandic), AApo-A-I (familial amyloidotic polyneuropathy—Iowa), AApo-A-II (accelerated senescence in mice), fibrinogen-associated amyloid; Parkinson's disease, systemic amyloidoses (e.g., AL-, AA-, ATTR-, A beta 2, microglobulin, IAPP/amylin amyloidosis) and Asor or Pr P-27 (scrapie, Creutzfeld jacob disease, Gertsmann-Straussler-Scheinker syndrome, bovine spongiform encephalitis) and subjects who are homozygous for the apolipoprotein E4 allele.

Other types of amyloid associated disorders include, for example AL amyloidosis, for example primary amyloidosis, secondary amyloidosis and hereditary amyloidosis. Without being bound by theory, in AL-amyloidosis are fibrils of AL amyloid deposits which are composed of monoclonal immunoglobulin light chains or fragments thereof. More specifically, the fragments are a region of the N-terminal region of the light chain (kappa or lambda), or derivatives thereof, and contain all or part of the variable (V_{L}) domain thereof. More specifically, the fragments do not contain a region of the heavy chain of the variable region (V_{H}). Deposits generally occur in the mesenchymal tissues, causing peripheral and autonomic neuropathy, carpal tunnel syndrome, macroglossia, restrictive cardiomyopathy, arthropathy of large joints, immune dyscrasias, multiple myelomas, as well as ocular dyscrasias. However, it should be noted that almost any tissue, particularly visceral organs such as the heart, may be involved. In light chain amyloidosis (AL-amyloidosis) a monoclonal immunoglobulin light chain forms the amyloid deposits. See Glenner et al., Amyloid Fibril Proteins: Proof of Homology with Immunoglobulin Light Chains by Sequence Analyses, Science 172:1150-1151, 1971. Amyloid fibrils from patients suffering AL-amyloidosis occasionally contain only intact light chains, but more often they are formed by proteolytic fragments of the light chains which contain the VL domain and varying amounts of the constant domain, or by a mixture of fragments and full-length light chains. Not all light chains from plasma cell dyscrasias form protein deposits; some circulate throughout the body at high concentrations and are excreted with the subject's urine without pathological deposition of the protein in vivo. See Solomon, Clinical Implications of Monoclonal Light Chains, Semin. OncoL 13:341-349, 1986; Buxbaum, Mechanisms of Disease: Monoclonal Immunoglobulin Deposition, Amyloidosis, Light Chain Deposition Disease, and Light and Heavy Chain Deposition Disease, Hematol./Oncol. Clinics of North America 6:323-346, 1992; and Eulitz, Amyloid Formation from Immunoglobulin Chains, Biol. Chef Hoppe-Seyler 373:629-633, 1992. Subjects suffering from AL amyloidosis can be recognized from methods known by a physician of ordinary skill, for example, typical symptoms of amyloidosis depend on the organ affected and include a wide range of symptoms, for example but are not limited to at least one of the following or combinations of; swelling of your ankles and legs, weakness, weight loss, shortness of breath, numbness or tingling in your hands or feet, diarrhea, severe fatigue, an enlarged tongue (macroglossia), skin changes, an irregular heartbeat, and difficulty swallowing. In some instances, the subject may not experience any of the symptoms listed but still has amyloidosis. In addition, a number of diagnostic tests are available for identifying subjects at risk of, or having AL amyloidosis which are commonly known by person skilled in the art, and are encompassed for use in the present invention. These include measurement of including blood and urine tests, though blood or urine tests may detect an abnormal protein, which could indicate amyloidosis, the only definitive test for amyloidosis is a tissue biopsy, in which the physical analyses a small sample of tissue. The tissue sample may be taken from one or more parts of the subject's body, for example abdominal fat, bone marrow or rectum, which is then examined under a microscope in a laboratory to check for signs of amyloid. Occasionally, tissue samples may be taken from other parts of your body, such as your liver or kidney, to help diagnose the specific organ affected by amyloidosis.

Primary Amyloidosis.

This most common form of amyloidosis primarily affects your heart, kidneys, tongue, nerves and intestines. Primary amyloidosis isn't associated with other diseases except for multiple myeloma, in a minority of cases. The cause of primary amyloidosis is unknown, but doctors do know that the disease begins in your bone marrow. In addition to producing red and white blood cells and platelets, your bone marrow makes antibodies, the proteins that protect you against infection and disease. After antibodies serve their function, your body breaks them down and recycles them. Amyloidosis occurs when cells in the bone marrow produce antibodies that can't be broken down. These antibodies then build up in your bloodstream. Ultimately, they leave your bloodstream and can deposit in your tissues as amyloid, interfering with normal function.

Secondary Amyloidosis.

This form occurs in association with chronic infectious or inflammatory diseases, such as tuberculosis, rheumatoid arthritis or osteomyelitis, a bone infection. It primarily affects your kidneys, spleen, liver and lymph nodes, though other organs may be involved. Treatment of the underlying disease may help stop this form of amyloidosis.

Hereditary Amyloidosis.

As the name implies, this form of amyloidosis is inherited. This type often affects the nerves, heart and kidneys.

There are a variety of other forms of amyloid associated disease and disorders that are normally manifest as localized deposits of amyloid. In general, these diseases are probably the result of the localized production and/or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid.

In some types of hereditary amyloidoses, single amino acid changes in normal human proteins are responsible for amyloid fibril formation See Natvig et al., Amyloid and Amyloidosis 1990. Dordrecht, The Netherlands: Kluwer Academic Publishers, 1991, and references cited therein. It is unlikely, however, that any single amino acid position or substitution will fully explain the many different immunoglobulin light chain sequences associated with AL-amyloidosis. Rather, several different regions of the light chain molecule may sustain one or more substitutions which affect a number of biophysical characteristics, such as dimer formation, exposure of hydrophobic residues, solubility, and stability.

Heavy chain diseases are neoplastic plasma cell dyscrasias characterized by the overproduction of homogenous α, γ, and mu Ig heavy chains. These disorders result in incomplete monoclonal Igs. The clinical picture is more like lymphoma than multiple myeloma.

In some embodiments, the present invention provides methods to treat a disease and/or disorder associated with an amyloidogenic disease or an amyloid-associated disorder. Amyloidogenic diseases and amyloid-associated disorders are diseases from the secretion of a protein and/or peptide that aggregates and forms a deposit and is characterized by amyloid deposits or fibril formation. The methods of the present invention provide use of anti-amyloid peptides for the treatment of such amyloidogenic diseases or amyloid-associated disorders. Such amyloidogenic diseases and amyloid-associated disorders include, for example but is not limited to, Alzheimer's disease, Parkinson's disease, Down's syndrome, vascular dementia or cognitive impairment, type II diabetes mellitus, amyloid A (reactive), secondary amyloidosis, familial mediterranean fever, systemic amyloidoses (e.g., AL, AA, ATTR, A beta 2 microglobulin, IAPP/amylin), familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heritiaty cerebral hemorrhage with amyloidosis (Icelandic), AApo-A-I (familial amyloidotic polyneuropathy—Iowa), AApo-A-II (accelerated senescence in mice), fibrinogen-associated amyloid; and Asor or Pr P-27 (scrapie, Creutzfeld jacob disease, Gertsmann-Straussler-Scheinker syndrome, bovine spongiform encephalitis) and person who are homozygous for the apolipoprotein E4 allele.

As used herein, the terms “prevent,” “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the complete avoidance or prevention of symptoms or markers, but also a reduced severity or degree of any one of those symptoms or markers, relative to those symptoms or markers arising in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

In some embodiments, a subject amenable for the methods as described herein or for the administration with a composition comprising at least one anti-amyloid peptide engineered bacteriophage is selected based on the desired treatment regime. For instance, a subject is selected for treatment if the subject suffers from, or is at risk of an amyloid associated disorder.

In some embodiments, a subject with an amyloid associated disorder is a subject having or likely to develop a bacterial infection where the bacteria form a biofilm, or where the subject has been non-responsive to prior therapy or administration with an anti-amyloid peptide.

Accordingly, in some embodiments, a subjects suffering from, or at risk of developing an amyloid-associated disorders is administered an anti-amyloid peptide engineered bacteriophage.

In some embodiments, a subject can be administered a composition comprising at an anti-amyloid peptide engineered bacteriophage which expresses, for example at least one, 2, 3, or 4 or as many of 10 different anti-amyloid peptides. In some embodiments, a subject is administered at least one anti-amyloid peptide engineered bacteriophage, as disclosed herein, or a plurality anti-amyloid peptide engineered bacteriophages, for example, for example at least 2, 3, or 4 or as many of 10 different anti-amyloid peptide engineered bacteriophage as disclosed herein. In some embodiments, the composition can comprise an anti-amyloid peptide engineered bacteriophage with at least one or a variety of different other bacteriophages, or different anti-amyloid peptide engineered bacteriophage. In alternative embodiments, the composition can comprise at least two, or at least 3, 4, 5 or as many of 10 different anti-amyloid peptide engineered bacteriophage, wherein each of the anti-amyloid peptide engineered bacteriophages comprise a nucleic acid which encodes at least different anti-amyloid peptide. Any combination and mixture of anti-amyloid peptide engineered bacteriophages are useful in the compositions and methods of the present invention.

In some embodiments, an anti-amyloid peptide engineered bacteriophage is administered to a subject at the same time, prior to, or after the administration of an additional agent. In some embodiments, an anti-amyloid peptide engineered bacteriophage can be formulated to a specific time-release for activity, such as the an anti-amyloid peptide engineered bacteriophage is present in a time-release capsule. In such embodiments, an anti-amyloid peptide that is formulated for time-release can be administered to a subject at the same time, concurrent with, or prior to, or after the administration of an additional agent, such as an additional therapeutic, anti-amyloid peptide or agent which inhibits fiber association. Methods of formulation of an anti-amyloid peptide engineered bacteriophage for release in a time-dependent manner are disclosed herein as “sustained release pharmaceutical compositions” in the section entitled “pharmaceutical formulations and compositions.” Accordingly, in such embodiments, a time-release an anti-amyloid peptide engineered bacteriophage can be administered to a subject at the same time (i.e. concurrent with), prior to or after the administration of an additional agent, such as an additional therapeutic agent or therapeutic agent.

In some embodiments, an additional agent administered at the same or different time as an anti-amyloid peptide engineered bacteriophage can be a pro-drug, where it is activated by a second agent. Accordingly, in such embodiments, a pro-drug agent can be administered to a subject at the same time, concurrent with, or prior to, or after the administration of an anti-amyloid peptide engineered bacteriophage, and administration of an agent which activates the pro-drug into its active form can be administered the same time, concurrent with, or prior to, or after the administration of the anti-amyloid peptide engineered bacteriophage.

In some embodiments, a subject is selected for the administration with the compositions comprising an anti-amyloid peptide engineered bacteriophage as disclosed herein by identifying a subject that needs a specific treatment regimen, and is administered an anti-amyloid peptide engineered bacteriophage concurrently with, or prior to, or after administration with an additional therapeutic agent.

Using a subject with cystic fibrosis as an exemplary example, a subject could be administered an anti-amyloid peptide engineered bacteriophage to avoid chronic endobronchial infections, such as those caused by pseudomonas aeruginosis or stentrophomonas

As disclosed in the Example 9, the inventors discovered that the nucleating sequence of CsgB (e.g. CsgB₁₃₄₋₁₄₀) as TAIVVQR (SEQ ID NO: 196) can inhibit formation of amyloid from amyloid-β nucleators, which a subset thereof contains a sequence, VVIA (SEQ ID NO: 198) exactly the reverse of the critical nucleating sequence of CsgB, AIVV (SEQ ID NO: 199). Thus, in a certain embodiment, an anti-amyloid peptide engineered bacteriophage expressing TAIVVQR (SEQ ID NO: 196) can be used for the treatment of Alzheimer's disease. In another embodiment, an anti-amyloid peptide engineered bacteriophage comprising an amino acid sequence of AIVV (SEQ ID NO: 199) can also be used for the treatment of Alzheimer's disease. In some embodiments, the treatment is prophylactic treatment.

### Alzheimer's Disease

Alzheimer's disease (AD) is a progressive disease resulting in senile dementia. See generally Selkoe, TINS 16, 403-409 (1993); Hardy et al., WO 92/13069; Selkoe, J. Neuropathol. Exp. Neurol. 53, 438-447 (1994); Duff et al., Nature 373, 476-477 (1995); Games et al., Nature 373, 523 (1995). Broadly speaking the disease falls into two categories: late onset, which occurs in old age (65+ years) and early onset, which develops well before the senile period, i.e., between 35 and 60 years. In both types of disease, the pathology is the same but the β abnormalities tend to be more severe and widespread in cases beginning at an earlier age. The disease is characterized at the macroscopic level by significant brain shrinkage away from the cranial vault as seen in MRI images as a direct result of neuronal loss and by two types of macroscopic lesions in the brain, senile plaques and neurofibrillary tangles. Senile plaques are areas comprising disorganized neuronal processes up to 150 μm across and extracellular amyloid deposits, which are typically concentrated at the center and visible by microscopic analysis of sections of brain tissue. Neurofibrillary tangles are intracellular deposits of tau protein consisting of two filaments twisted about each other in pairs.

The principal constituent of the plaques is a peptide termed Aβ or β-amyloid peptide. Aβ peptide is an internal fragment of 39-43 amino acids of a precursor protein termed amyloid precursor protein (APP). Several mutations within the APP protein have been correlated with the presence of Alzheimer's disease. See, e.g., Goate et al., Nature 349, 704) (1991) (valine⁷¹⁷ to isoleucine); Chartier Harlan et al. Nature 353, 844 (1991)) (valine⁷¹⁷ to glycine); Murrell et al., Science 254, 97 (1991) (valine⁷¹⁷ to phenylalanine); Mullan et al., glycine); Murrell et al., Science 254, 97 (1991) (valine⁷¹⁷ to phenylalanine); Mullan et al., Nature Genet. 1, 345 (1992) (a double mutation changing lysine⁵⁹⁵-methionine⁵⁹⁶ to asparagine⁵⁹⁵-leucine⁵⁹⁶). Such mutations are thought to cause Alzheimer's disease by increased or altered processing of APP to Aβ, particularly processing of APP to increased amounts of the long form of Aβ (i.e., Aβ 1-42 and Aβ 1-43). Mutations in other genes, such as the presenilin genes, PS1 and PS2, are thought indirectly to affect processing of APP to generate increased amounts of long form Aβ (see Hardy, TINS 20, 154 (1997)). These observations indicate that Aβ, and particularly its long form, is a causative element in Alzheimer's disease.

Aβ, also known as β-amyloid peptide, or A4 peptide (see U.S. Pat. No. 4,666,829; Glenner & Wong, Biochem. Biophys. Res. Commun. 120, 1131 (1984)) in the art, is a peptide of 39-43 amino acids, is the principal component of characteristic plaques of Alzheimer's disease. Aβ is generated by processing of a larger protein APP by two enzymes, termed β and γ secretases (see Hardy, TINS 20, 154 (1997)). Known mutations in APP associated with Alzheimer's disease occur proximate to the site of β or γ-secretase, or within Aβ. For example, position 717 is proximate to the site of γ-secretase cleavage of APP in its processing to Aβ, and positions 670/671 are proximate to the site of β-secretase cleavage. It is believed that the mutations cause AD disease by interacting with the cleavage reactions by which Aβ is formed so as to increase the amount of the 42/43 amino acid form of Aβ generated.

Aβ has the unusual property that it can fix and activate both classical and alternate complement cascades. In particular, it binds to Clq and ultimately to C3bi. This association facilitates binding to macrophages leading to activation of B cells. In addition, C3bi breaks down further and then binds to CR2 on B cells in a T cell dependent manner leading to a 10,000 increase in activation of these cells. This mechanism causes Aβ to generate an immune response in excess of that of other antigens.

Most therapeutic strategies for Alzheimer's disease are aimed at reducing or eliminating the deposition of Aβ42 in the brain, typically via reduction in the generation of Aβ42 from APP and/or some means of lowering existing Aβ42 levels from sources that directly contribute to the deposition of this peptide in the brain (De Felice and Ferreira, 2002). A partial list of aging-associated causative factors in the development of sporadic Alzheimer's disease includes a shift in the balance between Aβ peptide production and its clearance from neurons that favors intracellular accumulation, increased secretion of Aβ peptides by neurons into the surrounding extracellular space, increased levels of oxidative damage to these cells, and global brain hypoperfusion and the associated compensatory metabolic shifts in affected neurons (Cohen et al., 1988; Higgins et al., 1990; Kalaria, 2000; Nalivaevaa et al., 2004; Teller et al., 1996; Wen et al., 2004).

The Aβ42 that deposits within neurons and plaques could also originate from outside of the neurons (exogenous Aβ42) during Alzheimer's disease pathogenesis. Levels of soluble Aβ peptides in the blood are known to be much higher than in the interstitial space and CSF in the brains of healthy individuals (Seubert et al., 1992) with blood as a source of exogenous Aβ peptides that eventually deposit in the Alzheimer's disease brain (Zlokovic et al., 1993).

Genetic markers of risk toward Alzheimer's disease include mutations in the APP gene, particularly mutations at position 717 and positions 670 and 671 referred to as the Hardy and Swedish mutations respectively (see Hardy, TINS, supra). Other markers of risk are mutations in the presenilin genes, PS1 and PS2, and ApoE4, family history of Alzheimer's disease, hypercholesterolemia or atherosclerosis. Subjects presently suffering from Alzheimer's disease can be recognized from characteristic dementia, as well as the presence of risk factors described above. In addition, a number of diagnostic tests are available for identifying subjects who have Alzheimer's disease. These include measurement of CSF tau and Aβ42 levels. Elevated tau and increased Aβ42 levels signify the presence of Alzheimer's disease. Individuals suffering from Alzheimer's disease can also be diagnosed by MMSE or ADRDA criteria. The tissue sample for analysis is typically blood, plasma, serum, mucus or cerebral spinal fluid from the patient. The sample is analyzed for indicia of an immune response to any forms of Aβ peptide, typically Aβ42. The immune response can be determined from the presence of, e.g., antibodies or T-cells that specifically bind to Aβ peptide. ELISA methods of detecting antibodies specific to Aβ are described in the Examples section.

In asymptomatic patients, treatment can begin at any age (e.g., 10, 20, 30). Usually, however, it is not necessary to begin treatment until a patient reaches 40, 50, 60 or 70. Treatment typically entails at least one, or multiple dosages of a composition comprising an anti-amyloid engineered bacteriophage over a period of time. Treatment can be monitored by assaying the amount of Aβ peptide, or the amount of Aβ peptide in the CSF over time. If the Aβ peptide is still present in the CSF additional treatment with anti-amyloid engineered bacteriophages as disclosed herein are recommended, and/or treatment of additional therapies for Alzheimer's disease. In the case of potential Down's syndrome patients, treatment with an anti-amyloid engineered bacteriophage can begin antenatally by administering therapeutic agent to the mother or shortly after birth.

In some embodiments, anti-amyloid engineered bacteriophages as disclosed herein are also useful in the treatment of other neurodegenerative disorders with amyloid deposits, e.g., Creutzfeldt-Jakob or mad cow disease, Huntington's disease, multiple sclerosis, Parkinson's disease, Pick disease and other brain storage disorders (e.g., amyloidosis, gangliosidosis, lipid storage disorders, mucopolysaccharidosis). Thus, treatment with an anti-amyloid engineered bacteriophage as disclosed herein can be directed to a subject who is affected with, yet asymptomatic of a neurodegenerative disease characterized by amyloid deposits. The efficacy of treatment can be determined by measuring the presence and amount of Tau or Aβ in the CSF. Some methods entail determining a baseline value of, for example the level of beta amyloid in the CSF of a subject before administering a dosage of an anti-amyloid engineered bacteriophage, and comparing this with a value for beta amyloid in the CSF after treatment with an anti-amyloid engineered bacteriophages. A decrease, for example a 10% decrease in the level of beta amyloid in the CSF indicates a positive treatment outcome (i.e., that administration of the anti-amyloid engineered bacteriophage has achieved or augmented a decrease in the amount or level of beta amyloid in the CSF). If the value for level of beta amyloid in the CSF does not change significantly, or increases, a negative treatment outcome is indicated. In general, subjects undergoing an initial course of treatment with an anti-amyloid engineered bacteriophage are expected to show a decrease in beta amyloid in the CSF with successive dosages of an anti-amyloid engineered bacteriophage as described herein.

In other methods to determine efficacy of treatment, a control value (i.e., a mean and standard deviation) of beta amyloid is determined for a control population. Typically the individuals in the control population have not received prior treatment and do not suffer from Altzhiemer's disease. Measured values of beta amyloid in the CSF in a subject after administering an anti-amyloid engineered bacteriophages as disclosed herein are then compared with the control value. A decrease in the beta amyloid in the CSF of the subject relative to the control value (i.e. a decrease of at least 10% of beta amyloid in a subject) signals a positive treatment outcome. A lack of significant decrease signals a negative treatment outcome.

In other methods, a control value of, for example beta amyloid in the CSF is determined from a control population of subjects who have undergone treatment with a therapeutic agent that is effective at reducing beta amyloid in the CSF. Measured values of CSF beta amyloid in the subject are compared with the control value.

In other methods, a subject who is not presently receiving treatment with an anti-amyloid engineered bacteriophages as disclosed herein, but has undergone a previous course of treatment is monitored for beta amyloid in the CSF to determine whether a resumption of treatment is required. The measured value of CSF beta amyloid in the test subject can be compared with a level of the CSF beta amyloid in the previously achieved in the subject after a previous course of treatment. A significant decrease in CSF beta amyloid relative to the previous measurement (i.e., a decrease of at least 10%) is an indication that treatment can be resumed. Alternatively, the level of beta amyloid in the CSF in the subject can be compared with a control level of CSF beta amyloid determined in a population of subjects after undergoing a course of treatment. Alternatively, the level of CSF beta amyloid in a subject can be compared with a control value in populations of prophylatically treated subjects who remain free of symptoms of disease, or populations of therapeutically treated subjects who show amelioration of disease symptoms.

### Methods to Identify Subjects for Risk of or Having Alzheimer's Disease.

Subjects amenable to treatment using the methods as disclosed herein, such as for the administration of a composition comprising an anti-amyloid engineered bacteriophage, e.g., a bacteriophage expressing at least CsgB₁₃₃₋₁₄₂, e.g., expressing RRR-CsgB₁₃₃₋₁₄₂-PPP, include subjects at risk of a neurodegenerative disease, for example Alzheimer's Disease but not showing symptoms, as well as subjects showing symptoms of the neurodegenerative disease, for example subjects with symptoms of Alzheimer's Disease.

Subjects can be screened for their likelihood of having or developing Alzheimer's Disease based on a number of biochemical and genetic markers.

One can also diagnose a subject with increased risk of developing Alzheimer's Disease using genetic markers for Alzheimer's Disease. Genetic abnormality in a few families has been traced to chromosome 21 (St. George-Hyslop et al., Science 235:885-890, 1987). One genetic marker is, for example mutations in the APP gene, particularly mutations at position 717 and positions 670 and 671 referred to as the Hardy and Swedish mutations respectively (see Hardy, TINS, supra). Other markers of risk are mutations in the presenilin genes, PS1 and PS2, and ApoE4, family history of Alzheimer's Disease, hypercholesterolemia or atherosclerosis. Subjects with APP, PS1 or PS2 mutations are highly likely to develop Alzheimer's disease. ApoE is a susceptibility gene, and subjects with the e4 isoform of ApoE (ApoE4 isoform) have an increased risk of developing Alzheimer's disease. Test for subjects with ApoE4 isoform are disclosed in U.S. Pat. No. 6,027,896, which is incorporated in its entirety herein by reference. Other genetic links have been associated with increased risk of Alzheimer's disease, for example variances in the neuronal sortilin-related receptor SORL1 may have increased likelihood of developing late-onset Alzheimer's disease (Rogaeva at al, Nat Genet. 2007 February; 39(2):168-77). Other potential Alzheimer disease susceptibility genes, include, for example ACE, CHRNB2, CST3, ESR1, GAPDHS, IDE, MTHFR, NCSTN, PRNP, PSEN1, TF, TFAM and TNF and be used to identify subjects with increased risk of developing Alzheimer's disease (Bertram et al, Nat Genet. 2007 January; 39(1):17-23), as well as variences in the alpha-T catenin (VR22) gene (Bertram et al, J Med Genet. 2007 January; 44(1):e63) and Insulin-degrading enzyme (IDE) and Kim et al, J Biol Chem. 2007; 282:7825-32).

One can also diagnose a subject with increased risk of developing Alzheimer's disease on the basis of a simple eye test, where the presence of cataracts and/or Abeta in the lens identifies a subject with increased risk of developing Alzheimer's Disease. Methods to detect Alzheimer's disease include using a quasi-elastic light scattering device (Goldstein et al., Lancet. 2003; 12; 361:1258-65) from Neuroptix, using Quasi-Elastic Light Scattering (QLS) and Fluorescent Ligand Scanning (FLS) and a Neuroptix™ QEL scanning device, to enable non-invasive quantitative measurements of amyloid aggregates in the eye, to examine and measure deposits in specific areas of the lens as an early diagnostic for Alzheimer's disease. Method to diagnose a subject at risk of developing Alzheimers disease using such a method of non-invasive eye test are disclosed in U.S. Pat. No. 7,107,092, which is incorporated in its entirety herein by reference.

Individuals presently suffering from Alzheimer's disease can be recognized from characteristic dementia, as well as the presence of risk factors described above. In addition, a number of diagnostic tests are available for identifying individuals who have AD. These include measurement of CSF tau and Ax3b242 levels. Elevated tau and decreased Ax3b242 levels signify the presence of Alzheimer's Disease.

There are two alternative “criteria” which are utilized to clinically diagnose Alzheimer's Disease: the DSM-IIIR criteria and the NINCDS-ADRDA criteria (which is an acronym for National Institute of Neurological and Communicative Disorders and Stroke (NINCDS) and the Alzheimer's Disease and Related Disorders Association (ADRDA); see McKhann et al., Neurology 34:939-944, 1984). Briefly, the criteria for diagnosis of Alzheimer's Disease under DSM-IIIR include (1) dementia, (2) insidious onset with a generally progressive deteriorating course, and (3) exclusion of all other specific causes of dementia by history, physical examination, and laboratory tests. Within the context of the DSM-IIIR criteria, dementia is understood to involve “a multifaceted loss of intellectual abilities, such as memory, judgement, abstract thought, and other higher cortical functions, and changes in personality and behaviour.” (DSM-1IR, 1987).

In contrast, the NINCDS-ADRDA criteria sets forth three categories of Alzheimer's Disease, including “probable,” “possible,” and “definite” Alzheimer's Disease. Clinical diagnosis of “possible” Alzheimer's Disease may be made on the basis of a dementia syndrome, in the absence of other neurologic, psychiatric or systemic disorders sufficient to cause dementia. Criteria for the clinical diagnosis of “probable” Alzheimer's Disease include (a) dementia established by clinical examination and documented by a test such as the Mini-Mental test (Foldstein et al., J. Psych. Res. 12:189-198, 1975); (b) deficits in two or more areas of cognition; (c) progressive worsening of memory and other cognitive functions; (d) no disturbance of consciousness; (e) onset between ages 40 and 90, most often after age 65; and (f) absence of systemic orders or other brain diseases that could account for the dementia. The criteria for definite diagnosis of Alzheimer's Disease include histopathologic evidence obtained from a biopsy, or after autopsy. Since confirmation of definite Alzheimer's Disease requires histological examination from a brain biopsy specimen (which is often difficult to obtain), it is rarely used for early diagnosis of Alzheimer's Disease.

One can also use neuropathologic diagnosis of Alzheimer's Disease, where the numbers of plaques and tangles in the neurocortex (frontal, temporal, and parietal lobes), hippocampus and amygdala are analyzed (Khachaturian, Arch. Neurol. 42:1097-1105; Esiri, “Anatomical Criteria for the Biopsy diagnosis of Alzheimer's Disease,” Alzheimer's Disease, Current Research in Early Diagnosis, Becker and Giacobini (eds.), pp. 239-252, 1990).

One can also use quantitative electroencephalographic analysis (EEG) to diagnose Alzheimer's Disease. This method employs Fourier analysis of the beta, alpha, theta, and delta bands (Riekkinen et al., “EEG in the Diagnosis of Early Alzheimer's Disease,” Alzheimer's Disease, Current Research in Early Diagnosis, Becker and Giacobini (eds.), pp. 159-167, 1990) for diagnosis of Alzheimer's Disease.

One can also diagnose Alzheimer's Disease by quantifying the degree of neural atrophy, since such atrophy is generally accepted as a consequence of Alzheimer's Disease. Examples of these methods include computed tomographic scanning (CT), and magnetic resonance imaging (MRI) (Leedom and Miller, “CT, MRI, and NMR Spectroscopy in Alzheimer's Disease,” Alzheimer's Disease, Current Research in Early Diagnosis, Becker and Giacobini (eds.), pp. 297-313, 1990).

One can also diagnose Alzheimer's Disease by assessing decreased cerebral blood flow or metabolism in the posterior temporoparietal cerebral cortex by measuring decreased blood flow or metabolism by positron emission tomography (PET) (Parks and Becker, “Positron Emission Tomography and Neuropsychological Studies in Dementia,” Alzheimer's Disease's, Current Research in Early Diagnosis, Becker and Giacobini (eds.), pp. 315-327, 1990), single photon emission computed tomography (SPECT) (Mena et al., “SPECT Studies in Alzheimer's Type Dementia Patients,” Alzheimer's Disease, Current Research in Early Diagnosis, Becker and Giacobini (eds.), pp. 339-355, 1990), and xenon inhalation methods (Jagust et al., Neurology 38:909-912; Prohovnik et al., Neurology 38:931-937; and Waldemar et al., Senile Dementias: II International Symposium, pp. 399407, 1988).

One can also immunologically diagnose Alzheimer's disease (Wolozin, “Immunochemical Approaches to the Diagnosis of Alzheimer's Disease,” Alzheimer's Disease, Current Research in Early Diagnosis, Becker and Giacobini (eds.), pp. 217-235, 1990). Wolozin and coworkers (Wolozin et al., Science 232:648-650, 1986) produced a monoclonal antibody “Alz50,” that reacts with a 68-kDa protein “A68,” which is expressed in the plaques and neuron tangles of patients with Alzheimer's disease. Using the antibody Alz50 and Western blot analysis, A68 was detected in the cerebral spinal fluid (CSF) of some Alzheimer's patients and not in the CSF of normal elderly patients (Wolozin and Davies, Ann. Neurol. 22:521-526, 1987).

One can also diagnose Alzheimer's disease using neurochemical markers of Alzheimer's disease. Neurochemical markers which have been associated with Alzheimer's Disease include reduced levels of acetylcholinesterase (Giacobini and Sugaya, “Markers of Cholinergic Dysfunction in Alzheimer's Disease,” Alzheimer's Disease, Current Research in Early Diagnosis, Becker and Giacobini (eds.), pp. 137-156, 1990), reduced somatostatin (Tamming a et al., Neurology 37:161-165, 1987), a negative relation between serotonin and 5-hydroxyindoleacetic acid (Volicer et al., Arch Neurol. 42:127-129, 1985), greater probenecid-induced rise in homovanyllic acid (Gibson et al., Arch. Neurol. 42:489-492, 1985) and reduced neuron-specific enolase (Cutler et al., Arch. Neurol. 43:153-154, 1986).

Methods to Identify Subjects for Risk of or Having Dementia and/or Methods for Memory Assesment.

Current standard practice can be used to diagnose the various types of dementia and, once diagnosed, to monitor the progression of the disease, e.g., Alzheimer's disease over an extended period of time. One such method includes at least one of the following; (i) a memory assessment, (ii) an extensive neuropsychological exam, (iii) an examination by a geriatric neurologist and (iv) MRI imaging of the brain. Disease progression is documented by changes in these parameters over time. In some embodiments, changes in the parameters of at least one of these assessments can be used to assess the efficacy of an anti-amyloid engineered bacteriophage, e.g., a bacteriophage expressing at least CsgB₁₃₃₋₁₄₂, e.g., expressing RRR-CsgB₁₃₃₋₁₄₂-PPP in the subject over time.

A memory assessment can be used by one of ordinary skill in the art, which is used to assess adult patients with complaint of short term memory and/or cognitive decline are seen in the Memory Assessment Program, comprising evaluation by Geriatric Neurology, Neuropsychology and Social services. Patients can be both self-referred or directed from community clinicians and physicians on the suspicion of a possible or probable memory disorder or dementia. In such a memory assessment, at the time of the initial evaluation, all of the evaluations such as (i) memory assessment (ii) an extensive neuropsychological exam, (iii) an examination by a geriatric neurologist and (iv) MRI imaging of the brain are performed the same day. The neuropsychology assessment captures a broad inventory of cognitive function which aids in determining the array and severity of deficits. These include assessments of Judgement, Insight, Behavior, Orientation, Executive Control, General Intellectual Functioning, Visualspatial Function, Memory and New Learning Ability. Depression, if present, is identified. The neurological evaluation captures the history of cognitive alteration as well as the general medical history, and typically a complete neurological exam is performed. The neurological examination can also comprise laboratory studies to exclude reversible causes of dementia including Vitamin B12, Folate, Basic Metabolic Profile, CBC, TSH, ALT, AST, C-reactive protein, serum homocysteine, and RPR. The brain imaging provides a structural brain image, such as brain MRI, although one can use other brain imaging methods known by persons of ordinary skill in the art. The data matrix of history, neuropsychologic tests, neurologic examination, laboratory studies and neuroimaging is used to formulate the diagnosis.

Dementia diagnosis can be based the guidelines of the American Academy of Neurology Practice Parameter published in 2001. Diagnosis of Alzheimers disease can be based on the NINDS-ADRDA criteria. Diagnosis of vascular dementia can be based on State of California AD Diagnostic and Treatment Centers criteria. One can communicate the diagnostic conclusion to the patient and family at a subsequent meeting. If the diagnostic conclusion indicates that the patient or subject has or is likely to have dementia and/or memory loss, a clinician can advise treatment administration with an effective amount of an anti-amyloid engineered bacteriophage, e.g., a bacteriophage expressing at least CsgB₁₃₃₋₁₄₂, e.g., expressing RRR-CsgB₁₃₃₋₁₄₂-PPP as disclosed herein. Often presence of a social worker at the subsequent meeting can also aid and direct patient and their family with current and future needs.

### Assessment of Anti-Amyloid Engineered Bacteriophage, E.G., a Bacteriophage Expressing at Least CsgB₁₃₃₋₄₄₂, or a Modified Version E.G., Expressing RRR-CsgB₁₃₃₋₁₄₂-PPP in Models of Alzheimer's Disease.

In some embodiments, an anti-amyloid engineered bacteriophage, e.g., a bacteriophage expressing at least CsgB₁₃₃₋₁₄₂, e.g., expressing RRR-CsgB₁₃₃₋₁₄₂-PPP₂ (SEQ ID NO: 61) can be assessed in animal models for vascular dementia, permitting analysis of the effects of an anti-amyloid engineered bacteriophage, e.g., a bacteriophage expressing at least CsgB₁₃₃₋₁₄₂, e.g., expressing RRR-CsgB₁₃₃₋₁₄₂-PPP on vascular dementia development and treatment, as well as assessment of drug dosages on the development, prognosis and recovery from vascular dementia.

Animal models of vascular dementia includes, for example occlusion of carotid arteries in rats. See, e.g., Sarti et al., Persistent impairment of gait performances and working memory after bilateral common carotid artery occlusion in the adult Wistar rat, BEHAVIOURAL BRAIN RESEARCH 136: 13-20 (2002). Thus, cerebrovascular white matter lesions can be experimentally induced in the rat brain as a result of chronic cerebral hypoperfusion. This model is created by permanent occlusion of both common carotid arteries. For example, Wistar rats can be anesthetized, the bilateral common carotid arteries are exposed through a midline cervical incision and the common carotid arteries are double-ligated with silk sutures bilaterally. The cerebral blood flow (CBF) then initially decreases by about 30 to 50% of the control after ligation. The CBF values later range from 40 to 80% of control after about 1 week to about 1 month. Blood-brain barrier disruptions have also been observed as well as increased matrix metalloproteinase activity in white matter lesions. These changes appear very similar to those in human cerebrovascular white matter lesions. Moreover, these results suggest that inflammatory and immunologic reactions play a role in the pathogenesis of the white matter changes.

Such physiological changes are correlated with learning and memory problems in the occluded carotid artery rat model. Thus, the gait performance of rats with occluded arteries declines over time in comparison with baseline, for example, at and 90 days, rats with bilateral common carotid artery occlusion have decreased performances on object recognition and Y maze spontaneous alternation test in comparison with sham-operated rats. Thus, this rat model of experimental chronic cerebral hypoperfusion by permanent occlusion of the bilateral common carotid arteries is useful as a model for significant learning impairments along with rarefaction of the white matter. This model is a useful tool to assess the effectiveness of an anti-amyloid engineered bacteriophage, e.g., a bacteriophage expressing at least CsgB₁₃₃₋₁₄₂, e.g., expressing RRR-CsgB₁₃₃₋₁₄₂-PPP on the pathophysiology of chronic cerebral hypoperfusion, and to provide data for determining optimal dosages and dosage regimens for preventing the cognitive impairment and white matter lesions in patients with cerebrovascular disease.

The effectiveness of an anti-amyloid engineered bacteriophage, e.g., a bacteriophage expressing at least CsgB₁₃₃₋₁₄₂, e.g., expressing RRR-CsgB₁₃₃₋₁₄₂-PPP for treating or preventing vascular dementia can therefore be determined by observing the gait performance, memory, learning abilities and the incidence and severity of white matter lesions in rats with carotid artery occlusions. Similarly, the dosage and administration schedule of an anti-amyloid engineered bacteriophage, e.g., a bacteriophage expressing at least CsgB₁₃₃₋₁₄₂, e.g., expressing RRR-CsgB₁₃₃₋₁₄₂-PPP can be adjusted pursuant to the memory and learning abilities of human patients being treated for vascular dementia.

In other embodiments, the optimum dosage of an anti-amyloid engineered bacteriophage, e.g., a bacteriophage expressing at least CsgB₁₃₃₋₁₄₂, e.g., expressing RRR-CsgB₁₃₃₋₁₄₂-PPP is one generating the maximum beneficial effect on damaged tissue caused by arterial occlusion. An effective dosage causes at least a statistically or clinically significant attenuation of at least one marker, symptom, or histological evidence characteristic of vascular dementia. Markers, symptoms and histological evidence characteristic of vascular dementia include memory loss, confusion, disturbances in axonal transport, demyelination, induction of metalloproteinases (MMPs), activation of glial cells, infiltration of lymphocytes, edema and immunological reactions that lead to tissue damage and further vascular injury. Stabilization of symptoms or diminution of tissue damage, under conditions wherein control patients or animals experience a worsening of symptoms or tissue damage, is one indicator of efficacy of a suppressive treatment.

### Assessment of an Anti-Amyloid Engineered Bacteriophage, E.G., a Bacteriophage Expressing at Least CsgB₁₃₃₋₁₄₂, E.G., Expressing RRR-CsgB₁₃₃₋₁₄₂-PPP on Models of Neurodegenerative Diseases.

The suitability of an anti-amyloid engineered bacteriophage, e.g., a bacteriophage expressing at least CsgB₁₃₃₋₁₄₂, e.g., expressing RRR-CsgB₁₃₃₋₁₄₂-PPP for the treatment of a neurodegenerative disease involving amyloid or fibril accumulation can be assessed in any of a number of animal models for neurodegenerative disease. For example, mice transgenic for an expanded polyglutamine repeat mutant of ataxin-1 develop ataxia typical of spinocerebellar ataxia type 1 (SCA-1) are known (Burright et al., 1995, Cell 82: 937-948; Lorenzetti et al., 2000, Hum. Mol. Genet. 9: 779-785; Watase, 2002, Neuron 34: 905-919), and can be used to determine the efficacy of an anti-amyloid engineered bacteriophage, e.g., a bacteriophage expressing at least CsgB₁₃₃₋₁₄₂, e.g., expressing RRR-CsgB₁₃₃₋₁₄₂-PPP₂ for the treatment or prevention of neurodegenerative disease. Additional animal models, for example, for Huntington's disease (see, e.g., Mangiarini et al., 1996, Cell 87: 493-506, Lin et al., 2001, Hum. Mol. Genet. 10: 137-144), Alzheimer's disease (Hsiao, 1998, Exp. Gerontol, 33: 883-889; Hsiao et al., 1996, Science 274: 99-102), Parkinson's disease (Kim et al., 2002, Nature 418: 50-56), amyotrophic lateral sclerosis (Zhu et al., 2002, Nature 417: 74-78), Pick's disease (Lee & Trojanowski, 2001, Neurology 56 (Suppl. 4): S26-S30, and spongiform encephalopathies (He et al., 2003, Science 299: 710-712) can be used to evaluate the efficacy of an anti-amyloid engineered bacteriophage, e.g., a bacteriophage expressing at least CsgB₁₃₃₋₁₄₂, e.g., expressing RRR-CsgB₁₃₃₋₁₄₂-PPP as disclosed herein in a similar manner.

Animal models are not limited to mammalian models. For example, Drosophila strains provide accepted models for a number of neurodegenerative disorders (reviewed in Fortini & IBonini, 2000, Trends Genet. 16: 161-167; Zoghbi & Botas, 2002, Trends Genet. 18: 463-471). These models include not only flies bearing mutated fly genes, but also flies bearing human transgenes, optionally with targeted mutations. Among the Drosophila models available are, for example, spinocerebellar ataxias (e.g., SCA-1 (see, e.g., WO 02/058626), SCA-3 (Warrick et al., 1998, Cell 93: 939-949)), Huntington's disease (Kazemi-Esfarjani & Benzer, 2000, Science 287: 1837-1840), Parkinson's disease (Feany et al, 2000, Nature 404: 394-398; Auluck et al., 2002, Science 295: 809-8 10), age-dependent neurodegeneration (Genetics, 2002, 161:4208), Alzheimer's disease (Selkoe et al., 1998, Trends Cell Biol. 8: 447-453; Ye et al., 1999, J. Cell Biol. 146: 1351-1364), amyotrophic lateral sclerosis (Parkes et al., 1998, Nature Genet. 19: 171-174), and adrenoleukodystrophy.

The use of Drosophila as a model organism has proven to be an important tool in the elucidation of human neurodegenerative pathways, as the Drosophila genome contains many relevant human orthologs that are extremely well conserved in function (Rubin, G. M., et al., Science 287: 2204-2215 (2000)). For example, Drosophila melanogaster carries a gene that is homologous to human APP which is involved in nervous system function. The gene, APP-like (APPL), is approximately 40% identical to APP695, the neuronal isoform (Rosen et al., Proc. Natl. Acad. Sci. U.S.A. 86:2478-2482 (1988)), and like human APP695 is exclusively expressed in the nervous system. Flies deficient for the APPL gene show behavioral defects which can be rescued by the human APP gene, suggesting that the two genes have similar functions in the two organisms (Luo et al., Neuron 9:595-605 (1992)). Drosophila models for Alzhiemers disease are disclosed in U.S. Patent Applications 2004/0244064, 2005/0132425, 2005/0132424, 2005/0132423, 2005/0132422, 200/50132421, 2005/0108779, 2004/0255342, 2004/0255341, 2004/0250302 which are incorporated herein in their entirety by reference.

In addition, Drosophila models of polyglutamine repeat diseases (Jackson, G. R., et al., Neuron 21:633-642 (1998); Kazemi-Esfarani, P. and Benzer, S., Science 287:1837-1840 (2000); Fernandez-Funez et al., Nature 408:101-6 (2000)), Parkinson's disease (Feany, M. B. and Bender, W. W., Nature 404:394-398 (2000)) and other diseases have been established which closely mimic the disease state in humans at the cellular and physiological levels, and have been successfully employed in identifying other genes that can be involved in these diseases. The transgenic flies exhibit progressive neurodegeneration which can lead to a variety of altered phenotypes including locomotor phenotypes, behavioral phenotypes (e.g., appetite, mating behavior, and/or life span), and morphological phenotypes (e.g., shape, size, or location of a cell, organ, or appendage; or size, shape, or growth rate of the fly).

Animals administered a composition comprising an anti-amyloid engineered bacteriophage, e.g., a bacteriophage expressing at least CsgB₁₃₃₋₁₄₂, e.g., expressing RRR-CsgB₁₃₃₋₁₄₂-PPP can be evaluated for symptoms relative to animals not administered the compounds. A measurable change in the severity a symptom (i.e., a decrease in at least one symptom, i.e. 10% or greater decrease), or a delay in the onset of a symptom, in animals treated with an an anti-amyloid engineered bacteriophage, e.g., a bacteriophage expressing at least CsgB₁₃₃₋₁₄₂, e.g., expressing RRR-CsgB₁₃₃₋₁₄₂-PPP versus untreated animals is indicative of therapeutic efficacy.

One can assess the animals for memory and learning, for instance by performing behavioral testing. One can use any behavioral test for memory and learning commonly known by person of ordinary skill in the art, for but not limited to the Morris water maze test for rodent animal models. A measurable increase in the ability to perform the Morris water maze test in animals administered an anti-amyloid engineered bacteriophage, e.g., a bacteriophage expressing at least CsgB₁₃₃₋₁₄₂, e.g., expressing RRR-CsgB₁₃₃₋₁₄₂-PPP versus untreated animals is indicative of therapeutic efficacy.

The suitability of an anti-amyloid engineered bacteriophage, e.g., a bacteriophage expressing at least CsgB₁₃₃₋₁₄₂, e.g., expressing RRR-CsgB₁₃₃₋₁₄₂-PPP for the treatment of Altzhimer's disease can be assessed in any of a number of animal models. One method that can be used is to assess the ability of blood-borne components, such as Ig or amyloid beta (Aβ) peptides to cross the blood-brain barrier (BBB) and interact with neurons in the brain. One method useful in the methods as disclosed herein to assess blood-borne components, such as Ig or amyloid beta (Aβ) peptides crossing the BBB uses fluorescent labeled Abeta42, and is described in Clifford et al., 2007, Brain Research 1142: 223-236, which is incorporated herein in its entirety by reference. In this method, the ability of blood-borne Aβ peptides to cross a defective BBB was assessed using fluorescein isothiocyanate (FITC)-labeled Aβ42 and Aβ40 introduced via tail vein injection into mice with a BBB rendered permeable by treatment with pertussis toxin. Both Aβ40 and Aβ42 were shown to cross the permeabilized BBB and bound selectively to certain neuronal subtypes, but not glial cell, with widespead Aβ42-positive neurons in the brain 48 hrs post-injection. As a control, animals with intact BBB (saline-injected controls) blocked entry of blood-borne Aβ peptides into the brain. One can use such a animal model to assess the ability of an anti-amyloid engineered bacteriophage, e.g., a bacteriophage expressing at least CsgB₁₃₃₋₁₄₂, e.g., expressing RRR-CsgB₁₃₃₋₁₄₂-PPP on the reduction of Aβ42 accumulation by assessing Aβ42-positive neurons in the brain 48 hrs post-injection of pertussis toxin and FITC-labeled Aβ42 in the presence or absence of an anti-amyloid engineered bacteriophage, e.g., a bacteriophage expressing at least CsgB₁₃₃₋₁₄₂, e.g., expressing RRR-CsgB₁₃₃₋₁₄₂-PPP. A decrease in Aβ42-positive neurons in the brain in animals administered an anti-amyloid engineered bacteriophage, e.g., a bacteriophage expressing at least CsgB₁₃₃₋₁₄₂, e.g., expressing RRR-CsgB₁₃₃₋₁₄₂-PPP as compared to animals not administered an anti-amyloid engineered bacteriophage indicates that the anti-amyloid engineered bacteriophage, e.g., a bacteriophage expressing at least CsgB₁₃₃₋₁₄₂, e.g., expressing RRR-CsgB₁₃₃₋₁₄₂-PPP is a effective at treating and/or preventing Altzhiemer's disease.

Effective doses of the compositions of the present invention, for the treatment of the above described amyloid-associated disorders vary depending upon many different factors, including the type of disorder, means of administration, target site, physiological state of the subject, whether the subject is human or an animal, other medications administered, and whether treatment is prophylactic or therapeutic.

The dosage and frequency of administration to a subject can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In some embodiments, the subject is a human, and in alternative embodiments the subject is a non-human mammal. Treatment dosages need to be titrated to optimize safety and efficacy. The amount of immunogenic peptide expressed by the anti-amyloid peptide engineered bacteriophage depends on the anti-amyloid peptide being administered as well as the route of administration. Typically, an amount of an anti-amyloid peptide engineered bacteriophage is administered so that the concentration of anti-amyloid peptide varies from 1 μg-500 μg per subject and more usually from 5-500 μg per administration for human administration. Occasionally, an amount of an anti-amyloid peptide engineered bacteriophage such that the amount of anti-amyloid peptide is at a higher dose of 0.5-5 mg per administration. Typically an amount of anti-amyloid peptide engineered bacteriophage is administered such that the amount of anti-amyloid peptide is about 10, 20, 50 or 100 μg for administration to a human.

The timing of administration can vary significantly from once a day, to once a year, to once a decade. Generally, in accordance with the teachings provided herein, effective dosages can be monitored by obtaining a fluid sample from the subject, generally a blood serum sample, and determining the titer of the an anti-amyloid peptide engineered bacteriophage using methods well known in the art and readily adaptable to the specific bacteriophage measured. Additionally, the level of decrease in amyloid formation or maintenance can be monitored by methods commonly known in the art. Ideally, a sample is taken prior to initial dosing; subsequent samples are taken and titered after each immunization. Generally, a dose or dosing schedule which provides a detectable titer at least four times greater than control or “background” levels at a serum dilution of 1:100 is desirable, where background is defined relative to a control serum or relative to a plate background in ELISA assays. Titers of at least 1:1000 or 1:5000 are preferred in accordance with the present invention.

On any given day that a dosage of an anti-amyloid peptide engineered bacteriophage such that the amount of anti-amyloid peptide dosage is greater than about 1 μg/subject and usually greater than 10 μg/subject, and greater than 10 μg/subject and usually greater than 100 μg/subject in the absence of adjuvant. Doses for individual an anti-amyloid peptide engineered bacteriophage such that the amount of anti-amyloid peptide is effective is determined according to standard dosing and titering methods, taken in conjunction with the teachings provided herein. A typical regimen consists of an administration of an anti-amyloid peptide engineered bacteriophage followed by booster administration of an anti-amyloid peptide engineered bacteriophage at time intervals, such as 6 week intervals.

In some embodiments, efficacy of treatment can be measured as an improvement in morbidity or mortality (e.g., lengthening of survival curve for a selected population). Prophylactic methods (e.g., preventing or reducing the incidence of relapse) are also considered treatment.

Dosages, formulations, dosage volumes, regimens, and methods for analyzing results aimed at reducing the number of viable bacteria and/or activity can vary. Thus, minimum and maximum effective dosages vary depending on the method of administration. Suppression of the clinical changes associated with bacterial infections or infection with a microorganism can occur within a specific dosage range, which, however, varies depending on the organism receiving the dosage, the route of administration, whether other agents such as other anti-amyloid peptides or agents which inhibit fiber association are administered in conjunction with the anti-amyloid peptide engineered bacteriophages as disclosed herein, and in some embodiments with other co-stimulatory molecules, and the specific regimen administration. For example, in general, nasal administration requires a smaller dosage than oral, enteral, rectal, or vaginal administration.

For oral or enteral formulations for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. No. 4,704,295, “Enteric Film-Coating Compositions,” issued Nov. 3, 1987; U.S. Pat. No. 4,556,552, “Enteric Film-Coating Compositions,” issued Dec. 3, 1985; U.S. Pat. No. 4,309,404, “Sustained Release Pharmaceutical Compositions,” issued Jan. 5, 1982; and U.S. Pat. No. 4,309,406, “Sustained Release Pharmaceutical Compositions,” issued Jan. 5, 1982, which are incorporated herein in their entirety by reference.

Examples of solid carriers include starch, sugar, bentonite, silica, and other commonly used carriers. Further non-limiting examples of carriers and diluents which can be used in the formulations of the present invention include saline, syrup, dextrose, and water.

In some embodiments, the pharmaceutical compositions comprising an anti-amyloid peptide engineered bacteriophage as disclosed herein can be administered by parenteral, topical, intravenous, oral, subcutaneous, intraperitoneal, intranasal or intramuscular means for prophylactic and/or therapeutic treatment. Typical routes of administration of an immunogenic peptide are intramuscular (i.m.), intravenous (i.v.) or subcutaneous (s.c.), although other routes can be equally effective. Intramuscular injection is most typically performed in the arm or leg muscles. In some methods, the immunogenic peptides or other pharmaceutical compositions are injected directly into a particular tissue, for example a tumor tissue where the immunoglobulin producing cell is located. Such administration is termed intratumoral administration. In some methods, particular pharmaceutical compositions comprising the immunogenic peptides for the treatment of amyloidogenic diseases of the brain are administered directly to the head or brain via injection directly into the cranium. In some methods, antibodies are administered as a sustained release composition or device, such as a Medipad™ device.

An anti-amyloid peptide engineered bacteriophage as disclosed herein can optionally be administered in replacement of, or in combination with other agents, for example, agents which are commonly used for the treatment of amyloid associated disorders and amyloidosis. For example, but not limited to, the an anti-amyloid peptide engineered bacteriophage can be administered with agents that are at least partly effective in treatment of plasma cell malignancies, for example AL amyloidosis and/or multiple myeloma.

Other agents which can be administered in conjunction (i.e. prior to, at the same time, or following) with an anti-amyloid peptide engineered bacteriophage to a subject suffering from, or likely to have an amyloid-associated disorder as disclosed here include traditional therapies for treatment of amyloid-associated disorders, or amyloidosis, include, melphalan (ALKERAN®, ALKERAN IV®), a chemotherapy agent also used to treat certain types of cancer, and dexamethasone, a corticosteroid used for its anti-inflammatory effects, bortezomib (VELCADE®), thalidomide (THALOMID®), and a thalidomide derivative called lenalidomide (REVLIMID®), or peripheral blood stem cell transplantation. Peripheral blood stem cell transplantation involves using high-dose chemotherapy and transfusion of previously collected immature blood cells (stem cells) to replace diseased or damaged marrow. These cells may be one's own (autologous transplant) or from a donor (allogeneic transplant).

If the amyloid-associated disorder is Alzheimer's disease, agents which can be administered in conjunction (i.e. prior to, at the same time, or following) administration with anti-amyloid peptide engineered bacteriophage to a subject suffering from, or likely to have an amyloid-associated disorder which is Alzheimer's as include for example but are not limited to, gamma secretase inhibitors and modulators, and human beta-secretase (BACE) inhibitors. Disease modifying agents also are, for example but not limited to gamma secretase inhibitors and modulators, beta-secretase (BACE) inhibitors and any other anti-amyloid approaches including active and passive immunization, for example agents identified by the methods as disclosed in U.S. Patent Application 2005/0170359, as well as agents as disclosed in International Patent Applications WO05/07277, WO03/104466 and WO07/028,133, and U.S. Pat. Nos. 6,866,849, 6,913,745, which are incorporated in their entirety herein by reference.

An anti-amyloid peptide engineered bacteriophages as disclosed herein can also be administered in conjunction with other agents that increase passage of the anti-amyloid peptides of the present invention across the blood-brain barrier, for example, where the anti-amyloid peptide inhibits the formation and maintenance of β-amyloid plaques in Alzheimer's disease.

### Industrial/Environmental Use

The inventors have demonstrated the effectiveness of an anti-amyloid peptide engineered bacteriophage to inhibit biofilm formation on solid surfaces or in fluid samples, as disclosed herein in the Examples. Accordingly, the present invention also contemplates the use of the anti-amyloid peptide engineered bacteriophages as discussed herein to treat biofilm infections on various environmental surfaces, or in fluid samples.

Environmental surfaces in which the engineered bacteriophage is useful to reduce biofilm infection include, but are not limited to, slaughterhouses, meat processing facilities, feedlots, vegetable processing facilities, medical facilities and devices, military facilities, veterinary offices, animal husbandry facilities, public and private restrooms, and nursing and nursing home facilities. The invention further contemplates the use of an anti-amyloid peptide engineered bacteriophage for the battlefield decontamination of food products, the environment, and personnel and equipment, both military and non-military.

Effective dose of the compositions comprising an anti-amyloid peptide engineered bacteriophage for the treatment of the above-described bacterial biofilm vary depending upon many different factors, including the type of bacterial biofilm, environmental surface, administration site, and mode and frequency of administration.

An anti-amyloid peptide engineered bacteriophage can be administered at a concentration effective to inhibit the formation of amyloids, and/or inhibit the presence of bacterial biofilms on environmental surfaces or in fluid samples. In some embodiments, the concentration of anti-amyloid peptide engineered bacteriophages, for example, to prevent biofilm formation on medical devices, can be about at least 1×10⁷ PFU/ml-1×10¹⁰ PFU/mL, for example about at least 1×10⁷ PFU/ml, or about at least 1×10⁸ PFU/ml, or about at least 1×10⁸ PFU/ml, or about at least 10⁹ PFU/ml, or about at least 10¹⁰ PFU/ml, or more than about at least 1×10¹⁰ PFU/ml. One of skill in the art is capable of ascertaining bacteriophage concentrations using widely known bacteriophage assay techniques (Adams, M. H. (1959). Methods of study bacterial viruses. Bacteriophages. London, Interscience Publishers, Ltd.: 443-519.).

An anti-amyloid peptide bacteriophage as discussed herein can be useful alone or in combination with other bacteriophages expressing other amyloid peptides and/or other chemical compounds, for example, detergents, soaps, etc., for preventing the formation of biofilms, or controlling the growth of biofilms, in various environments. Aqueous embodiments of the engineered bacteriophage are available in solutions that include, but are not limited to, phosphate buffered saline, Luria-Bertani Broth or chlorine-free water.

Practice of the present invention will employ, unless indicated otherwise, conventional techniques of cell biology, cell culture, molecular biology, microbiology, recombinant DNA, protein chemistry, and immunology, which are within the skill of the art. Such techniques are described in the literature. See, for example, Molecular Cloning: A Laboratory Manual, 2nd edition. (Sambrook, Fritsch and Maniatis, eds.), Cold Spring Harbor Laboratory Press, 1989; DNA Cloning, Volumes I and II (D. N. Glover, ed), 1985; Oligonucleotide Synthesis, (M. J. Gait, ed.), 1984; U.S. Pat. No. 4,683,195 (Mullis et al.,); Nucleic Acid Hybridization (B. D. Hames and S. J. Higgins, eds.), 1984; Transcription and Translation (B. D. Hames and S. J. Higgins, eds.), 1984; Culture of Animal Cells (R. I. Freshney, ed). Alan R. Liss, Inc., 1987; Immobilized Cells and Enzymes, IRL Press, 1986; A Practical Guide to Molecular Cloning (B. Perbal), 1984; Methods in Enzymology, Volumes 154 and 155 (Wu et al., eds), Academic Press, New York; Gene Transfer Vectors for Mammalian Cells (J. H. Miller and M. P. Calos, eds.), 1987, Cold Spring Harbor Laboratory; Immunochemical Methods in Cell and Molecular Biology (Mayer and Walker, eds.), Academic Press, London, 1987; Handbook of Experiment Immunology, Volumes I-IV (D. M. Weir and C. C. Blackwell, eds.), 1986; Manipulating the Mouse Embryo, Cold Spring Harbor Laboratory Press, 1986.

The following Examples are provided to illustrate the present invention, and should not be construed as limiting thereof.

In some embodiments of the present invention may be defined in any of the following numbered paragraphs:

### Claims:

1. An engineered bacteriophage comprising a nucleic acid operatively linked to a promoter, wherein the nucleic acid encodes at least one anti-amyloid peptide.
2. The bacteriophage of paragraph 1, wherein the anti-amyloid peptide is a peptide between at least 5 and 50 amino acid long whose sequence comprises at least 5 and no more than 50 contiguous amino acids of the sequence of a first amyloidogenic polypeptide which is capable of nucleating amyloid formation by a second amyloidogenic polypeptide.
3. The bacteriophage of paragraph 1, wherein the anti-amyloid peptide is a peptide between at least 5 and 50 amino acid long whose sequence comprises at least 5 and no more than 50 contiguous amino acids of the sequence of a second amyloidogenic polypeptide, wherein a second amyloidogenic polypeptide forms an amyloid formation with a first amyloidogenic polypeptide.
4. The bacteriophage of any of paragraphs 1 to 3, wherein the anti-amyloid peptide is a peptide between least 8 and no more than 30 contiguous amino acids of the sequence of a first amyloidogenic polypeptide.
5. The bacteriophage of any of paragraphs 1 to 3, wherein the anti-amyloid peptide is a peptide between least 8 and no more than 30 contiguous amino acids of the sequence of a second amyloidogenic polypeptide.
6. The bacteriophage of any of paragraphs 1 to 5, wherein the first and second amyloidogenic polypeptides are no more than 50% identical.
7. The bacteriophage of any of paragraphs 1 to 6, wherein at least one of the amyloidogenic polypeptides is a component of a naturally occurring amyloid or a component of a high order aggregate comprising at least two different polypeptides.
8. The bacteriophage of any of paragraphs 1 to 7, wherein at least one of the amyloidogenic polypeptides is a component of a biofilm generated by a bacterium.
9. The bacteriophage of any of paragraphs 1 to 8, wherein the bacterium is a human or animal pathogen.
10. The bacteriophage of any of paragraphs 1 to 9, wherein the bacterium is a gram-negative bacterium.
11. The bacteriophage of any of paragraphs 1 to 10, wherein the bacterium is a gram-negative rod.
12. The bacteriophage of any of paragraphs 1 to 11, wherein the bacterium is an enterobacterium.
13. The bacteriophage of any of paragraphs 1 to 12, wherein the bacterium is a member of a genus selected from Escherichia, Klebsiella, Salmonella, and Shigella.
14. The bacteriophage of any of paragraphs 1 to 13, wherein the first amyloidogenic polypeptide is a CsgB polypeptide.
15. The bacteriophage of any of paragraphs 1 to 14, wherein the second amyloidogenic polypeptide is a CsgA polypeptide.
16. The bacteriophage of any of paragraphs 1 to 15, wherein the first and second amyloidogenic polypeptides are a CsgB polypeptide and a CsgA polypeptide, respectively.
17. The bacteriophage of any of paragraphs 1 to 16, wherein the anti-amyloid peptide is between 10 and 30 amino acids in length.
18. The bacteriophage of any of paragraphs 1 to 17, wherein the anti-amyloid peptide is between 15 and 25 amino acids in length.
19. The bacteriophage of any of paragraphs 1 to 18, wherein the sequence of the anti-amyloid peptide comprises or consists of a sequence selected from SEQ ID NO: 1 or SEQ ID NO: 2 and orthologs thereof.
20. The bacteriophage of any of paragraphs 1 to 19, wherein the sequence of the anti-amyloid peptide comprises or consists of a sequence selected from SEQ ID NO: 1 or SEQ ID NO: 2 and orthologs thereof.
21. The bacteriophage of any of paragraphs 1 to 20, wherein the anti-amyloid peptide is CsgA peptide.
22. The bacteriophage of any of paragraphs 1 to 21, wherein the anti-amyloid peptide is a CsgB peptide.
23. The bacteriophage of any of paragraphs 1 to 22, wherein the CsgA peptide is selected from the group comprising: SEQ ID NO; 11-18, CsgA III class of peptides (SEQ ID NO: 52-53), CsgAIIb class of peptides (SEQ ID NOs:35, 36, 39-41, 45, 49-51), CsgAIIa class of peptides (SEQ ID NO: 11 and 12) and CsgAI class of peptides (SEQ ID NOs: 42, 44, 46, 57 and 58) or orthologs thereof.
24. The bacteriophage of any of paragraphs 1 to 23, wherein the CsgA peptide is selected from the group comprising: SEQ ID NOs: 52 and 53) or orthologs thereof.
25. The bacteriophage of any of paragraphs 1 to 22, wherein the CsgB peptide is selected from the group comprising: SEQ ID NO; 27-34, CsgBIII class of peptides (SEQ ID NOs: 61-65), CsgBIIb class of peptides (SEQ ID NOs: 59, 60, 69, 75, 81, 93 and 94), CsgBIIa class of peptides (SEQ ID NO: 29) and CsgBI class of peptides (SEQ ID NOs: 66-68 and 70-72) or orthologs thereof.
26. The bacteriophage of any of paragraphs 1 to 22, wherein the CsgB peptide is selected from the group comprising: SEQ ID NOs: 61-65 or orthologs thereof.
27. The bacteriophage of any of paragraphs 1 to 26, wherein the anti-amyloid peptide sequence differs by not more than 4 amino acid insertions, deletions, or substitutions from that of the peptides of paragraph 23 or 24.
28. The bacteriophage of any of paragraphs 1 to 27, wherein the anti-amyloid peptide sequence differs by not more than 4 amino acid insertions, deletions, or substitutions from that of the peptides of paragraphs 25 or 26.
29. The bacteriophage of any of paragraphs 1 to 28, wherein the N-terminus and/or C-terminus of the anti-amyloid peptide sequence comprise at least one additional amino acid residue.
30. The bacteriophage of any of paragraphs 1 to 29, wherein the N-terminus or C-terminus of the anti-amyloid peptide sequence comprises a charged amino acid residue or at least one bulky amino acid.
31. The bacteriophage of any of paragraphs 1 to 30, wherein the amino acid is an arginine or a proline amino acid residue.
32. The bacteriophage of any of paragraphs 1 to 31, wherein the N-terminal amino acid is at least one arginine amino acid residue.
33. The bacteriophage of any of paragraphs 1 to 31, wherein the N-terminal amino acid is at least two arginine amino acid residue.
34. The bacteriophage of any of paragraphs 1 to 31, wherein the N-terminal amino acid is at least three arginine amino acid residue.
35. The bacteriophage of any of paragraphs 1 to 31, wherein the C-terminal amino acid is at least one proline amino acid residue.
36. The bacteriophage of any of paragraphs 1 to 31, wherein the C-terminal amino acid is at least two proline amino acid residue.
37. The bacteriophage of any of paragraphs 1 to 31, wherein the C-terminal amino acid is at least three proline amino acid residue.
38. The bacteriophage of any of paragraphs 1 to 37, wherein the anti-amyloid peptide is expressed on the surface of the engineered bacteriophage from which it is expressed.
39. The bacteriophage of any of paragraphs 1 to 38, wherein the anti-amyloid peptide is released from a bacterial host cell infected by the engineered bacteriophage.
40. The bacteriophage of any of paragraphs 1 to 39, wherein the anti-amyloid peptide is released from a bacterial host cell infected by the engineered bacteriophage by lysis of the bacterial cell.
41. The bacteriophage of any of paragraphs 1 to 40, wherein the antimicrobial peptide is released from a bacterial host cell infected by the engineered bacteriophage by secretion by the bacterial host cell.
42. The bacteriophage of any of paragraphs 1 to 41, wherein the nucleic acid encoding at least one anti-amyloid peptide agent also encodes a signal sequence.
43. The bacteriophage of any of paragraphs 1 to 42, wherein the signal sequence is a secretory sequence.
44. The bacteriophage of any of paragraphs 1 to 43, wherein the secretory sequence is cleaved from the anti-amyloid peptide or antimicrobial peptide.
45. A method to reduce protein aggregate formation in a subject comprising administering to a subject at least one bacteriophage comprising a nucleic acid operatively linked to a promoter, wherein the nucleic acid encodes at least one anti-amyloid peptide.
46. The method of paragraph 45, wherein the subject suffers or is at risk of amyloid associated disorder.
47. The method of paragraphs 45 or 46 wherein the subject suffers from or is at increased risk of an infection by a bacterium.
48. The method of any of paragraphs 45 to 47, wherein the bacterium is associated with biofilm formation.
49. The method of any of paragraphs 45 to 48, wherein the subject is a mammal.
50. The method of any of paragraphs 45 to 49, wherein the mammal is a human.
51. The method of any of paragraphs 45 to 50, further comprising adding an additional agent to the subject.
52. The method of any of paragraphs 45 to 51, wherein the anti-amyloid peptide is a peptide between at least 5 and 50 amino acid long whose sequence comprises at least 5 and no more than 50 contiguous amino acids of the sequence of a first amyloidogenic polypeptide or a second amyloidogenic polypeptide, wherein a first amyloidogenic polypeptide is capable of nucleating amyloid formation by a second amyloidogenic polypeptide.
53. The method of any of paragraphs 45 to 52, wherein the anti-amyloid peptide is a peptide between least 8 and no more than 30 contiguous amino acids of the sequence of a first amyloidogenic polypeptide or a second amyloidogenic polypeptide.
54. The method of any of paragraphs 45 to 53, wherein the first and second amyloidogenic polypeptides are no more than 50% identical.
55. The method of any of paragraphs 45 to 54, wherein the anti-amyloid peptide inhibits the formation of at least one of the amyloidogenic polypeptides that is a component of a naturally occurring amyloid or a component of a high order aggregate comprising at least two different polypeptides.
56. The method of any of paragraphs 45 to 55, wherein the high order aggregate comprises a fiber.
57. The method of any of paragraphs 45 to 56, wherein the first amyloidogenic polypeptide is a CsgB polypeptide.
58. The method of paragraphs 45 to 57, wherein the second amyloidogenic polypeptide is a CsgA polypeptide.
59. The method of any of paragraphs 45 to 58, wherein the anti-amyloid peptide is between 10 and 30 amino acids in length.
60. The method of any of paragraphs 45 to 59, wherein the anti-amyloid peptide is between 15 and 25 amino acids in length.
61. The method of any of paragraphs 45 to 60, wherein the sequence of the anti-amyloid peptide comprises or consists of a sequence of at least 8 contagious amino acids selected from any in SEQ ID NO: 1 or SEQ ID NO: 2 and orthologs thereof.
62. The method of any of paragraphs 45 to 61, wherein the anti-amyloid peptide is CsgA peptide.
63. The method of any of paragraphs 45 to 62, wherein the anti-amyloid peptide is a CsgB peptide.
64. The method of any of paragraphs 45 to 63, wherein the CsgA peptide is selected from the group comprising: SEQ ID NO; 11-18, SEQ ID NO; 11-18, CsgA III class of peptides (SEQ ID NO: 52-53), CsgAIIb class of peptides (SEQ ID NOs:35, 36, 39-41, 45, 49-51), CsgAIIa class of peptides (SEQ ID NO: 11 and 12) and CsgAI class of peptides (SEQ ID NOs: 42, 44, 46, 57 and 58) or orthologs thereof.
65. The method of any of paragraphs 45 to 64, wherein the CsgA peptide is selected from the group comprising: SEQ ID NOs: 52 and 53) or orthologs thereof.
66. The method of any of paragraphs 45 to 63 wherein the CsgB peptide is selected from the group comprising: SEQ ID NO; 27-34, CsgBIII class of peptides (SEQ ID NOs: 61-65), CsgBIIb class of peptides (SEQ ID NOs: 59, 60, 69, 75, 81, 93 and 94), CsgBIIa class of peptides (SEQ ID NO: 29) and CsgBI class of peptides (SEQ ID NOs: 66-68 and 70-72) or orthologs thereof.
67. The method of any of paragraphs 45 to 66, wherein the CsgB peptide is selected from the group comprising: SEQ ID NOs: 61-65 or orthologs thereof.
68. The method of any of paragraphs 45 to 67, wherein the anti-amyloid peptide sequence differs by not more than 4 amino acid insertions, deletions, or substitutions from that of the peptides of paragraph 56 or 57.
69. The method of any of paragraphs 45 to 68, wherein the anti-amyloid peptide sequence differs by not more than 4 amino acid insertions, deletions, or substitutions from that of the peptides of paragraphs 57 or 59.
70. The method of any of paragraphs 45 to 69, wherein the anti-amyloid peptide is expressed on the surface of the engineered bacteriophage from which it is expressed.
71. The method of any of paragraphs 45 to 70, wherein the anti-amyloid peptide is released from a bacterial host cell infected by the engineered bacteriophage.
72. The method of any of paragraphs 45 to 72, wherein the subject is administered a plurality of bacteriophages, wherein each bacteriophage comprises a nucleic acid which encodes one or more different anti-amyloid peptides.
73. The method of any of paragraphs 45 to 73, wherein the plurality of bacteriophages express one or more different anti-amyloid peptides from the same amyloidogenic polypeptide or a different amyloidogenic polypeptide.
74. The method of any of paragraphs 45 to 74, wherein at least one bacteriophage in a plurality of bacteriophages express one or more anti-amyloid peptides from a first amyloidogenic polypeptide, and at least one bacteriophage in a plurality of bacteriophages expresses one or more anti-amyloid peptides from a second amyloidogenic polypeptide.
75. The method of any of paragraphs 45 to 74, wherein the first amyloidogenic polypeptide is a CsgA polypeptide and a second amyloidogenic polypeptide is a CsgB polypeptide.
76. The method of any of paragraphs 45 to 75, wherein the N-terminus and/or C-terminus of the anti-amyloid peptide sequence comprise at least one additional amino acid residue.
77. The method of any of paragraphs 45 to 76, wherein the N-terminus or C-terminus of the anti-amyloid peptide sequence comprises a charged amino acid residue or at least one bulky amino acid.
78. The method of any of paragraphs 45 to 77, wherein the amino acid is an arginine or a proline amino acid residue.
79. The method of any of paragraphs 45 to 75, wherein the N-terminal amino acid is at least one arginine amino acid residue, or at least two arginine amino acid residues, or at least three arginine amino acid residues.
80. The method of any of paragraphs 45 to 75, wherein the C-terminal amino acid is at least one proline amino acid residue, or at least two proline amino acid residues, or at least three proline amino acid residues.
81. A method to inhibit protein aggregate formation on a surface, or in a fluid sample comprising administering to the surface or fluid sample a composition comprising at least one bacteriophage comprising a nucleic acid operatively linked to a promoter, wherein the nucleic acid encodes at least one anti-amyloid peptide.
82. A method to promote protein aggregate formation on a surface, or in a fluid sample comprising administering to the surface or fluid sample a composition comprising at least one bacteriophage comprising a nucleic acid operatively linked to a promoter, wherein the nucleic acid encodes at least one amyloid peptide.
83. The method of paragraph 81 or 82, wherein the surface is a solid surface.
84. The method of any of paragraphs 81 to 83, wherein the solid surface is the surface of a medical device.
85. The method of paragraphs 81 to 84, wherein the solid surface is the work surface of a facility.
86. The method of paragraphs 81 to 85, wherein the surface is infected with, or likely to be infected with a bacterial infection an infection.
87. The method of any of paragraphs 81 to 86, wherein the bacterium is associated with biofilm formation.
88. The method of any of paragraphs 81 to 87, wherein the composition further comprises an additional agent.
89. The method any of paragraphs 81 to 88, wherein the additional agent is a different engineered bacteriophage.
90. The method any of paragraphs 81 to 89, wherein the additional agent is a chemical.
91. The method of any of paragraphs 81 to 90, wherein the anti-amyloid peptide or amyloid peptide sequence is a peptide between at least 5 and 50 amino acid long whose sequence comprises at least 5 and no more than 50 contiguous amino acids of the sequence of a first amyloidogenic polypeptide or a second amyloidogenic polypeptide, wherein a first amyloidogenic polypeptide is capable of nucleating amyloid formation by a second amyloidogenic polypeptide.
92. The method of any of paragraphs 81 to 91, wherein the anti-amyloid peptide or amyloid peptide sequence is a peptide between least 8 and no more than 30 contiguous amino acids of the sequence of a first amyloidogenic polypeptide or a second amyloidogenic polypeptide.
93. The method of any of paragraphs 81 to 92, wherein the first and second amyloidogenic polypeptides are no more than 50% identical.
94. The method of any of paragraphs 81 and 83 to 93, wherein the anti-amyloid peptide inhibits the formation of at least one of the amyloidogenic polypeptides that is a component of a naturally occurring amyloid or a component of a high order aggregate comprising at least two different polypeptides.
95. The method of any of paragraphs 81 and 83 to 94, wherein the anti-amyloid peptide inhibits the formation of at least one of the amyloidogenic polypeptides that is a component of a non-naturally occurring amyloid or a component of a high order aggregate comprising at least two different polypeptides.
96. The method of any of paragraphs 82 to 93, wherein the amyloid peptide promotes the formation of at least one of the amyloidogenic polypeptides that is a component of a naturally occurring amyloid or a component of a high order aggregate comprising at least two different polypeptides.
97. The method of any of paragraphs 82 to 93, wherein the amyloid peptide promotes the formation of at least one of the amyloidogenic polypeptides that is a component of a non-naturally occurring amyloid or a component of a high order aggregate comprising at least two different polypeptides.
98. The method of any of paragraphs 81 to 97, wherein the high order aggregate comprises a fiber.
99. The method of any of paragraphs 81 to 98, wherein the high order aggregate is a biofilm plaque.
100. The method of any of paragraphs 81 to 99, wherein the first amyloidogenic polypeptide is a CsgB polypeptide.
101. The method of any of paragraphs 81 to 100, wherein the second amyloidogenic polypeptide is a CsgA polypeptide.
102. The method of any of paragraphs 81 to 101, wherein the anti-amyloid peptide or amyloid peptide sequence is between 10 and 30 amino acids in length.
103. The method of any of paragraphs 81 to 102, wherein the anti-amyloid peptide or amyloid peptide sequence is between 15 and 25 amino acids in length.
104. The method of any of paragraphs 81 to 103, wherein the sequence of the anti-amyloid peptide or amyloid peptide sequence comprises or consists of a sequence of at least 8 contagious amino acids selected from any in SEQ ID NO: 1 or SEQ ID NO: 2 and orthologs thereof.
105. The method of any of paragraphs 81 to 104, wherein the anti-amyloid peptide or amyloid peptide sequence is a CsgA peptide.
106. The method of any of paragraphs 81 to 105, wherein the anti-amyloid peptide or amyloid peptide sequence is a CsgB peptide.
107. The method of any of paragraphs 81 to 106, wherein the CsgA peptide is selected from the group comprising: SEQ ID NO; 11-18, CsgA III class of peptides (SEQ ID NO: 52-53), CsgAIIb class of peptides (SEQ ID NOs:35, 36, 39-41, 45, 49-51), CsgAIIa class of peptides (SEQ ID NO: 11 and 12) and CsgAI class of peptides (SEQ ID NOs: 42, 44, 46, 57 and 58) or orthologs thereof.
108. The method of any of paragraphs 81 to 107, wherein the CsgA peptide is selected from the group comprising: SEQ ID NOs: 52 and 53) or orthologs thereof.
109. The method of any of paragraphs 81 to 108, wherein the CsgB peptide is selected from the group comprising: SEQ ID NO; 27-34, CsgBIII class of peptides (SEQ ID NOs: 61-65), CsgBIIb class of peptides (SEQ ID NOs: 59, 60, 69, 75, 81, 93 and 94), CsgBIIa class of peptides (SEQ ID NO: 29) and CsgBI class of peptides (SEQ ID NOs: 66-68 and 70-72) or orthologs thereof.
110. The method of any of paragraphs 81 to 109, wherein the CsgB peptide is selected from the group comprising: SEQ ID NOs: 61-65 or orthologs thereof.
111. The method of any of paragraphs 81 to 110, wherein the anti-amyloid peptide or amyloid peptide sequence differs by not more than 4 amino acid insertions, deletions, or substitutions from that of the peptides of paragraph 93 or 94.
112. The method of any of paragraphs 81 to 111, wherein the anti-amyloid peptide or amyloid peptide sequence differs by not more than 4 amino acid insertions, deletions, or substitutions from that of the peptides of paragraphs 95 or 96.
113. The method of any of paragraphs 82 to 112, wherein the amyloid peptide is selected from the group comprising RRR-CsgB(132-142)-GGG (SEQ ID NO: 88) or a ortholog thereof.
114. The method of any of paragraphs 81 to 113, wherein the anti-amyloid peptide is expressed on the surface of the engineered bacteriophage from which it is expressed.
115. The method of any of paragraphs 81 to 114, wherein the anti-amyloid peptide is released from a bacterial host cell infected by the engineered bacteriophage.
116. The method of any of paragraphs 81 to 115, wherein the non-living matter is administered a plurality of bacteriophages, wherein each bacteriophage comprises a nucleic acid which encodes one or more different anti-amyloid peptides.
117. The method of any of paragraphs 81 to 116, wherein the plurality of bacteriophages express one or more different anti-amyloid peptides from the same amyloidogenic polypeptide or a different amyloidogenic polypeptide.
118. The method of any of paragraphs 81 to 117, wherein at least one bacteriophage in a plurality of bacteriophages express one or more anti-amyloid peptides from a first amyloidogenic polypeptide and at least one bacteriophage in a plurality of bacteriophages expresses one or more anti-amyloid peptides from a second amyloidogenic polypeptide.
119. The method of any of paragraphs 81 to 118, wherein the first amyloidogenic polypeptide is a CsgA polypeptide and a second amyloidogenic polypeptide is a CsgB polypeptide.
120. The method of any of paragraphs 81 to 119, wherein the N-terminus and/or C-terminus of the anti-amyloid peptide sequence comprise at least one additional amino acid residue.
121. The method of any of paragraphs 81 to 120, wherein the N-terminus or C-terminus of the anti-amyloid peptide sequence comprises a charged amino acid residue or at least one bulky amino acid.
122. The method of any of paragraphs 81 to 123, wherein the amino acid is an arginine or a proline amino acid residue.
123. The method of any of paragraphs 81 to 124, wherein the N-terminal amino acid is at least one arginine amino acid residue, or at least two arginine amino acid residues, or at least three arginine amino acid residues.
124. The method of any of paragraphs 81 to 125, wherein the C-terminal amino acid is at least one proline amino acid residues.
125. A composition comprising the bacteriophage of paragraph 1.
126. The composition of paragraph 125, further comprising a pharmaceutical acceptable carrier.
127. The composition of paragraphs 125 or 126, further comprising an additional agent.
128. The composition of any of paragraphs 125 to 127, wherein the additional agent is an anti-amyloid peptide or an agent which inhibits fiber aggregation.
129. A kit comprising a bacteriophage comprising the nucleic acid operatively linked to a promoter, wherein the nucleic acid encodes at least one anti-amyloid peptide.
130. Use of an engineered bacteriophage of any of paragraphs 1 to 44 for reducing the formation or maintenance of protein aggregates.
131. The use of paragraph 130, wherein the protein aggregate is a naturally forming amyloid or a high order aggregate comprising of at least two different polypeptides.
132. The use of paragraphs 130 or 131, wherein the naturally forming amyloid comprises a first amyloidogenic polypeptide which is capable of nucleating amyloid formation by a second amyloidogenic polypeptide.
133. The use of any of paragraphs 130 to 132, wherein the protein aggregates are present in a subject.
134. The use of any of paragraphs 130 to 133, wherein the protein aggregates are present on a surface of a support, or in a fluid sample.
135. The use of any of paragraphs 130 to 134, wherein the surface is a solid surface.
136. The use of any of paragraphs 130 to 135, wherein the surface is a work surface of a facility.
137. The use of any of paragraphs 130 to 136, wherein the protein aggregates are in a bacterial biofilms.
138. Use of an engineered bacteriophage of any of paragraphs 1 to 44 for sterilizing a medical device or surfaces of a medical facility.
139. Use of an engineered bacteriophage of any of paragraphs 1 to 44 for personal hygiene.
140. A method for the treatment of Alzheimer's disease comprises administering to a subject a composition comprising an anti-amyloid engineered bacteriophage comprising comprising at least one bacteriophage comprising a nucleic acid operatively linked to a promoter, wherein the nucleic acid encodes at least one anti-amyloid peptide.
141. The method of paragraph 140, wherein the subject suffers or at risk of Alzheimer's disease or diseases associated with A□ peptides.
142. The method of paragraphs 140 to 141, wherein the subject is a mammal.
143. The method of paragraphs 140 to 142, wherein the mammal is a human.
144. The method of any of paragraphs 140 to 143, wherein the composition further comprises an additional agent.
145. The method any of paragraphs 140 to 144, wherein the additional agent is a different engineered bacteriophage.
146. The method any of paragraphs 140 to 145, wherein the additional agent is an additional therapeutics used for treatment of Alzheimer's disease.
147. The method of any of paragraphs 140 to 146, wherein the anti-amyloid peptide is a peptide between at least 4 and 50 amino acid long whose sequence comprises at least 5 and no more than 50 contiguous amino acids of the reverse sequence of the A□ peptide or variants thereof.
148. The method of any of paragraphs 140 to 147, wherein the anti-amyloid peptide is a peptide between least 4 and no more than 20 contiguous amino acids of the sequence of the reverse sequence of the A□ peptide or variants thereof.
149. The method of any of paragraphs 140 to 148, wherein the anti-amyloid peptide inhibits the formation of at least one of the A□ polypeptides that is a component of a naturally occurring amyloid or a component of a high order aggregate comprising at least two different polypeptides.
150. The method of any of paragraphs 140 to 149, wherein the anti-amyloid peptide inhibits the formation of at least one of the A□ polypeptides that is a component of a non-naturally occurring amyloid or a component of a high order aggregate comprising at least two different polypeptides.
151. The method of any of paragraphs 140 to 150, wherein the high order aggregate comprises at least one A□ polypeptide.
152. The method of any of paragraphs 140 to 151, wherein the anti-amyloid peptide comprises AIVV (SEQ ID NO: 192).
153. The method of any of paragraphs 140 to 152, wherein the anti-amyloid peptide is between 15 and 25 amino acids in length.
154. The method of any of paragraphs 140 to 153, wherein the sequence of the anti-amyloid peptide comprises or consists of a sequence of at least 8 contagious amino acids selected from any in SEQ ID NO: 2 and orthologs thereof.
155. The method of any of paragraphs 140 to 154, wherein the anti-amyloid peptide is CsgB peptide.
156. The method of any of paragraphs 140 to 155, wherein the CsgB peptide is selected from the group comprising: SEQ ID NO; 27-34, CsgA III class of peptides (SEQ ID NO: 52-53), CsgAIIb class of peptides (SEQ ID NOs: 35, 36, 39-41, 45, 49-51), CsgAIIa class of peptides (SEQ ID NO: 11 and 12) and CsgAI class of peptides (SEQ ID NOs: 42, 44, 46, 57 and 58) or orthologs thereof.
157. The method of any of paragraphs 140 to 156, wherein the CsgB peptide is selected from the group comprising: SEQ ID NOs: 61-65 or orthologs thereof.
158. The method of any paragraphs 140 to 157, wherein the CsgB peptide comprises the nucleating AIVV (SEQ ID NO: 199) sequence of the CsgB peptide.
159. The method of any of paragraphs 140 to 158, wherein the anti-amyloid peptide sequence differs by more than 4 amino acid insertions, deletions, or substitutions from that of the peptides of paragraphs 156-158.
160. The method of any of paragraphs 140 to 158, wherein the anti-amyloid peptide sequence differs by not more than 4 amino acid insertions, deletions, or substitutions from that of the peptides of paragraphs 156-158.
161. The method of any of paragraphs 140 to 160, wherein the anti-amyloid peptide is expressed on the surface of the engineered bacteriophage from which it is expressed.
162. The method of any of paragraphs 140 to 161, wherein the anti-amyloid peptide is released from a bacterial host cell infected by the engineered bacteriophage.
163. The method of any of paragraphs 140 to 162, wherein the plurality of bacteriophages express one or more different anti-amyloid peptides from the same amyloidogenic polypeptide or a different amyloidogenic polypeptide.
164. The method of any of paragraphs 140 to 163, wherein at least one bacteriophage in a plurality of bacteriophages express one or more anti-amyloid peptides against a Aβ polypeptide.

### EXAMPLES

The examples presented herein relate to the methods and compositions comprising anti-amyloid peptide engineered bacteriophages for the inhibition or disruption of the formation or maintenance of protein aggregates which comprises of two or more different polypeptides. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

The inventors have genetically engineered M13mp18 filamentous and T7 lytic bacteriophages (phages) to give them properties of blocking curli formation and inhibiting amyloid formation by curli and NM (the prion-determining region of the yeast protein Sup35p) by inducing the expression and secretion of anti-amyloid peptides from the host bacteria. The anti-amyloid peptide engineered phage show improved killing activity against bacteria, for example when the bacteria are in solution, e.g a fluid sample.

Anti-amyloid peptides are small peptides, typically composed of contiguous 7 to 30 amino acids from a polypeptide which forms aggregates and high order aggregates. The inventors have combined the broad activity spectrum of anti-amyloid peptides with advantages such as exponential growth and low toxicity of bacteriophages such that the bacteriophages function as a bioreactor to produce high amounts of anti-amyloid peptides at a required site, for example at a site where protein aggregates or high order aggregates occur. Use of bacteriophages to express the anti-amyloid peptides is advantageous because phages multiply and replicate in the presence of host cells, whereas typical administration of anti-amyloid peptide therapies would require that the correct amount of anti-amyloid peptide be delivered systemically such appropriate therapeutic concentration are reached the site of infection; this poses toxicity issues for anti-amyloid peptide. An anti-amyloid peptide as disclosed herein include engineered bacteriophages which comprise at least one DNA sequence inducing the expression (and secretion in some case) of different anti-amyloid peptide such that anti-amyloid peptides are synthesized and delivered at the site of the protein aggregation. In some embodiments, for example, where the anti-amyloid peptide engineered bacteriophages comprises a nucleic acid encoding an anti-amyloid peptide which is a CsgA peptide and/or a CsgB peptide, the expression of the CsgA peptide and/or a CsgB peptides occurs at the location of the bacteria to block curli formation in E. coli.

This approach is extremely advantageous for future therapeutic applications and the inventors show that these engineered bacteriophages have increased bacterial killing activity in solution.

The inventors have engineered bacteriophages to induce expression of the anti-amyloid peptides of the CsgA polypeptide (SEQ ID NO:1) and CsgB polypeptide (SEQ ID NO:2). The inventors generated engineered bacteriophages expressing peptides derived from the CsgA polypeptide are shown in Table 3 (SEQ ID NOs: 11-18) or derived from the CsgB polypeptide (SEQ ID NOs: 27-34) (see Table 4). The inventors also generated anti-amyloid engineered bacteriophages expressing modified peptides from CsgA and CsgB (see Table 5) (SEQ ID NOs: 11, 12, 29 and 35-90). The engineering of the genome was carried out using conventional genetic engineering techniques.

Strains, Bacteriophage, and Chemicals.

Escherichia coli O1:K1:H7 (ATCC#11775) was obtained from the American Type Culture Collection (Manassas, Va.). Bacteriophage kits for peptide expression were obtained from Novagen Inc. (San Diego, Calif.). Wild-type T7 phage (ATCC #BAA-1025-B2) were purchased from ATCC (Manassas, Va.). M13mp18 phage, T4 DNA ligase, restriction enzymes, and PCR reagents were obtained from NEW England Biolabs, Inc. (Ipswich, Mass.). PCR reactions and restriction digests was carried out with the QIAquick Gel Extraction or PCR Purification kits (Qiagen, Valencia, Calif.). All other chemicals were purchased from Fisher Scientific, Inc. (Hampton, N.H.) or as noted in the text.

Mutational Analysis of CsgA and CsgB.

CsgA or CsgB mutants were expressed from plasmids located in cells with their endogenous CsgA or CsgB genes knocked out.

Phage Display of Curli-Blocking Peptides.

The T7select415-1 kit (Novagen) was used for high-copy expression of peptides on phage capsids (415 peptides per capsid). The T7select10-3b kit (Novagen) was used for medium-copy expression of peptides on phage capsids (5-15 peptides per capsid). DNA inserts were cloned in the EcoRI and HindIII sites of T7select phage genomes and packaged in vitro according to kit instructions. Phages constructed using the T7select415-1 system were amplified in E. coli BL21 cells whereas phages constructed using T7select10-3b were amplified in E. coli BLT5403 cells. As a negative control, an S•Tag insert was cloned into the EcoRI and HindIII sites to construct T7-con. All phages concentrations were determined via plaque assay on BL21 cells and were equalized to the same concentrations.

In vitro Curli Assembly Assays.

Phage were added at concentrations from 10¹ to 10⁶ PFU/mL. Amyloid fiber formation by CsgA was monitored using ThT fluorescence in a plate reader. Average ThT fluorescence was calculated from three independent experiments.

Biofilm Assays.

Biofilm levels were assessed using a crystal violet assay as previously described¹⁴. Briefly, E. coli bacteria were grown overnight in LB media at 37° C. and 300 rpm (model G25 incubator shaker, New Brunswick Scientific). Bacteria were collected via centrifugation at 3,700 g for 5 minutes and resuspended in fresh YESCA media to an optical density at 600 nm (OD₆₀₀ₙₘ) of 1.0. Phage were added to the cells at various concentrations. Lids with plastic pegs (MBEC Physiology and Genetics Assay, Edmonton, Calif.) were placed in 96-well plates containing 150 μL of bacteria±phage. Plates were then inserted into plastic bags to minimize evaporation and shaken at 28° C. and 150 rpm in a Minitron shaker (Infors HT, Bottmingen, Switzerland). After 24 hours, pegs were washed three times in 200 μL of 1× phosphate-buffered saline (PBS) in 96-well plates. Pegs were then stained with 200 μL of 1% crystal violet for 15 minutes followed by three additional washing steps with 1×PBS. To quantify biofilm levels, crystal violet was solubilized in 200 μL of 33% acetic acid and the resulting absorbance (OD₆₀₀ₙₘ) was measured with a TECAN SpectraFluor Plus plate reader (Zurich, Switzerland). Crystal violet OD₆₀₀ₙₘ of all samples was normalized to the OD600 nm of untreated samples. For all conditions, n=8 samples were collected except for the untreated, wild-type T7 (T7-wt), and control phage (T7-con), for which n=16 samples were obtained.

Mammalian Cell Invasion Assay.

HEK 293 cells were grown overnight in 24-well plates to 80% confluence (2×10⁵ cells per well). Bacteria were inoculated in LB broth and grown at 37° C. for 16 h, diluted 100-fold, and grown to mid-exponential phase (OD₆₀₀ₙₘ=0.6). Bacteria were then diluted to OD₆₀₀ₙₘ=0.3 in DMEM media and 2 mL were added to each well with epithelial monolayer for 4 h incubation at 37° C. in the presence or in the absence of 10⁹ phage particles. Cells were washed once with PBS (pH 7.3), and PBS containing 40 μg/mL gentamicin was added. After 1 h incubation, the cells were washed twice in PBS, and lysed by 10 min incubation with ice-cold 0.5% triton X-100. Appropriate bacterial dilutions [2000-5000 fold dilutions] were plated to determine the number of viable internalized bacteria. E. coli 11775 containing pTrc99a was maintained on Luria-Bertani (LB) agar containing 50 μg/mL ampicillin.

In Vitro ThT Fluorescence Assay.

For ThT fluorescent assays involving NM, the NM protein was prepared as in Scheibel, et al., Current Biology, 2001, 11(5), 36-369. The NM stock solution was diluted to a final concentration of 2.5 uM and ThT binding studies were carried out in the presence and absence of phage. For the ThT fluorescent assays involving the amyloid-b1-42 peptide, lyophilized amyloid-b1-42 (0.5 mg, Bachem) was resuspended in 200 uL 1,1,1,3,3,3-hexafluoro-2-propanol (HFIP, Aldrich). The solution was vortexed for up to 1 hour to allow the peptide to fully dissolve. Subsequently, the HFIP was evaporated using argon and the peptide was resuspended in 200 uL of dimethyl sulfoxide (DMSO, Aldrich). This stock solution was diluted to a final concentration of 2.5 uM and ThT binding studies were carried out in the presence and absence of phage.

Phage Display of Curli-Blocking Peptides.

The T7select415-1 kit (Novagen) was used for high-copy expression of peptides on phage capsids (415 peptides per capsid). The T7select10-3b kit (Novagen) was used for medium-copy expression of peptides on phage capsids (5-15 peptides per capsid). Oligonucleotide pairs designated with prefix N in Table 6 were annealed and digested with EcoRI and HindIII. Oligonucleotide pairs designated with prefix D in Table 6 were used as PCR primers on the DNA template composed of annealing N79 and N127 together; the result of these PCR reactions were gel-purified and digested with EcoRI and HindIII. The cut inserts were cloned in the EcoRI and HindIII sites of T7select phage genome followed by in vitro packaging according to kit instructions.

Preincubation of Biofilm Plates with Phage to Prevent Biofilms.

Lids with plastic pegs (MBEC Physiology and Genetics Assay, Edmonton, Calif.) were placed in 96-well plates containing 200 μL of phage for 4 hours at 28° C. Lids were then moved in 96-well plates containing 150 μL of E. coli. Plates were then inserted into plastic bags to minimize evaporation and shaken at 28° C. and 150 rpm in a Minitron shaker (Infors HT, Bottmingen, Switzerland). After 24 hours, pegs were washed three times in 200 μL of 1× phosphate-buffered saline (PBS) in 96-well plates. Pegs were then stained with 200 μL of 1% crystal violet for 15 minutes followed by three additional washing steps with 1×PBS. To quantify biofilm levels, crystal violet was solubilized in 200 μL of 33% acetic acid and the resulting absorbance (OD₆₀₀ₙₘ) was measured with a TECAN SpectraFluor Plus plate reader (Zurich, Switzerland). Crystal violet OD₆₀₀ₙₘ of all samples was normalized to the OD₆₀₀ₙₘ of untreated samples. For all conditions, n=8 samples were collected except for the untreated cases, for which n=11 samples were obtained.

Computational Predictions of Amyloid Structure.

The computer program, herein referred to as “AmyloidMutants” is useful to predict amyloid structure by calculating a pseudo-energetic score for the exponential set of possible amyloid structures. From this calculated ensemble, a representative set of structures can be sampled, clustered, and the most likely conformations can be used as a prediction. At the core of this tool is the ability to compute the Boltzmann partition function Z, where Z=Σₛe^{E}^{_{z}}^{/RT} given temperature RT, and the energy of every possible structure Eₛ. Although calculating Z for a biomolecule using a true 3-dimensional representation is considered computationally intractable¹, polynomial-time calculations have been shown feasible when restricting the structural representation via a grammatical model^{2,3}. The AmyloidMutants algorithm uses a similar philosophy of domain restriction, but with a more expressive framework than grammatical models, allowing for the definition of amyloid structure (either β-solenoidal or β-sandwiched). The summation of Z itself is performed via an efficient, parallel dynamic programming traversal of recursively-defined structure space.

The energy potential of any fibril state Eₛ is derived in terms of the likelihood of observing a sub-structural state pₛ_{ᵢ}, Eₛ=Σᵢ(−RT log(pₛ_{ᵢ})−RT log(Z)). Sub-structural states include the likelihood that two residues pair within a β-sheet^{4,5} (p(i|j)), conditioned on amphipathicity, and whether those residues are in the middle, sides, or edge of a β-sheet, the statistical potential of two consecutive residues forming a coil (p(i,j)), and a simple hydropathic propensity score for two residues packing between β-sheet faces⁶. The relative influence these terms can be scaled independently, allowing specific facets of structural interaction to be investigated. These frequencies are computed from structures in the Protein Data Bank, conditioned on sub-structural elements with similar microenvironments. While computing the partition function, the appropriate frequency is chosen for a specific conditional/microenvironment based the structural location within the recursive search of all possible structures.

To efficiently sample representative structures from the exponential space of all possible structures, a table of intermediate sub-structural energies is constructed during the dynamic programming traversal calculating the partition function. By stochastically backtracking over these intermediate values, full structural conformations can be sampled according to their Boltzmann distributed energy score'. Populations of similar structures are identified and separated via hierarchical clustering, taking as input the number of clusters, and relying on a distant metric that combines secondary structure, hydrogen bond registration, coil location, and β-strand overlap. Using these intermediate values many other structural properties can be calculated, such as a stochastic contact map, which describes the Boltzmann-weighted likelihood p(i, j) that two residues i and j will form a β-sheet. Each p(i, j) reflects the precise exact β-sheet composition at that location across the entire structural landscape, and can be used to identify high-likelihood common substructures between possibly disparate full structures⁸.

Calculating Amyloid Ensembles.

At the core of the AmyloidMutabnt algorithm lies the ability to compute the Boltzmann partition function Z for any given protein sequence, a key distinction from prior methodologies. The thermodynamic normalization constant Z encodes the statistical variation of a system in equilibrium, defined here as the set of all feasible structural conformations a protein can achieve (an ensemble), with a Boltzmann-distributed energy score Es assigned to each conformation s. Given temperature T, and the physical constant R, Z is the sum: ∀s, Z=Σs^{e-Es/RT}.

AmyloidMutants extends this notion of a structural ensemble v, to analyze sequence/structure ensembles vi: the set of all feasible combination of structural ensembles across a set of related sequence mutants. The partition function Z of a sequence/structure ensemble is therefore the sum: ∀ω, ∀s, Z=ΣωΣs e−Es/RT, given sequences ω and structures s. This encodes not only statistical variations in protein structure, but variations in protein sequence, distributed according to the energetic likelihood of that sequence's conformations. With this one can not only predict the most energetically favorable structure and sequence assignment, but a single quantitative energetic score can be used to measure the difference between two sequences, between two structures, or between both.

Although calculating the partition function of a biomolecule using a 3-dimensional representation is considered computationally intractable 36, such a computation has been shown feasible for structural RNA by heavily restricting the representation using context-free-grammar (CFG) models and applying dynamic programming 37. However, protein structures generally exhibit far too complex interactions to tractably apply this same approach. The only polynomial-time calculation of Z derived for protein structure thus far relies on a multitape CFG model, and is achieved by restricting the prediction problem to only the family of transmembrane β-barrel proteins 38. Similarly, the calculation of sequence/structure ensembles have been long considered too computationally expensive to be done. To date, the only algorithm that has considered this problem computes k-neighbor sequence/structure landscapes of the restrictive CFG RNA model previously reported 39.

The AmyloidMutant algorithm for computing the sequence/structure landscape of an amyloid fibril uses a similar philosophy of domain restriction, but permits any number of different structural and mutational restrictions, as defined by a schema, and is not limited to CFGs. Each schema outlines structure space using a recursive definition of allowed β-strand/β-strand or β-sheet/β-sheet interactions, and outline sequence space as a set of allowed mutations of a base sequence. This is implemented as a C++ template (defining structure space) and a mutational protocol (defining sequence space), separate and interchangeable from the core algorithms of the tool. An analysis is performed on this input, and a dynamic programming procedure is constructed that traverses and scores all possible subsequence/substructure conformations and stores these in a table. From this Z can be calculated via a simple traversal.

Defining Amyloid Schemas.

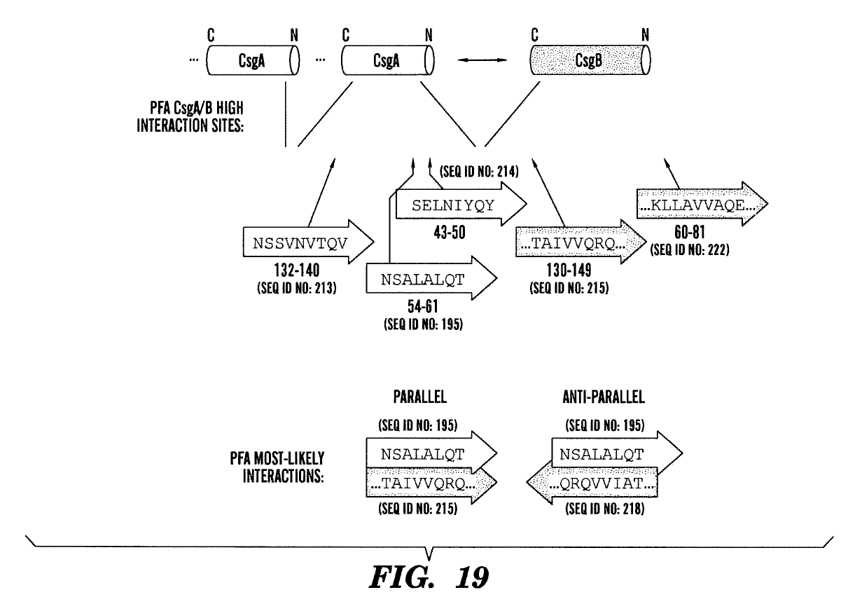

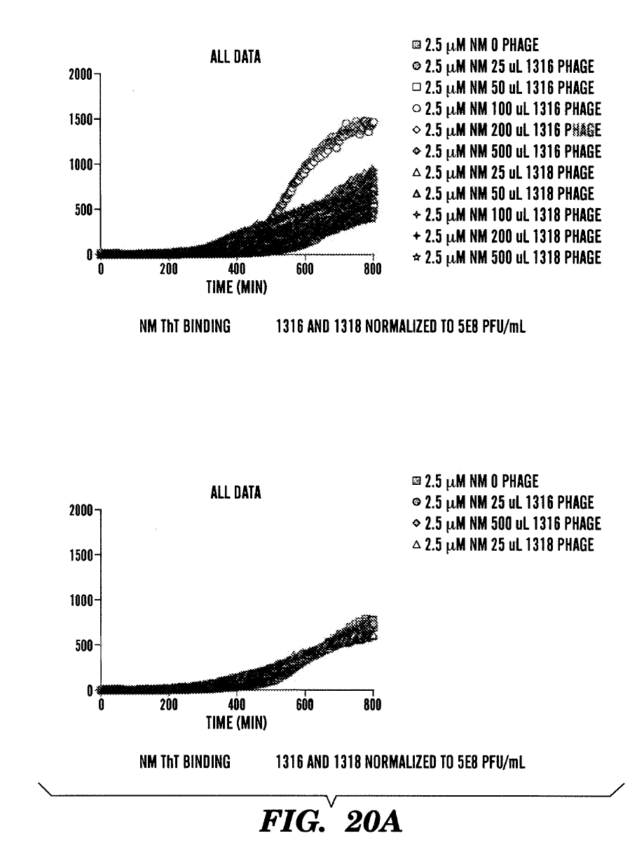

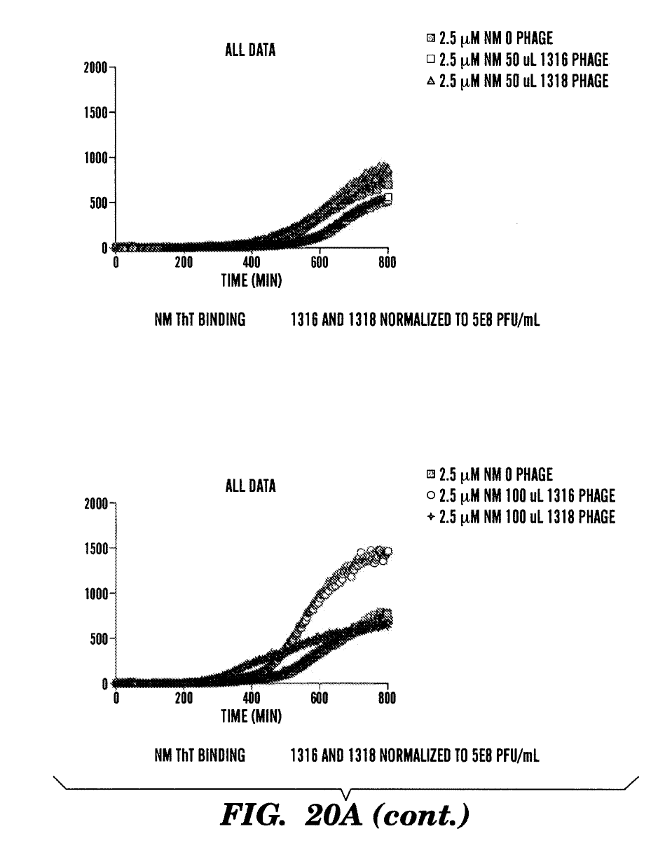

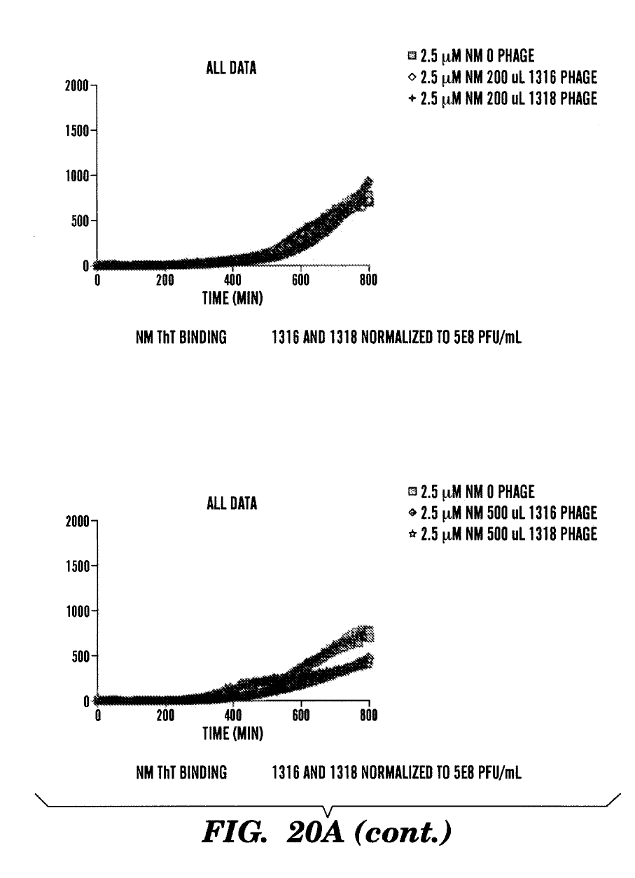

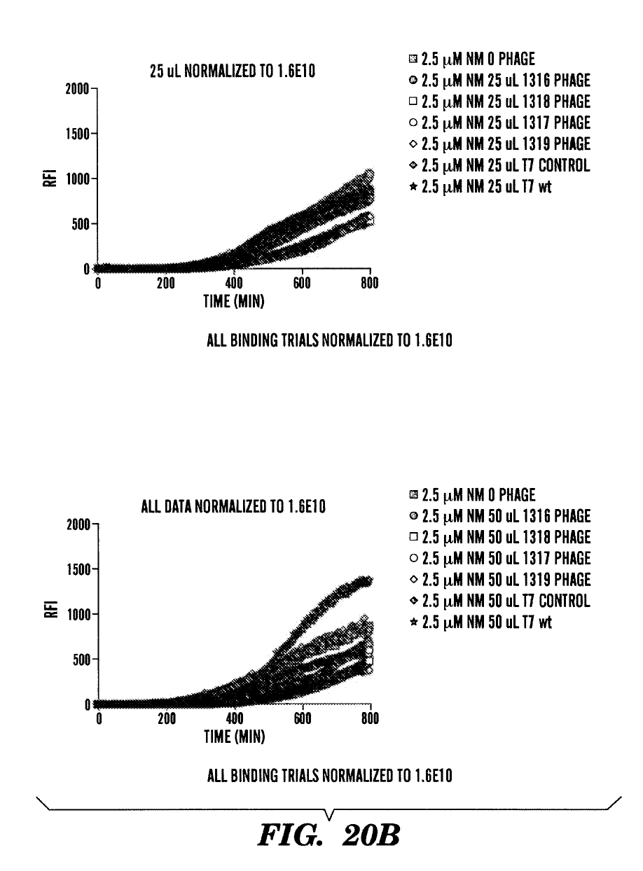

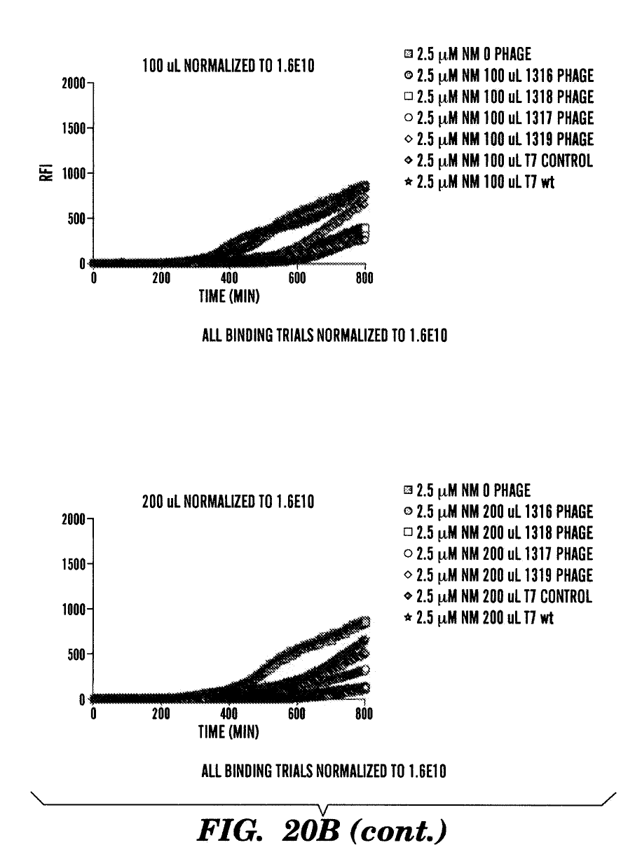

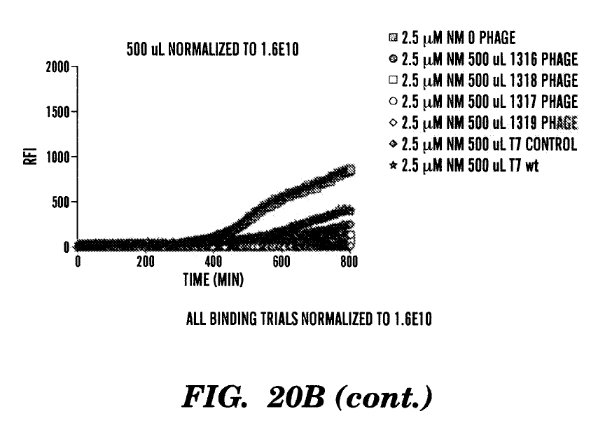

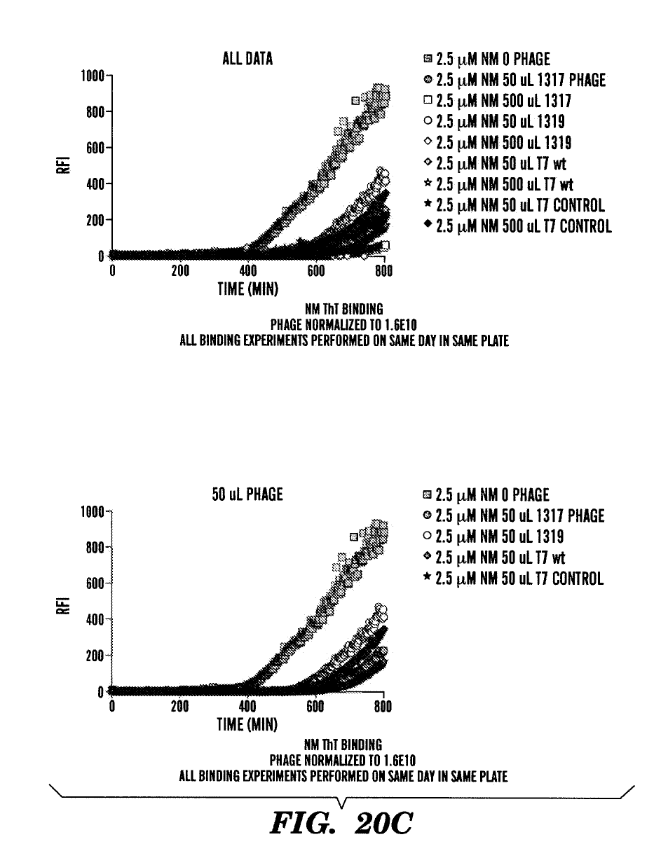

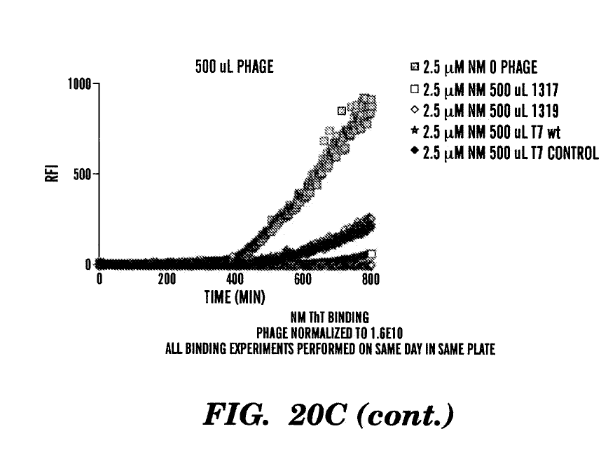

Schemas are defined in two parts, a recursive encoding of structure space that is compatible with a chosen energy model, and a protocol giving a list of all allowed sequence mutations. To represent amyloid fibril structures, which can amass thousands of peptide chains down their length, a schema formally defines only the possible conformations of a single peptide chain and its two immediate axial neighbors (see FIG. 19). This representation models a theoretical fibril slice that is repeated indefinitely along the axis (e.g. if peptides ABCDE are adjacent in a longer fibril, then a schema defines the identical conformational landscapes of ABC, BCD, and CDE). The inclusion of axial neighbors in our model is necessary to ensure a realistic conformational symmetry between peptides—a property shown highly important in protein modeling 40. Heterogeneous fibrils with relaxed symmetry constraints, and amyloidal interaction sites between different types of proteins have also been modeled by our schemas, though are not discussed here.

Structure space is defined as putative geometric arrangement of β-sheets at the resolution of (1) intra-peptide strand-to-strand hydrogen bonding interactions along the fibril axis; (2) β-sheet to-β-sheet packing arrangements perpendicular to the fibril axis (e.g. steric-zipper sites, etc.); and (3) symmetry found between peptide chains, including inter-peptide strand-to-strand hydrogen bonds. This representation indicates whether a residue is in a β-sheet or coil region, which other residue(s) it forms a hydrogen bonding pair with, which specific β-sheet it is in, and what is the overall β-sheet architecture of the amyloid slice (the number of sheets and their arrangement in two dimensions). Using this, the inventors have implemented schemas P, A, and S—however, the inventors note that our technique allows more complicated architectures to be constructed, such as N-sheet β-helices, heterogeneous-peptide fibrils, β-sheet donor-strand-exchange substructures 41, and variants with non-symmetrical restrictions. The inventors choice in resolution strikes a practical compromise between the accuracy of energetic models, the efficiency of computation, the ease of physical interpretation, and the ability to incorporate experimental knowledge or intuition.

Sequence space is defined simply by a set of allowed mutations off a base sequence, per-sequence position, per-residue. For example, one mutation in the set might specify “position index 0 can either be Ala, Leu, or Val.” For ease of use, short-hand definitions are supported such as “all it Val can be Val or Ala.” Specification of both index and allowable mutant residues is required to avoid an exponential computation, as there are 20N residue permutations in a sequence of length N. At runtime, an analysis is performed to determine the minimum dynamic programming table dimension required to fit each possible mutation. Presently, deletion and insertion mutations are not supported due to limitations of the energy models.

To improve runtime speed, the inventors permit (but do not require) an algorithmic parameter that limits the ensemble analysis to only the top N % of β-strand/β-strand interactions, as defined by the energy model. Such a thresholding approach has been applied successfully in similar RNA 42 and protein 43 structure analyses, and has the benefit of dramatically improving runtime speed while maintaining a truncated, but otherwise very similar distribution of energetic states. Further, optional schema-dependent parameters can also be set: (1) limits on the length of β-strands or coils; (2) enabling or disabling β-sheet “kinks” (which permit a single residue deviation in the standard in/out sidechain orientation of β-strands); (3) requiring a minimum/maximum total-fibril β-sheet concentration; (4) enabling or disabling fibril twist (implemented via axially-adjacent β-strands “slipping” registration in a symmetrically consistent matter); (5) permitting N- and C-terminal coil asymmetries; and (6) allowing investigator-defined residue/residue hydrogen bond interactions to be fixed. These parameters effect both the running time and accuracy of ensemble calculations, and allow specific point knowledge to be accounted for in the ensemble, enabling a more profitable back-and-forth between predictions and experimentation.

Additionally (although not treated in this article), schemas can be extended by specific experimental knowledge such as fibril width, flexibility, or known residue interactions—as much or as little a priori knowledge as desired. This facility allows iterative tool re-use, to enhance the predictive accuracy of the model, or to use speculative predictions to help guide further experimentation.

Energy Models for Amyloid-Like Interaction.

Driving the high sensitivity of AmyloidMutants is a potential-energy scoring function derived from residue/residue interaction frequencies observed in known protein structures in the PDB 44, and conditioned on specific microenvironments. To permit Boltzmann ensemble calculations, a fibril's energy must decompose into independent substructure energy scores that recombine according to the schema. Formally, the energy of each fibril structural state s is defined to be Es=−RT log(ps)−RT log(Z), and we make the assumption that Es can be linearly decomposed into i parts such that Es=Σi−RT log(psi)−RT log(Z)45. The probability psk thus represents the likelihood of observing a substructural state k, such as the propensity for two residues to pair within a β-sheet, and log(Z) serves as a statistical centering constant.

The energy scoring function combines the statistical potential that two residues pair within a β-sheet 46, 47 (p(i|j)) and the statistical potential of two consecutive residues forming a coil (p(i, j)). An optional hydropathic packing score can be added, describing the propensity for two residues to pack between two β-sheet faces 48. The relative influence of each of these terms can be scaled independently so one can investigate multiple facets of structural interactions. To best reflect amyloid specific energetics within β-sheets, the inventors examine all non-homolog structures in the PDB (<50% sequence identity) and compute separate frequencies for substructures with similar microenvironments, such as amphipathicity and solvent accessibility, β-strand edge proximity, residue-stacking ladders, β-sheet edges, and β-sheet twist. Energies are derived conditioned on each separate environment, and the appropriate energy is chosen at each step of the search through schema space. There is no explicit cost for performing a mutation ab initio, the mutated sequences simply impact the possible structural scores. The inventors note that although the analysis of amyloid fibrils uses only pairwise likelihoods, the framework incorporates other formulations for specific problem domains, such as incorporation of position-specific scoring matrices 18, energetic models based on stacked residue-pairs 38 and quasi-chemical interaction propensities 49.

Finally, a key feature of this algorithm is the ability to include a wide range of amyloid potential scoring metrics. Indeed, a number of published metrics were substituted or combined with ours 18, 38, although no predictive improvements were seen.

Sampling.

The principal output of AmyloidMutants is list of sequences and structural conformations that are statistically representative of the full ensemble. This is achieved via a sampling procedure that stochastically backtracks over the table of subsequence/substructure conformation scores that were generated when computing Z. To maintain a proper distribution of samples, backtracking steps must be weighted energetically 50. For convenience, AmyloidMutants also can enforce that only unique samples be generated during the backtracking steps (maintaining the same proper distribution). Populations of similar structures are identified and separated via PAM clustering, taking as input the number of clusters, and relying on a distant metric that combines secondary structure, energy score, hydrogen bond registration, coil location, and β-strand overlap. For each cluster a mediod vii is selected to represent that population. This clustering choice highlights sequence differences that arise between structures. Alternately, samples can be clustered according to sequence, presenting the inverse, or clustered according to both structure and sequence. The AmyloidMutant program was used to identify the following sequences: CsgA sequences SALALQ SEQ ID NO: 385) and SELNIY (SEQ ID NO: 386), NSSVN (SEQ ID NO: 387) and NNATAH (SEQ ID NO: 389). CsgB sequences TAIVV (SEQ ID NO: 390) and SQMAIRTV (SEQ ID NO: 391) AAIIGQ/SAQLRQ (SEQ ID NO: 392/SEQ ID NO: 393) and NSDLTITQ (SEQ ID NO: 394.

The minimum energy sequence/structure combination can also be output by AmyloidMutants, by performing backtracking steps which choose minimum energy paths instead of a Boltzmann weighted random selection. However, ensemble mediods have shown to have a higher predictive accuracy than minimum energy structures 38, (data not shown).

Stochastic Contact Maps and Other Calculable Properties.

Through the construction a stochastic contact map, AmyloidMutants can identify small β-strand interaction motifs within the ensemble that may be otherwise hard to discern from full-conformation sampling. A stochastic contact map describes the Boltzmann-weighted likelihood p_{i,j} (normalized by Z) that any two residues i and j will form a β-sheet hydrogen bond given all of the conformations in the ensemble. In addition to local motif identification, contact maps offer a gross metric of overall ensemble makeup and disorder. This can be calculated exactly by expanding the dynamic program to compute sub-structural pair energies, or estimated by extracting pair frequencies from a set of full conformational samples. Further, knowing the partition function Z of a system (even one conditioned on a schema), enables AmyloidMutants to predict a number of other useful properties. For example, per-residue peptide flexibility can be estimated akin to X-ray crystallography B-values 38. The value of Z can also be used on its own to abstractly estimate thermodynamic variables such as entropy (S=∂/∂T(RT ln Z)) and heat capacity (C=1/RT2(∂2Z/∂β2)).

### Example 1

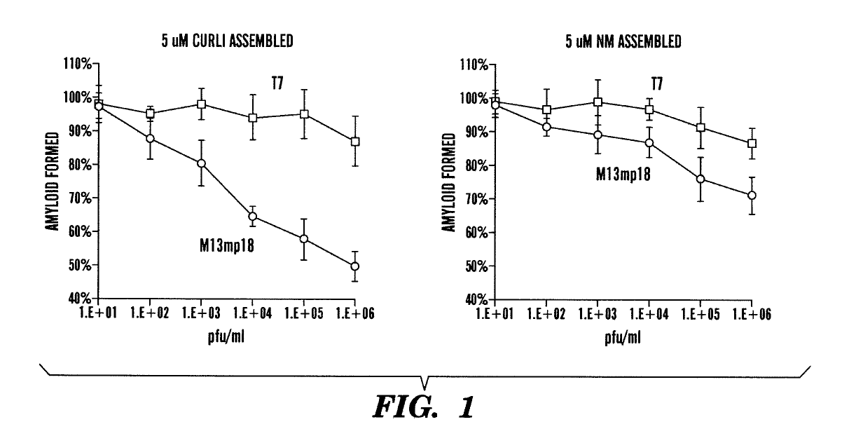

There are amyloids found in humans, yeast, and bacteria. Curli protein in E. coli constitute amyloids (Chapman, M. R. et al. Science 295 (2002)). The inventors first tested whether unmodified phage could block amyloid formation in the absence of capsid-bound peptide-based curli modulators. The inventors monitored in vitro CsgA fiber assembly using ThT fluorescence. As shown in FIG. 1, wild-type T7 phage (T7-wt) exhibited minimal inhibition of curli and Sup35-NM amyloid fiber formation (<15%) while unmodified M13mp18 phage was effective at inhibiting curli amyloids (˜50%) and Sup35-NM amyloids (˜25%). The inventors demonstrated that bacteriophage alone (i.e. non-engineered bacteriophage) was able to block curli formation in vitro. The inventors determined that M13mp18, a filamentous and lysogenic bacteriophage, was more effective than T7, a lytic bacteriophages, at preventing amyolid formation by curli.

### Example 2

CsgA is the major curli subunit and is nucleated by CsgB and CsgF (Chapman, M. R. et al. Science 295 (2002); Loferer, H. et al. Mol Microbiol 26 (1997); Hammar, et al. Mol Microbiol 18 (1995)). The inventors designed potential peptide-inhibitors for curli based off of the native amino acid sequences of CsgA (SEQ ID NO: 1) and CsgB (SEQ ID NO:2).

The inventors designed and expressed specific peptide sequences derived from CsgA polypeptide sequence (SEQ ID NO: 1), as shown in Table 3 and cloned them into the EcoRI and HindIII sites of T7select-415 plasmid from Novagen.

TABLE 3
Sequences derived from CsgA that were cloned into T7select-415 plasmid between EcoRI and
HindIII restriction sites, the CsgA sequence is highlighted in bold between the EcoRI
and HindIII restriction sites (not bold). The nucleic acid sequences
(SEQ ID NO: 3-10) encode polypeptides SEQ ID NOs 11-18 respectively.

| | | Relevant Peptide |
|---|---|---|
| NUMBER | DNA Sequence | Sequence |
| 17 | GGGGATCCGAATTCGTCTGAGCTGAACATTTACCAGTACGGTGGCAA | SELNIYQYGG |
| | GCTTGCGGCC (SEQ ID NO: 3) | (SEQ ID NO: 11) |
| 18 | GGGGATCCGAATTCGTCTGCACTTGCTCTGCAAACTGATGCCCGTAA | SALALQTDAR |
| | GCTTGCGGCC (SEQ ID NO: 4) | (SEQ ID NO: 12) |
| 19 | GGGGATCCGAATTCGAACTCCTCCGTCAACGTGACTCAGGTTGGCAA | NSSVNVTQVG |
| | GCTTGCGGCC (SEQ ID NO: 5) | (SEQ ID NO: 13) |
| 20 | GGGGATCCGAATTCGTTTGGTAACAACGCGACCGCTCATCAGTACAA | FGNNATAHQY |
| | GCTTGCGGCC (SEQ ID NO: 6) | (SEQ ID NO: 14) |
| 21 | GGGGATCCGAATTCGCCGTCTGAGCTGAACATTTACCAGTACGGTGG | PSELNIYQYGG |
| | CAAGCTTGCGGCC (SEQ ID NO: 7) | (SEQ ID NO: 15) |
| 22 | GGGGATCCGAATTCGTCTGCACTTGCTCTGCAAACTGATGCCCGTCG | SALALQTDARR |
| | GAAGCTTGCGGCC (SEQ ID NO: 8) | (SEQ ID NO: 16) |
| 23 | GGGGATCCGAATTCGAACTCCTCCGTCAACGTGACTCAGGTTGGCCC | NSSVNVTQVGP |
| | GAAGCTTGCGGCC (SEQ ID NO: 9) | (SEQ ID NO: 17) |
| 24 | GGGGATCCGAATTCGTTTGGTAACAACGCGACCGCTCATCAGTACCG | FGNNATAHQYR |
| | GAAGCTTGCGGCC (SEQ ID NO: 10) | (SEQ ID NO: 18) |

The inventors designed and expressed specific peptide sequences derived from CsgB polypeptide sequence (SEQ ID NO: 2), as shown in Table 4 and cloned them into the EcoRI and HindIII sites of T7select-415 plasmid from Novagen.

TABLE 4
Sequences derived from CsgB that were cloned into T7select-415 plasmid between EcoRI and
HindIII restriction sites, the CsgB sequence is highlighted in bold between the EcoRI
and HindIII restriction sites (not bold). The nucleic acid sequences (SEQ ID NO: 19-26)
encode polypeptides SEQ ID NOs 27-34 respectively.

| | DNA Sequence encoding CsgB peptides | |
|---|---|---|
| # | Sequence | CsgB Peptide |
| 25 | GGGGATCCGAATTCGAATCAGGCAGCCATAATTGGTCAAGCTGGGAAGC | NQAAIIGQAG |
| | TTGCGGCC (SEQ ID NO: 19) | (SEQ ID NO: 27) |
| 26 | GGGGATCCGAATTCGAATAGTGCTCAGTTACGGCAGGGAGGCTCAAAGC | NSAQLRQGGS |
| | TTGCGGCC (SEQ ID NO: 20) | (SEQ ID NO: 28) |
| 27 | GGGGATCCGAATTCGAAAACGGCAATTGTAGTGCAGAGACAGTCGAAGC | KTAIVVQRQS |
| | TTGCGGCC (SEQ ID NO: 21) | (SEQ ID NO: 29) |
| 28 | GGGGATCCGAATTCGTCGCAAATGGCTATTCGCGTGACACAACGTAAGC | SQMAIRVTQR |
| | TTGCGGCC (SEQ ID NO: 22) | (SEQ ID NO: 30) |
| 29 | GGGGATCCGAATTCGAATCAGGCAGCCATAATTGGTCAAGCTGGGCGGA | NQAAIIGQAGR |
| | AGCTTGCGGCC (SEQ ID NO: 23) | (SEQ ID NO: 31) |
| 30 | GGGGATCCGAATTCGAATAGTGCTCAGTTACGGCAGGGAGGCTCACCGA | NSAQLRQGGSP |
| | AGCTTGCGGCC (SEQ ID NO: 24) | (SEQ ID NO: 32) |
| 31 | GGGGATCCGAATTCGAAAACGGCAATTGTAGTGCAGAGACAGTCGCCGA | KTAIVVQRQSP |
| | AGCTTGCGGCC (SEQ ID NO: 25) | (SEQ ID NO: 33) |
| 32 | GGGGATCCGAATTCGTCGCAAATGGCTATTCGCGTGACACAACGTCGGA | SQMAIRVTQRR |
| | AGCTTGCGGCC (SEQ ID NO: 26) | (SEQ ID NO: 34) |

The inventors expressed the anti-amyloid peptides shown in Tables 3 and 4 by bacteriophages to generate a library of anti-amyloid peptide engineered bacteriophages or amyloid-inhibiting agents. In some embodiments, the peptides listed in Tables 3 and 4 (SEQ ID NO: 11-18 and 27-34 respectively) were expressed on the surface of bacteriophages via display technology. The potential advantages for doing so includes: 1) increased efficacy due to combination of amyloid-inhibiting effects from peptides and bacteriophages; 2) decreased production costs since producing engineered bacteriophages is easier than synthesizing peptides; 3) enhanced delivery of bacteriophages and peptides conjugated to each other given that bacteriophages could be specific for bacterial hosts which express curli and peptides could have affinity for specific amyloids; 4) potential for enhanced clearance of amyloid aggregates if peptides intercalate into amyloids and immune systems target bacteriophages and the associated aggregate for clearance.

In other experiments, the peptides listed in Tables 3 and 4 (SEQ ID NO: 11-18 and 27-34 respectively) are expressed intracellularly during infection instead of displaying them on bacteriophages surfaces. These peptides would be released into the extracellular space during bacterial lysis (Lu, T. K. et al. Proc Natl Acad Sci USA 104 (2007)). In some embodiments, the CsgA and CsgB peptides are secreted from bacteria infected with the bacteriophages. These strategies could potentially enable greater amounts of anti-amyloid peptides to be produced.

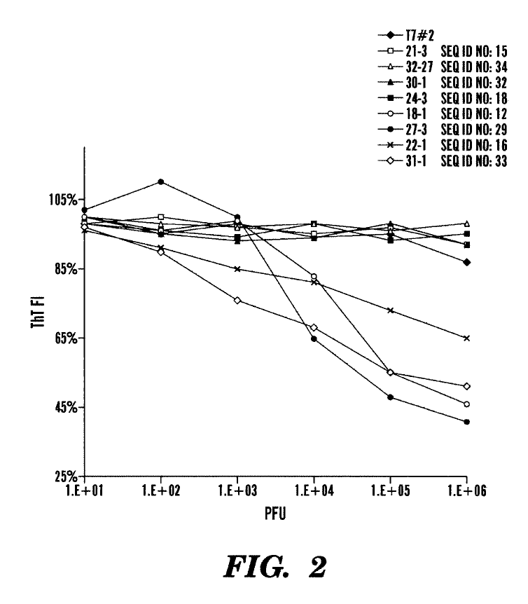

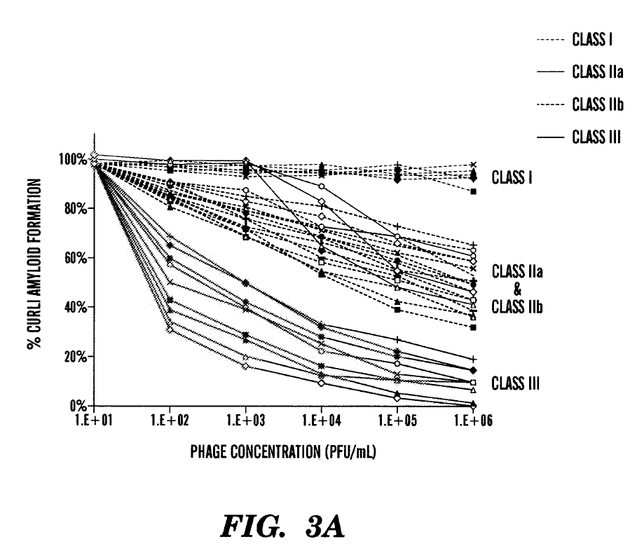

The inventors discovered that four sequences from Table 3 (SEQ ID NO: 11-18) and Table 4 (SEQ ID NO 24-34) which were cloned into T7select-415 bacteriophages produced particularly effective inhibitors of curli assembly (FIG. 2). The levels of inhibition observed with engineered bacteriophages expressing curli-inhibiting peptides were greater than with unmodified control T7select-415 bacteriophage (FIG. 2). The most effective engineered bacteriophages were the ones which expressed CsgA peptides #18 (SEQ ID NO:12) or #22 (SEQ ID NO: 16) or CsgB peptides #27 (SEQ ID NO: 29) and #31 (SEQ ID NO: 33).

### Example 3

Next the inventors generated a new set of CsgA and CsgB peptide sequences expressed by bacteriophages for enhanced anti-amyloid activity. The new peptide sequences, shown in Table 5, are variant sequences (i.e. one or more changes amino acid) from the peptides shown in Tables 3 and 4. In particular, the inventors modified (i.e. added, deleted or substituted) one or more amino acid of the CsgA peptides (SEQ ID NOs: 11 or 12), or modified (i.e. added, deleted or substituted) one or more amino acid of the CsgB peptides (SEQ ID NO: 29).

Further, to see if charged mutations could enhance blocking of CsgA fiber assembly, the inventors mutated key residues within CsgA₄₃₋₅₂ (SEQ ID NO: 11), CsgA₅₅₋₆₄ (SEQ ID NO: 12), and CsgB₁₃₃₋₁₄₂ (SEQ ID NO: 29) to lysines (Table 5). The inventors also constructed charged mutations in CsgB₁₄₂₋₁₅₁ (SEQ ID NO: 30) since the peptide arrays showed that the peptides CsgB₁₃₀₋₁₄₉ was important for nucleation (Table 5). In addition, the inventors constructed another set of peptides by introducing charged residues, such as lysines and arginines, flanking CsgA₄₃₋₅₂, CsgA₅₅₋₆₄, CsgB₁₁₃₋₁₄₂, and CsgB₁₄₂₋₁₅₁ sequences (Table 5). Finally, the inventors created a set of β-breaker peptides by flanking CsgA₄₃₋₅₂, CsgA₅₅₋₆₄, CsgB₁₃₃₋₁₄₂, and CsgB₁₄₂₋₁₅₁ sequences with proline residues (Table 5)¹².

TABLE 5
Recombinant phages constructed from DNA encoding peptide sequences derived from CsgA
or CsgB cloned in between EcoR1 and HindIII in T7select vectors. Mutations or
flanking sequences are bolded either in red or black. Each primer pair contains a
forward and reverse primer. When the sequence of a forward primer or a reverse primer
is known (shown in Table 6), one of skill in the art will readily be able to
determine the sequence of another, which is the reverse of the complementary
sequence to the known primer.

| | Phage | Sequence | | Primer |
|---|---|---|---|---|
| Phage Name | Background | Based On | Actual Sequence | Pairs |
| T7-CsgA₄₃₋₅₂ | T7select415 | CsgA₄₃₋₅₂ | SELNIYQYGG | N17, N33 |
| | | | (SEQ ID NO: 11)* | |
| T7-CsgA₅₅₋₆₄ | T7select415 | CsgA₅₅₋₆₄ | SALALQTDAR | N18, N34 |
| | | | (SEQ ID NO: 12) * | |
| T7-CsgB₁₃₃₋₁₄₂ | T7select415 | CsgB₁₃₃₋₁₄₂ | KTAIVVQRQS | N27, N43 |
| | | | (SEQ ID NO: 29) * | |
| T7-RRR-CsgA₄₃₋₅₂ | T7select415 | CsgA₄₃₋₅₂ | RRRSELNIYQYGG | N49, N97 |
| | | | (SEQ ID NO: 35) * | |
| T7-PPP-CsgA₄₃₋₅₂ | T7select415 | CsgA₄₃₋₅₂ | PPPSELNIYQYGG | N50, N98 |
| | | | (SEQ ID NO: 36) * | |
| T7-RRR-CsgA₄₃₋₅₂-RRR | T7select415 | CsgA₄₃₋₅₂ | RRRSELNIYQYGGRRR | N51 |
| | | | (SEQ ID NO: 37) | |
| T7-PPP-CsgA₄₃₋₅₂-PPP | T7select415 | CSgA₄₃₋₅₂ | PPPSELNIYQYGGPPP | N52 |
| | | | (SEQ ID NO: 38) | |
| T7-PPP-CsgA₄₃₋₅₂-RRR | T7select415 | CsgA₄₃₋₅₂ | PPPSELNIYQYGGRRR | N53, |
| | | | (SEQ ID NO: 39) * | N101 |
| T7-CsgA₄₃₋₅₂-RRR | T7select415 | CsgA₄₃₋₅₂ | SELNIYQYGGRRR | N54, |
| | | | (SEQ ID NO: 40) * | N102 |
| T7-CsgA₄₃₋₅2-PPP | T7select415 | CsgA₄₃₋₅₂ | SELNIYQYGGPPP | N55, |
| | | | (SEQ ID NO: 41) | N103 |
| | T7select415 | CsgA₄₃₋₅₂ | SEKNKYQYGG | N56 |
| | | | (SEQ ID NO: 42) | |
| T7-CsgA₄₃₋₅₂(I47K-Q49K) | T7select415 | CsgA₄₃₋₅₂ | SELNKYKYGG | N57, |
| | | | (SEQ ID NO: 43) | N105 |
| T7-CsgA₄₃₋₅₂(I47K-Y48K) | T7select415 | CsgA₄₃₋₅₂ | SELNKKQYGG | N58, |
| | | | (SEQ ID NO: 44) | N106 |
| T7-CsgA₄₃₋₅₂(S43K-G52K) | T7select415 | CsgA₄₃₋₅₂ | KELNIYQYGK | N59, |
| | | | (SEQ ID NO: 45) * | N107 |
| T7-CsgA₄₃₋₅₂(S43K-I47K- | T7select415 | CsgA₄₃₋₅₂ | KELNKYQYGK | N60, |
| G52K) | | | (SEQ ID NO: 46) | N108 |
| T7-RRR-CsgA₅₅₋₆₄ | T7select415 | CsgA₅₅₋₆₄ | RRRSALALQTDAR | N61, |
| | | | (SEQ ID NO: 47) | N109 |
| T7-PPP-CsgA₅₅₋₆₄ | T7select415 | CsgA₅₅₋₆₄ | PPPSALALQTDAR | N62 |
| | | | (SEQ ID NO: 48) | |
| T7-CsgA₅₅₋₆₄-RRR | T7select415 | CsgA₅₅₋₆₄ | SALALQTDARRRR | N63, |
| | | | (SEQ ID NO: 49) * | N111 |
| T7-CsgA₅₅₋₆₄-PPP | T7select415 | CsgA₅₅₋₆₄ | SALALQTDARPPP | N64, |
| | | | (SEQ ID NO: 50) * | N112 |
| T7-RRR-CsgA₅₅₋₆₄-RRR | T7select415 | CsgA₅₅₋₆₄ | RRRSALALQTDARRRR | N65, |
| | | | (SEQ ID NO: 51) * | N113 |
| T7-PPP-CsgA₅₅₋₆₄-PPP | T7select415 | CsgA₅₅₋₆₄ | PPPSALALQTDARPPP | N66, |
| | | | (SEQ ID NO: 52) * | N114 |
| T7-PPP-CsgA₅₅₋₆₄-RRR | T7select415 | CsgA₅₅₋₆₄ | PPPSALALQTDARRRR | N67, |
| | | | (SEQ ID NO: 53) * | N115 |
| | T7select415 | CsgA₅₅₋₆₄ | kSALAkQTDARk | N68 |
| | | | (SEQ ID NO: 54) | |
| | T7select415 | CsgA₅₅₋₆₄ | kSALALQTDARk | N69 |
| | | | (SEQ ID NO: 55) | |
| | T7select415 | CsgA₅₅₋₆₄ | SKLKLQTDAR | N70 |
| | | | (SEQ ID NO: 56) | |
| T7-CsgA₅₅₋₆₄(Q60K-T61K) | T7select415 | CsgA₅₅₋₆4 | SALALKKDAR | N71, |
| | | | (SEQ ID NO: 57) | N119 |
| T7-CsgA₅₅₋₆₄(A58K-Q60K) | T7select415 | CsgA₅₅₋₆₄ | SALKLKTDAR | N72, |
| | | | (SEQ ID NO: 58) | N120 |
| T7-RRR-CsgB₁₃₃₋₁₄₂ | T7select415 | CsgB₁₃₃₋₁₄₂ | RRRKTAIVVQRQS | N73, |
| | | | (SEQ ID NO: 59) * | N121 |
| T7-PPP-CsgB₁₃₃₋₁₄₂ | T7select415 | CsgB₁₃₃₋₁₄₂ | PPPKTAIVVQRQS | N74, |
| | | | (SEQ ID NO: 60) * | N122 |
| T7-RRR-CsgB₁₃₃₋₁₄₂-PPP | T7select415 | CsgB₁₃₃₋₁₄₂ | RRRKTAIVVQRQSPPP | N75, or |
| | | | (SEQ ID NO: 61) | N79, |
| | | | | N127 |
| T7-RRR-CsgB₁₃₃₋₁₄₂-RRR | T7select415 | CsgB₁₃₃₋₁₄₂ | RRRKTAIVVQRQSRRR | N76 or |
| | | | (SEQ ID NO: 62) * | N77, |
| | | | | N125 |
| T7-PPP-CsgB₁₃₃₋₁₄₂-PPP | T7select415 | CsgB₁₃₃₋₁₄₂ | PPPKTAIVVQRQSPPP | N77 or |
| | | | (SEQ ID NO: 63) | N78, |
| | | | | N126 |
| T7-CsgB₁₃₃₋₁₄₂-RRR | T7select415 | CsgB₁₃₃₋₁₄₂ | KTAIVVQRQSRRR | N78 or |
| | | | (SEQ ID NO: 64) | N75, |
| | | | | N123 |
| T7-CsgB₁₃₃₋₁₄₂-PPP | T7select415 | CsgB₁₃₃₋₁₄₂ | KTAIVVQRQSPPP | N79 or |
| | | | (SEQ ID NO: 65) | N76, |
| | | | | N124 |
| T7-CsgB₁₃₃₋₁₄₂(A135K-V137K) | T7select415 | CsgB₁₃₃₋₁₄₂ | KTKIKVQRQS | N80, |
| | | | (SEQ ID NO: 66) | N128 |
| T7-CsgB₁₃₃₋₁₄₂(I136K-V138K) | T7select415 | CsgB₁₃₃₋₁₄₂ | KTAKVKQRQS | N81, |
| | | | (SEQ ID NO: 67) | N129 |
| T7-CsgB₁₃₃₋₁₄₂(I136K-V137K) | T7select415 | CsgB₁₃₃₋₁₄₂ | KTAKKVQRQS | N82, |
| | | | (SEQ ID NO: 68) | N130 |
| T7-K-CsgB₁₃₃₋₁₄₂-K | T7select415 | CsgB₁₃₃₋₁₄₂ | KKTAIVVQRQSK | N83, |
| | | | (SEQ ID NO: 69) | N131 |
| T7-K-CsgB₁₃₃₋₁₄₂(V137K)-K | T7select415 | CsgB₁₃₃₋₁₄₂ | KKTAIKVQRQSK | N84, |
| | | | (SEQ ID NO: 70) | N132 |
| T7-PPP-CsgB₁₄₂₋₁₅₁ | T7select415 | CsgB₁₄₂₋₁₅₁ | PPPSQMAIRVTQR | N85, |
| | | | (SEQ ID NO: 71) | N133 |
| T7-RRR-CsgB₁₄₂₋₁₅₁ | T7select415 | CsgB₁₄₂₋₁₅₁ | RRRSQMAIRVTQR | N86, |
| | | | (SEQ ID NO: 72) | N134 |
| T7-CsgB₁₄₂₋₁₅₁-PPP | T7select415 | CsgB₁₄₂₋₁₅₁ | SQMAIRVTQRPPP | N87, |
| | | | (SEQ ID NO: 73) | N135 |
| T7-CsgB₁₄₂₋₁₅₁-RRR | T7select415 | CsgB₁₄₂₋₁₅₁ | SQMAIRVTQRRRR | N88, |
| | | | (SEQ ID NO: 74) | N136 |
| T7-PPP-CsgB₁₄₂₋₁₅₁-PPP | T7select415 | CsgB₁₄₂₋₁₅₁ | PPPSQMAIRVTQRPPP | N89, |
| | | | (SEQ ID NO: 75) | N137 |
| T7-RRR-CsgB₁₄₂₋₁₅₁-RRR | T7select415 | CsgB₁₄₂₋₁₅₁ | RRRSQMAIRVTQRRRR | N90 |
| | | | (SEQ ID NO: 76) | |
| T7-PPP-CsgB₁₄₂₋₁₅₁-RRR | T7select415 | CsgB₁₄₂₋₁₅₁ | PPPSQMAIRVTQRRRR | N91, |
| | | | (SEQ ID NO: 77) | N139 |
| T7-CsgB₁₄₂₋₁₅₁(A145K-I146K) | T7select415 | CsgB₁₄₂₋₁₅₁ | SQMKKRVTQR | N92, |
| | | | (SEQ ID NO: 78) | N140 |
| T7-CsgB₁₄₂₋₁₅₁(M144K-I146K) | T7select415 | CsgB₁₄₂₋₁₅₁ | SQKAKRVTQR | N93, |
| | | | (SEQ ID NO: 79) | N141 |
| T7-CsgB₁₄₂₋₁₅₁(V148K-Q150K) | T7select415 | CsgB₁₄₂₋₁₅₁ | SQMAIRKTKR | N94, |
| | | | (SEQ ID NO: 80) | N142 |
| T7-K-CsgB₁₄₂₋₁₅₁-K | T7select415 | CsgB₁₄₂₋₁₅₁ | KSQMAIRVTQRK | N95, |
| | | | (SEQ ID NO: 81) | N143 |
| T7_{med}-RRR-CsgB₁₃₃₋₁₄₂-PPP | T7select10- | CsgB₁₃₃₋₁₄₂ | RRRKTAIVVQRQSPPP | N79, |
| | 3b | | (SEQ ID NO: 82) | N127 |
| | T7select415 | CsgB₁₄₂₋₁₅₁ | KSQMAKRVTQRK | N96 |
| | | | (SEQ ID NO: 83) | |
| T7-RRRRR-CsgB₁₃₃₋₁₄₂- | T7select415 | CsgB₁₃₃₋₁₄₂ | RRRRRKTAIVVQRQSPPPP | D1013, |
| PPPPP | | | P | D1016 |
| | | | (SEQ ID NO: 84) | |
| T7-RRR-CsgB₁₃₃₋₁₄₂-PPPPP | T7select415 | CsgB₁₃₃₋₁₄₂ | RRRKTAIVVQRQSPPPPP | D1018, |
| | | | (SEQ ID NO: 85) | D1016 |
| T7-RRRRR-CsgB₁₃₃₋₁₄₂-PPP | T7select415 | CsgB₁₃₃₋₁₄₂ | RRRRRKTAIVVQRQSPPP | D1013, |
| | | | (SEQ ID NO: 86) | D1014 |
| T7-GGG-CsgB₁₃₃₋₁₄₂-PPP | T7select415 | CsgB₁₃₃₋₁₄₂ | GGGKTAIVVQRQSPPP | D1019, |
| | | | (SEQ ID NO: 87) | D1014 |
| T7-RRR-CsgB₁₃₃₋₁₄₂-GGG | T7select415 | CsgB₁₃₃₋₁₄₂ | RRRKTAIVVQRQSGGG | D1018, |
| | | | (SEQ ID NO: 88) | D1021 |
| T7-RR-CsgB₁₃₃₋₁₄₂-PP | T7select415 | CsgB₁₃₃₋₁₄₂ | RRKTAIVVQRQSPP | D1063, |
| | | | (SEQ ID NO: 89) | D1064 |
| T7-R-CsgB₁₃₃₋₁₄₂-P | T7select415 | CsgB₁₃₃₋₁₄₂ | RKTAIVVQRQSP | D1066, |
| | | | (SEQ ID NO: 90) | D1067 |
| T7-con | T7select415 | S•Tag | KETAAAKFERQHMDS | Positive |
| | | | (SEQ ID NO: 91) | control |
| | | | | insert |

TABLE 6
DNA oligonucleotide sequences of the primers used in construction of recombinant
phages in Table 5.

| Oligo | |
|---|---|
| Name | Oligonucleotide Sequence |
| N17 | GGGGATCCGAATTCGTCTGAGCTGAACATTTACCAGTACGGTGGCAAGCTTGCGGCC |
| | (SEQ ID NO: 3) |
| N33 | GGCCGCAAGCTTGCCACCGTACTGGTAAATGTTCAGCTCAGACGAATTCGGATCCCC |
| | (SEQ ID NO: 92) |
| N18 | GGGGATCCGAATTCGTCTGCACTTGCTCTGCAAACTGATGCCCGTAAGCTTGCGGCC |
| | (SEQ ID: 4) |
| N34 | GGCCGCAAGCTTACGGGCATCAGTTTGCAGAGCAAGTGCAGACGAATTCGGATCCCC |
| | (SEQ ID NO: 93) |
| N27 | GGGGATCCGAATTCGAAAACGGCAATTGTAGTGCAGAGACAGTCGAAGCTTGCGGCC |
| | (SEQ ID NO: 21) |
| N43 | GGCCGCAAGCTTCGACTGTCTCTGCACTACAATTGCCGTTTTCGAATTCGGATCCCC |
| | (SEQ ID NO: 94) |
| N49 | GGGGATCCGAATTCGcgccgtcggTCTGAGCTGAACATTTACCAGTACGGTGGCAAGCTTGCG |
| | GCC |
| | (SEQ ID NO: 95) |
| N97 | GGCCGCAAGCTTGCCACCGTACTGGTAAATGTTCAGCTCAGAccgacggcgCGAATTCGGATC |
| | CCC |
| | (SEQ ID NO: 96) |
| N50 | GGGGATCCGAATTCGccgccaccaTCTGAGCTGAACATTTACCAGTACGGTGGCAAGCTTGCG |
| | GCC |
| | (SEQ ID NO: 97) |
| N98 | GGCCGCAAGCTTGCCACCGTACTGGTAAATGTTCAGCTCAGAtggtggcggCGAATTCGGATC |
| | CCC |
| | (SEQ ID NO: 98) |
| N51 | GGGGATCCGAATTCGcgccgtcgcTCTGAGCTGAACATTTACCAGTACGGTGGCcgccgtcgcAAG |
| | CTTGCGGCC (SEQ ID NO: 99) |
| N52 | GGGGATCCGAATTCGccgccaccaTCTGAGCTGAACATTTACCAGTACGGTGGCccaccaccgAA |
| | GCTTGCGGCC (SEQ ID NO: 100) |
| N53 | GGGGATCCGAATTCGccgccaccaTCTGAGCTGAACATTTACCAGTACGGTGGCcgccgtcgcAA |
| | GCTTGCGGCC (SEQ ID NO: 101) |
| N101 | GGCCGCAAGCTTgcgacggcgGCCACCGTACTGGTAAATGTTCAGCTCAGAtggtggcggCGAATT |
| | CGGATCCCC(sEQ ID NO: 102) |
| N54 | GGGGATCCGAATTCGTCTGAGCTGAACATTTACCAGTACGGTGGCcgccgtcggAAGCTTGCG |
| | GCC |
| | (SEQ ID NO: 103) |
| N102 | GGCCGCAAGCTTccgacggcgGCCACCGTACTGGTAAATGTTCAGCTCAGACGAATTCGGATC |
| | CCC |
| | (SEQ ID NO: 104) |
| N55 | GGGGATCCGAATTCGTCTGAGCTGAACATTTACCAGTACGGTGGCccgccaccaAAGCTTGCG |
| | GCC |
| | (SEQ ID NO: 105) |
| N56 | GGGGATCCGAATTCGTCTGAGaaaAACaaaTACCAGTACGGTGGCAAGCTTGCGGCC |
| | (SEQ ID NO: 106) |
| N103 | GGCCGCAAGCTTtggtggcggGCCACCGTACTGGTAAATGTTCAGCTCAGACGAATTCGGATC |
| | CCC |
| | (SEQ ID NO: 107) |
| N57 | GGGGATCCGAATTCGTCTGAGCTGAACaaaTACaaaTACGGTGGCAAGCTTGCGGCC |
| | (SEQ ID NO: 108) |
| N105 | GGCCGCAAGCTTGCCACCGTAtttGTAtttGTTCAGCTCAGACGAATTCGGATCCCC |
| | (SEQ ID NO: 109) |
| N58 | GGGGATCCGAATTCGTCTGAGCTGAACaaaaagCAGTACGGTGGCAAGCTTGCGGCC |
| | (SEQ ID NO: 110) |
| N106 | GGCCGCAAGCTTGCCACCGTACTGctttttGTTCAGCTCAGACGAATTCGGATCCCC |
| | (SEQ ID NO: 111) |
| N59 | GGGGATCCGAATTCGaaaTCTGAGCTGAACATTTACCAGTACGGTGGCaagAAGCTTGCGGC |
| | C |
| | (SEQ ID NO: 112) |
| N107 | GGCCGCAAGCTTcttGCCACCGTACTGGTAAATGTTCAGCTCAGAtttCGAATTCGGATCCCC |
| | (SEQ ID NO: 113) |
| N60 | GGGGATCCGAATTCGaaaGAGCTGAACaaaTACCAGTACGGTaaaAAGCTTGCGGCC |
| | (SEQ ID NO: 114) |
| N108 | GGCCGCAAGCTTtttACCGTACTGGTAtttGTTCAGCTCtttCGAATTCGGATCCCC |
| | (SEQ ID NO: 115) |
| N61 | GGGGATCCGAATTCGcgccgtcggTCTGCACTTGCTCTGCAAACTGATGCCCGTAAGCTTGCG |
| | GCC |
| | (SEQ ID NO: 116) |
| N62 | GGGGATCCGAATTCGccgccaccaTCTGCACTTGCTCTGCAAACTGATGCCCGTAAGCTTGCG |
| | GCC (SEQ ID NO: 117) |
| N109 | GGCCGCAAGCTTACGGGCATCAGTTTGCAGAGCAAGTGCAGAccgacggcgCGAATTCGGAT |
| | CODIC (SEQ ID NO: 118) |
| N63 | GGGGATCCGAATTCGTCTGCACTTGCTCTGCAAACTGATGCCCGTcgccgtcggAAGCTTGCG |
| | GCC |
| | (SEQ ID NO: 119) |
| N111 | GGCCGCAAGCTTccgacggcgACGGGCATCAGTTTGCAGAGCAAGTGCAGACGAATTCGGAT |
| | CODIC (SEQ ID NO: 120) |
| N64 | GGGGATCCGAATTCGTCTGCACTTGCTCTGCAAACTGATGCCCGTccgccaccaAAGCTTGCG |
| | GCC |
| | (SEQ ID NO: 121) |
| N112 | GGCCGCAAGCTTtggtggcggACGGGCATCAGTTTGCAGAGCAAGTGCAGACGAATTCGGATC |
| | CCC |
| | (SEQ ID NO: 122) |
| N65 | GGGGATCCGAATTCGcgccgtcggTCTGCACTTGCTCTGCAAACTGATGCCCGTcgccgtcggAAG |
| | CTTGCGGCC (SEQ ID NO: 123) |
| N113 | GGCCGCAAGCTTccgacggcgACGGGCATCAGTTTGCAGAGCAAGTGCAGAccgacggcgCGAA |
| | TTCGGATCCCC (SEQ ID NO: 124) |
| N66 | GGGGATCCGAATTCGccgccaccaTCTGCACTTGCTCTGCAAACTGATGCCCGTccgccaccaAA |
| | GCTTGCGGCC (SEQ ID NO: 125) |
| N114 | GGCCGCAAGCTTtggtggcggACGGGCATCAGTTTGCAGAGCAAGTGCAGAtggtggcggCGAATT |
| | CGGATCCCC (SEQ ID NO: 126) |
| N67 | GGGGATCCGAATTCGcgccgtcggTCTGCACTTGCTCTGCAAACTGATGCCCGTccgccaccaAAG |
| | CTTGCGGCC (SEQ ID NO: 127) |
| N68 | GGGGATCCGAATTCGaaaTCTGCACTTGCTaaaCTGCAAACTGATGCCCGTaaaAAGCTTGC |
| | GGCC (SEQ ID NO: 128) |
| N69 | GGGGATCCGAATTCGaaaTCTGCACTTGCTCTGCAAACTGATGCCCGTaaaAAGCTTGCGGC |
| | C (SEQ ID NO: 129) |
| N70 | GGGGATCCGAATTCGTCTaagCTTaaaCTGCAAACTGATGCCCGTAAGCTTGCGGCC |
| | (SEQ ID NO: 130) |
| N115 | GGCCGCAAGCTTtggtggcggACGGGCATCAGTTTGCAGAGCAAGTGCAGAccgacggcgCGAAT |
| | TCGGATCCCC (SEQ ID NO: 131) |
| N71 | GGGGATCCGAATTCGTCTGCACTTGCTCTGaagaaaGATGCCCGTAAGCTTGCGGCC |
| | (SEQ ID NO: 132) |
| N119 | GGCCGCAAGCTTACGGGCATCtttcttCAGAGCAAGTGCAGACGAATTCGGATCCCC |
| | (SEQ ID NO: 133) |
| N72 | GGGGATCCGAATTCGTCTGCACTTaaaCTGaaaACTGATGCCCGTAAGCTTGCGGCC |
| | (SEQ ID NO: 134) |
| N120 | GGCCGCAAGCTTACGGGCATCAGTtttCAGtttAAGTGCAGACGAATTCGGATCCCC |
| | (SEQ ID NO: 135) |
| N73 | GGGGATCCGAATTCGcgccgtcggAAAACGGCAATTGTAGTGCAGAGACAGTCGAAGCTTGCG |
| | GCC |
| | (SEQ ID NO: 135) |
| N121 | GGCCGCAAGCTTCGACTGTCTCTGCACTACAATTGCCGTTTTccgacggcgCGAATTCGGATC |
| | CCC |
| | (SEQ ID NO: 136) |
| N74 | GGGGATCCGAATTCGccgccaccaAAAACGGCAATTGTAGTGCAGAGACAGTCGAAGCTTGCG |
| | GCC (SEQ ID NO: 137) |
| N122 | GGCCGCAAGCTTCGACTGTCTCTGCACTACAATTGCCGTTTTtggtggcggCGAATTCGGATCC |
| | CC |
| | (SEQ ID NO: 138) |
| N75 | GGGGATCCGAATTCGAAAACGGCAATTGTAGTGCAGAGACAGTCGcgccgtcggAAGCTTGCG |
| | GCC |
| | (SEQ ID NO: 139) |
| N123 | GGCCGCAAGCTTccgacggcgCGACTGTCTCTGCACTACAATTGCCGTTTTCGAATTCGGATC |
| | CCC |
| | (SEQ ID NO: 140) |
| N76 | GGGGATCCGAATTCGAAAACGGCAATTGTAGTGCAGAGACAGTCGccgccaccaAAGCTTGCG |
| | GCC (SEQ ID NO: 141) |
| N124 | GGCCGCAAGCTTtggtggcggCGACTGTCTCTGCACTACAATTGCCGTTTTCGAATTCGGATCC |
| | CC |
| | (SEQ ID NO: 142) |
| N77 | GGGGATCCGAATTCGcgccgtcggAAAACGGCAATTGTAGTGCAGAGACAGTCGcgccgtcggAA |
| | GCTTGCGGCC(sEQ ID NO: 143) |
| N125 | GGCCGCAAGCTTccgacggcgCGACTGTCTCTGCACTACAATTGCCGTTTTccgacggcgCGAATT |
| | CGGATCCCC(sEQ ID NO: 144) |
| N78 | GGGGATCCGAATTCGccgccaccaAAAACGGCAATTGTAGTGCAGAGACAGTCGccgccaccaAA |
| | GCTTGCGGCC (SEQ ID NO: 145) |
| N126 | GGCCGCAAGCTTtggtggcggCGACTGTCTCTGCACTACAATTGCCGTTTTtggtggcggCGAATTC |
| | GGATCCCC (SEQ ID NO: 146) |
| N79 | GGGGATCCGAATTCGcgccgtcggAAAACGGCAATTGTAGTGCAGAGACAGTCGccgccaccaAA |
| | GCTTGCGGCC (SEQ ID NO: 147) |
| N127 | GGCCGCAAGCTTtggtggcggCGACTGTCTCTGCACTACAATTGCCGTTTTccgacggcgCGAATT |
| | CGGATCCCC (SEQ ID NO: 148) |
| N80 | GGGGATCCGAATTCGaaaAAAaagATTGTAGTGCAGAGACAGTCGAAGCTTGCGGCC |
| | (SEQ ID NO: 149) |
| N128 | GGCCGCAAGCTTCGACTGTCTCTGCACTACAATcttTTTtttCGAATTCGGATCCCC |
| | (SEQ ID NO: 150) |
| N81 | GGGGATCCGAATTCGAAAACGGCAaagATTaaaCAGAGACAGTCGAAGCTTGCGGCC |
| | (SEQ ID NO: 151) |
| N129 | GGCCGCAAGCTTCGACTGTCTCTGtttAATcttTGCCGTTTTCGAATTCGGATCCCC |
| | (SEQ ID NO: 152) |
| N82 | GGGGATCCGAATTCGAAAACGGCAaaaaagGTGCAGAGACAGTCGAAGCTTGCGGCC |
| | (SEQ ID NO: 153) |
| N130 | GGCCGCAAGCTTCGACTGTCTCTGCACctttttTGCCGTTTTCGAATTCGGATCCCC |
| | (SEQ ID NO: 154) |
| N83 | GGGGATCCGAATTCGaagAAAACGGCAATTGTAGTGCAGAGACAGTCGaagAAGCTTGCGG |
| | CC |
| | (SEQ ID NO: 155) |
| N131 | GGCCGCAAGCTTcttCGACTGTCTCTGCACTACAATTGCCGTTTTcttCGAATTCGGATCCCC |
| | (SEQ ID NO: 156) |
| N84 | GGGGATCCGAATTCGaagAAAACGGCAATTaaaGTAGTGCAGAGACAGTCGaagAAGCTTGC |
| | GGCC (SEQ ID NO: 157) |
| N132 | GGCCGCAAGCTTcttCGACTGTCTCTGCACTACtttAATTGCCGTTTTcttCGAATTCGGATCCCC |
| | (SEQ ID NO: 158) |
| N85 | GGGGATCCGAATTCGcgccgtcggTCGCAAATGGCTATTCGCGTGACACAACGTAAGCTTGCG |
| | GCC (SEQ ID NO: 158) |
| N133 | GGCCGCAAGCTTACGTTGTGTCACGCGAATAGCCATTTGCGAccgacggcgCGAATTCGGATC |
| | CCC(SEQ ID NO: 160) |
| N86 | GGGGATCCGAATTCGccgccaccaTCGCAAATGGCTATTCGCGTGACACAACGTAAGCTTGCG |
| | GCC (SEQ ID NO: 161) |
| N134 | GGCCGCAAGCTTACGTTGTGTCACGCGAATAGCCATTTGCGAtggtggcggCGAATTCGGATC |
| | CCC(SEQ ID NO: 162) |
| N87 | GGGGATCCGAATTCGTCGCAAATGGCTATTCGCGTGACACAACGTcgccgtcggAAGCTTGCG |
| | GCC (SEQ ID NO: 163) |
| N135 | GGCCGCAAGCTTccgacggcgACGTTGTGTCACGCGAATAGCCATTTGCGACGAATTCGGATC |
| | CCC(SEQ ID NO: 164) |
| N88 | GGGGATCCGAATTCGTCGCAAATGGCTATTCGCGTGACACAACGTccgccaccaAAGCTTGCG |
| | GCC (SEQ ID NO: 165) |
| N136 | GGCCGCAAGCTTtggtggcggACGTTGTGTCACGCGAATAGCCATTTGCGACGAATTCGGATC |
| | CCC (SEQ ID NO: 166) |
| N89 | GGGGATCCGAATTCGcgccgtcggTCGCAAATGGCTATTCGCGTGACACAACGTcgccgtcggAAG |
| | CTTGCGGCC (SEQ ID NO: 167) |
| N90 | GGGGATCCGAATTCGccgccaccaTCGCAAATGGCTATTCGCGTGACACAACGTccgccaccaAA |
| | GCTTGCGGCC (SEQ ID NO: 168) |
| N137 | GGCCGCAAGCTTccgacggcgACGTTGTGTCACGCGAATAGCCATTTGCGAccgacggcgCGAAT |
| | TCGGATCCCC ( SEQ ID NO: 169) |
| N91 | GGGGATCCGAATTCGcgccgtcggTCGCAAATGGCTATTCGCGTGACACAACGTccgccaccaAA |
| | GCTTGCGGCC (SEQ ID NO: 170) |
| N139 | GGCCGCAAGCTTtggtggcggACGTTGTGTCACGCGAATAGCCATTTGCGAccgacggcgCGAATT |
| | CGGATCCCC (SEQ ID NO: 171) |
| N92 | GGGGATCCGAATTCGTCGCAAATGaagaaaCGCGTGACACAACGTAAGCTTGCGGCC |
| | (SEQ ID NO: 172) |
| N140 | GGCCGCAAGCTTACGTTGTGTCACGCGtttcttCATTTGCGACGAATTCGGATCCCC |
| | (SEQ ID NO: 173) |
| N93 | GGGGATCCGAATTCGTCGCAAaaaGCTaaaCGCGTGACACAACGTAAGCTTGCGGCC |
| | (SEQ ID NO: 174) |
| N141 | GGCCGCAAGCTTACGTTGTGTCACGCGtttAGCtttTTGCGACGAATTCGGATCCCC |
| | (SEQ ID NO: 175) |
| N94 | GGGGATCCGAATTCGTCGCAAATGGCTATTCGCaagGTGaaaCGTAAGCTTGCGGCC |
| | (SEQ ID NO: 176) |
| N142 | GGCCGCAAGCTTACGtttCACcttGCGAATAGCCATTTGCGACGAATTCGGATCCCC |
| | (SEQ ID NO: 177) |
| N95 | GGGGATCCGAATTCGaaaTCGCAAATGGCTATTCGCGTGACACAACGTaaaAAGCTTGCGGC |
| | C (SEQ ID NO: 178) |
| N96 | GGGGATCCGAATTCGaaaTCGCAAATGGCTaaaCGCGTGACACAACGTaaaAAGCTTGCGGC |
| | C (SEQ ID NO: 179) |
| N143 | GGCCGCAAGCTTtttACGTTGTGTCACGCGAATAGCCATTTGCGAtttCGAATTCGGATCCCC |
| | (SEQ ID NO: 180) |
| D1013 | aa GAATTC G cgt cgc cgc cgt cgc AAAACGGCAA ( SEQ ID NO: 181) |
| D1014 | aata AAGCTT cgg tgg cgg CGACTGTCT ( SEQ ID NO: 182) |
| D1016 | aata AAGCTT agg cgg cgg tgg cgg CGACTGTCT (SEQ ID NO: 183) |
| D1018 | aa GAATTC G cgc cgt cgc AAAACGGCAA ( SEQ ID NO: 184) |
| D1019 | aa GAATTC G ggc ggt ggc AAAACGGCAATTGTAGTGCAG ( SEQ ID NO: 185) |
| D1021 | aatg AAGCTT gcc gcc acc CGACTGTCTCTGCACTACA ( SEQ ID NO: 186) |
| D1063 | aa GAATTC G cgt cgc AAAACGGCAATTGT ( SEQ ID NO: 188) |
| D1064 | aatg AAGCTT tgg cgg CGACTGTCTCT (SEQ ID NO: 189) |
| D1066 | aa GAATTC G cgc AAAACGGCAATTGTAGTG ( SEQ ID NO: 191) |
| D1067 | aatg AAGCTT cgg CGACTGTCTCTGC ( SEQ ID NO: 192) |

The inventors discovered that several peptide sequences from Table 5 (SEQ ID NO: 11, 12, 29 and 35-91) which were cloned into T7select-415 bacteriophages produced effective inhibitors of curli assembly (FIG. 3A). The levels of inhibition observed with engineered bacteriophages expressing curli-inhibiting peptides were greater than with unmodified control T7select-415 bacteriophage (FIG. 3A). Further, the inventors identified three classes of phages which could be grouped together based on their inhibitory effectiveness against in vitro CsgA fiber formation, as shown in Table 7 (specific peptide sequences), Table 8 (numerical representation) and FIG. 3A (graphical representation).

The most effective engineered bacteriophages expressing modified CsgA peptides were the ones which are categorized as Class CsgAIII as shown in Table 7 (e.g., SEQ ID NO: 52 and 53). Next most effective modified CsgA peptides were those categorized as Class CsgAIIb and next most effective was those categorized as Class CsgA IIa. CsgAIII group of peptides are SEQ ID NO: 52 and 53 are more effective at inhibiting curli amyloid formation than which are more the CsgAIIb class of peptides (SEQ ID NOs: 35, 36, 39-41, 45, 49-51), which are more effective at inhibiting curli amyloid formation than the CsgAIIa class of peptide (SEQ ID NO: 11 and 12) which are more effective at inhibiting curli amyloid formation than the CsgAI class of peptides (SEQ ID NOs: 42, 44, 46, 57 and 58).
The most effective engineered bacteriophages expressing modified CsgB peptides were the ones which are categorized as Class III CsgB as shown in Table 7. Next most effective modified CsgB peptides were those categorized as Class CsgBIIb and next most effective was those categorized as Class Csg BIIa. The CsgBIII group (SEQ ID NOs: 61-65) are more effective at inhibiting curli amyloid formation than the CsgBIIb peptide group (SEQ ID NOs: 59, 60, 69, 75, 81, 93 and 94) which are more effective at inhibiting curli amyloid formation than the CsgB Class IIa group (SEQ ID NO: 29) which are more effective at inhibiting curli amyloid formation than the CsgB Class I peptide group (SEQ ID NOs: 66-68 and 70-72).

TABLE 7
Sequences modified from CsgA or CsgB peptides that were cloned into T7select-415
bacteriophage between EcoRI and HindIII restriction sites, and categorized according
to their effectiveness of inhibiting curli amyloid formation. The most
effective at inhibiting the curli assembly are in the following order
(of most effective to least effective) Class III > Class IIb > Class Ha > Class I.
Csg A peptides

| phage | SEQUENCE CLONED | SEQ ID NO: | Class | CsgA |
|---|---|---|---|---|
| 66-6 | PPPSALALQTDARPPP | SEQ ID NO: 52 | CsgA III | csgA |
| 67-3 | PPPSALALQTDARRRR | SEQ ID NO: 53 | CsgA III | csgA |
| 49-5 | RRRSELNIYQYGG | SEQ ID NO: 35 | CsgA IIb | csgA |
| 50-3 | PPPSELNIYQYGG | SEQ ID NO: 36 | CsgA IIb | csgA |
| 53-5 | PPPSELNIYQYGGRRR | SEQ ID NO: 39 | CsgA IIb | csgA |
| 54-4 | SELNIYQYGGRRR | SEQ ID NO: 40 | CsgA IIb | csgA |
| 55-2 | SELNIYQYGGPPP | SEQ ID NO: 41 | CsgA IIb | csgA |
| 59-3 | KELNIYQYGK | SEQ ID NO: 45 | CsgA IIb | csgA |
| 63-3 | SALALQTDARRRR | SEQ ID NO: 49 | CsgA IIb | csgA |
| 64-3 | SALALQTDARPPP | SEQ ID NO: 50 | CsgA IIb | csgA |
| 65-12 or 61-3 | RRRSALALQTDARRRR | SEQ ID NO: 51 | CsgA Ilb | csgA |
| 18-1 | SALALQTDAR | SEQ ID NO: 12 | CsgA IIa | csgA |
| 17-3 | SELNIYQYGG | SEQ ID NO: 11 | CsgA IIa | csgA |
| 57-1 | SEKNKYQYGG | SEQ ID NO: 42 | CsgA I | csgA |
| 58-2 | SELNKKQYGG | SEQ ID NO: 44 | CsgA I | csgA |
| 60-1 | KELNKYQYGK | SEQ ID NO: 46 | CsgA I | csgA |
| 71-4 | SALALKKDAR | SEQ ID NO: 57 | CsgA I | csgA |
| 72-5 | SALKLKTDAR | SEQ ID NO: 58 | CsgA I | csgA |

CsgB peptides

| phage | SEQUENCE CLONED | SEQ ID NO: | Class | Csg B |
|---|---|---|---|---|
| 75-5 | RRRKTAIVVQRQSPPP | SEQ ID NO: 61 | CsgB III | csgB |
| 76-1 or 76-2 | RRRKTAIVVQRQSRRR | SEQ ID NO: 62 | CsgB III | csgB |
| 78-4 or 78-6 | KTAIVVQRQSRRR | SEQ ID NO: 64 | CsgB III | csgB |
| 77-7 | PPPKTAIVVQRQSPPP | SEQ ID NO: 63 | CsgB III | csgB |
| 79-1 | KTAIVVQRQSPPP | SEQ ID NO: 65 | CsgB III | csgB |
| 73-9 | RRRKTAIVVQRQS | SEQ ID NO: 59 | CsgB IIb | csgB |
| 74-7 | PPPKTAIVVQRQS | SEQ ID NO: 60 | CsgB IIb | csgB |
| 83-4 | KKTAIVVQRQSK | SEQ ID NO: 69 | CsgB IIb | csgB |
| 95-7 | KSQMAIRVTQRK | SEQ ID NO: 81 | CsgB IIb | csgB |
| 85-8 | PPPSQMAIRVTQRPPP | SEQ ID NO: 75 | CsgB IIb | csgB |
| 87-1 | SQMAIRRVTQRPPP | SEQ ID NO: 193 | CsgB IIb | csgB |
| 88-1 | SQMAIRVTQRRRR | SEQ ID NO: 194 | CsgB IIb | csgB |
| 27-3 | KTAIVVQRQS | SEQ ID NO: 29 | CsgB IIa | csgB |
| 80-3 | KTKIKVQRQS | SEQ ID NO: 66 | CsgB I | csgB |
| 81-1 | KTAKVKQRQS | SEQ ID NO: 67 | CsgB I | csgB |
| 82-1 | KTAKKVQRQS | SEQ ID NO: 68 | CsgB I | csgB |
| 84-3 | KKTAIKVQRQSK | SEQ ID NO: 70 | CsgB I | csgB |
| 86-2 | RRRSQMAIRVTQR | SEQ ID NO: 72 | CsgB I | csgB |
| 89-3 and 91-1 | PPPSQMAIRVTQRPPP | SEQ ID NO: 75 | CsgB I | csgB |
| 92-1 | SQMKKRVTQR | SEQ ID NO: 78 | CsgB I | csgB |
| 93-8 | SQKAKRVTQR | SEQ ID NO: 79 | CsgB I | csgB |
| 94-4 | SQMAIRKTKR | SEQ ID NO: 80 | CsgB I | csgB |

TABLE 8
ThT fluorescence of curli assembly in the presence of engineering phage expressing
curli-inhibiting peptides at various phage concentrations. The engineered phages of
Class I, IIa, IIb and III are shown.

| | 10¹ | | 10³ | 10⁴ | 10⁵ | 10⁶ | |
|---|---|---|---|---|---|---|---|
| | PFU/ | 10² PFU/ | PFU/ | PFU/ | PFU/ | PFU/ | Phage |
| Phage Name | mL | mL | mL | mL | mL | mL | Class |
| T7-wt | 0.99 | 0.97 | 0.96 | 0.95 | 0.95 | 0.95 | Class I |
| T7-CsgA₄₃₋₅₂(I47K-Q49K) | 0.98 | 0.95 | 0.98 | 0.94 | 0.95 | 0.87 | Class I |
| T7-CsgA₄₃₋₅₂(I47K-Y48K) | 0.98 | 1 | 0.97 | 0.95 | 0.97 | 0.92 | Class I |
| T7-CsgA₄₃₋₅₂(S43K-I47K-G52K) | 1 | 0.98 | 0.97 | 0.98 | 0.96 | 0.98 | Class I |
| T7-CsgA₅₅₋₆₄(Q60K-T61K) | 1 | 0.95 | 0.93 | 0.94 | 0.98 | 0.92 | Class I |
| T7-CsgA₅₅₋₆₄(A58K-Q60K) | 0.96 | 0.96 | 0.94 | 0.98 | 0.93 | 0.95 | Class I |
| T7-CsgB₁₃₃₋₁₄₂(A135K-V137K) | 0.96 | 0.95 | 0.96 | 0.93 | 0.93 | 0.92 | Class I |
| T7-CsgB₁₃₃₋₁₄₂(I136K-V138K) | 0.98 | 0.96 | 0.97 | 0.94 | 0.94 | 0.94 | Class I |
| T7-CsgB₁₃₃₋₁₄₂(I136K-V137K) | 0.96 | 0.96 | 0.95 | 0.94 | 0.94 | 0.93 | Class I |
| T7-K-CsgB₁₃₃₋₁₄₂(V137K)-K | 0.96 | 0.97 | 0.96 | 0.95 | 0.95 | 0.95 | Class I |
| T7-RRR-CsgB₁₄₂₋₁₅₁ | 0.96 | 0.98 | 0.96 | 0.96 | 0.95 | 0.95 | Class I |
| T7-PPP-CsgB₁₄₂₋₁₅₁-PPP | 0.98 | 0.97 | 0.96 | 0.96 | 0.95 | 0.95 | Class I |
| T7-PPP-CsgB₁₄₂₋₁₅₁-RRR | 0.98 | 0.97 | 0.96 | 0.97 | 0.95 | 0.95 | Class I |
| T7-CsgB₁₄₂₋₁₅₁(A145K-I146K) | 0.98 | 0.98 | 0.98 | 0.95 | 0.94 | 0.92 | Class I |
| T7-CsgB₁₄₂₋₁₅₁(M144K-I146K) | 0.99 | 0.98 | 0.96 | 0.95 | 0.92 | 0.92 | Class I |
| T7-CsgB₁₄₂₋₁₅₁(V148K-Q150K) | 0.99 | 0.97 | 0.94 | 0.95 | 0.95 | 0.94 | Class I |
| T7-CsgA₄₃₋₅₂ | 1 | 0.98 | 0.98 | 0.89 | 0.69 | 0.61 | Class IIa |
| T7-CsgA₅₅₋₆₄ | 1 | 0.96 | 0.99 | 0.83 | 0.55 | 0.46 | Class IIa |
| T7-CsgB₁₃₃₋₁₄₂ | 1.02 | 1 | 1 | 0.65 | 0.48 | 0.41 | Class IIa |
| T7-RRR-CsgA₄₃₋₅₂ | 0.96 | 0.91 | 0.88 | 0.73 | 0.69 | 0.53 | Class IIb |
| T7-PPP-CsgA₄₃₋₅₂ | 0.98 | 0.89 | 0.79 | 0.68 | 0.58 | 0.47 | Class IIb |
| T7-PPP-CsgA₄₃₋₅₂-RRR | 0.96 | 0.91 | 0.85 | 0.81 | 0.73 | 0.65 | Class IIb |
| T7-CsgA₄₃₋₅₂-RRR | 0.97 | 0.9 | 0.76 | 0.68 | 0.55 | 0.51 | Class IIb |
| T7-CsgA₄₃₋₅₂-PPP | 0.99 | 0.81 | 0.69 | 0.55 | 0.42 | 0.37 | Class IIb |
| T7-CsgA₄₃₋₅₂(S43K-G52K) | 0.99 | 0.83 | 0.72 | 0.53 | 0.39 | 0.32 | Class IIb |
| T7-RRR-CsgA₅₅₋₆₄ | 0.99 | 0.81 | 0.69 | 0.58 | 0.51 | 0.43 | Class IIb |
| T7-CsgA₅₅₋₆₄-RRR | 0.98 | 0.85 | 0.76 | 0.6 | 0.53 | 0.38 | Class IIb |
| T7-CsgA₅₅₋₆₄-PPP | 0.98 | 0.85 | 0.68 | 0.55 | 0.48 | 0.36 | Class IIb |
| T7-RRR-CsgA₅₅₋₆₄-RRR | 0.99 | 0.87 | 0.8 | 0.72 | 0.65 | 0.59 | Class IIb |
| T7-RRR-CsgB₁₃₃₋₁₄₂ | 0.99 | 0.91 | 0.83 | 0.77 | 0.68 | 0.63 | Class IIb |
| T7-PPP-CsgB₁₃₃₋₁₄₂ | 0.99 | 0.85 | 0.73 | 0.66 | 0.61 | 0.49 | Class IIb |
| T7-K-CsgB₁₃₃₋₁₄₂-K | 0.98 | 0.86 | 0.71 | 0.63 | 0.54 | 0.43 | Class IIb |
| T7-PPP-CsgB₁₄₂₋₁₅₁ | 0.98 | 0.9 | 0.83 | 0.77 | 0.66 | 0.58 | Class IIb |
| T7-CsgB₁₄₂₋₁₅₁-PPP | 0.99 | 0.86 | 0.81 | 0.71 | 0.62 | 0.56 | Class IIb |
| T7-CsgB₁₄₂₋₁₅₁-RRR | 0.98 | 0.83 | 0.72 | 0.66 | 0.57 | 0.49 | Class IIb |
| T7-K-CsgB₁₄₂₋₁₅₁-K | 0.99 | 0.89 | 0.78 | 0.69 | 0.59 | 0.49 | Class IIb |
| T7-PPP-CsgA₅₅₋₆₄-PPP | 0.97 | 0.69 | 0.5 | 0.33 | 0.27 | 0.19 | Class III |
| T7-PPP-CsgA₅₅₋₆₄-RRR | 0.98 | 0.65 | 0.5 | 0.32 | 0.22 | 0.15 | Class III |
| T7-CsgB₁₃₃₋₁₄₂-RRR | 0.99 | 0.57 | 0.4 | 0.22 | 0.17 | 0.09 | Class III |
| T7-CsgB₁₃₃₋₁₄₂-PPP Clone #1 | 0.99 | 0.6 | 0.42 | 0.28 | 0.2 | 0.14 | Class III |
| T7-CsgB₁₃₃₋₁₄₂-PPP Clone #2 | 0.99 | 0.43 | 0.29 | 0.16 | 0.11 | 0.09 | Class III |
| T7-RRR-CsgB₁₃₃₋₁₄₂-RRR | 0.99 | 0.5 | 0.39 | 0.25 | 0.13 | 0.09 | Class III |
| T7-PPP-CsgB₁₃₃₋₁₄₂-PPP Clone | 0.98 | 0.39 | 0.27 | 0.13 | 0.1 | 0.06 | Class III |
| #4 | | | | | | | |
| T7-PPP-CsgB₁₃₃₋₁₄₂-PPP Clone | 0.99 | 0.31 | 0.16 | 0.09 | 0.03 | 0 | Class III |
| #6 | | | | | | | |
| T7-RRR-CsgB₁₃₃₋₁₄₂-PPP | 0.99 | 0.34 | 0.2 | 0.12 | 0.05 | 0.01 | Class III |

The inventors discovered that class I peptide-expressing phages were ineffective at blocking curli fiber formation and were mostly composed of sequences based on CsgB₁₄₂₋₁₅₁ as well as lysine substitution mutants of CsgA₄₃₋₅₂, CsgA₅₅₋₆₄, CsgB₁₃₃₋₁₄₂, and CsgB₁₄₂₋₁₅₁ sequences (FIG. 3B and Table 8). Class I also included wild-type T7 (T7-wt). Class IIb phages were moderately effective at blocking fiber assembly (ranging from 35% to 68% inhibition) (FIG. 3B and Table 8). Although Class IIb phages were about as effective as phages displaying wild-type CsgA and CsgB sequences (Class IIa), they did not stimulate fiber assembly at low concentrations as Class IIa phages did. Class IIb phages contained CsgA₄₃₋₅₂, CsgA₅₅₋₆₄, CsgB₁₃₃₋₁₄₂, and CsgB₁₄₂₋₁₅₁ sequences flanked by lysine, arginine, and/or proline residues. Class III phages strongly reduced amyloid fiber formation (ranging from 91% to >99% inhibition) and contained sequences such as PPP-CsgA₅₅₋₆₄-PPP, PPP-CsgA₅₅₋₆₄-RRR, and CsgB₁₃₃₋₁₄₂ flanked by PPP and/or RRR (FIG. 3B and Table 8). The most effective peptides within Class III were modified sequences based off of CsgB₁₃₃₋₁₄₂, which is consistent with the identification of a major nucleating sequence within CsgB₁₃₄₋₁₄₀ using peptide arrays and using the computational program “AmyloidMutant” as disclosed herein.

The anti-amyloid peptide engineered bacteriophages can also be used in specific products and services. For example, the anti-amyloid peptide engineered bacteriophages can be formulated in liquid or tablet forms for medical, food processing, agricultural, sanitization and defense purposes. The engineered phages can also be packaged in tablets sold for sterilization of water storage tanks or in liquid forms used for various sterilization purposes ranging from open wounds, sites of surgery in patients or even the clinical operating rooms. Such anti-amyloid peptide engineered bacteriophages can be used in the farming industry to replace current antibiotics and prevent the rise of drug resistant bacteria in food stocks. Similarly the anti-amyloid peptide engineered bacteriophages can be used to prevent bacterial contamination by food borne pathogens of crops or food products and would be used in food processing plants for meat, dairies and fresh vegetables.

TABLE 9
Examples of bacteriophages which can be engineered to be an anti-amyloid peptide
bacteriophage, inhibitor-engineered bacteriophage, or a repressor-engineered bacteriophage
or a susceptibility-engineered bacteriophage as disclosed herein.
Table 9: Examples of bacteriophages which can be engineered to be an anti-amyloid peptide
bacteriophage, inhibitor-engineered bacteriophage, or a repressor-engineered bacteriophage
or a susceptibility-engineered bacteriophage as disclosed herein.

| organism | accession | length | proteins | RNAs | genes |
|---|---|---|---|---|---|
| Acholeplasma phage L2 | NC_001447 | 11965 nt | 14 | 0 | 14 |
| Acholeplasma phage MV-L1 | NC_001341 | 4491 nt | 4 | 0 | 4 |
| Acidianus bottle-shaped virus | NC_009452 | 23814 nt | 57 | 0 | 57 |
| Acidianus filamentous virus 1 | NC_005830 | 20869 nt | 40 | 0 | 40 |
| Acidianus filamentous virus 2 | NC_009884 | 31787 nt | 52 | 1 | 53 |
| Acidianus filamentous virus 3 | NC_010155 | 40449 nt | 68 | 0 | 68 |
| Acidianus filamentous virus 6 | NC_010152 | 39577 nt | 66 | 0 | 66 |
| Acidianus filamentous virus 7 | NC_010153 | 36895 nt | 57 | 0 | 57 |
| Acidianus filamentous virus 8 | NC_010154 | 38179 nt | 61 | 0 | 61 |
| Acidianus filamentous virus 9 | NC_010537 | 41172 nt | 73 | 0 | 73 |
| Acidianus rod-shaped virus 1 | NC_009965 | 24655 nt | 41 | 0 | 41 |
| Acidianus two-tailed virus | NC_007409 | 62730 nt | 72 | 0 | 72 |
| Acinetobacter phage AP205 | NC_002700 | 4268 nt | 4 | 0 | 4 |
| Actinomyces phage Av-1 | NC_009643 | 17171 nt | 22 | 1 | 23 |
| Actinoplanes phage phiAsp2 | NC_005885 | 58638 nt | 76 | 0 | 76 |
| Acyrthosiphon pisum secondary | NC_000935 | 36524 nt | 54 | 0 | 54 |
| endosymbiont phage 1 | | | | | |
| Aeromonas phage 25 | NC_008208 | 161475 nt | 242 | 13 | 242 |
| Aeromonas phage 31 | NC_007022 | 172963 nt | 247 | 15 | 262 |
| Aeromonas phage 44RR2.8t | NC_005135 | 173591 nt | 252 | 17 | 269 |
| Aeromonas phage Aeh1 | NC_005260 | 233234 nt | 352 | 23 | 375 |
| Aeromonas phage phiO18P | NC_009542 | 33985 nt | 45 | 0 | 45 |
| Archaeal BJ1 virus | NC_008695 | 42271 nt | 70 | 1 | 71 |
| Azospirillum phage Cd | NC_010355 | 62337 nt | 95 | 0 | 95 |
| Bacillus phage 0305phi8-36 | NC_009760 | 218948 nt | 246 | 0 | 246 |
| Bacillus phage AP50 | NC_011523 | 14398 nt | 31 | 0 | 31 |
| Bacillus phage B103 | NC_004165 | 18630 nt | 17 | 0 | 17 |
| Bacillus phage BCJA1c | NC_006557 | 41092 nt | 58 | 0 | 58 |
| Bacillus phage Bam35c | NC_005258 | 14935 nt | 32 | 0 | 32 |
| Bacillus phage Cherry | NC_007457 | 36615 nt | 51 | 0 | 51 |
| Bacillus phage Fah | NC_007814 | 37974 nt | 50 | 0 | 50 |
| Bacillus phage GA-1 | NC_002649 | 21129 nt | 35 | 1 | 52 |
| Bacillus phage GIL16c | NC_006945 | 14844 nt | 31 | 0 | 31 |
| Bacillus phage Gamma | NC_007458 | 37253 nt | 53 | 0 | 53 |
| Bacillus phage IEBH | NC_011167 | 53104 nt | 86 | 0 | 86 |
| Bacillus phage SPBc2 | NC_001884 | 134416 nt | 185 | 0 | 185 |
| Bacillus phage SPO1 | NC_011421 | 132562 nt | 204 | 5 | 209 |
| Bacillus phage SPP1 | NC_004166 | 44010 nt | 101 | 0 | 101 |
| Bacillus phage TP21-L | NC_011645 | 37456 nt | 56 | 0 | 56 |
| Bacillus phage WBeta | NC_007734 | 40867 nt | 53 | 0 | 53 |
| Bacillus phage phBC6A51 | NC_004820 | 61395 nt | 75 | 0 | 75 |
| Bacillus phage phBC6A52 | NC_004821 | 38472 nt | 49 | 0 | 49 |
| Bacillus phage phi105 | NC_004167 | 39325 nt | 51 | 0 | 51 |
| Bacillus phage phi29 | NC_011048 | 19282 nt | 27 | 0 | 27 |
| Bacillus virus 1 | NC_009737 | 35055 nt | 54 | 0 | 54 |
| Bacterio phage APSE-2 | NC_011551 | 39867 nt | 41 | 1 | 42 |
| Bacteroides phage B40-8 | NC_011222 | 44929 nt | 46 | 0 | 46 |
| Bdellovibrio phage phiMH2K | NC_002643 | 4594 nt | 11 | 0 | 11 |
| Bordetella phage BIP-1 | NC_005809 | 42638 nt | 48 | 0 | 48 |
| Bordetella phage BMP-1 | NC_005808 | 42663 nt | 47 | 0 | 47 |
| Bordetella phage BPP-1 | NC_005357 | 42493 nt | 49 | 0 | 49 |
| Burkholderia ambifaria ge BcepF1 | NC_009015 | 72415 nt | 127 | 0 | 127 |
| Burkholderia phage Bcep1 | NC_005263 | 48177 nt | 71 | 0 | 71 |
| Burkholderia phage Bcep176 | NC_007497 | 44856 nt | 81 | 0 | 81 |
| Burkholderia phage Bcep22 | NC_005262 | 63879 nt | 81 | 1 | 82 |
| Burkholderia phage Bcep43 | NC_005342 | 48024 nt | 65 | 0 | 65 |
| Burkholderia phage Bcep781 | NC_004333 | 48247 nt | 66 | 0 | 66 |
| Burkholderia phage BcepB1A | NC_005886 | 47399 nt | 73 | 0 | 73 |
| Burkholderia phage BcepC6B | NC_005887 | 42415 nt | 46 | 0 | 46 |
| Burkholderia phage BcepGomr | NC_009447 | 52414 nt | 75 | 0 | 75 |
| Burkholderia phage BcepMu | NC_005882 | 36748 nt | 53 | 0 | 53 |
| Burkholderia phage BcepNY3 | NC_009604 | 47382 nt | 70 | 1 | 70 |
| Burkholderia phage BcepNazgul | NC_005091 | 57455 nt | 73 | 0 | 73 |
| Burkholderia phage KS10 | NC_011216 | 37635 nt | 49 | 0 | 49 |
| Burkholderia phage phi1026b | NC_005284 | 54865 nt | 83 | 0 | 83 |
| Burkholderia phage phi52237 | NC_007145 | 37639 nt | 47 | 0 | 47 |
| Burkholderia phage phi644-2 | NC_009235 | 48674 nt | 71 | 0 | 71 |
| Burkholderia phage phiE12-2 | NC_009236 | 36690 nt | 50 | 0 | 50 |
| Burkholderia phage phiE125 | NC_003309 | 53373 nt | 71 | 0 | 71 |
| Burkholderia phage phiE202 | NC_009234 | 35741 nt | 48 | 0 | 48 |
| Burkholderia phage phiE255 | NC_009237 | 37446 nt | 55 | 0 | 55 |
| Chlamydia phage 3 | NC_008355 | 4554 nt | 8 | 0 | 8 |
| Chlamydia phage 4 | NC_007461 | 4530 nt | 8 | 0 | 8 |
| Chlamydia phage CPAR39 | NC_002180 | 4532 nt | 7 | 0 | 7 |
| Chlamydia phage Chp1 | NC_001741 | 4877 nt | 12 | 0 | 12 |
| Chlamydia phage Chp2 | NC_002194 | 4563 nt | 8 | 0 | 7 |
| Chlamydia phage phiCPG1 | NC_001998 | 4529 nt | 9 | 0 | 9 |
| Clostridium phage 39-O | NC_011318 | 38753 nt | 62 | 0 | 62 |
| Clostridium phage c-st | NC_007581 | 185683 nt | 198 | 0 | 198 |
| Clostridium phage phi CD119 | NC_007917 | 53325 nt | 79 | 0 | 79 |
| Clostridium phage phi3626 | NC_003524 | 33507 nt | 50 | 0 | 50 |
| Clostridium phage phiC2 | NC_009231 | 56538 nt | 82 | 0 | 82 |
| Clostridium phage phiCD27 | NC_011398 | 50930 nt | 75 | 0 | 75 |
| Clostridium phage phiSM101 | NC_008265 | 38092 nt | 53 | 1 | 54 |
| Corynebacterium phage BFK20 | NC_009799 | 42969 nt | 54 | 0 | 54 |
| Corynebacterium phage P1201 | NC_009816 | 70579 nt | 97 | 4 | 101 |
| Enterobacteria phage 13a | NC_011045 | 38841 nt | 55 | 0 | 55 |
| Enterobacteria phage 933W | NC_000924 | 61670 nt | 80 | 4 | 84 |
| Enterobacteria phage BA14 | NC_011040 | 39816 nt | 52 | 0 | 52 |
| Enterobacteria phage BP-4795 | NC_004813 | 57930 nt | 85 | 0 | 85 |
| Enterobacteria phage BZ13 | NC_001426 | 3466 nt | 4 | 0 | 4 |
| Enterobacteria phage EPS7 | NC_010583 | 111382 nt | 170 | 0 | 171 |
| Enterobacteria phage ES18 | NC_006949 | 46900 nt | 79 | 0 | 79 |
| Enterobacteria phage EcoDS1 | NC_011042 | 39252 nt | 53 | 0 | 53 |
| Enterobacteria phage FI sensu lato | NC_004301 | 4276 nt | 4 | 0 | 4 |
| Enterobacteria phage Felix 01 | NC_005282 | 86155 nt | 131 | 22 | 153 |
| Enterobacteria phage Fels-2 | NC_010463 | 33693 nt | 47 | 0 | 48 |
| Enterobacteria phage G4 sensu lato | NC_001420 | 5577 nt | 11 | 0 | 13 |
| Enterobacteria phage HK022 | NC_002166 | 40751 nt | 57 | 0 | 57 |
| Enterobacteria phage HK620 | NC_002730 | 38297 nt | 58 | 0 | 58 |
| Enterobacteria phage HK97 | NC_002167 | 39732 nt | 61 | 0 | 62 |
| Enterobacteria phage I2-2 | NC_001332 | 6744 nt | 9 | 0 | 9 |
| Enterobacteria phage ID18 sensu lato | NC_007856 | 5486 nt | 11 | 0 | 11 |
| Enterobacteria phage ID2 Moscow/ID/2001 | NC_007817 | 5486 nt | 11 | 0 | 11 |
| Enterobacteria phage If1 | NC_001954 | 8454 nt | 10 | 0 | 10 |
| Enterobacteria phage Ike | NC_002014 | 6883 nt | 10 | 0 | 10 |
| Enterobacteria phage JK06 | NC_007291 | 46072 nt | 82 | 0 | 82 |
| Enterobacteria phage JS98 | NC_010105 | 170523 nt | 266 | 3 | 269 |
| Enterobacteria phage K1-5 | NC_008152 | 44385 nt | 52 | 0 | 52 |
| Enterobacteria phage K1E | NC_007637 | 45251 nt | 62 | 0 | 62 |
| Enterobacteria ge K1F | NC_007456 | 39704 nt | 43 | 0 | 41 |
| Enterobacteria phage M13 | NC_003287 | 6407 nt | 10 | 0 | 10 |
| Enterobacteria phage MS2 | NC_001417 | 3569 nt | 4 | 0 | 4 |
| Enterobacteria phage Min27 | NC_010237 | 63395 nt | 83 | 3 | 86 |
| Enterobacteria phage Mu | NC_000929 | 36717 nt | 55 | 0 | 55 |
| Enterobacteria phage N15 | NC_001901 | 46375 nt | 60 | 0 | 60 |
| Enterobacteria phage N4 | NC_008720 | 70153 nt | 72 | 0 | 72 |
| Enterobacteria phage P1 | NC_005856 | 94800 nt | 110 | 4 | 117 |
| Enterobacteria phage P2 | NC_001895 | 33593 nt | 43 | 0 | 43 |
| Enterobacteria phage P22 | NC_002371 | 41724 nt | 72 | 2 | 74 |
| Enterobacteria phage P4 | NC_001609 | 11624 nt | 14 | 5 | 19 |
| Enterobacteria phage PRD1 | NC_001421 | 14927 nt | 31 | 0 | 31 |
| Enterobacteria phage Phi1 | NC_009821 | 164270 nt | 276 | 0 | 276 |
| Enterobacteria phage PsP3 | NC_005340 | 30636 nt | 42 | 0 | 42 |
| Enterobacteria phage Qbeta | NC_001890 | 4215 nt | 4 | 0 | 4 |
| Enterobacteria phage RB32 | NC_008515 | 165890 nt | 270 | 8 | 270 |
| Enterobacteria phage RB43 | NC_007023 | 180500 nt | 292 | 1 | 292 |
| Enterobacteria phage RB49 | NC_005066 | 164018 nt | 279 | 0 | 279 |
| Enterobacteria phage RB69 | NC_004928 | 167560 nt | 273 | 2 | 275 |
| Enterobacteria phage RTP | NC_007603 | 46219 nt | 75 | 0 | 75 |
| Enterobacteria phage SP6 | NC_004831 | 43769 nt | 52 | 0 | 52 |
| Enterobacteria phage ST104 | NC_005841 | 41391 nt | 63 | 0 | 63 |
| Enterobacteria phage ST64T | NC_004348 | 40679 nt | 65 | 0 | 65 |
| Enterobacteria phage Sf6 | NC_005344 | 39043 nt | 66 | 2 | 70 |
| Enterobacteria phage SfV | NC_003444 | 37074 nt | 53 | 0 | 53 |
| Enterobacteria phage T1 | NC_005833 | 48836 nt | 78 | 0 | 78 |
| Enterobacteria phage T3 | NC_003298 | 38208 nt | 55 | 0 | 56 |
| Enterobacteria phage T4 | NC_000866 | 168903 nt | 278 | 10 | 288 |
| Enterobacteria phage T5 | NC_005859 | 121750 nt | 162 | 33 | 195 |
| Enterobacteria phage T7 | NC_001604 | 39937 nt | 60 | 0 | 60 |
| Enterobacteria phage TLS | NC_009540 | 49902 nt | 87 | 0 | 87 |
| Enterobacteria phage VT2-Sakai | NC_000902 | 60942 nt | 83 | 3 | 86 |
| Enterobacteria phage WA13 sensu lato | NC_007821 | 6068 nt | 10 | 0 | 10 |
| Enterobacteria phage YYZ-2008 | NC_011356 | 54896 nt | 75 | 0 | 75 |
| Enterobacteria phage alpha3 | NC_001330 | 6087 nt | 10 | 0 | 10 |
| Enterobacteria phage epsilon15 | NC_004775 | 39671 nt | 51 | 0 | 51 |
| Enterobacteria phage lambda | NC_001416 | 48502 nt | 73 | 0 | 92 |
| Enterobacteria phage phiEco32 | NC_010324 | 77554 nt | 128 | 1 | 128 |
| Enterobacteria phage phiEcoM-GJ1 | NC_010106 | 52975 nt | 75 | 1 | 76 |
| Enterobacteria phage phiP27 | NC_003356 | 42575 nt | 58 | 2 | 60 |
| Enterobacteria phage phiV10 | NC_007804 | 39104 nt | 55 | 0 | 55 |
| Enterobacteria phage phiX174 sensu lato | NC_001422 | 5386 nt | 11 | 0 | 11 |
| Enterococcus phage phiEF24C | NC_009904 | 142072 nt | 221 | 5 | 226 |
| Erwinia phage Era103 | NC_009014 | 45445 nt | 53 | 0 | 53 |
| Erwinia phage phiEa21-4 | NC_011811 | 84576 nt | 118 | 26 | 144 |
| Escherichia phage rv5 | NC_011041 | 137947 nt | 233 | 6 | 239 |
| Flavobacterium phage 11b | NC_006356 | 36012 nt | 65 | 0 | 65 |
| Geobacillus phage GBSV1 | NC_008376 | 34683 nt | 54 | 0 | 54 |
| Geobacillus virus E2 | NC_009552 | 40863 nt | 71 | 0 | 71 |
| Haemophilus phage Aaphi23 | NC_004827 | 43033 nt | 66 | 0 | 66 |
| Haemophilus phage HP1 | NC_001697 | 32355 nt | 42 | 0 | 42 |
| Haemophilus phage HP2 | NC_003315 | 31508 nt | 37 | 0 | 37 |
| Haloarcula phage SH1 | NC_007217 | 30889 nt | 56 | 0 | 56 |
| Halomonas phage phiHAP-1 | NC_010342 | 39245 nt | 46 | 0 | 46 |
| Halorubrum phage HF2 | NC_003345 | 77670 nt | 114 | 5 | 119 |
| Halovirus HF1 | NC_004927 | 75898 nt | 102 | 4 | 106 |
| His1 virus | NC_007914 | 14462 nt | 35 | 0 | 35 |
| His2 virus | NC_007918 | 16067 nt | 35 | 0 | 35 |
| Iodobacteriophage phiPLPE | NC_011142 | 47453 nt | 84 | 0 | 84 |
| Klebsiella phage K11 | NC_011043 | 41181 nt | 51 | 0 | 51 |
| Klebsiella phage phiKO2 | NC_005857 | 51601 nt | 64 | 0 | 63 |
| Kluyvera phage Kvp1 | NC_011534 | 39472 nt | 47 | 1 | 48 |
| Lactobacillus johnsonii prophage Lj771 | NC_010179 | 40881 nt | 56 | 0 | 56 |
| Lactobacillus phage A2 | NC_004112 | 43411 nt | 61 | 0 | 64 |
| Lactobacillus phage KC5a | NC_007924 | 38239 nt | 61 | 0 | 61 |
| Lactobacillus phage LL-H | NC_009554 | 34659 nt | 51 | 0 | 51 |
| Lactobacillus phage LP65 | NC_006565 | 131522 nt | 165 | 14 | 179 |
| Lactobacillus phage Lc-Nu | NC_007501 | 36466 nt | 51 | 0 | 51 |
| Lactobacillus phage Lrm1 | NC_011104 | 39989 nt | 54 | 0 | 54 |
| Lactobacillus phage Lv-1 | NC_011801 | 38934 nt | 47 | 0 | 47 |
| Lactobacillus phage phiAT3 | NC_005893 | 39166 nt | 55 | 0 | 55 |
| Lactobacillus phage phiJL-1 | NC_006936 | 36674 nt | 46 | 0 | 46 |
| Lactobacillus phage phiadh | NC_000896 | 43785 nt | 63 | 0 | 63 |
| Lactobacillus phage phig1e | NC_004305 | 42259 nt | 50 | 0 | 62 |
| Lactobacillus prophage Lj928 | NC_005354 | 38384 nt | 50 | 1 | 50 |
| Lactobacillus prophage Lj965 | NC_005355 | 40190 nt | 46 | 4 | 46 |
| Lactococcus phage 1706 | NC_010576 | 55597 nt | 76 | 0 | 76 |
| Lactococcus phage 712 | NC_008370 | 30510 nt | 55 | 0 | 55 |
| Lactococcus phage BK5-T | NC_002796 | 40003 nt | 63 | 0 | 63 |
| Lactococcus phage KSY1 | NC_009817 | 79232 nt | 130 | 3 | 131 |
| Lactococcus phage P008 | NC_008363 | 28538 nt | 58 | 0 | 58 |
| Lactococcus phage P335 sensu lato | NC_004746 | 36596 nt | 49 | 0 | 49 |
| Lactococcus phage Q54 | NC_008364 | 26537 nt | 47 | 0 | 47 |
| Lactococcus phage TP901-1 | NC_002747 | 37667 nt | 56 | 0 | 56 |
| Lactococcus phage Tuc2009 | NC_002703 | 38347 nt | 56 | 0 | 56 |
| Lactococcus phage asccphi28 | NC_010363 | 18762 nt | 28 | 0 | 27 |
| Lactococcus phage bIBB29 | NC_011046 | 29305 nt | 54 | 0 | 54 |
| Lactococcus phage bIL170 | NC_001909 | 31754 nt | 64 | 0 | 64 |
| Lactococcus phage bIL285 | NC_002666 | 35538 nt | 62 | 0 | 62 |
| Lactococcus phage bIL286 | NC_002667 | 41834 nt | 61 | 0 | 61 |
| Lactococcus phage bIL309 | NC_002668 | 36949 nt | 56 | 0 | 56 |
| Lactococcus phage bIL310 | NC_002669 | 14957 nt | 29 | 0 | 29 |
| Lactococcus phage bIL311 | NC_002670 | 14510 nt | 22 | 0 | 22 |
| Lactococcus phage bIL312 | NC_002671 | 15179 nt | 27 | 0 | 27 |
| Lactococcus phage bIL67 | NC_001629 | 22195 nt | 37 | 0 | 0 |
| Lactococcus phage c2 | NC_001706 | 22172 nt | 39 | 2 | 41 |
| Lactococcus phage jj50 | NC_008371 | 27453 nt | 49 | 0 | 49 |
| Lactococcus phage phiLC3 | NC_005822 | 32172 nt | 51 | 0 | 51 |
| Lactococcus phage r1t | NC_004302 | 33350 nt | 50 | 0 | 50 |
| Lactococcus phage sk1 | NC_001835 | 28451 nt | 56 | 0 | 56 |
| Lactococcus phage ul36 | NC_004066 | 36798 nt | 61 | 0 | 61 |
| Leuconostoc phage L5 | NC_009534 | 2435 nt | 0 | 0 | 0 |
| Listeria phage 2389 | NC_003291 | 37618 nt | 59 | 1 | 58 |
| Listeria phage A006 | NC_009815 | 38124 nt | 62 | 0 | 62 |
| Listeria phage A118 | NC_003216 | 40834 nt | 72 | 0 | 72 |
| Listeria phage A500 | NC_009810 | 38867 nt | 63 | 0 | 63 |
| Listeria phage A511 | NC_009811 | 137619 nt | 199 | 16 | 215 |
| Listeria phage B025 | NC_009812 | 42653 nt | 65 | 0 | 65 |
| Listeria phage B054 | NC_009813 | 48172 nt | 80 | 0 | 80 |
| Listeria phage P35 | NC_009814 | 35822 nt | 56 | 0 | 56 |
| Listeria phage P40 | NC_011308 | 35638 nt | 62 | 0 | 62 |
| Listonella phage phiHSIC | NC_006953 | 37966 nt | 47 | 0 | 47 |
| Mannheimia phage phiMHaA1 | NC_008201 | 34525 nt | 49 | 0 | 50 |
| Methanobacterium phage psiM2 | NC_001902 | 26111 nt | 32 | 0 | 32 |
| Methanothermobacter phage psiM100 | NC_002628 | 28798 nt | 35 | 0 | 35 |
| Microbacterium phage Min1 | NC_009603 | 46365 nt | 77 | 0 | 77 |
| Microcystis phage Ma-LMM01 | NC_008562 | 162109 nt | 184 | 2 | 186 |
| Morganella phage MmP1 | NC_011085 | 38233 nt | 47 | 0 | 47 |
| Mycobacterium phage 244 | NC_008194 | 74483 nt | 142 | 2 | 144 |
| Mycobacterium phage Adjutor | NC_010763 | 64511 nt | 86 | 0 | 86 |
| Mycobacterium phage BPs | NC_010762 | 41901 nt | 63 | 0 | 63 |
| Mycobacterium phage Barnyard | NC_004689 | 70797 nt | 109 | 0 | 109 |
| Mycobacterium phage Bethlehem | NC_009878 | 52250 nt | 87 | 0 | 87 |
| Mycobacterium phage Boomer | NC_011054 | 58037 nt | 105 | 0 | 105 |
| Mycobacterium phage Brujita | NC_011291 | 47057 nt | 74 | 0 | 74 |
| Mycobacterium phage Butterscotch | NC_011286 | 64562 nt | 86 | 0 | 86 |
| Mycobacterium phage Bxb1 | NC_002656 | 50550 nt | 86 | 0 | 86 |
| Mycobacterium phage Bxz1 | NC_004687 | 156102 nt | 225 | 28 | 253 |
| Mycobacterium phage Bxz2 | NC_004682 | 50913 nt | 86 | 3 | 89 |
| Mycobacterium phage Cali | NC_011271 | 155372 nt | 222 | 35 | 257 |
| Mycobacterium phage Catera | NC_008207 | 153766 nt | 218 | 34 | 253 |
| Mycobacterium phage Chah | NC_011284 | 68450 nt | 104 | 0 | 104 |
| Mycobacterium phage Che12 | NC_008203 | 52047 nt | 98 | 3 | 101 |
| Mycobacterium phage Che8 | NC_004680 | 59471 nt | 112 | 0 | 112 |
| Mycobacterium phage Che9c | NC_004683 | 57050 nt | 84 | 1 | 85 |
| Mycobacterium phage Che9d | NC_004686 | 56276 nt | 111 | 0 | 111 |
| Mycobacterium phage Cjw1 | NC_004681 | 75931 nt | 141 | 1 | 142 |
| Mycobacterium phage Cooper | NC_008195 | 70654 nt | 99 | 0 | 99 |
| Mycobacterium phage Corndog | NC_004685 | 69777 nt | 122 | 0 | 122 |
| Mycobacterium phage D29 | NC_001900 | 49136 nt | 79 | 5 | 84 |
| Mycobacterium phage DD5 | NC_011022 | 51621 nt | 87 | 0 | 87 |
| Mycobacterium phage Fruitloop | NC_011288 | 58471 nt | 102 | 0 | 102 |
| Mycobacterium phage Giles | NC_009993 | 54512 nt | 79 | 1 | 80 |
| Mycobacterium phage Gumball | NC_011290 | 64807 nt | 88 | 0 | 88 |
| Mycobacterium phage Halo | NC_008202 | 42289 nt | 65 | 0 | 65 |
| Mycobacterium phage Jasper | NC_011020 | 50968 nt | 94 | 0 | 94 |
| Mycobacterium phage KBG | NC_011019 | 53572 nt | 89 | 0 | 89 |
| Mycobacterium phage Konstantine | NC_011292 | 68952 nt | 95 | 0 | 95 |
| Mycobacterium phage Kostya | NC_011056 | 75811 nt | 143 | 2 | 145 |
| Mycobacterium phage L5 | NC_001335 | 52297 nt | 85 | 3 | 88 |
| Mycobacterium phage Llij | NC_008196 | 56852 nt | 100 | 0 | 100 |
| Mycobacterium phage Lockley | NC_011021 | 51478 nt | 90 | 0 | 90 |
| Mycobacterium phage Myrna | NC_011273 | 164602 nt | 229 | 41 | 270 |
| Mycobacterium phage Nigel | NC_011044 | 69904 nt | 94 | 1 | 95 |
| Mycobacterium phage Omega | NC_004688 | 110865 nt | 237 | 2 | 239 |
| Mycobacterium phage Orion | NC_008197 | 68427 nt | 100 | 0 | 100 |
| Mycobacterium phage PBI1 | NC_008198 | 64494 nt | 81 | 0 | 81 |
| Mycobacterium phage PG1 | NC_005259 | 68999 nt | 100 | 0 | 100 |
| Mycobacterium phage PLot | NC_008200 | 64787 nt | 89 | 0 | 89 |
| Mycobacterium phage PMC | NC_008205 | 56692 nt | 104 | 0 | 104 |
| Mycobacterium phage Pacc40 | NC_011287 | 58554 nt | 101 | 0 | 101 |
| Mycobacterium phage Phaedrus | NC_011057 | 68090 nt | 98 | 0 | 98 |
| Mycobacterium phage Pipefish | NC_008199 | 69059 nt | 102 | 0 | 102 |
| Mycobacterium phage Porky | NC_011055 | 76312 nt | 147 | 2 | 149 |
| Mycobacterium phage Predator | NC_011039 | 70110 nt | 92 | 0 | 92 |
| Mycobacterium phage Pukovnik | NC_011023 | 52892 nt | 88 | 1 | 89 |
| Mycobacterium phage Qyrzula | NC_008204 | 67188 nt | 81 | 0 | 81 |
| Mycobacterium phage Ramsey | NC_011289 | 58578 nt | 108 | 0 | 108 |
| Mycobacterium phage Rizal | NC_011272 | 153894 nt | 220 | 35 | 255 |
| Mycobacterium phage Rosebush | NC_004684 | 67480 nt | 90 | 0 | 90 |
| Mycobacterium phage ScottMcG | NC_011269 | 154017 nt | 221 | 36 | 257 |
| Mycobacterium phage Solon | NC_011267 | 49487 nt | 86 | 0 | 86 |
| Mycobacterium phage Spud | NC_011270 | 154906 nt | 222 | 35 | 257 |
| Mycobacterium phage TM4 | NC_003387 | 52797 nt | 89 | 0 | 89 |
| Mycobacterium phage Troll4 | NC_011285 | 64618 nt | 84 | 0 | 84 |
| Mycobacterium phage Tweety | NC_009820 | 58692 nt | 109 | 0 | 109 |
| Mycobacterium phage U2 | NC_009877 | 51277 nt | 81 | 0 | 81 |
| Mycobacterium phage Wildcat | NC_008206 | 78441 nt | 148 | 23 | 171 |
| Mycoplasma phage MAV1 | NC_001942 | 15644 nt | 15 | 0 | 15 |
| Mycoplasma phage P1 | NC_002515 | 11660 nt | 11 | 0 | 11 |
| Mycoplasma phage phiMFV1 | NC_005964 | 15141 nt | 15 | 0 | 17 |
| Myxococcus phage Mx8 | NC_003085 | 49534 nt | 86 | 0 | 85 |
| Natrialba phage PhiCh1 | NC_004084 | 58498 nt | 98 | 0 | 98 |
| Pasteurella phage F108 | NC_008193 | 30505 nt | 44 | 0 | 44 |
| Phage Gifsy-1 | NC_010392 | 48491 nt | 58 | 1 | 59 |
| Phage Gifsy-2 | NC_010393 | 45840 nt | 55 | 0 | 56 |
| Phage cdtI | NC_009514 | 47021 nt | 60 | 0 | 60 |
| Phage phiJL001 | NC_006938 | 63649 nt | 90 | 0 | 90 |
| Phormidium phage Pf-WMP3 | NC_009551 | 43249 nt | 41 | 0 | 41 |
| Phormidium phage Pf-WMP4 | NC_008367 | 40938 nt | 45 | 0 | 45 |
| Prochlorococcus phage P-SSM2 | NC_006883 | 252401 nt | 329 | 1 | 330 |
| Prochlorococcus phage P-SSM4 | NC_006884 | 178249 nt | 198 | 0 | 198 |
| Prochlorococcus phage P-SSP7 | NC_006882 | 44970 nt | 53 | 0 | 53 |
| Propionibacterium phage B5 | NC_003460 | 5804 nt | 10 | 0 | 10 |
| Propionibacterium phage PA6 | NC_009541 | 29739 nt | 48 | 0 | 48 |
| Pseudoalteromonas phage PM2 | NC_000867 | 10079 nt | 22 | 0 | 22 |
| Pseudomonas phage 119X | NC_007807 | 43365 nt | 53 | 0 | 53 |
| Pseudomonas phage 14-1 | NC_011703 | 66235 nt | 90 | 0 | 90 |
| Pseudomonas phage 201phi2-1 | NC_010821 | 316674 nt | 461 | 1 | 462 |
| Pseudomonas phage 73 | NC_007806 | 42999 nt | 52 | 0 | 52 |
| Pseudomonas phage B3 | NC_006548 | 38439 nt | 59 | 0 | 59 |
| Pseudomonas phage D3 | NC_002484 | 56425 nt | 95 | 4 | 99 |
| Pseudomonas phage D3112 | NC_005178 | 37611 nt | 55 | 0 | 55 |
| Pseudomonas phage DMS3 | NC_008717 | 36415 nt | 52 | 0 | 52 |
| Pseudomonas phage EL | NC_007623 | 211215 nt | 201 | 0 | 201 |
| Pseudomonas phage F10 | NC_007805 | 39199 nt | 63 | 0 | 63 |
| Pseudomonas phage F116 | NC_006552 | 65195 nt | 70 | 0 | 70 |
| Pseudomonas phage F8 | NC_007810 | 66015 nt | 91 | 0 | 91 |
| Pseudomonas phage LBL3 | NC_011165 | 64427 nt | 87 | 0 | 87 |
| Pseudomonas phage LKA1 | NC_009936 | 41593 nt | 56 | 0 | 56 |
| Pseudomonas phage LKD16 | NC_009935 | 43200 nt | 53 | 0 | 53 |
| Pseudomonas phage LMA2 | NC_011166 | 66530 nt | 93 | 0 | 93 |
| Pseudomonas phage LUZ19 | NC_010326 | 43548 nt | 54 | 0 | 54 |
| Pseudomonas phage LUZ24 | NC_010325 | 45625 nt | 68 | 0 | 68 |
| Pseudomonas phage M6 | NC_007809 | 59446 nt | 85 | 0 | 85 |
| Pseudomonas phage MP22 | NC_009818 | 36409 nt | 51 | 0 | 51 |
| Pseudomonas phage MP29 | NC_011613 | 36632 nt | 51 | 0 | 51 |
| Pseudomonas phage MP38 | NC_011611 | 36885 nt | 51 | 0 | 51 |
| Pseudomonas phage PA11 | NC_007808 | 49639 nt | 70 | 0 | 70 |
| Pseudomonas phage PAJU2 | NC_011373 | 46872 nt | 79 | 0 | 79 |
| Pseudomonas phage PB1 | NC_011810 | 65764 nt | 93 | 0 | 94 |
| Pseudomonas phage PP7 | NC_001628 | 3588 nt | 4 | 0 | 4 |
| Pseudomonas phage PRR1 | NC_008294 | 3573 nt | 4 | 0 | 4 |
| Pseudomonas phage PT2 | NC_011107 | 42961 nt | 54 | 0 | 54 |
| Pseudomonas phage PT5 | NC_011105 | 42954 nt | 52 | 0 | 52 |
| Pseudomonas phage PaP2 | NC_005884 | 43783 nt | 58 | 0 | 58 |
| Pseudomonas phage PaP3 | NC_004466 | 45503 nt | 71 | 4 | 75 |
| Pseudomonas phage Pf1 | NC_001331 | 7349 nt | 14 | 0 | 14 |
| Pseudomonas phage Pf3 | NC_001418 | 5833 nt | 9 | 0 | 9 |
| Pseudomonas phage SN | NC_011756 | 66390 nt | 92 | 0 | 92 |
| Pseudomonas phage YuA | NC_010116 | 58663 nt | 77 | 0 | 77 |
| Pseudomonas phage gh-1 | NC_004665 | 37359 nt | 42 | 0 | 42 |
| Pseudomonas phage phi12 | NC_004173 | 6751 nt | 6 | 0 | 6 |
| Pseudomonas phage phi12 | NC_004175 | 4100 nt | 5 | 0 | 5 |
| Pseudomonas phage phi12 | NC_004174 | 2322 nt | 4 | 0 | 4 |
| Pseudomonas phage phi13 | NC_004172 | 6458 nt | 4 | 0 | 4 |
| Pseudomonas phage phi13 | NC_004171 | 4213 nt | 5 | 0 | 5 |
| Pseudomonas phage phi13 | NC_004170 | 2981 nt | 4 | 0 | 4 |
| Pseudomonas phage phi6 | NC_003715 | 6374 nt | 4 | 0 | 4 |
| Pseudomonas phage phi6 | NC_003716 | 4063 nt | 4 | 0 | 4 |
| Pseudomonas phage phi6 | NC_003714 | 2948 nt | 5 | 0 | 5 |
| Pseudomonas phage phi8 | NC_003299 | 7051 nt | 7 | 0 | 7 |
| Pseudomonas phage phi8 | NC_003300 | 4741 nt | 6 | 0 | 6 |
| Pseudomonas phage phi8 | NC_003301 | 3192 nt | 6 | 0 | 6 |
| Pseudomonas phage phiCTX | NC_003278 | 35580 nt | 47 | 0 | 47 |
| Pseudomonas phage phiKMV | NC_005045 | 42519 nt | 49 | 0 | 49 |
| Pseudomonas phage phiKZ | NC_004629 | 280334 nt | 306 | 0 | 306 |
| Pyrobaculum spherical virus | NC_005872 | 28337 nt | 48 | 0 | 48 |
| Pyrococcus abyssi virus 1 | NC_009597 | 18098 nt | 25 | 0 | 25 |
| Ralstonia phage RSB1 | NC_011201 | 43079 nt | 47 | 0 | 47 |
| Ralstonia phage RSL1 | NC_010811 | 231256 nt | 345 | 2 | 346 |
| Ralstonia phage RSM1 | NC_008574 | 8999 nt | 15 | 0 | 15 |
| Ralstonia phage RSM3 | NC_011399 | 8929 nt | 14 | 0 | 14 |
| Ralstonia phage RSS1 | NC_008575 | 6662 nt | 12 | 0 | 12 |
| Ralstonia phage p12J | NC_005131 | 7118 nt | 9 | 0 | 9 |
| Ralstonia phage phiRSA1 | NC_009382 | 38760 nt | 51 | 0 | 51 |
| Rhizobium phage 16-3 | NC_011103 | 60195 nt | 110 | 0 | 109 |
| Rhodothermus phage RM378 | NC_004735 | 129908 nt | 146 | 0 | 146 |
| Roseobacter phage SIO1 | NC_002519 | 39898 nt | 34 | 0 | 34 |
| Salmonella phage E1 | NC_010495 | 45051 nt | 51 | 0 | 52 |
| Salmonella phage Fels-1 | NC_010391 | 42723 nt | 52 | 0 | 52 |
| Salmonella phage KS7 | NC_006940 | 40794 nt | 59 | 0 | 59 |
| Salmonella phage SE1 | NC_011802 | 41941 nt | 67 | 0 | 67 |
| Salmonella phage SETP3 | NC_009232 | 42572 nt | 53 | 0 | 53 |
| Salmonella phage ST64B | NC_004313 | 40149 nt | 56 | 0 | 56 |
| Salmonella phage phiSG-JL2 | NC_010807 | 38815 nt | 55 | 0 | 55 |
| Sinorhizobium phage PBC5 | NC_003324 | 57416 nt | 83 | 0 | 83 |
| Sodalis phage phiSG1 | NC_007902 | 52162 nt | 47 | 0 | 47 |
| Spiroplasma kunkelii virus SkV1_CR2-3x | NC_009987 | 7870 nt | 13 | 0 | 13 |
| Spiroplasma phage 1-C74 | NC_003793 | 7768 nt | 13 | 0 | 13 |
| Spiroplasma phage 1-R8A2B | NC_001365 | 8273 nt | 12 | 0 | 12 |
| Spiroplasma phage 4 | NC_003438 | 4421 nt | 9 | 0 | 9 |
| Spiroplasma phage SVTS2 | NC_001270 | 6825 nt | 13 | 0 | 13 |
| Sputnik virophage | NC_011132 | 18343 nt | 21 | 0 | 21 |
| Staphylococcus aureus phage P68 | NC_004679 | 18227 nt | 22 | 0 | 22 |
| Staphylococcus phage 11 | NC_004615 | 43604 nt | 53 | 0 | 53 |
| Staphylococcus phage 187 | NC_007047 | 39620 nt | 77 | 0 | 77 |
| Staphylococcus phage 2638A | NC_007051 | 41318 nt | 57 | 0 | 57 |
| Staphylococcus phage 29 | NC_007061 | 42802 nt | 67 | 0 | 67 |
| Staphylococcus phage 37 | NC_007055 | 43681 nt | 70 | 0 | 70 |
| Staphylococcus phage 3A | NC_007053 | 43095 nt | 67 | 0 | 67 |
| Staphylococcus phage 42E | NC_007052 | 45861 nt | 79 | 0 | 79 |
| Staphylococcus phage 44AHJD | NC_004678 | 16784 nt | 21 | 0 | 21 |
| Staphylococcus phage 47 | NC_007054 | 44777 nt | 65 | 0 | 65 |
| Staphylococcus phage 52A | NC_007062 | 41690 nt | 60 | 0 | 60 |
| Staphylococcus phage 53 | NC_007049 | 43883 nt | 74 | 0 | 74 |
| Staphylococcus phage 55 | NC_007060 | 41902 nt | 77 | 0 | 77 |
| Staphylococcus phage 66 | NC_007046 | 18199 nt | 27 | 0 | 27 |
| Staphylococcus phage 69 | NC_007048 | 42732 nt | 69 | 0 | 69 |
| Staphylococcus phage 71 | NC_007059 | 43114 nt | 67 | 0 | 67 |
| Staphylococcus phage 77 | NC_005356 | 41708 nt | 69 | 0 | 69 |
| Staphylococcus phage 80alpha | NC_009526 | 43864 nt | 73 | 0 | 73 |
| Staphylococcus phage 85 | NC_007050 | 44283 nt | 71 | 0 | 71 |
| Staphylococcus phage 88 | NC_007063 | 43231 nt | 66 | 0 | 66 |
| Staphylococcus phage 92 | NC_007064 | 42431 nt | 64 | 0 | 64 |
| Staphylococcus phage 96 | NC_007057 | 43576 nt | 74 | 0 | 74 |
| Staphylococcus phage CNPH82 | NC_008722 | 43420 nt | 65 | 0 | 65 |
| Staphylococcus phage EW | NC_007056 | 45286 nt | 77 | 0 | 77 |
| Staphylococcus phage G1 | NC_007066 | 138715 nt | 214 | 0 | 214 |
| Staphylococcus phage K | NC_005880 | 127395 nt | 115 | 0 | 115 |
| Staphylococcus phage PH15 | NC_008723 | 44041 nt | 68 | 0 | 68 |
| Staphylococcus phage PT1028 | NC_007045 | 15603 nt | 22 | 0 | 22 |
| Staphylococcus phage PVL | NC_002321 | 41401 nt | 62 | 0 | 62 |
| Staphylococcus phage ROSA | NC_007058 | 43155 nt | 74 | 0 | 74 |
| Staphylococcus phage SAP-2 | NC_009875 | 17938 nt | 20 | 0 | 20 |
| Staphylococcus phage Twort | NC_007021 | 130706 nt | 195 | 0 | 195 |
| Staphylococcus phage X2 | NC_007065 | 43440 nt | 77 | 0 | 77 |
| Staphylococcus phage phi 12 | NC_004616 | 44970 nt | 49 | 0 | 49 |
| Staphylococcus phage phi13 | NC_004617 | 42722 nt | 49 | 0 | 49 |
| Staphylococcus phage phi2958PVL | NC_011344 | 47342 nt | 60 | 0 | 59 |
| Staphylococcus phage phiETA | NC_003288 | 43081 nt | 66 | 0 | 66 |
| Staphylococcus phage phiETA2 | NC_008798 | 43265 nt | 69 | 0 | 69 |
| Staphylococcus phage phiETA3 | NC_008799 | 43282 nt | 68 | 0 | 68 |
| Staphylococcus phage phiMR11 | NC_010147 | 43011 nt | 67 | 0 | 67 |
| Staphylococcus phage phiMR25 | NC_010808 | 44342 nt | 70 | 0 | 70 |
| Staphylococcus phage phiN315 | NC_004740 | 44082 nt | 65 | 0 | 64 |
| Staphylococcus phage phiNM | NC_008583 | 43128 nt | 64 | 0 | 64 |
| Staphylococcus phage phiNM3 | NC_008617 | 44061 nt | 65 | 0 | 65 |
| Staphylococcus phage phiPVL108 | NC_008689 | 44857 nt | 59 | 0 | 59 |
| Staphylococcus phage phiSLT | NC_002661 | 42942 nt | 61 | 0 | 61 |
| Staphylococcus phage phiSauS-IPLA35 | NC_011612 | 45344 nt | 62 | 0 | 62 |
| Staphylococcus phage phiSauS-IPLA88 | NC_011614 | 42526 nt | 60 | 0 | 61 |
| Staphylococcus phage tp310-1 | NC_009761 | 41407 nt | 59 | 0 | 59 |
| Staphylococcus phage tp310-2 | NC_009762 | 45710 nt | 67 | 0 | 67 |
| Staphylococcus phage tp310-3 | NC_009763 | 41966 nt | 58 | 0 | 58 |
| Staphylococcus prophage phiPV83 | NC_002486 | 45636 nt | 65 | 0 | 65 |
| Stenotrophomonas phage S1 | NC_011589 | 40287 nt | 48 | 0 | 48 |
| Stenotrophomonas phage phiSMA9 | NC_007189 | 6907 nt | 7 | 0 | 7 |
| Streptococcus phage 2972 | NC_007019 | 34704 nt | 44 | 0 | 44 |
| Streptococcus phage 7201 | NC_002185 | 35466 nt | 46 | 0 | 46 |
| Streptococcus phage 858 | NC_010353 | 35543 nt | 46 | 0 | 46 |
| Streptococcus phage C1 | NC_004814 | 16687 nt | 20 | 0 | 20 |
| Streptococcus phage Cp-1 | NC_001825 | 19343 nt | 25 | 0 | 25 |
| Streptococcus phage DT1 | NC_002072 | 34815 nt | 45 | 0 | 45 |
| Streptococcus phage EJ-1 | NC_005294 | 42935 nt | 73 | 0 | 73 |
| Streptococcus phage MM1 | NC_003050 | 40248 nt | 53 | 0 | 53 |
| Streptococcus phage O1205 | NC_004303 | 43075 nt | 57 | 0 | 57 |
| Streptococcus phage P9 | NC_009819 | 40539 nt | 53 | 0 | 53 |
| Streptococcus phage PH15 | NC_010945 | 39136 nt | 60 | 0 | 60 |
| Streptococcus phage SM1 | NC_004996 | 34692 nt | 56 | 0 | 56 |
| Streptococcus phage SMP | NC_008721 | 36216 nt | 48 | 0 | 48 |
| Streptococcus phage Sfi11 | NC_002214 | 39807 nt | 53 | 0 | 53 |
| Streptococcus phage Sfi19 | NC_000871 | 37370 nt | 45 | 0 | 45 |
| Streptococcus phage Sfi21 | NC_000872 | 40739 nt | 50 | 0 | 50 |
| Streptococcus phage phi3396 | NC_009018 | 38528 nt | 64 | 0 | 64 |
| Streptococcus pyogenes phage 315.1 | NC_004584 | 39538 nt | 56 | 0 | 56 |
| Streptococcus pyogenes phage 315.2 | NC_004585 | 41072 nt | 60 | 1 | 61 |
| Streptococcus pyogenes phage 315.3 | NC_004586 | 34419 nt | 52 | 0 | 52 |
| Streptococcus pyogenes phage 315.4 | NC_004587 | 41796 nt | 64 | 0 | 64 |
| Streptococcus pyogenes phage 315.5 | NC_004588 | 38206 nt | 55 | 0 | 55 |
| Streptococcus pyogenes phage 315.6 | NC_004589 | 40014 nt | 51 | 0 | 51 |
| Streptomyces phage VWB | NC_005345 | 49220 nt | 61 | 0 | 61 |
| Streptomyces phage mu1/6 | NC_007967 | 38194 nt | 52 | 0 | 52 |
| Streptomyces phage phiBT1 | NC_004664 | 41831 nt | 55 | 1 | 56 |
| Streptomyces phage phiC31 | NC_001978 | 41491 nt | 53 | 1 | 54 |
| Stx1 converting phage | NC_004913 | 59866 nt | 167 | 0 | 166 |
| Stx2 converting phage I | NC_003525 | 61765 nt | 166 | 0 | 166 |
| Stx2 converting phage II | NC_004914 | 62706 nt | 170 | 0 | 169 |
| Stx2-converting phage 1717 | NC_011357 | 62147 nt | 77 | 0 | 81 |
| Stx2-converting phage 86 | NC_008464 | 60238 nt | 81 | 3 | 80 |
| Sulfolobus islandicus filamentous virus | NC_003214 | 40900 nt | 73 | 0 | 73 |
| Sulfolobus islandicus rod-shaped virus 1 | NC_004087 | 32308 nt | 45 | 0 | 45 |
| Sulfolobus islandicus rod-shaped virus 2 | NC_004086 | 35450 nt | 54 | 0 | 54 |
| Sulfolobus spindle-shaped virus 4 | NC_009986 | 15135 nt | 34 | 0 | 34 |
| Sulfolobus spindle-shaped virus 5 | NC_011217 | 15330 nt | 34 | 0 | 34 |
| Sulfolobus turreted icosahedral virus | NC_005892 | 17663 nt | 36 | 0 | 36 |
| Sulfolobus virus 1 | NC_001338 | 15465 nt | 32 | 0 | 33 |
| Sulfolobus virus 2 | NC_005265 | 14796 nt | 34 | 0 | 34 |
| Sulfolobus virus Kamchatka 1 | NC_005361 | 17385 nt | 31 | 0 | 31 |
| Sulfolobus virus Ragged Hills | NC_005360 | 16473 nt | 37 | 0 | 37 |
| Sulfolobus virus STSV1 | NC_006268 | 75294 nt | 74 | 0 | 74 |
| Synechococcus phage P60 | NC_003390 | 47872 nt | 80 | 0 | 80 |
| Synechococcus phage S-PM2 | NC_006820 | 196280 nt | 236 | 1 | 238 |
| Synechococcus phage Syn5 | NC_009531 | 46214 nt | 61 | 0 | 61 |
| Synechococcus phage syn9 | NC_008296 | 177300 nt | 226 | 6 | 232 |
| Temperate phage phiNIH1.1 | NC_003157 | 41796 nt | 55 | 0 | 55 |
| Thalassomonas phage BA3 | NC_009990 | 37313 nt | 47 | 0 | 47 |
| Thermoproteus tenax spherical virus 1 | NC_006556 | 20933 nt | 38 | 0 | 38 |
| Thermus phage IN93 | NC_004462 | 19603 nt | 40 | 0 | 32 |
| Thermus phage P23-45 | NC_009803 | 84201 nt | 117 | 0 | 117 |
| Thermus phage P74-26 | NC_009804 | 83319 nt | 116 | 0 | 116 |
| Thermus phage phiYS40 | NC_008584 | 152372 nt | 170 | 3 | 170 |
| Vibrio phage K139 | NC_003313 | 33106 nt | 44 | 0 | 44 |
| Vibrio phage KSF-1phi | NC_006294 | 7107 nt | 12 | 0 | 12 |
| Vibrio phage KVP40 | NC_005083 | 244834 nt | 381 | 29 | 415 |
| Vibrio phage VGJphi | NC_004736 | 7542 nt | 13 | 0 | 13 |
| Vibrio phage VHML | NC_004456 | 43198 nt | 57 | 0 | 57 |
| Vibrio phage VP2 | NC_005879 | 39853 nt | 47 | 0 | 47 |
| Vibrio phage VP5 | NC_005891 | 39786 nt | 48 | 0 | 48 |
| Vibrio phage VP882 | NC_009016 | 38197 nt | 71 | 0 | 71 |
| Vibrio phage VSK | NC_003327 | 6882 nt | 14 | 0 | 14 |
| Vibrio phage Vf12 | NC_005949 | 7965 nt | 7 | 0 | 7 |
| Vibrio phage Vf33 | NC_005948 | 7965 nt | 7 | 0 | 7 |
| Vibrio phage VfO3K6 | NC_002362 | 8784 nt | 10 | 0 | 10 |
| Vibrio phage VfO4K68 | NC_002363 | 6891 nt | 8 | 0 | 8 |
| Vibrio phage fs1 | NC_004306 | 6340 nt | 15 | 0 | 15 |
| Vibrio phage fs2 | NC_001956 | 8651 nt | 9 | 0 | 9 |
| Vibrio phage kappa | NC_010275 | 33134 nt | 45 | 0 | 45 |
| Vibrio phage VP4 | NC_007149 | 39503 nt | 31 | 0 | 31 |
| Vibrio phage VpV262 | NC_003907 | 46012 nt | 67 | 0 | 67 |
| Xanthomonas phage Cf1c | NC_001396 | 7308 nt | 9 | 0 | 9 |
| Xanthomonas phage OP1 | NC_007709 | 43785 nt | 59 | 0 | 59 |
| Xanthomonas phage OP2 | NC_007710 | 46643 nt | 62 | 0 | 62 |
| Xanthomonas phage Xop411 | NC_009543 | 44520 nt | 58 | 0 | 58 |
| Xanthomonas phage Xp10 | NC_004902 | 44373 nt | 60 | 0 | 60 |
| Xanthomonas phage Xp15 | NC_007024 | 55770 nt | 84 | 0 | 84 |
| Yersinia pestis phage phiA1122 | NC_004777 | 37555 nt | 50 | 0 | 50 |
| Yersinia phage Berlin | NC_008694 | 38564 nt | 45 | 0 | 45 |
| Yersinia phage L-413C | NC_004745 | 30728 nt | 40 | 0 | 40 |
| Yersinia phage PY54 | NC_005069 | 46339 nt | 67 | 0 | 66 |
| Yersinia phage Yepe2 | NC_011038 | 38677 nt | 46 | 0 | 46 |
| Yersinia phage phiYeO3-12 | NC_001271 | 39600 nt | 59 | 0 | 59 |

TABLE 10
Examples of promoters which can be operatively linked to the nucleic acid
in the engineered bacteriophages.
Table 10: Examples of promoters which can be operatively linked to the nucleic
acid in the engineered bacteriophages.

| Name | Description | Length |
|---|---|---|
| BBa_I0500 | Inducible pBad/araC promoter | 1210 |
| BBa_I13453 | Pbad promoter | 130 |
| BBa_I712004 | CMV promoter | 654 |
| BBa_I712074 | T7 promoter (strong promoter from T7 bacteriophage) | 46 |
| BBa_I714889 | OR21 of PR and PRM | 101 |
| BBa_I714924 | RecA_DlexO_DLacO1 | 862 |
| BBa_I714927 | RecA_S_WTlexO_DLacO | 862 |
| BBa_I714929 | RecA_S_WTlexO_DLacO3 | 862 |
| BBa_I714930 | RecA_D_consenLexO_lacO1 | 862 |
| BBa_I714933 | WT_sulA_Single_LexO_double_LacO1 | 884 |
| BBa_I714935 | WT_sulA_Single_LexO_double_LacO2 | 884 |
| BBa_I714936 | WT_sulA_Single_LexO_double_LacO3 | 884 |
| BBa_I714937 | sluA_double_lexO_LacO1 | 884 |
| BBa_I714938 | sluA_double_lexO_LacO2 | 884 |
| BBa_I714939 | sluA_double_lexO_LacO3 | 884 |
| BBa_I715038 | pLac-RBS-T7 RNA Polymerase | 2878 |
| BBa_I716014 | yfbE solo trial 2 | 302 |
| BBa_I716102 | pir (Induces the R6K Origin) | 918 |
| BBa_I719005 | T7 Promoter | 23 |
| BBa_I732205 | NOT Gate Promoter Family Member (D001O55) | 124 |
| BBa_J13002 | TetR repressed POPS/RIPS generator | 74 |
| BBa_J13023 | 3OC6HSL + LuxR dependent POPS/RIPS generator | 117 |
| BBa_J23100 | constitutive promoter family member | 35 |
| BBa_J23101 | constitutive promoter family member | 35 |
| BBa_J23102 | constitutive promoter family member | 35 |
| BBa_J23103 | constitutive promoter family member | 35 |
| BBa_J23104 | constitutive promoter family member | 35 |
| BBa_J23105 | constitutive promoter family member | 35 |
| BBa_J23106 | constitutive promoter family member | 35 |
| BBa_J23107 | constitutive promoter family member | 35 |
| BBa_J23108 | constitutive promoter family member | 35 |
| BBa_J23109 | constitutive promoter family member | 35 |
| BBa_J23110 | constitutive promoter family member | 35 |
| BBa_J23111 | constitutive promoter family member | 35 |
| BBa_J23112 | constitutive promoter family member | 35 |
| BBa_J23113 | constitutive promoter family member | 35 |
| BBa_J23114 | constitutive promoter family member | 35 |
| BBa_J23115 | constitutive promoter family member | 35 |
| BBa_J23116 | constitutive promoter family member | 35 |
| BBa_J23117 | constitutive promoter family member | 35 |
| BBa_J23118 | constitutive promoter family member | 35 |
| BBa_J44002 | pBAD reverse | 130 |
| BBa_J52010 | NFkappaB-dependent promoter | 814 |
| BBa_J52034 | CMV promoter | 654 |
| BBa_J61043 | [fdhF2] Promoter | 269 |
| BBa_J63005 | yeast ADH1 promoter | 1445 |
| BBa_J63006 | yeast GAL1 promoter | 549 |
| BBa_K082017 | general recombine system | 89 |
| BBa_K091110 | LacI Promoter | 56 |
| BBa_K091111 | LacIQ promoter | 56 |
| BBa_K094120 | pLacI/ara-1 | 103 |
| BBa_K100000 | Natural Xylose Regulated Bi-Directional Operator | 303 |
| BBa_K100001 | Edited Xylose Regulated Bi-Directional Operator 1 | 303 |
| BBa_K100002 | Edited Xylose Regulated Bi-Directional Operator 2 | 303 |
| BBa_K118011 | PcstA (glucose-repressible promoter) | 131 |
| BBa_K135000 | pCpxR (CpxR responsive promoter) | 55 |
| BBa_K137029 | constitutive promoter with (TA)10 between-10 and -35 elements | 39 |
| BBa_K137030 | constitutive promoter with (TA)9 between-10 and -35 elements | 37 |
| BBa_K137046 | 150 bp inverted tetR promoter | 150 |
| BBa_K137047 | 250 bp inverted tetR promoter | 250 |
| BBa_K137048 | 350 bp inverted tetR promoter | 350 |
| BBa_K137049 | 450 bp inverted tetR promoter | 450 |
| BBa_K137050 | 650 bp inverted tetR promoter | 650 |
| BBa_K137051 | 850 bp inverted tetR promoter | 850 |
| BBa_R0010 | promoter (lacI regulated) | 200 |
| BBa_R0011 | Promoter (lacI regulated, lambda pL hybrid) | 55 |
| BBa_R0053 | Promoter (p22 cII regulated) | 54 |
| BBa_I1010 | cI(1) fused to tetR promoter | 834 |
| BBa_I1051 | Lux cassette right promoter | 68 |
| BBa_I12006 | Modified lamdba Prm promoter (repressed by 434 cI) | 82 |
| BBa_I12036 | Modified lamdba Prm promoter (cooperative repression by 434 cI) | 91 |
| BBa_I12040 | Modified lambda P(RM) promoter: -10 region from P(L) and cooperatively | 91 |
| | repressed by 434 cI | |
| BBa_I13005 | Promoter R0011 w/ YFP (-LVA) TT | 920 |
| BBa_I13006 | Promoter R0040 w/ YFP (-LVA) TT | 920 |
| BBa_I14015 | P(Las) TetO | 170 |
| BBa_I14016 | P(Las) CIO | 168 |
| BBa_I14017 | P(Rhl) | 51 |
| BBa_I14018 | P(Bla) | 35 |
| BBa_I14033 | P(Cat) | 38 |
| BBa_I14034 | P(Kat) | 45 |
| BBa_I714890 | OR321 of PR and PRM | 121 |
| BBa_I714925 | RecA_DlexO_DLacO2 | 862 |
| BBa_I714926 | RecA_DlexO_DLacO3 | 862 |
| BBa_I714928 | RecA_S_WTlexO_DLacO2 | 862 |
| BBa_I714931 | RecA_D_consenLexO_lacO2 | 862 |
| BBa_I718018 | dapAp promoter | 81 |
| BBa_I720001 | AraBp->rpoN | 1632 |
| BBa_I720002 | glnKp->lacI | 1284 |
| BBa_I720003 | NifHp->cI (lambda) | 975 |
| BBa_I720005 | NifA lacI RFP | 3255 |
| BBa_I720006 | GFP glnG cI | 2913 |
| BBa_I720007 | araBp->rpoN (leucine landing pad) | 51 |
| BBa_I720008 | Ara landing pad (pBBLP 6) | 20 |
| BBa_I720009 | Ara landing pad (pBBLP 7) | 23 |
| BBa_I720010 | Ara landing pad (pBBLP 8) | 20 |
| BBa_I721001 | Lead Promoter | 94 |
| BBa_I723020 | Pu | 320 |
| BBa_I728456 | MerRT: Mercury-Inducible Promoter + RBS (MerR + part of MerT) | 635 |
| BBa_I741018 | Right facing promoter (for xylF) controlled by xylR and CRP-cAMP | 221 |
| BBa_I742124 | Reverse complement Lac promoter | 203 |
| BBa_I746104 | P2 promoter in agr operon from S. aureus | 96 |
| BBa_I746360 | PF promoter from P2 phage | 91 |
| BBa_I746361 | PO promoter from P2 phage | 92 |
| BBa_I746362 | PP promoter from P2 phage | 92 |
| BBa_I746364 | Psid promoter from P4 phage | 93 |
| BBa_I746365 | PLL promoter from P4 phage | 92 |
| BBa_I748001 | Putative Cyanide Nitrilase Promoter | 271 |
| BBa_I752000 | Riboswitch(theophylline) | 56 |
| BBa_I761011 | CinR, CinL and glucose controlled promotor | 295 |
| BBa_I761014 | cinr + cinl (RBS) with double terminator | 1661 |
| BBa_I764001 | Ethanol regulated promoter AOX1 | 867 |
| BBa_I765000 | Fe promoter | 1044 |
| BBa_I765001 | UV promoter | 76 |
| BBa_I765007 | Fe and UV promoters | 1128 |
| BBa_J13210 | pOmpR dependent POPS producer | 245 |
| BBa_J22106 | rec A (SOS) Promoter | 192 |
| BBa_J23119 | constitutive promoter family member | 35 |
| BBa_J24669 | Tri-Stable Toggle (Arabinose induced component) | 3100 |
| BBa_J3902 | PrFe (PI + PII rus operon) | 272 |
| BBa_J58100 | AND-type promoter synergistically activated by cI and CRP | 106 |
| BBa_J61051 | [Psal1] | 1268 |
| BBa_K085005 | (lacI)promoter->key3c->Terminator | 405 |
| BBa_K088007 | GlnRS promoter | 38 |
| BBa_K089004 | phaC Promoter (−663 from ATG) | 663 |
| BBa_K089005 | −35 to Tc start site of phaC | 49 |
| BBa_K089006 | −663 to Tc start site of phaC | 361 |
| BBa_K090501 | Gram-Positive IPTG-Inducible Promoter | 107 |
| BBa_K090504 | Gram-Positive Strong Constitutive Promoter | 239 |
| BBa_K091100 | pLac_lux hybrid promoter | 74 |
| BBa_K091101 | pTet_Lac hybrid promoter | 83 |
| BBa_K091104 | pLac/Mnt Hybrid Promoter | 87 |
| BBa_K091105 | pTet/Mnt Hybrid Promoter | 98 |
| BBa_K091106 | LsrA/cI hybrid promoter | 141 |
| BBa_K091107 | pLux/cI Hybrid Promoter | 57 |
| BBa_K091114 | LsrAR Promoter | 248 |
| BBa_K091115 | LsrR Promoter | 100 |
| BBa_K091116 | LsrA Promoter | 126 |
| BBa_K091117 | pLas promoter | 126 |
| BBa_K091143 | pLas/cI Hybrid Promoter | 164 |
| BBa_K091146 | pLas/Lux Hybrid Promoter | 126 |
| BBa_K091184 | pLux/cI + RBS + LuxS + RBS + Mnt + TT + pLac/Mnt + RBS + LuxS + RBS + cI + TT | 2616 |
| BBa_K093000 | pRecA with LexA binding site | 48 |
| BBa_K101017 | MioC Promoter (DNAa-Repressed Promoter) | 319 |
| BBa_K101018 | MioC Promoter (regulating tetR) | 969 |
| BBa_K105020 | tetR - operator | 29 |
| BBa_K105021 | cI - operator | 27 |
| BBa_K105022 | lex A - operator | 31 |
| BBa_K105023 | lac I - operator | 25 |
| BBa_K105024 | Gal4 - operator | 27 |
| BBa_K105026 | Gal1 promoter | 549 |
| BBa_K105027 | cyc100 minimal promoter | 103 |
| BBa_K105028 | cyc70 minimal promoter | 103 |
| BBa_K105029 | cyc43 minimal promoter | 103 |
| BBa_K105030 | cyc28 minimal promoter | 103 |
| BBa_K105031 | cyc16 minimal promoter | 103 |
| BBa_K108014 | PR | 234 |
| BBa_K108016 | PP | 406 |
| BBa_K108025 | Pu | 200 |
| BBa_K109200 | AraC and TetR promoter (hybrid) | 132 |
| BBa_K110005 | Alpha-Cell Promoter MF(ALPHA)2 | 500 |
| BBa_K110006 | Alpha-Cell Promoter MF(ALPHA)1 | 501 |
| BBa_K110016 | A-Cell Promoter STE2 (backwards) | 500 |
| BBa_K112118 | rrnB P1 promoter | 503 |
| BBa_K112318 | {<bolA promoter>} in BBb format | 436 |
| BBa_K112319 | {<ftsQ promoter>} in BBb format | 434 |
| BBa_K112320 | {<ftsAZ promoter>} in BBb format | 773 |
| BBa_K112322 | {Pdps} in BBb format | 348 |
| BBa_K112323 | {H-NS!} in BBb format | 414 |
| BBa_K112400 | Promoter for grpE gene - Heat Shock and Ultrasound Sensitive | 98 |
| BBa_K112401 | Promoter for recA gene - SOS and Ultrasound Sensitive | 286 |
| BBa_K112402 | promoter for FabA gene - Membrane Damage and Ultrasound Senstitive | 256 |
| BBa_K112405 | Promoter for CadA and CadB genes | 370 |
| BBa_K112406 | cadC promoter | 2347 |
| BBa_K112407 | Promoter for ygeF psuedogene | 494 |
| BBa_K113009 | pBad/araC | 1210 |
| BBa_K116001 | nhaA promoter, that can be regulated by pH and nhaR protein. | 274 |
| BBa_K116401 | external phosphate sensing promoter | 506 |
| BBa_K116500 | OmpF promoter that is activated or repressesed by OmpR according to | 126 |
| | osmolarity. | |
| BBa_K116603 | pRE promoter from λ phage | 48 |
| BBa_K117002 | LsrA promoter (indirectly activated by AI-2) | 102 |
| BBa_K117004 | pLacI-GFP | 1086 |
| BBa_K117005 | pLacI-RBS | 220 |
| BBa_K119002 | RcnR operator (represses RcnA) | 83 |
| BBa_K122000 | pPGK1 | 1497 |
| BBa_K122002 | pADH1 (truncated) | 701 |
| BBa_K123002 | LacIQ ERE TetR | 742 |
| BBa_K123003 | ER | 1849 |
| BBa_K125110 | nir promoter + rbs (0.6) | 111 |
| BBa_K128006 | L. bulgaricus LacS Promoter | 197 |
| BBa_K133044 | TetR(RBS) | 35 |
| BBa_K136006 | flgA promoter followed by its natural RBS | 202 |
| BBa_K136008 | flhB promoter followed by its natural RBS | 203 |
| BBa_K136009 | fliL promoter followed by its natural RBS | 154 |
| BBa_K136010 | fliA promoter | 345 |
| BBa_K137031 | constitutive promoter with (C)10 between-10 and -35 elements | 62 |
| BBa_K137032 | constitutive promoter with (C)12 between-10 and -35 elements | 64 |
| BBa_K137125 | LacI-repressed promoter B4 | 103 |
| BBa_K145150 | Hybrid promoter: HSL-LuxR activated, P22 C2 repressed | 66 |
| BBa_K149001 | Prp22 promoter | 1006 |
| BBa_K165001 | pGAL1 + w/XhoI sites | 672 |
| BBa_K165011 | Zif268-HIV binding sites (3) | 46 |
| BBa_K165012 | Gli1 binding sites | 127 |
| BBa_K165013 | YY1 binding sites | 51 |
| BBa_K165016 | mCYC1 minimal yeast promoter | 245 |
| BBa_K165030 | mCYC promoter plus Zif268-HIV binding sites | 307 |
| BBa_K165031 | mCYC promoter plus LexA binding sites | 403 |
| BBa_K165032 | mCYC promoter plus Gli1 binding sites | 411 |
| BBa_K165033 | YY1 binding sites + mCYC promoter | 304 |
| BBa_K165034 | Zif268-HIV bs + LexA bs + mCYC promoter | 457 |
| BBa_K165035 | Gli1 bs + Zif268-HIV bs + mCYC promoter | 442 |
| BBa_K165036 | Gli1 bs + LexA bs + mCYC promoter | 538 |
| BBa_K165038 | Gli1 binding sites + ADH1 constitutive yeast promoter | 1580 |
| BBa_K165039 | Zif268-HIV binding sites + ADH1 yeast promoter | 1499 |
| BBa_K165040 | Gli1 binding sites + TEF constitutive yeast promoter | 538 |
| BBa_K165041 | Zif268-HIV binding sites + TEF constitutive yeast promoter | 457 |
| BBa_K165042 | Gli1 binding sites + MET25 inducible yeast promoter | 522 |
| BBa_K165043 | Zif268-HIV binding sites + MET25 constitutive yeast promoter | 441 |
| BBa_K165045 | pGAL1 + LexA bindingsites | 785 |
| BBa_K165048 | LexA op8 mCYC1 | 393 |
| BBa_R0050 | Promoter (HK022 cI regulated) | 55 |
| BBa_R0052 | Promoter (434 cI regulated) | 46 |
| BBa_R0061 | Promoter (HSL-mediated luxR repressor) | 30 |
| BBa_R0063 | Promoter (luxR & HSL regulated -- lux pL) | 151 |
| BBa_R0065 | Promoter (lambda cI and luxR regulated -- hybrid) | 97 |
| BBa_R0071 | Promoter (RhlR & C4-HSL regulated) | 53 |
| BBa_R0073 | Promoter (Mnt regulated) | 67 |
| BBa_R0074 | Promoter (PenI regulated) | 77 |
| BBa_R0075 | Promoter (TP901 cI regulated) | 117 |
| BBa_R0077 | Promoter (cinR and HSL regulated, RBS+) | 231 |
| BBa_R0078 | Promoter (cinR and HSL regulated) | 225 |
| BBa_R0081 | Inhibitor (AraC loop attachment with O2 site) | 183 |
| BBa_R0082 | Promoter (OmpR, positive) | 108 |
| BBa_R0083 | Promoter (OmpR, positive) | 78 |
| BBa_R0084 | Promoter (OmpR, positive) | 108 |
| BBa_R1050 | Promoter, Standard (HK022 cI regulated) | 56 |
| BBa_R1051 | Promoter, Standard (lambda cI regulated) | 49 |
| BBa_R1052 | Promoter, Standard (434 cI regulated) | 46 |
| BBa_R1053 | Promoter, Standard (p22 cII regulated) | 55 |
| BBa_R1062 | Promoter, Standard (luxR and HSL regulated -- lux pR) | 56 |
| BBa_R2000 | Promoter, Zif23 regulated, test: between | 45 |
| BBa_R2001 | Promoter, Zif23 regulated, test: after | 52 |
| BBa_R2002 | Promoter, Zif23 regulated, test: between and after | 52 |
| BBa_R2109 | Promoter with operator site for C2003 | 72 |
| BBa_R2114 | Promoter with operator site for C2003 | 72 |
| BBa_I10498 | Oct-4 promoter | 1417 |
| BBa_I12001 | Promoter (PRM+) | 96 |
| BBa_I12003 | Lambda Prm Promoter | 88 |
| BBa_I12005 | lambda Prm Inverted Antisense (No start codon) | 85 |
| BBa_I12008 | Barkai-Leibler design experiment part A (p22cII) | 1154 |
| BBa_I12010 | Modified lamdba Prm promoter (repressed by p22 cII) | 78 |
| BBa_I12014 | Repressor, 434 cI (RBS-LVA-) | 636 |
| BBa_I12021 | Inducible Lambda cI Repressor Generator (Controlled by IPTG and LacI) | 2370 |
| BBa_I12031 | Barkai-Leibler design experiment Part A (Lambda cI) wth cooperativity | 1159 |
| BBa_I12032 | Modified lamdba Prm promoter (repressed by p22 cI with cooperativity) | 106 |
| | RBS+ | |
| BBa_I12034 | Modified lamdba Prm promoter (repressed by 434 cI with cooperativity) | 102 |
| | RBS+ | |
| BBa_I12035 | Modified lamdba Prm promoter (repressed by p22 cI without cooperativity) | 106 |
| | RBS+ | |
| BBa_I12037 | Reporter 3 for Barkai-Leibler oscillator | 1291 |
| BBa_I12044 | Activator for BL oscillator with reporter protein, (cooperativity) | 2112 |
| BBa_I12045 | BL oscillator, cooperativity, reporter protein, kickstart | 4139 |
| BBa_I12046 | Activator for BL oscillator with reporter protein, (cooperativity and L-strain- | 2112 |
| | 10 region) | |
| BBa_I12047 | BL oscillator, cooperativity + replaced-10 region (Llac), reporter protein, | 4139 |
| | kickstart | |
| BBa_I12210 | plac Or2-62 (positive) | 70 |
| BBa_I12212 | TetR—TetR-4C heterodimer promoter (negative) | 61 |
| BBa_I12219 | Wild-type TetR(B) promoter (negative) | 71 |
| BBa_I13062 | LuxR QPI | 822 |
| BBa_I13267 | Intermediate part from assembly 317 | 1769 |
| BBa_I13406 | Pbad/AraC with extra REN sites | 1226 |
| BBa_I14021 | plTetO1.RBS.CinI | 810 |
| BBa_I20255 | Promoter-RBS | 57 |
| BBa_I20256 | Promoter-RBS | 56 |
| BBa_I20258 | Promoter-RBS | 56 |
| BBa_I714932 | RecA_D_consenLexO_lacO3 | 862 |
| BBa_I715003 | hybrid pLac with UV5 mutation | 55 |
| BBa_I715052 | Trp Leader Peptide and anti-terminator/terminator | 134 |
| BBa_I715053 | Trp Leader Peptide and anti-terminator/terminator with hixC insertion | 159 |
| BBa_I717002 | Pr from lambda switch | 177 |
| BBa_I723011 | pDntR (estimated promoter for DntR) | 26 |
| BBa_I723013 | pDntA (estimated promoter for DntA) | 33 |
| BBa_I723018 | Pr (promoter for XylR) | 410 |
| BBa_I731004 | FecA promoter | 90 |
| BBa_I732021 | Template for Building Primer Family Member | 159 |
| BBa_I732200 | NOT Gate Promoter Family Member (D001O1wt1) | 125 |
| BBa_I732201 | NOT Gate Promoter Family Member (D001O11) | 124 |
| BBa_I732202 | NOT Gate Promoter Family Member (D001O22) | 124 |
| BBa_I732203 | NOT Gate Promoter Family Member (D001O33) | 124 |
| BBa_I732204 | NOT Gate Promoter Family Member (D001O44) | 124 |
| BBa_I732206 | NOT Gate Promoter Family Member (D001O66) | 124 |
| BBa_I732207 | NOT Gate Promoter Family Member (D001O77) | 124 |
| BBa_I732270 | Promoter Family Member with Hybrid Operator (D001O12) | 124 |
| BBa_I732271 | Promoter Family Member with Hybrid Operator (D001O16) | 124 |
| BBa_I732272 | Promoter Family Member with Hybrid Operator (D001O17) | 124 |
| BBa_I732273 | Promoter Family Member with Hybrid Operator (D001O21) | 124 |
| BBa_I732274 | Promoter Family Member with Hybrid Operator (D001O24) | 124 |
| BBa_I732275 | Promoter Family Member with Hybrid Operator (D001O26) | 124 |
| BBa_I732276 | Promoter Family Member with Hybrid Operator (D001O27) | 124 |
| BBa_I732277 | Promoter Family Member with Hybrid Operator (D001O46) | 124 |
| BBa_I732278 | Promoter Family Member with Hybrid Operator (D001O47) | 124 |
| BBa_I732279 | Promoter Family Member with Hybrid Operator (D001O61) | 124 |
| BBa_I732301 | NAND Candidate (U073O26D001O16) | 120 |
| BBa_I732302 | NAND Candidate (U073O27D001O17) | 120 |
| BBa_I732303 | NAND Candidate (U073O22D001O46) | 120 |
| BBa_I732304 | NAND Candidate (U073O22D001O47) | 120 |
| BBa_I732305 | NAND Candidate (U073O22D059O46) | 178 |
| BBa_I732306 | NAND Candidate (U073O11D002O22) | 121 |
| BBa_I732351 | NOR Candidate (U037O11D002O22) | 85 |
| BBa_I732352 | NOR Candidate (U035O44D001O22) | 82 |
| BBa_I732400 | Promoter Family Member (U097NUL + D062NUL) | 165 |
| BBa_I732401 | Promoter Family Member (U097O11 + D062NUL) | 185 |
| BBa_I732402 | Promoter Family Member (U085O11 + D062NUL) | 173 |
| BBa_I732403 | Promoter Family Member (U073O11 + D062NUL) | 161 |
| BBa_I732404 | Promoter Family Member (U061O11 + D062NUL) | 149 |
| BBa_I732405 | Promoter Family Member (U049O11 + D062NUL) | 137 |
| BBa_I732406 | Promoter Family Member (U037O11 + D062NUL) | 125 |
| BBa_I732407 | Promoter Family Member (U097NUL + D002O22) | 125 |
| BBa_I732408 | Promoter Family Member (U097NUL + D014O22) | 137 |
| BBa_I732409 | Promoter Family Member (U097NUL + D026O22) | 149 |
| BBa_I732410 | Promoter Family Member (U097NUL + D038O22) | 161 |
| BBa_I732411 | Promoter Family Member (U097NUL + D050O22) | 173 |
| BBa_I732412 | Promoter Family Member (U097NUL + D062O22) | 185 |
| BBa_I732413 | Promoter Family Member (U097O11 + D002O22) | 145 |
| BBa_I732414 | Promoter Family Member (U097O11 + D014O22) | 157 |
| BBa_I732415 | Promoter Family Member (U097O11 + D026O22) | 169 |
| BBa_I732416 | Promoter Family Member (U097O11 + D038O22) | 181 |
| BBa_I732417 | Promoter Family Member (U097O11 + D050O22) | 193 |
| BBa_I732418 | Promoter Family Member (U097O11 + D062O22) | 205 |
| BBa_I732419 | Promoter Family Member (U085O11 + D002O22) | 133 |
| BBa_I732420 | Promoter Family Member (U085O11 + D014O22) | 145 |
| BBa_I732421 | Promoter Family Member (U085O11 + D026O22) | 157 |
| BBa_I732422 | Promoter Family Member (U085O11 + D038O22) | 169 |
| BBa_I732423 | Promoter Family Member (U085O11 + D050O22) | 181 |
| BBa_I732424 | Promoter Family Member (U085O11 + D062O22) | 193 |
| BBa_I732425 | Promoter Family Member (U073O11 + D002O22) | 121 |
| BBa_I732426 | Promoter Family Member (U073O11 + D014O22) | 133 |
| BBa_I732427 | Promoter Family Member (U073O11 + D026O22) | 145 |
| BBa_I732428 | Promoter Family Member (U073O11 + D038O22) | 157 |
| BBa_I732429 | Promoter Family Member (U073O11 + D050O22) | 169 |
| BBa_I732430 | Promoter Family Member (U073O11 + D062O22) | 181 |
| BBa_I732431 | Promoter Family Member (U061O11 + D002O22) | 109 |
| BBa_I732432 | Promoter Family Member (U061O11 + D014O22) | 121 |
| BBa_I732433 | Promoter Family Member (U061O11 + D026O22) | 133 |
| BBa_I732434 | Promoter Family Member (U061O11 + D038O22) | 145 |
| BBa_I732435 | Promoter Family Member (U061O11 + D050O22) | 157 |
| BBa_I732436 | Promoter Family Member (U061O11 + D062O22) | 169 |
| BBa_I732437 | Promoter Family Member (U049O11 + D002O22) | 97 |
| BBa_I732438 | Promoter Family Member (U049O11 + D014O22) | 109 |
| BBa_I732439 | Promoter Family Member (U049O11 + D026O22) | 121 |
| BBa_I732440 | Promoter Family Member (U049O11 + D038O22) | 133 |
| BBa_I732441 | Promoter Family Member (U049O11 + D050O22) | 145 |
| BBa_I732442 | Promoter Family Member (U049O11 + D062O22) | 157 |
| BBa_I732443 | Promoter Family Member (U037O11 + D002O22) | 85 |
| BBa_I732444 | Promoter Family Member (U037O11 + D014O22) | 97 |
| BBa_I732445 | Promoter Family Member (U037O11 + D026O22) | 109 |
| BBa_I732446 | Promoter Family Member (U037O11 + D038O22) | 121 |
| BBa_I732447 | Promoter Family Member (U037O11 + D050O22) | 133 |
| BBa_I732448 | Promoter Family Member (U037O11 + D062O22) | 145 |
| BBa_I732450 | Promoter Family Member (U073O26 + D062NUL) | 161 |
| BBa_I732451 | Promoter Family Member (U073O27 + D062NUL) | 161 |
| BBa_I732452 | Promoter Family Member (U073O26 + D062O61) | 181 |
| BBa_I735008 | ORE1X Oleate response element | 273 |
| BBa_I735009 | ORE2X oleate response element | 332 |
| BBa_I735010 | This promoter encoding for a thiolase involved in beta-oxidation of fatty | 850 |
| | acids. | |
| BBa_I739101 | Double Promoter (constitutive/TetR, negative) | 83 |
| BBa_I739102 | Double Promoter (cI, negative/TetR, negative) | 97 |
| BBa_I739103 | Double Promoter (lacI, negative/P22 cII, negative) | 87 |
| BBa_I739104 | Double Promoter (LuxR/HSL, positive/P22 cII, negative) | 101 |
| BBa_I739105 | Double Promoter (LuxR/HSL, positive/cI, negative) | 99 |
| BBa_I739106 | Double Promoter (TetR, negative/P22 cII, negative) | 84 |
| BBa_I739107 | Double Promoter (cI, negative/LacI, negative) | 78 |
| BBa_I741015 | two way promoter controlled by XylR and Crp-CAmp | 301 |
| BBa_I741017 | dual facing promoter controlled by xylR and CRP-cAMP (I741015 reverse | 302 |
| | complement) | |
| BBa_I741019 | Right facing promoter (for xylA) controlled by xylR and CRP-cAMP | 131 |
| BBa_I741020 | promoter to xylF without CRP and several binding sites for xylR | 191 |
| BBa_I741021 | promoter to xylA without CRP and several binding sites for xylR | 87 |
| BBa_I741109 | Lambda Or operator region | 82 |
| BBa_I742126 | Reverse lambda cI-regulated promoter | 49 |
| BBa_I746363 | PV promoter from P2 phage | 91 |
| BBa_I746665 | Pspac-hy promoter | 58 |
| BBa_I751500 | pcI (for positive control of pcI-lux hybrid promoter) | 77 |
| BBa_I751501 | plux-cI hybrid promoter | 66 |
| BBa_I751502 | plux-lac hybrid promoter | 74 |
| BBa_I756002 | Kozak Box | 7 |
| BBa_I756014 | LexAoperator-MajorLatePromoter | 229 |
| BBa_I756015 | CMV Promoter with lac operator sites | 663 |
| BBa_I756016 | CMV-tet promoter | 610 |
| BBa_I756017 | U6 promoter with tet operators | 341 |
| BBa_I756018 | Lambda Operator in SV-40 intron | 411 |
| BBa_I756019 | Lac Operator in SV-40 intron | 444 |
| BBa_I756020 | Tet Operator in SV-40 intron | 391 |
| BBa_I756021 | CMV promoter with Lambda Operator | 630 |
| BBa_I760005 | Cu-sensitive promoter | 16 |
| BBa_I761000 | cinr + cinl (RBS) | 1558 |
| BBa_I761001 | OmpR binding site | 62 |
| BBa_I766200 | pSte2 | 1000 |
| BBa_I766214 | pGal1 | 1002 |
| BBa_I766555 | pCyc (Medium) Promoter | 244 |
| BBa_I766556 | pAdh (Strong) Promoter | 1501 |
| BBa_I766557 | pSte5 (Weak) Promoter | 601 |
| BBa_I766558 | pFig1 (Inducible) Promoter | 1000 |
| BBa_I9201 | lambda cI operator/binding site | 82 |
| BBa_J01005 | pspoIIE promoter (spo0A J01004, positive) | 206 |
| BBa_J01006 | Key Promoter absorbs 3 | 59 |
| BBa_J03007 | Maltose specific promotor | 206 |
| BBa_J03100 | --No description-- | 847 |
| BBa_J04700 | Part containing promoter, riboswitch mTCT8-4 theophylline aptamer | 258 |
| | (J04705), and RBS | |
| BBa_J04705 | Riboswitch designed to turn “ON” a protein | 38 |
| BBa_J04800 | J04800 (RevAptRibo) contains a theophylline aptamer upstream of the RBS | 258 |
| | that should act as a riboswi | |
| BBa_J04900 | Part containing promoter, 8 bp, RBS, and riboswitch mTCT8-4 theophylline | 258 |
| | aptamer (J04705) | |
| BBa_J05209 | Modifed Pr Promoter | 49 |
| BBa_J05210 | Modifed Prm + Promoter | 82 |
| BBa_J05215 | Regulator for R1-CREBH | 41 |
| BBa_J05216 | Regulator for R3-ATF6 | 41 |
| BBa_J05217 | Regulator for R2-YAP7 | 41 |
| BBa_J05218 | Regulator for R4-cMaf | 41 |
| BBa_J05221 | Tripple Binding Site for R3-ATF6 | 62 |
| BBa_J05222 | ZF-2*e2 Binding Site | 37 |
| BBa_J05500 | Sensing Device A (cI) | 2371 |
| BBa_J05501 | Sensing Device B (cI + LVA) | 2337 |
| BBa_J06403 | RhIR promoter repressible by CI | 51 |
| BBa_J07007 | ctx promoter | 145 |
| BBa_J07010 | ToxR_inner (aa's 1-198; cytoplasm + TM) | 594 |
| BBa_J07019 | FecA Promoter (with Fur box) | 86 |
| BBa_J07041 | POPS/RIPS generator (R0051::B0030) | 72 |
| BBa_J07042 | POPS/RIPS generator (R0040::B0030) | 77 |
| BBa_J11003 | control loop for PI controller with BBa_J11002 | 961 |
| BBa_J13211 | R0040.B0032 | 75 |
| BBa_J13212 | R0040.B0033 | 73 |
| BBa_J15301 | Pars promoter from Escherichia coli chromosomal ars operon. | 127 |
| BBa_J15502 | copA promoter | 287 |
| BBa_J16101 | BanAp—Banana-induced Promoter | 19 |
| BBa_J16105 | HelPp—“Help” Dependant promoter | 26 |
| BBa_J16400 | Iron sensitive promoter (test delete later) | 26 |
| BBa_J21002 | Promoter + LuxR | 998 |
| BBa_J21003 | Promoter + TetR | 904 |
| BBa_J21004 | Promoter + LacL | 1372 |
| BBa_J21006 | LuxR, TetR Generator | 1910 |
| BBa_J21007 | LuxR, TetR, LacL Generator | 3290 |
| BBa_J22052 | Pcya | 65 |
| BBa_J22086 | pX (DnaA binding site) | 125 |
| BBa_J22126 | Rec A (SOS) promoter | 186 |
| BBa_J23150 | 1bp mutant from J23107 | 35 |
| BBa_J23151 | 1bp mutant from J23114 | 35 |
| BBa_J24000 | CafAp (Cafeine Dependant promoter) | 14 |
| BBa_J24001 | WigLp (Wiggle-dependent Promotor) | 46 |
| BBa_J24670 | Tri-Stable Toggle (Lactose induced component) | 1877 |
| BBa_J24671 | Tri-Stable Toggle (Tetracycline induced component) | 2199 |
| BBa_J24813 | URA3 Promoter from S. cerevisiae | 137 |
| BBa_J26003 | Mushroom Activated Promoter | 23 |
| BBa_J31013 | pLac Backwards [cf. BBa_R0010] | 200 |
| BBa_J31014 | crRNA | 38 |
| BBa_J3102 | pBad:RBS | 153 |
| BBa_J31020 | produces taRNA | 295 |
| BBa_J31022 | comK transcription activator from B. subtilis | 578 |
| BBa_J33100 | ArsR and Ars Promoter | 472 |
| BBa_J34800 | Promoter tetracyclin inducible | 94 |
| BBa_J34806 | promoter lac induced | 112 |
| BBa_J34809 | promoter lac induced | 125 |
| BBa_J34814 | T7 Promoter | 28 |
| BBa_J45503 | hybB Cold Shock Promoter | 393 |
| BBa_J45504 | htpG Heat Shock Promoter | 405 |
| BBa_J45992 | Full-length stationary phase osmY promoter | 199 |
| BBa_J45993 | Minimal stationary phase osmY promoter | 57 |
| BBa_J45994 | Exponential phase transcriptional control device | 1109 |
| BBa_J48103 | Iron promoter | 140 |
| BBa_J48104 | NikR promoter, a protein of the ribbon helix-helix family of trancription | 40 |
| | factors that repress expre | |
| BBa_J48106 | vnfH | 891 |
| BBa_J48107 | UGT008-3 Promoter/Met32p | 588 |
| BBa_J48110 | Fe Promoter + mRFP1 | 1009 |
| BBa_J48111 | E. coli NikR | 926 |
| BBa_J48112 | vnfH: vanadium promoter | 1816 |
| BBa_J49000 | Roid Rage | 4 |
| BBa_J49001 | Testosterone dependent promoter for species Bicyclus Bicyclus | 89 |
| BBa_J49006 | Nutrition Promoter | 3 |
| BBa_J4906 | WrooHEAD2 (Wayne Rooney's Head dependent promoter) | 122 |
| BBa_J54015 | Protein Binding Site_LacI | 42 |
| BBa_J54016 | promoter_lacq | 54 |
| BBa_J54017 | promoter_always | 98 |
| BBa_J54018 | promoter_always | 98 |
| BBa_J54101 | deltaP-GFP(A) | |
| BBa_J54102 | DeltaP-GFP(A) | 813 |
| BBa_J54110 | MelR_regulated promoter | 76 |
| BBa_J54120 | EmrR_regulated promoter | 46 |
| BBa_J54130 | BetI_regulated promoter | 46 |
| BBa_J54200 | lacq_Promoter | 50 |
| BBa_J54210 | RbsR_Binding_Site | 37 |
| BBa_J54220 | FadR_Binding_Site | 34 |
| BBa_J54230 | TetR_regulated | 38 |
| BBa_J54250 | LacI_Binding_Site | 42 |
| BBa_J56012 | Invertible sequence of dna includes Ptrc promoter | 409 |
| BBa_J56015 | lacIQ—promoter sequence | 57 |
| BBa_J61045 | [spv] spv operon (PoPS out) | 1953 |
| BBa_J61054 | [HIP-1] Promoter | 53 |
| BBa_J61055 | [HIP-1fnr] Promoter | 53 |
| BBa_J64000 | rhlI promoter | 72 |
| BBa_J64001 | psicA from Salmonella | 143 |
| BBa_J64010 | lasI promoter | 53 |
| BBa_J64065 | cI repressed promoter | 74 |
| BBa_J64067 | LuxR + 3OC6HSL independent R0065 | 98 |
| BBa_J64068 | increased strength R0051 | 49 |
| BBa_J64069 | R0065 with lux box deleted | 84 |
| BBa_J64700 | Trp Operon Promoter | 616 |
| BBa_J64712 | LasR/LasI Inducible & RHLR/RHLI repressible Promoter | 157 |
| BBa_J64750 | SPI-1 TTSS secretion-linked promoter from Salmonella | 167 |
| BBa_J64800 | RHLR/RHLI Inducible & LasR/LasI repressible Promoter | 53 |
| BBa_J64804 | The promoter region (inclusive of regulator binding sites) of the B. subtilis | 135 |
| | RocDEF operon | |
| BBa_J64931 | glnKp promoter | 147 |
| BBa_J64951 | E. Coli CreABCD phosphate sensing operon promoter | 81 |
| BBa_J64979 | glnAp2 | 151 |
| BBa_J64980 | OmpR-P strong binding, regulatory region for Team Challenge03-2007 | |
| BBa_J64981 | OmpR-P strong binding, regulatory region for Team Challenge03-2007 | 82 |
| BBa_J64982 | OmpR-P strong binding, regulatory region for Team Challenge 03-2007 | 25 |
| BBa_J64983 | Strong OmpR Binding Site | 20 |
| BBa_J64986 | LacI Consensus Binding Site | 20 |
| BBa_J64987 | LacI Consensus Binding Site in sigma 70 binding region | 32 |
| BBa_J64991 | TetR | 19 |
| BBa_J64995 | Phage-35 site | 6 |
| BBa_J64997 | T7 consensus-10 and rest | 19 |
| BBa_J64998 | consensus-10 and rest from SP6 | 19 |
| BBa_J70025 | Promoter for tetM gene, from pBOT1 plasmid, pAMbeta1 | 345 |
| BBa_J72005 | {Ptet} promoter in BBb | 54 |
| BBa_K076017 | Ubc Promoter | 1219 |
| BBa_K078101 | aromatic compounds regulatory pcbC promoter | 129 |
| BBa_K079017 | Lac symmetric - operator library member | 20 |
| BBa_K079018 | Lac 1 - operator library member | 21 |
| BBa_K079019 | Lac 2 - operator library member | 21 |
| BBa_K079036 | Tet O operator library member | 15 |
| BBa_K079037 | TetO-4C - operator library member | 15 |
| BBa_K079038 | TetO-wt/4C5G - operator library member | 15 |
| BBa_K079039 | LexA 1 - operator library member | 16 |
| BBa_K079040 | LexA 2 - opeartor library member | 16 |
| BBa_K079041 | Lambda OR1 - operator library member | 17 |
| BBa_K079042 | Lambda OR2 - operator library member | 17 |
| BBa_K079043 | Lambda OR3 - operator library member | 17 |
| BBa_K079045 | Lac operator library | 78 |
| BBa_K079046 | Tet operator library | 61 |
| BBa_K079047 | Lambda operator library | 67 |
| BBa_K079048 | LexA operator library | 40 |
| BBa_K080000 | TCFbs-BMP4 | 1582 |
| BBa_K080001 | A20/alpha cardiac actin miniPro-BMP4 | 1402 |
| BBa_K080003 | CMV-rtTA | 1413 |
| BBa_K080005 | TetO (TRE)-nkx2.5-fmdv2A-dsRed | 2099 |
| BBa_K080006 | TetO (TRE)-gata4-fmdv2A-dsRed | 2447 |
| BBa_K080008 | TetO (TRE)-nkx-2.5-fmdv2A-gata4-fmdv2A-dsRed | 3497 |
| BBa_K085004 | riboswitch system with GFP | 1345 |
| BBa_K085006 | pTet->lock3d->GFP->Ter | 932 |
| BBa_K086017 | unmodified Lutz-Bujard LacO promoter | 55 |
| BBa_K086018 | modified Lutz-Bujard LacO promoter, with alternative sigma factor σ24 | 55 |
| BBa_K086019 | modified Lutz-Bujard LacO promoter, with alternative sigma factor σ24 | 55 |
| BBa_K086020 | modified Lutz-Bujard LacO promoter, with alternative sigma factor σ24 | 55 |
| BBa_K086021 | modified Lutz-Bujard LacO promoter, with alternative sigma factor σ24 | 55 |
| BBa_K086022 | modified Lutz-Bujard LacO promoter, with alternative sigma factor σ28 | 55 |
| BBa_K086023 | modified Lutz-Bujard LacO promoter, with alternative sigma factor σ28 | 55 |
| BBa_K086024 | modified Lutz-Bujard LacO promoter, with alternative sigma factor σ28 | 55 |
| BBa_K086025 | modified Lutz-Bujard LacO promoter, with alternative sigma factor σ28 | 55 |
| BBa_K086026 | modified Lutz-Bujard LacO promoter, with alternative sigma factor σ32 | 55 |
| BBa_K086027 | modified Lutz-Bujard LacO promoter, with alternative sigma factor σ32 | 55 |
| BBa_K086028 | modified Lutz-Bujard LacO promoter, with alternative sigma factor σ32 | 55 |
| BBa_K086029 | modified Lutz-Bujard LacO promoter, with alternative sigma factor σ32 | 55 |
| BBa_K086030 | modified Lutz-Bujard LacO promoter, with alternative sigma factor σ38 | 55 |
| BBa_K086031 | modified Lutz-Bujard LacO promoter, with alternative sigma factor σ38 | 55 |
| BBa_K086032 | modified Lutz-Bujard LacO promoter, with alternative sigma factor σ38 | 55 |
| BBa_K086033 | modified Lutz-Bujard LacO promoter, with alternative sigma factor σ38 | 55 |
| BBa_K090502 | Gram-Positive Xylose-Inducible Promoter | 126 |
| BBa_K090503 | Gram-Positive General Constitutive Promoter | 91 |
| BBa_K091112 | pLacIQ1 promoter | 56 |
| BBa_K091156 | pLux | 55 |
| BBa_K091157 | pLux/Las Hybrid Promoter | 55 |
| BBa_K093008 | reverse BBa_R0011 | 55 |
| BBa_K094002 | plambda P(O-R12) | 100 |
| BBa_K094140 | pLacIq | 80 |
| BBa_K100003 | Edited Xylose Regulated Bi-Directional Operator 3 | 303 |
| BBa_K101000 | Dual-Repressed Promoter for p22 mnt and TetR | 61 |
| BBa_K101001 | Dual-Repressed Promoter for LacI and LambdacI | 116 |
| BBa_K101002 | Dual-Repressed Promoter for p22 cII and TetR | 66 |
| BBa_K102909 | TA11 gate from synthetic algorithm v1.1 | 134 |
| BBa_K102910 | TA12 gate from synthetic algorithm v1.1 | 107 |
| BBa_K102911 | TA13 gate from synthetic algorithm v1.2 | 90 |
| BBa_K102912 | TA12 plus pause sequence | 108 |
| BBa_K102950 | TA0In null anti-sense input | 175 |
| BBa_K102951 | TA1In anti-sense input to TA1 (BBa_K102901) | 157 |
| BBa_K102952 | TA2In anti-sense input to BBa_K102952 | 168 |
| BBa_K102953 | TA13n anti-sense input to TA3 (BBa_K102903) | 168 |
| BBa_K102954 | TA6In anti-sense input to BBa_K102904 | 169 |
| BBa_K102955 | TA7In anti-sense input to BBa_K102905 | 168 |
| BBa_K102956 | TA8In anti-sense input to BBa_K102906 | 168 |
| BBa_K102957 | TA9In anti-sense input to BBa_K102907 | 173 |
| BBa_K102958 | TA10In anti-sense input to BBa_K102908 | 183 |
| BBa_K102959 | TA11In anti-sense input to BBa_K102909 | 178 |
| BBa_K102960 | TA12In anti-sense input to anti-terminator BBa_K102910 | 173 |
| BBa_K102961 | TA13In anti-sense input to BBa_K102911 | 171 |
| BBa_K102962 | TA14In anti-sense input to BBa_K102912 | 180 |
| BBa_K103021 | modified T7 promoter with His-Tag | 166 |
| BBa_K103022 | Plac with operator and RBS | 279 |
| BBa_K106673 | 8xLexAops-Cyc1p | 418 |
| BBa_K106680 | 8xLexAops-Fig1P | 1169 |
| BBa_K106694 | Adh1P! (Adh1 Promoter, A! end) | 1511 |
| BBa_K106699 | Gal1 Promoter | 686 |
| BBa_K109584 | | |
| BBa_K110004 | Alpha-Cell Promoter Ste3 | 501 |
| BBa_K110007 | A-Cell Promoter MFA2 | 501 |
| BBa_K110008 | A-Cell Promoter MFA1 | 501 |
| BBa_K110009 | A-Cell Promoter STE2 | 501 |
| BBa_K110014 | A-Cell Promoter MFA2 (backwards) | 550 |
| BBa_K110015 | A-Cell Promoter MFA1 (RtL) | 436 |
| BBa_K112139 | oriR6K conditional replication origin | 408 |
| BBa_K112148 | phoPp1 magnesium promoter | 81 |
| BBa_K112149 | PmgtCB Magnesium promoter from Salmonella | 280 |
| BBa_K112321 | {H-NS!} using MG1655 reverse oligo in BBb format | 414 |
| BBa_K112701 | hns promoter | 669 |
| BBa_K112706 | Pspv2 from Salmonella | 474 |
| BBa_K112707 | Pspv from Salmonella | 1956 |
| BBa_K112708 | PfhuA | 210 |
| BBa_K112711 | rbs.spvR! | 913 |
| BBa_K112900 | Pbad | 1225 |
| BBa_K112904 | PconB5 | 41 |
| BBa_K112905 | PconC5 | 41 |
| BBa_K112906 | PconG6 | 41 |
| BBa_K112907 | Pcon | 41 |
| BBa_K113010 | overlapping T7 promoter | 40 |
| BBa_K113011 | more overlapping T7 promoter | 37 |
| BBa_K113012 | weaken overlapping T7 promoter | 40 |
| BBa_K116201 | ureD promoter from P mirabilis | |
| BBa_K119000 | Constitutive weak promoter of lacZ | 38 |
| BBa_K119001 | Mutated LacZ promoter | 38 |
| BBa_K120010 | Triple_lexO | 114 |
| BBa_K120023 | lexA_DBD | 249 |
| BBa_K121011 | promoter (lacI regulated) | 232 |
| BBa_K121014 | promoter (lambda cI regulated) | 90 |
| BBa_K124000 | pCYC Yeast Promoter | 288 |
| BBa_K124002 | Yeast GPD (TDH3) Promoter | 681 |
| BBa_K125100 | nir promoter from Synechocystis sp. PCC6803 | 88 |
| BBa_K131017 | p_qrr4 from Vibrio harveyi | 275 |
| BBa_K137085 | optimized (TA) repeat constitutive promoter with 13 bp between-10 and -35 | 31 |
| | elements | |
| BBa_K137086 | optimized (TA) repeat constitutive promoter with 15 bp between-10 and -35 | 33 |
| | elements | |
| BBa_K137087 | optimized (TA) repeat constitutive promoter with 17 bp between-10 and -35 | 35 |
| | elements | |
| BBa_K137088 | optimized (TA) repeat constitutive promoter with 19 bp between-10 and -35 | 37 |
| | elements | |
| BBa_K137089 | optimized (TA) repeat constitutive promoter with 21 bp between-10 and -35 | 39 |
| | elements | |
| BBa_K137090 | optimized (A) repeat constitutive promoter with 17 bp between-10 and -35 | 35 |
| | elements | |
| BBa_K137091 | optimized (A) repeat constitutive promoter with 18 bp between-10 and -35 | 36 |
| | elements | |
| BBa_K137124 | LacI-repressed promoter A81 | 103 |
| BBa_K143010 | Promoter ctc for B. subtilis | 56 |
| BBa_K143011 | Promoter gsiB for B. subtilis | 38 |
| BBa_K143012 | Promoter veg a constitutive promoter for B. subtilis | 97 |
| BBa_K143013 | Promoter 43 a constitutive promoter for B. subtilis | 56 |
| BBa_K143014 | Promoter Xyl for B. subtilis | 82 |
| BBa_K143015 | Promoter hyper-spank for B. subtilis | 101 |
| BBa_K145152 | Hybrid promoter: P22 c2, LacI NOR gate | 142 |
| BBa_K157042 | Eukaryotic CMV promoter | 654 |
| BBa_K165000 | MET 25 Promoter | 387 |
| BBa_K165015 | pADH1 yeast constituative promoter | 1445 |
| BBa_K165017 | LexA binding sites | 393 |
| BBa_K165037 | TEF2 yeast constitutive promoter | 403 |
| BBa_M13101 | M13K07 gene I promoter | 47 |
| BBa_M13102 | M13K07 gene II promoter | 48 |
| BBa_M13103 | M13K07 gene III promoter | 48 |
| BBa_M13104 | M13K07 gene IV promoter | 49 |
| BBa_M13105 | M13K07 gene V promoter | 50 |
| BBa_M13106 | M13K07 gene VI promoter | 49 |
| BBa_M13108 | M13K07 gene VIII promoter | 47 |
| BBa_M13110 | M13110 | 48 |
| BBa_M31201 | Yeast CLB1 promoter region, G2/M cell cycle specific | 500 |
| BBa_M31232 | Redesigned M13K07 Gene III Upstream | 79 |
| BBa_M31252 | Redesigned M13K07 Gene V Upstream | 72 |
| BBa_M31272 | Redesigned M13K07 Gene VII Upstream | 50 |
| BBa_M31282 | Redesigned M13K07 Gene VIII Upstream | 146 |
| BBa_M31292 | Redesigned M13K07 Gene IX Upstream | 69 |
| BBa_M31302 | Redesigned M13K07 Gene X Upstream | 115 |
| BBa_M31370 | tacI Promoter | 68 |
| BBa_M31519 | Modified promoter sequence of g3. | 60 |
| BBa_R0001 | HMG-CoA Dependent RBS Blocking Segment | 53 |
| BBa_R00100 | Tet promoter and sRBS | 67 |
| BBa_R00101 | VM1.0 to RiPS converter | 36 |
| BBa_R0085 | T7 Consensus Promoter Sequence | 23 |
| BBa_R0180 | T7 RNAP promoter | 23 |
| BBa_R0181 | T7 RNAP promoter | 23 |
| BBa_R0182 | T7 RNAP promoter | 23 |
| BBa_R0183 | T7 RNAP promoter | 23 |
| BBa_R0184 | T7 promoter (lacI repressible) | 44 |
| BBa_R0185 | T7 promoter (lacI repressible) | 44 |
| BBa_R0186 | T7 promoter (lacI repressible) | 44 |
| BBa_R0187 | T7 promoter (lacI repressible) | 44 |
| BBa_R1028 | Randy Rettberg Standardillator | |
| BBa_R1074 | Constitutive Promoter I | 49 |
| BBa_R1075 | Constitutive Promoter II | 49 |
| BBa_R2108 | Promoter with operator site for C2003 | 72 |
| BBa_R2110 | Promoter with operator site for C2003 | 72 |
| BBa_R2111 | Promoter with operator site for C2003 | 72 |
| BBa_R2112 | Promoter with operator site for C2003 | 72 |
| BBa_R2113 | Promoter with operator site for C2003 | 72 |
| BBa_R2182 | RiPS generator | 44 |
| BBa_R2201 | C2006-repressible promoter | 45 |
| BBa_R6182 | RiPS generator | 36 |
| BBa_S03331 | | 30 |
| BBa_S03385 | Cold-sensing promoter (hybB) | |
| BBa_Z0251 | T7 strong promoter | 35 |
| BBa_Z0252 | T7 weak binding and processivity | 35 |
| BBa_Z0253 | T7 weak binding promoter | 35 |
| BBa_Z0294 | A1, A2, A3, boxA | 435 |

### Example 4

Identification and Targeted Modulation of Nucleation Domains in Curli and Amyloid-Beta.

Effective therapeutics are urgently needed to treat diseases that involve amyloids. To create effective anti-amyloid therapeutics, structural insights that confer amyloidogenic properties must be well understood. One example of a disease-causing amyloid is curli, an extracellular amyloid that enables bacteria to bind surfaces and form difficult-to-treat biofilms. The inventors herein have used high-throughput peptide arrays to identify nucleation sites within the curli nucleator, CsgB, and demonstrated that that nucleation of CsgA is facilitated by two hydrophobic regions in CsgB. With a statistical energy minimization algorithm of the AmlyiodMutant software, the inventors identified several regions within CsgA that interact with the CsgB nucleation sites and validated these predictions with mutational analysis. Using this structural data, the inventors designed a library of peptides that were targeted at the interacting sequences in CsgA and CsgB and expressed these peptides on the surface of T7 phage. The inventors demonstrate that anti-amyloid peptide engineered bacteriophages significantly reduced in vitro curli assembly, decreased Escherichia coli biofilm formation, blocked E. coli invasion of mammalian cells, and retarded E. coli colony growth. In contrast, other discovered peptides displayed on the phages were able to increase biofilm formation. Furthermore, the inventors discovered that curli-blocking phage also inhibited amyloid-β aggregation, demonstrating that there are similarities underlying amyloid fiber formation across species and functionality that can be rationally targeted. The inventors herein have discovered specific therapeutic anti-amyloid peptides for the inhibition of curli and amyloid-β amyloids, in addition to a general strategy for analyzing amyloidogenic proteins using experimental and computational methods to design effective amyloid-modulating agents.

Amyloids play an integral role in a broad range of human illnesses including prion diseases, neurodegenerative conditions such as Alzheimer's disease and Parkinson's disease, and systemic amyloidoses¹. Curli fibers are functional amyloids that are important components for the physiology of Escherichia coli and other enteric bacteria². Functional amyloids also have been found in other organisms, including Bacillus subtilis³. Curli is localized to bacterial cell surfaces and mediates cell-cell and cell-surface contacts important in biofilm formation². Curli are also involved in adhesion and invasion of mammalian cells². Functional amyloid formation by curli is a controlled process that is regulated by many factors². The major curli subunit, CsgA, is secreted as a soluble protein to cell surfaces where it polymerized into amyloid fibrils by CsgB, an outer-membrane associated protein². CsgA and CsgB form a cross-13 sheet complex on the surface of the bacterial membrane². Conversion of CsgA and other amyloidogenic proteins into amyloid fibers involves transient intermediate structures^{4,5}.

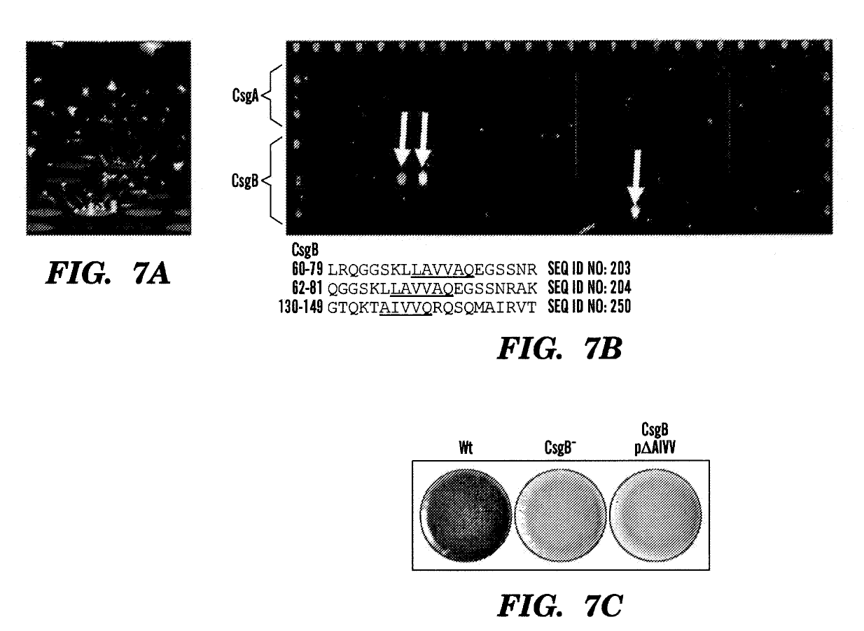

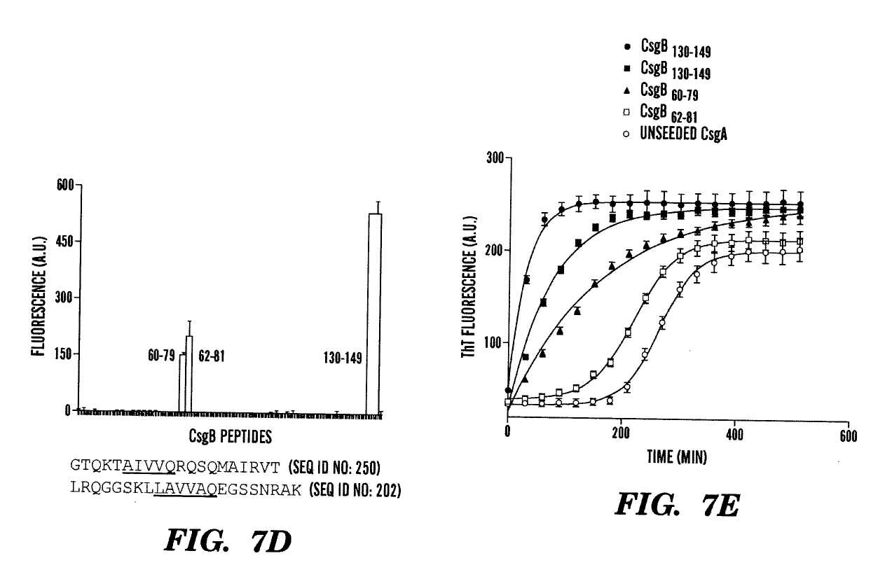

Despite the identification of amyloidogenic domains in CsgA and CsgB^{4,6}, the nucleation sequences in CsgB are still unknown. To identify the key nucleation sequences in CsgB, the inventors created peptide arrays composed of 20-residue peptides spanning the entire sequences of CsgA and CsgB (FIG. 7B)⁷. Surface-bound peptide arrays are useful for elucidating important sequences in amyloid formation since short amyloidogenic peptides are often poorly soluble. Soluble fluorescently labelled CsgA was added to the peptide arrays followed by stringent washing. When labelled CsgA was applied, no spots with CsgA peptides on the array produced significant fluorescence (FIG. 7B). However, three spots with CsgB peptides produced high levels of fluorescence (FIG. 7B). The inventors discovered that one peptide which had the strongest signal contained amino acids 130-149 in CsgB (FIGS. 7B and 7D). Two other amino acid regions in CsgB also showed substantial but lower fluorescence—amino acids 60-79 and 62-81—demonstrating the presence of a weaker nucleating site within amino acids 62-79 (FIGS. 7B and 7D). Using ThT fluorescence, the inventors validated that CsgB₆₂₋₈₁ and CsgB₁₃₀₋₁₄₉ could nucleate CsgA fiber assembly in vitro. CsgB₁₃₀₋₁₄₉ facilitated CsgA amyloid fiber formation with first-order kinetics consistent with seeded assembly (FIG. 7E). In contrast, both CsgB₆₂₋₈₁ and unseeded CsgA exhibited lag phases (FIG. 7E). The inventors' discovery of a weaker nucleating sequence in CsgB₆₂₋₇₉ and a stronger nucleating sequence in CsgB₁₃₀₋₁₄₉ are consistent with previous reports discussing that CsgB with a C-terminal 19 amino acid deletion (CsgBIII₁₃₂) was able to nucleate CsgA, with lower efficiency than full-length CsgB⁶. However, this report did not specifically demonstrate which region of CsgB₁₋₁₃₂ was able to nucleate CsgA. In some instances, the inventors used a structure & mutation prediction tool, referred to as “AmyloidMutants” as disclosed herein, which uses an algorithum to calculate the likehood of interaction sites between CsgA and CsgB (FIG. 19), to assist in predicting CsgA and CsgB interation sites and help identify CsgB peptides with a high likelihood of interation with residues of the CsgA sequence. AmyloidMutants' accuracy exceeds that of other published algorithms, and predicts full amyloid fiber structures at the resolution of β-strand backbone hydrogen contact-pairs, identifying energetically likely sets of sterically consistent β-sheet forming β-strands. This ability differs from other tools that do not distinguish whether predicted β-strand regions can be assembled consistently into fibrils and is crucial for the accurate modelling of heterogeneous fiber structures such as those formed by a CsgA/CsgB interface. Furthermore, AmyloidMutants incorporates a mutational analysis within the prediction objective function itself, which allows the rapid construction of point mutations to confirm which residues may be beneficial or detrimental to fiber formation. AmyloidMutants has been evaluated against other state-of-the-art predictors, such as Zyggregator⁸, and on five proteins with known NMR chemical shift data (amyloid-β, HET-s, Amylin, α-synuclein, and tau). AmyloidMutants demonstrates dramatically improved sensitivity in β-strand assignment (81% versus 42%) at a higher specificity (97% versus 90%). Furthermore, AmyloidMutants offers high sensitivity to even single-point mutations, as demonstrated on mutant variants of Aβ and HET-s.

To model putative CsgA/CsgA and CsgA/CsgB interfaces within an amyloid fiber, AmyloidMutants was used to explore all β-solenoidal and β-sandwiched amyloid fiber structures that the peptide sequences could attain (including parallel and anti-parallel β-strand interactions). Algorithmically, this is achieved via a Boltzmann statistical mechanical scoring function, log-odds potentials derived from the Protein Data Bank, and an efficient dynamic programming algorithm. The predicted set of most likely β-solenoidal CsgA/CsgB interfaces identified 8-12 intra- and inter-chain β-strand/β-strand interactions comprised of 6-10 (3-regions per chain. To identify only the most significant β-strand/β-strand interaction sites, the inventors applied a scalar multiplier to the scoring potentials, artificially reducing the likelihood of predicting β-strand structure by 8-fold. The resulting predictions found the same inter-chain β-strand/β-strand interactions as before, but none of the intra-chain β-regions. Given that there is no a priori bias for such a split in predicted outcome, this supports the notion that these inter-chain interactions are important in the formation or stabilization of amyloid structure.

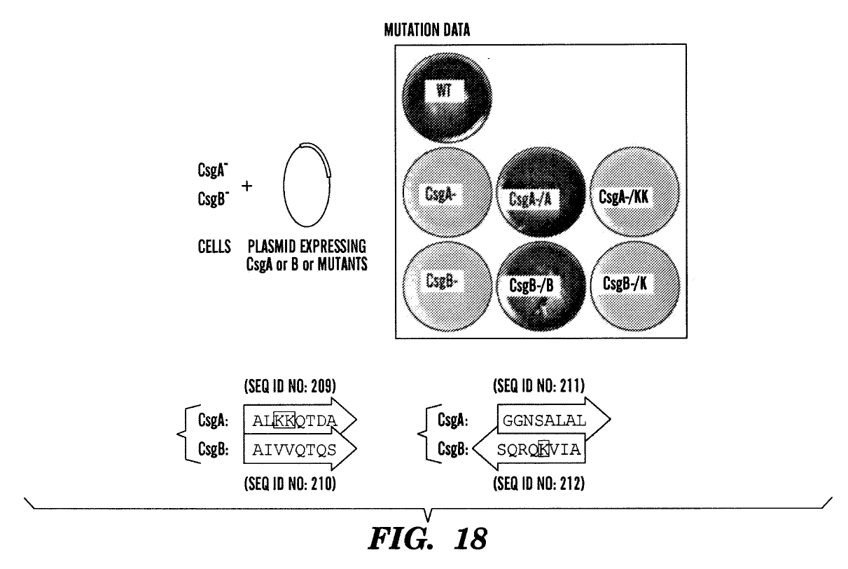

Within CsgB, two sequence regions around positions 60-81, and 130-149 were predicted to form inter-chain β-strands, aligning with CsgB peptide sequences shown to nucleate CsgA within the peptide array (FIG. 7D and FIG. 19). Pairing partners within CsgA were predicted at regions 43-61 (with two distinct likelihoods at 43-50 and 54-61), and 132-140 (FIG. 19). Thus, AmyloidMutants' was used to predict structural models of homogeneous CsgA fiber regions and some of the CsgA/CsgB interfaces and recapitulates the relative importance of the five known peptide repeat regions within CsgA, identifying repeats R1 and R5 as crucial to fiber structure^{4,9}. Based on the AmyloidMutant's pseudo-energy scores, amoung the top individual β-strand/β-strand interaction core which was identified included peptide sequences which centered around CsgA₅₄₋₆₁ pairing to CsgB₁₃₄₋₁₄₀ (NSALALQT/TAIVVQR) (SEQ ID NO: 195/SEQ ID NO: 196). Since the interaction between CsgA and CsgB introduces a putative asymmetry along the fiber axis in the (3-solenoidal model, the inventors' predictions were re-run assuming all four possible N-terminal/C-terminal orientations that may arise, presenting similar top-scoring cores. To confirm AmyloidMutant's predictions, the inventors created site-specific mutations in CsgA and CsgB. The ability of mutations in these regions to abolish curli formation was assayed by Congo red binding on agar plates (FIG. 18).

Based on the identification of nucleation domains in CsgB and interacting sequences in CsgA, the inventors displayed peptides on phage capsids targeted against CsgA and CsgB sequences to assess their ability to modulate amyloid assembly and function. Phage display of amyloid-modulating peptides has several advantages. First, the cost of constructing recombinant phage using synthetic DNA primers is lower than the cost of peptide synthesis. Second, the construction and validation of recombinant phage is relatively faster than peptide synthesis. Third, additional recombinant phage can be generated much more rapidly and cheaply than additional peptides. Fourth, peptides that contain amyloidogenic sequences are often poorly soluble and difficult to express or be functional both in vitro or in vivo. By expressing amyloidogenic sequences on phage capsids, the inventors were able to avoid issues with of the anti-amyloid peptide solubility. Finally, phage may be a useful delivery vehicle for in vivo use of amyloid-modulating peptides. For example, phages injected intravenously into mice have been shown to distribute throughout the body and can be targeted to various organs¹⁰. Furthermore, filamentous phages can be delivered into the brain intranasally¹¹.

The inventors demonstrated in Example 1 that unmodified M13 phage was more effective in inhibiting amyloid formation than T7 phage. Thus, the inventors expressed peptides on the capsids of T7 phage instead of M13 phage to isolate the amyloid-blocking effects of peptide modulators from the effects of phage. The inventors used a high-copy phage-display system that expresses 415 peptide copies on the phage surface. First, the inventors constructed two phages expressing wild-type sequences from CsgA (CsgA₄₃₋₅₂ (SEQ ID NO: 11) and CsgA₅₅₋₆₄ (SEQ ID NO: 12) predicted to interact with the major nucleating sequence of CsgB (named T7-CsgA₄₃₋₅₂ and T7-CsgA₅₅₋₆₄, respectively). The inventors also constructed a phage expressing the major wild-type nucleating sequence of CsgB (T7-CsgB₁₃₃₋₁₄₂) (SEQ ID NO: 29). At low phage concentrations (<10³ plaque-forming-units/mL (PFU/mL)), T7-CsgA₄₃₋₅₂, T7-CsgA₅₅₋₆₄, and T7-CsgB₁₃₃₋₁₄₂ slightly stimulated amyloid fiber assembly by CsgA (FIGS. 3A and 3B). Both T7-CsgA₅₅₋₆₄ and T7-CsgB₁₃₃₋₁₄₂ decreased the lag time of CsgA fiber assembly, with T7-CsgB₁₃₃₋₁₄₂ exceeding T7-CsgA₅₅₋₆₄ in amyloid-stimulating efficacy at phage concentrations of 500 PFU/mL (FIG. 3B). However, at concentrations higher than 10³ PFU/mL, these anti-amylod peptide engineered bacteriophages inhibited fiber assembly by up to 59% (FIG. 3A). Thus, T7-CsgA₄₃₋₅₂, T7-CsgA₅₅₋₆₄, and T7-CsgB₁₃₃₋₁₄₂ constitute an effective class of anti-amyloid peptide bacteriophages that enhance in vitro curli aggregation at low concentrations and inhibit aggregation at high concentrations (defined as Class IIa in FIG. 3A).

### Example 5

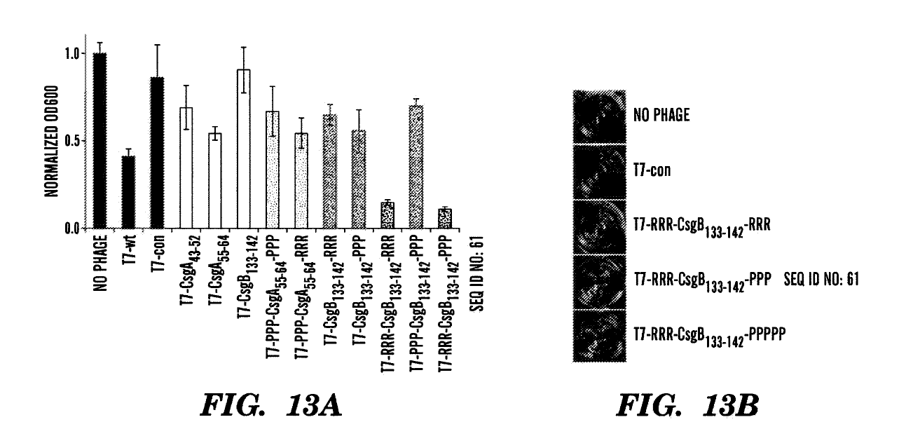

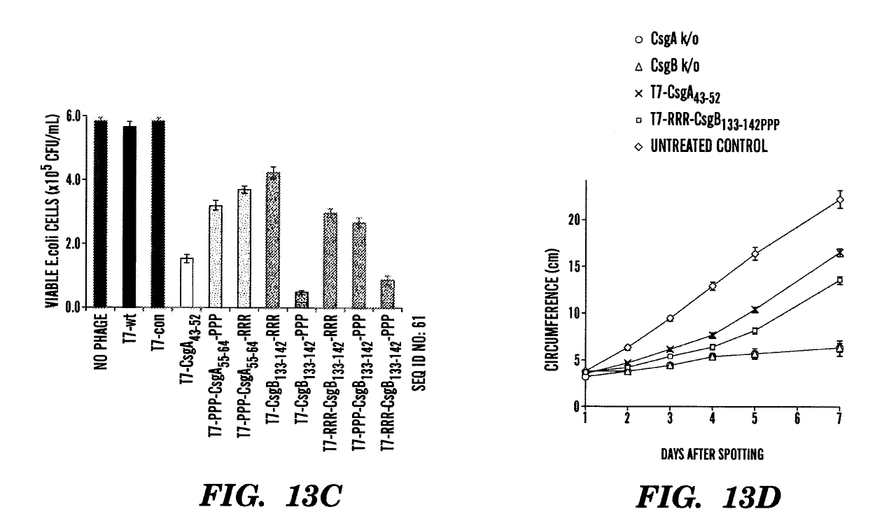

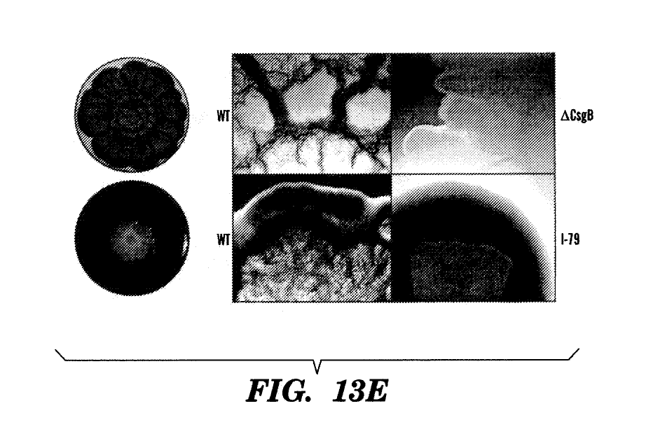

The inventors tested whether effective modulators of biofilm formation could be found within the most effective class of phages based on the disclosed in vitro assays. The inventors used Escherichia coli O1:K1:H7 (ATCC #11775), a urinary isolate, to grow biofilms on polystyrene pegs. This strain expresses curli and cannot be infected by T7 phage due to the K1 capsule, thus allowing us to examine the effects of peptide modulators without the influence of phage infection¹³. Quantification of biofilm formation was determined via crystal violet staining (FIGS. 13A and 13B)¹⁴. Control T7 (T7-con) (SEQ ID NO: 91) expressing an S•Tag peptide reduced biofilm formation by about 13%. In contrast, T7-PPP-CsgA₅₅₋₆₄-PPP (SEQ ID NO: 52), T7-RRR-CsgA₅₅₋₆₄-PPP, T7-CsgB₁₃₃₋₁₄₂-RRR (SEQ ID NO: 64), T7-CsgB₁₃₃₋₁₄₂-PPP (SEQ ID NO: 65), and T7-PPP-CsgB₁₃₃₋₁₄₂-PPP (SEQ ID NO: 63) moderately decreased biofilm formation (ranging from 30-50% inhibition) (FIG. 13A). Discrepancies between the inhibition of curli assembly in vitro and biofilm formation may be due to other extracellular components that can mediate cell attachment and biofilm formation in E. coli¹⁵. The most effective inhibitors of biofilm formation were T7-RRR-CsgB₁₃₃₋₁₄₂-PPP (89% inhibition) and T7-RRR-CsgB₁₃₃₋₁₄₂-RRR (SEQ ID NO: 61) (85% inhibition) (FIGS. 13A and 13B).

In addition to enhancing biofilm formation, curli plays an important role in mediating cell invasion, colony growth, and colony morphology². The inventors quantified cell invasion using a gentamicin protection assay with HEK 293 cells and E. coli¹⁸. Bacteria were preincubated with phage for two hours prior to addition of gentamicin. This assay showed that T7-CsgB₁₃₃₋₁₄₂-PPP (SEQ ID NO: 65) and T7-RRR-CsgB₁₃₃₋₁₄₂-PPP (SEQ ID NO: 61) were most effective at inhibiting cell invasion (FIG. 13C). Furthermore, the inventors found that adding T7-RRR-CsgB₁₃₃₋₁₄₂-PPP (SEQ ID NO: 61) to E. coli or knocking out csgA and csgB in E. coli reduced colony growth rates in YESCA soft agar plates (FIG. 13D). Finally, phage-treated cells, ΔcsgA E. coli, and ΔcsgB E. coli exhibited decreased Congo red binding and loss of rough morphologies compared with wild-type bacteria (FIG. 13E). The inventors therefore have demonstrated that disrupting curli with phage-displayed peptide modulators have a variety of biological effects that may be useful in treating and preventing surface-associated bacterial infections.

### Example 6

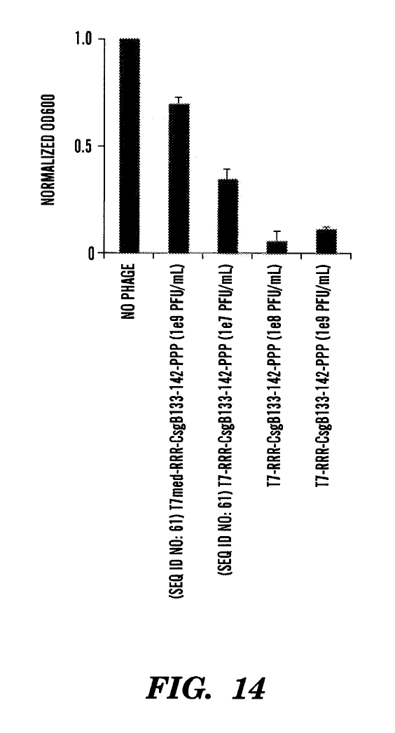

The most effective inhibitors of biofilm formation were T7-RRR-CsgB₁₃₃₋₁₄₂-PPP (SEQ ID NO: 61) (89% inhibition) and T7-RRR-CsgB₁₃₃₋₁₄₂-RRR (SEQ ID NO: 62) (85% inhibition) (FIGS. 13A and 13B). The inventors chose to characterize the biofilm-inhibiting activity of the most effective engineered phage, T7-RRR-CsgB₁₃₃₋₁₄₂-PPP (SEQ ID NO: 61), further. The inventors discovered that biofilm inhibition was dependent on phage concentration, with 10⁸ PFU/mL and 10⁷ PFU/mL exhibiting 95% and 66% inhibition, respectively (FIG. 14). Furthermore, expressing RRR-CsgB₁₃₃₋₁₄₂-PPP from a medium-copy phage cloning system with 5-15 peptides on the phage surface (T7_{med}-RRR-CsgB₁₃₃₋₁₄₂-PPP) resulted in decreased biofilm-inhibiting efficacy (from 89% to 30% inhibition) (FIG. 14).

### Example 7

The inventors also investigated the structural requirements that confer biofilm-inhibiting activity upon T7-RRR-CsgB₁₃₃₋₁₄₂-PPP (SEQ ID NO: 61) (FIG. 15). Independently increasing the number of N-terminal arginines or C-terminal prolines from three to five did not affect biofilm inhibition dramatically (90% and 80% biofilm inhibition for T7-RRR-CsgB133-142-PPPPP (SEQ ID NO: 85) and T7-RRRRR-CsgB133-142-PPP (SEQ ID NO: 86) respectively). Similarly, increasing the number of both N-terminal arginines and C-terminal prolines from three to five in the same phage T7-RRRRR-CsgB₁₃₃₋₁₄₂-PPPPP (SEQ ID NO: 84) had only small effects (from 89% to 77% inhibition). However, simultaneously decreasing the number of both N-terminal arginines and C-terminal prolines from three to two (T7-RR-CsgB₁₃₃₋₁₄₂-PP; (SEQ ID NO: 89)) or from three to one (T7-R-CsgB₁₃₃₋₁₄₂-P) (SEQ ID NO: 90) had detrimental effects on biofilm-blocking activity (from 89% to 43% and 56%, respectively). Substituting glycine residues for the N-terminal arginines (T7-GGG-CsgB₁₃₃₋₁₄₂-PPP) (SEQ ID NO: 87) did not affect biofilm inhibition greatly (from 89% to 81%). However, substituting gylcine residues for the C-terminal prolines (T7-RRR-CsgB₁₃₃₋₁₄₂-GGG (SEQ ID NO: 88) enhanced biofilm formation (from 80% inhibition to 53% stimulation). Improved biofilm formation may be beneficial for bioremediation and biotechnology applications¹⁶. Thus, the inventors have demonstrated that C-terminal PPP residues are critical for biofilm-inhibiting efficacy. Furthermore, by identifying amyloid nucleation domains and designing rational peptide-based modulators, the biological effects of amyloids can be enhanced or inhibited.

### Example 8

Surfaces coated with anti-amyloid peptide engineered bacteriophages or peptides which inhibit biofilm formation as disclosed herein are useful for reducing biofilm infections on medical devices. The inventors assessed whether preincubation of surfaces with anti-amyloid peptide engineered bacteriophages could prevent biofilm formation. The inventors demonstrated that T7-RRR-CsgB₁₃₃₋₁₄₂-PPP (SEQ ID NO: 61) and T7-RRR-CsgB₁₃₃₋₁₄₂-PPPPP (SEQ ID NO: 85) decreased biofilm formation by 35% and 52%, respectively (FIG. 16). The effectiveness of this approach could be enhanced by controlled release of curli-inhibiting phage or peptides as well as other strategies such as covalent attachment, or co-display of surface-binding peptides on curli-inhibiting phage¹⁷, which are encompassed for use in the methods and compositions as disclosed herein.

### Example 9

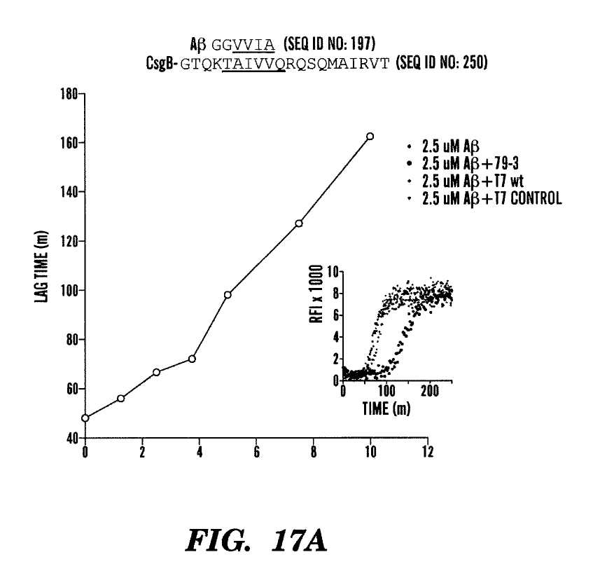

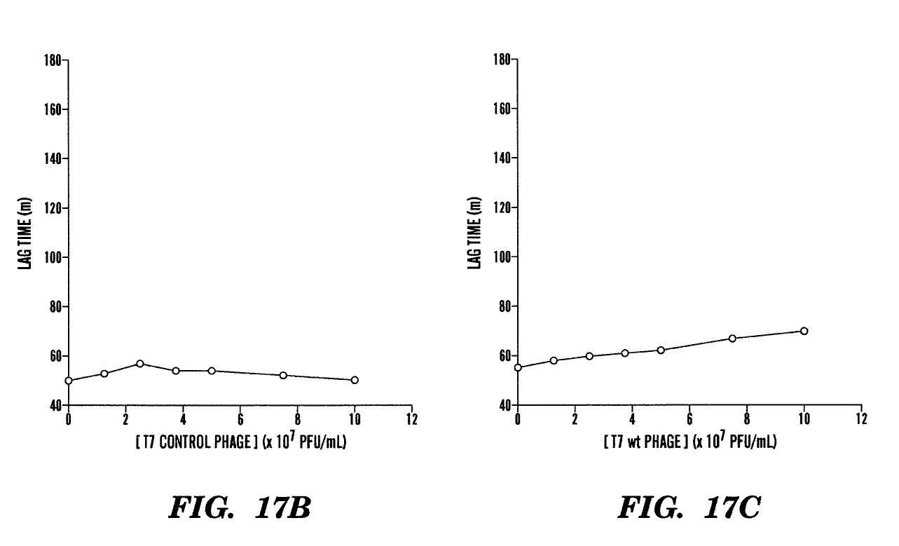

The inventors herein have identified the major nucleating sequence of CsgB as TAIVVQR (CsgB₁₃₄₋₁₄₀) (SEQ ID NO: 196). Amyloid-13 (Aβ) another amyloid-forming protein, is known to have a nucleating sequence Aβ₃₇₋₄₂, GGVVIA (SEQ ID NO: 197)¹⁹. Since a subset of this Aβ nucleator, VVIA (SEQ ID NO: 198), is exactly the reverse of the critical nucleating sequence of CsgB (AIVV) (SEQ ID NO: 199), the inventors assessed if T7-RRR-CsgB₁₃₃₋₁₄₂-PPP (SEQ ID NO: 61) has an efficacy against Aβ aggregation (FIG. 17A). Using an in vitro ThT fluorescence assay with 2.5 μM Aβ, the inventors demostrated that T7-RRR-CsgB₁₃₃₋₁₄₂-PPP (SEQ ID NO: 61) increased the lag time for Aβ fiber formation compared with T7-con (FIG. 17B) and T7-wt (FIG. 17C). This effect was dependent on the concentration of phage. A doubling of the lag time was achieved at 5×10⁷ PFU/mL of T7-RRR-CsgB₁₃₃₋₁₄₂-PPP (SEQ ID NO: 61), which translates into an Aβ:peptide molar ratio of greater than 70,000. These results implicate similarities between Aβ and curli aggregation and demonstrate that that replacing the interaction domains of CsgA and the nucleation domains of CsgB identified herein with the crucial amyloidogenic interaction domain of other amyloids is extremely useful for studying other amyloid systems and designing peptide modulators. Furthermore, the anti-amyloid peptide engineered bacteriophage that express amyloid-inhibiting peptides can be useful as a therapeutic platform for developing anti-amyloid engineered bacteriophages for inhibiting amyloid formation for other protein-misfolding diseases²⁰.

Bacterial biofilms are an important source of intractable infections in medical and industrial settings²¹. The curli-inhibiting phage and peptides that the inventors have demonstrated herein are extremely useful as new therapeutics to inhibit and prevent biofilm formation. The inventors used T7 phage, which is unable to infect E. coli O1:K1:H7, to express the anti-amyloid peptide to isolate the anti-biofilm effect of phage-displayed peptides from the anti-biofilm effect of phage infection. Thus, anti-biofilm efficacy can be further enhanced by one of ordinary skill in the art using amyloid-inhibiting phage that also productively infect target bacteria. Furthermore, the inventors used T7 instead of M13 phage, which exhibited greater amyloid-inhibiting efficacy in vitro, to identify the anti-biofilm effect due to the anti-amyloid peptides expressed from the phage, as compared to the anti-biofilm effect of the phage itself. Expression of anti-amyloid peptides on the surface of M13 can be used to yield even greater suppression of amyloid formation since M13 bacteriophage possesses some level of anti-amyloid activity (FIG. 1). Moreover, expressing cyclic peptides instead of linear peptides on phage capsids can be used to yield enhanced in vivo efficacy at blocking amyloid formation. Finally, the anti-curli or anti-amyloid peptides the inventors have identified herein can be useful as surface coatings to prevent biofilm formation, or in fluid samples to prevent bacterial infection and biofilm formation.

Combination therapies of the anti-amyloid peptide engineered bacteriophage and other engineered phages, e.g. biofilm-degrading phage, antibiotic-resistance-suppressing phage, or other agents, e.g. antibiotics, small-molecule amyloid inhibitors, and D-amino acids can also be used by one of ordinary skill in the art for enhanced efficacy against biofilms. For example, engineered phage that express biofilm-degrading enzymes (see U.S. patent Ser. Nos. 12/337, 677, 11/662, 551 and International Application WO06/137847, which are incorporated herin in their entirety by reference), or repressors of important antibiotic-resistance gene networks (e.g. as disclosed in WO 2009/108406) during infection enhance biofilm destruction and bacterial killing, especially when used in combination with antibiotics^{14,22}. Recently, D-amino acids have also been reported to inhibit biofilms by releasing amyloid fibers from cells²³. Small-molecule inhibitors of amyloid formation by curli¹⁵ and the yeast prion protein, Sup35²⁴, have also been reported. β-breaker peptides targeted against CsgA have also been reported to block curli amyloids²⁵. However, none of these therapeutics have been specifically targeted against the nucleation domains of CsgB as demonstrated herein by the inventors. Furthermore, the very low molar ratio required between amyloidogenic proteins and amyloid-blocking peptides demonstrates a high anti-amyloid efficiency of the anti-amyloid engineered bacteriophage as disclosed herein.

The inventors have discovered a major nucleating sequence of CsgB is the reverse of an Aβ nucleating sequence. Based on this discovery, the inventors demonstrated inhibition of both curli and Aβ aggregation by the same anti-amyloid engineered bacteriophage. Furthermore, the inventors have also demonstrated that both curli and Sup35-NM amyloid formation could be suppressed by unmodified M13mp18 phage. Thus, the inventors have demonstrated similarities between different amyloid systems in bacteria to yeast to humans. Though there are specific recognition elements that determine species-specific seeding for yeast prions^{5,7}, it has been reported that murine amyloid protein A aggregation can be accelerated by natural amyloid fibrils, such as silk, Sup35, and curli²⁶. Based on the inventors' discovery herein that a nucleating sequence for CsgB also inhibits Aβ amyloid formation, other amyloid systems can be dissected using peptide arrays and computational algorithms followed by probing and modulation using phage-based peptide expression. Thus, the inventors' discovery can be used as a general strategy to investigate and develop treatments for other important protein-misfolding diseases.

### REFERENCES

All references cited herein, in the specification and Examples are incorporated in their entirety by reference.
1. Arkin, M. R. & Wells, J. A. Small-molecule inhibitors of protein-protein interactions: progressing towards the dream. Nat Rev Drug Discov 3, 301-317, (2004).
2. Chapman, M. R. et al. Role of Escherichia coli curli operons in directing amyloid fiber formation. Science 295, 851-855, (2002).
3. Loferer, H., Hammar, M. & Normark, S. Availability of the fibre subunit CsgA and the nucleator protein CsgB during assembly of fibronectin-binding curli is limited by the intracellular concentration of the novel lipoprotein CsgG. Mol Microbiol 26, 11-23 (1997).
4. Hammar, M., Arnqvist, A., Bian, Z., Olsen, A. & Normark, S. Expression of two csg operons is required for production of fibronectin- and congo red-binding curli polymers in Escherichia coli K-12. Mol Microbiol 18, 661-670 (1995).
5. Lu, T. K. & Collins, J. J. Dispersing biofilms with engineered enzymatic bacteriophage. Proc Natl Acad Sci USA 104, 11197-11202, (2007).
   All references below cited herein, indicated by superscript numbers in the Examples, are incorporated in their entirety by reference.
1 Chiti, F. & Dobson, C. M. Protein misfolding, functional amyloid, and human disease. Annu Rev Biochem 75, 333-366, doi:10.1146/annurev.biochem.75.101304.123901 (2006).
2 Barnhart, M. M. & Chapman, M. R. Curli biogenesis and function. Annu Rev Microbiol 60, 131-147, doi:10.1146/annurev.micro.60.080805.142106 (2006).
3 Romero, D., Aguilar, C., Losick, R. & Kolter, R. Amyloid fibers provide structural integrity to Bacillus subtilis biofilms. Proc Natl Acad Sci USA 107, 2230-2234, doi:0910560107 [pii]10.1073/pnas.0910560107 (2010).
4 Wang, X., Smith, D. R., Jones, J. W. & Chapman, M. R. In vitro polymerization of a functional Escherichia coli amyloid protein. J Biol Chem 282, 3713-3719, doi:M609228200 [pii]10.1074/jbc.M609228200 (2007).
5 Krishnan, R. & Lindquist, S. L. Structural insights into a yeast prion illuminate nucleation and strain diversity. Nature 435, 765-772, doi:nature03679 [pii]10.1038/nature03679 (2005).
6 Hammer, N. D., Schmidt, J. C. & Chapman, M. R. The curli nucleator protein, CsgB, contains an amyloidogenic domain that directs CsgA polymerization. Proc Natl Acad Sci USA 104, 12494-12499, doi:0703310104 [pii]10.1073/pnas.0703310104 (2007).
7 Tessier, P. M. & Lindquist, S. Prion recognition elements govern nucleation, strain specificity and species barriers. Nature 447, 556-561, doi:nature05848 [pii]10.1038/nature05848 (2007).
8 Calamai, M., Tartaglia, G. G., Vendruscolo, M., Chiti, F. & Dobson, C. M. Mutational analysis of the aggregation-prone and disaggregation-prone regions of acylphosphatase. J Mol Biol 387, 965-974, doi:S0022-2836(08)01119-4 [pii]10.1016/j.jmb.2008.09.003 (2009).
9 Wang, X., Hammer, N. D. & Chapman, M. R. The molecular basis of functional bacterial amyloid polymerization and nucleation. J Biol Chem 283, 21530-21539, doi:M800466200 [pii]10.1074/jbc.M800466200 (2008).
10 Pasqualini, R. & Ruoslahti, E. Organ targeting in vivo using phage display peptide libraries. Nature 380, 364-366, doi:10.1038/380364a0 (1996).
11 Frenkel, D. & Solomon, B. Filamentous phage as vector-mediated antibody delivery to the brain. Proc Natl Acad Sci USA 99, 5675-5679, doi:10.1073/pnas.07202719999/8/5675 [pii] (2002).
12 Soto, C. et al. Beta-sheet breaker peptides inhibit fibrillogenesis in a rat brain model of amyloidosis: implications for Alzheimer's therapy. Nat Med 4, 822-826 (1998).
13 Scholl, D., Adhya, S. & Merril, C. Escherichia coli K1's capsule is a barrier to bacteriophage T7. Appl Environ Microbiol 71, 4872-4874, doi:71/8/4872 [pii]10.1128/AEM.71.8.4872-4874.2005 (2005).
14 Lu, T. K. & Collins, J. J. Dispersing biofilms with engineered enzymatic bacteriophage. Proc Natl Acad Sci USA 104, 11197-11202, doi:0704624104 [pii]10.1073/pnas.0704624104 (2007).
15 Cegelski, L. et al. Small-molecule inhibitors target Escherichia coli amyloid biogenesis and biofilm formation. Nat Chem Biol 5, 913-919, doi:nchembio.242 [pii]10.1038/nchembio.242 (2009).
16 Singh, R., Paul, D. & Jain, R. K. Biofilms: implications in bioremediation. Trends Microbiol 14, 389-397, doi:S0966-842X(06)00156-9 [pii]10.1016/j.tim.2006.07.001 (2006).
17 Adey, N. B., Mataragnon, A. H., Rider, J. E., Carter, J. M. & Kay, B. K. Characterization of phage that bind plastic from phage-displayed random peptide libraries. Gene 156, 27-31, doi:037811199500058E [pii] (1995).
18 Gophna, U. et al. Curli fibers mediate internalization of Escherichia coli by eukaryotic cells. Infect Immun 69, 2659-2665, doi:10.1128/IAI.69.4.2659-2665.2001 (2001).
19 Sawaya, M. R. et al. Atomic structures of amyloid cross-beta spines reveal varied steric zippers. Nature 447, 453-457, doi:nature05695 [pii]10.1038/nature05695 (2007).
20 Lowe, T. L., Strzelec, A., Kiessling, L. L. & Murphy, R. M. Structure-function relationships for inhibitors of beta-amyloid toxicity containing the recognition sequence KLVFF. Biochemistry 40, 7882-7889, doi:bi002734u [pii] (2001).
21 Costerton, J. W., Stewart, P. S. & Greenberg, E. P. Bacterial biofilms: a common cause of persistent infections. Science 284, 1318-1322 (1999).
22 Lu, T. K. & Collins, J. J. Engineered bacteriophage targeting gene networks as adjuvants for antibiotic therapy. Proc Natl Acad Sci USA 106, 4629-4634, doi:0800442106 [pii]10.1073/pnas.0800442106 (2009).
23 Kolodkin-Gal, I. et al. D-amino acids trigger biofilm disassembly. Science 328, 627-629, doi:328/5978/627 [pii]10.1126/science.1188628 (2010).
24 Wang, H. et al. Direct and selective elimination of specific prions and amyloids by 4,5-dianilinophthalimide and analogs. Proc Natl Acad Sci USA 105, 7159-7164, doi:0801934105 [pii]110.1073/pnas.0801934105 (2008).
25 Cherny, I. et al. The formation of Escherichia coli curli amyloid fibrils is mediated by prion-like peptide repeats. J Mol Biol 352, 245-252, doi:S0022-2836(05)00816-8 [pii]10.1016/j.jmb.2005.07.028 (2005).
26 Lundmark, K., Westermark, G. T., Olsen, A. & Westermark, P. Protein fibrils in nature can enhance amyloid protein A amyloidosis in mice: Cross-seeding as a disease mechanism. Proc Natl Acad Sci USA 102, 6098-6102, doi:0501814102 [pii]10.1073/pnas.0501814102 (2005).
   References for the Computational methods are as follows, and are incorporated herein in their entirety by reference.
1. Dobson, C. M. Protein folding and misfolding. Nature 426, 884-890 (2003).
2. Chiti, F. & Dobson, C. M. Protein Misfolding, Functional Amyloid, and Human Disease. Annu. Rev. Biochem. 75, 333-366 (2006).
3. Maji, S. K., et al. Functional Amyloids As Natural Storage of Peptide Hormones in Pituitary Secretory Granules. Science 325, 328-332 (2009).
4. Barnhart, M. M. & Chapman, M. Curli biogenesis and function. Annu. Rev. Microbiol. 60, 131-147 (2006).
5. Fowler, D. M. & Kelly, J. W. Aggregating knowledge about prions and amyloid. Cell 137, 146-158 (2009).
6. Pedersen, J. S. & Otzen, D. E. Amyloid—a state in many guises: Survival of the fittest fibril fold. Protein Sci. 17, 2-10 (2009).
7. Lie, J., et al. Toxicity of Familial ALS-Linked SOD1 Mutants from Selective Recruitment to Spinal Mitochondria. Neuron 43, 5-17 (2004).
8. Mukrasch, M. D., et al. Structural Polymorphism of 441-Residue Tau at Single Residue Resolution. PLoS Biol. 7, e1000034 (2009).
9. L″uhrs, T., et al. 3D structure of Alzheimer's amyloid-β(1-42) fibrils. Proc. Natl. Acad. Sci. 102, 17342-17347 (2005).
10. Wasmer, C., et al. Amyloid Fibrils of the HET-s(218-289) Prion Form a β Solenoid with a Triangular Hydrophobic Core. Science 219, 1523-1526 (2008).
11. Luca, S., Yau, W., Leapman, R. & Tycko, R. Peptide Conformation and Supramolecular Organization in Amylin Fibrils: Constraints from Solid State NMR. Biochemistry 46, 13505-13522 (2007).
12. Vilar, M., et al. The fold of α-synuclein fibrils. Proc. Natl. Acad. Sci. 105, 8637-8642 (2007).
13. Alberti, S., Halfmann, R., King, O., Kapila, A. & Lindquist, S. A Systematic Survey Identifies Prions and Illuminates Sequence Features of Prionogenic Proteins. Cell 147, 146-158 (2009).
14. Bryan A. W. Jr., Menke M., Cowen L. J., Lindquist S., Berger B. BETASCAN: Probable β-amyloids Identified by Pairwise Probabilistic Analysis. PLoS Comput. Biol. 5, e1000333 (2009).
15. Tartaglia, G. G. & Vendruscolo, M. The Zyggregator method for predicting protein aggregation propensities. Chem. Soc. Rev. 37, 1395-1401 (2008).
16. Trovato, A., Seno, F. & Tosatto, S. C. The PASTA server for protein aggregation prediction. Protein Eng., Des. Sel. 20, 521-523 (2007).
17. Fernandez-Escamilla, A., Rousseau, F., Schymkowitz, J. & Serrano, L. Prediction of sequence-dependent and mutational effects on the aggregation of peptides and proteins. Nat. Biotechnol. e-pub (2004).
18. Maurer-Stroh, S. et al. Exploring the sequence determinants of amyloid structure using position-specific scoring matrices. Nat. Methods 7, 237-242 (2010).
19. Morel, B., Casares, S. & Conejero-Lara, F. A Single Mutation Induces Amyloid Aggregation in the α-Spectrin SH3 Domain: Analysis of the Early Stages of Fibril Formation. J. Mol. Biol. 356, 453-468 (2006).
20. Tycko, R., Sciarretta, K. L., Orgel, J. P. R. O. & Meredith, S. C. Evidence for Novel β-Sheet Structures in Iowa Mutant β-Amyloid Fibrils. Biochemistry 48, 6074-6084 (2009).
21. Couthouis, J., et al. Screening for Toxic Amyloid in Yeast Exemplifies the Role of Alternative Pathway Responsible for Cytotoxicity. PLoS ONE 4, e4539 (2009).
22. Kajava, A. V., Aebi, U. & Steven, A. C. The Parallel Superpleated Beta-structure as a Model for Amyloid Fibrils of Human Amylin. J. Mol. Biol. 348, 247-252 (2005).
23. Heise, H., et al. Molecular-level secondary structure, polymorphism, and dynamics of full-length α-synuclein fibrils studied by solid-state NMR. Proc. Natl. Acad. Sci. 102, 15871-15876 (2005).
24. von Bergen, M., et al. Assembly of τ protein into Alzheimer paired helical filaments depends on a local sequence motif (306VQIVYK311) forming β structure. Proc. Natl. Acad. Sci. 97, 5129-5134 (2000).
25. Petkova, A. T., et al. Self-Propagating, Molecular-Level Polymorphism in Alzheimer's β-Amyloid Fibrils. Science 307, 262-265 (2005).
26. Petkova, A. T., et al. A structural model for alzheimer's beta-amyloid fibrils based on experimental constraints from solid state nmr. Proc. Natl. Acad. Sci. 100, 383-385 (2003).
27. Kim, W. & Hecht, M. H. Mutations Enhance the Aggregation Propensity of the Alzheimer's Aβ Peptide. J. Mol. Biol. 377, 565-574 (2008).
28. Berthelot, K., et al. Driving amyloid toxicity in a yeast model by structural changes: a molecular approach. FASEB J. 23, 2254-2263 (2009).
29. Tycko, R. & Ishii, Y. Constraints on supra-molecular structure in amyloid fibrils from two-dimensional solid state NMR spectroscopy with uniform isotopic labeling. J. Am. Chem. Soc. 125, 6606-6607 (2003).
30. Petkova, A. T., et al. Solid State NMR Reveals a pH-dependent Antiparallel β-sheet Registry in Fibrils Formed by a β-Amyloid Peptide. J. Mol. Biol. 335, 27-260 (2004).
31. Williams, A. D., et al. Mapping Aβ Amyloid Fibril Secondary Structure Using Scanning Proline Mutagenesis. J. Mol. Biol. 335, 833-842 (2004).
32. Coustou, V., Deleu, C., Saupe, S. J. & B´egueret, J. Mutational Analysis of the [HET-s] Prion Analog of Podospora anserina: a Short N-Terminal Peptide Allows Prion Propagation. Genetics 153, 1629-1640 (1999).
33. Wurth, C., Guimard, N. K. & Hecht, M. H. Mutations that Reduce Aggregation of the Alzheimer's Aβ42 Peptide: an Unbiased Search for the Sequence Determinants of Aβ Amyloidogenesis. J. Mol. Biol. 319, 1279-1290 (2002).
34. Kim, W. & Hecht, M. H. Generic hydrophobic residues are sufficient to promote aggregation of the Alzheimer's Aβ42 peptide. Proc. Nat. Acad. Sci. 103, 15824-15829 (2006).
35. Ortlund, E. A., Bridgham, J. T., Redinbo, M. R. & Thornton, J. W. Crystal Structure of an Ancient Protein Evolution by Conformational Epistasis. Science 317, 1544-1548 (2007).
36. Istrail, S. Statistical mechanics, three-dimensionality and NP-completeness: I. Universality of intractability of the partition functions of the ising model across non-planar lattices. In Press, A. (ed.) Proceedings of the 32nd ACM Symposium on the Theory of Computing (STOC00), 87-96 (2000).
37. McCaskill, J. The equilibrium partition function and base pair binding probabilities for RNA secondary structure. Biopolymers 29, 1105-1119 (1990).
38. Waldisp\ddot{}uhl, J., O'Donnell, C. W., Devadas, S., Clote, P. & Berger, B. Modeling Ensembles of Transmembrane β-barrel Proteins. Proteins: Struct., Funct., Bioinf. 71, 1097-1112 (2008).
39. Waldisp\ddot{}uhl, J., Devadas, S., Berger, B. & Clote, P. Efficient Algorithms for Probing the RNA Mutation Landscape. PLoS Comput. Biol. 4, e1000124 (2008).
40. Andr´e, I., Strauss, C. E. M., Kaplan, D. B., Bradley, P. & Baker, D. Emergence of symmetry in homooligomeric biological assemblies. Proc. Natl. Acad. Sci. 105, 16148-16152 (2008).
41. Yang, S., Levine, H., Onuchic, J. & Cox, D. L. Structure of infectious prions: stabilization by domain swapping. FASEB J. 19, 1778-1782 (2005).
42. Hofacker, I. L., Bernhart, S. H. F. & Stadler, P. F. Alignment of RNA Base Pairing Probability Matrices. Bioinformatics 20, 2222-2227 (2004).
43. Waldisp\ddot{}uhl, J., et al. Simultaneous Alignment and Folding of Protein Sequences. RECOMB 2009 (2009).
44. Berman, H., et al. The protein data bank. Nucleic Acids Res. 28, 235-242 (2000).
45. Clote, P. & Backofen, R. Computational Molecular Biology: An Introduction (John Wiley & Sons, 2000). 279 pages.
46. Bradley, P., Cowen, L., Menke, M., King, J. & Berger, B. BETAWRAP: Successful prediction of parallel Beta-helices from primary sequence reveals an association with many microbial pathogens. Proc. Nat. Acad. Sci. 98, 14819-14824 (2001).
47. Waldisp\ddot{}uhl, J., Berger, B., Clote, P. & Steyaer, J.-M. Predicting Transmembrane β-barrels and Inter-strand Residue Interactions from Sequence. Proteins: Struct., Funct., Bioinf. 65, 61-74 (2006).
48. Kyte, J. & Doolittle, R. F. A simple method for displaying the hydropathic character of a protein. J. Mol. Biol. 157, 105 (1982).
49. Miyazawa, S. & Jernigan, R. L. Estimation of Effective Interresidue Contact Energies from Protein Crystal Structures: Quasi-Chemical Approximation. Macromolecules 18, 534-552 (1985).
50. Ding, Y. & Lawrence, C. A statistical sampling algorithm for RNA secondary structure prediction. Nucleic Acids Res. 31(24), 7280-7301 (2003).

1. An engineered bacteriophage comprising a nucleic acid operatively linked to a promoter, wherein the nucleic acid encodes at least one anti-amyloid peptide.

2. The bacteriophage of claim 1, wherein the anti-amyloid peptide is a peptide between at least 5 and 50 amino acids long whose sequence comprises at least 5 and no more than 50 contiguous amino acids of the sequence of a first amyloidogenic polypeptide which is capable of nucleating amyloid formation by a second amyloidogenic polypeptide.

3. (canceled)

4. (canceled)

5. (canceled)

6. The bacteriophage of claim 2, wherein the first and second amyloidogenic polypeptides are no more than 50% identical.

7. The bacteriophage of claim 1, wherein at least one of the amyloidogenic polypeptides is a component of a naturally occurring amyloid or a component of a high order aggregate comprising at least two different polypeptides.

8. The bacteriophage of claim 1, wherein at least one of the amyloidogenic polypeptides is a component of a biofilm generated by a bacterium.

9. (canceled)

10. (canceled)

11. (canceled)

12. (canceled)

13. (canceled)

14. The bacteriophage of claim 2, wherein the first amyloidogenic polypeptide is a CsgB polypeptide and/or the second amyloidogenic polypeptide is a CsgA polypeptide.

15. (canceled)

16. (canceled)

17. (canceled)

18. (canceled)

19. The bacteriophage of claim 1, wherein the sequence of the anti-amyloid peptide comprises or consists of a sequence selected from SEQ ID NO: 1 or SEQ ID NO: 2 and orthologs thereof.

20. (canceled)

21. (canceled)

22. (canceled)

23. The bacteriophage of claim 14, wherein the CsgA peptide is selected from the group comprising: SEQ ID NO; 11-18, CsgA III class of peptides (SEQ ID NO: 52-53), CsgAIIb class of peptides (SEQ ID NOs:35, 36, 39-41, 45, 49-51), CsgAIIa class of peptides (SEQ ID NO: 11 and 12) and CsgAI class of peptides (SEQ ID NOs: 42, 44, 46, 57 and 58) or orthologs thereof.

24. (canceled)

25. The bacteriophage of claim 14, wherein the CsgB peptide is selected from the group comprising: SEQ ID NO; 27-34, CsgBIII class of peptides (SEQ ID NOs: 61-65), CsgBIIb class of peptides (SEQ ID NOs: 59, 60, 69, 75, 81, 93 and 94), CsgBIIa class of peptides (SEQ ID NO: 29) and CsgBI class of peptides (SEQ ID NOs: 66-68 and 70-72) or orthologs thereof.

26. (canceled)

27. (canceled)

28. (canceled)

29. The bacteriophage of claim 1, wherein the N-terminus and/or C-terminus of the anti-amyloid peptide sequence comprise at least one additional amino acid residue.

30. The bacteriophage of claim 29, wherein the N-terminus or C-terminus of the anti-amyloid peptide sequence comprises a charged amino acid residue or at least one bulky amino acid.

31.-37. (canceled)

38. The bacteriophage of claim 1, wherein the anti-amyloid peptide is expressed on the surface of the engineered bacteriophage from which it is expressed, or wherein the anti-amyloid peptide is released from a bacterial host cell infected by the engineered bacteriophage.

39. (canceled)

40. (canceled)

41. (canceled)

42. The bacteriophage of claim 1, wherein the nucleic acid encoding at least one anti-amyloid peptide agent also encodes a signal sequence.

43. (canceled)

44. (canceled)

45. A method to reduce protein aggregate formation in a subject comprising administering to a subject at least one bacteriophage comprising a nucleic acid operatively linked to a promoter, wherein the nucleic acid encodes at least one anti-amyloid peptide.

46. The method of claim 45, wherein the subject suffers or is at risk of amyloid associated disorder, or wherein the subject suffers from or is at increased risk of an infection by a bacterium.

47.-79. (canceled)

81. A method to inhibit protein aggregate formation on a surface, or in a fluid sample comprising administering to the surface or fluid sample a composition comprising at least one bacteriophage comprising a nucleic acid operatively linked to a promoter, wherein the nucleic acid encodes at least one anti-amyloid peptide.

82.-124. (canceled)

125. A composition comprising the bacteriophage of claim 1.

126. The composition of claim 125, further comprising a pharmaceutical acceptable carrier.

127.-164. (canceled)
